(19) United States
(12) Patent Application Publication
(10) Pub. No.: US 2007/0059799 A1
(43) Pub. Date: Mar. 15, 2007

(54) Inducing cellular immune responses to hepatitis B virus using peptide and nucleic acid compositions

(75) Inventors: Alessandro Sette, La Jolla, CA (US); John Sidney, La Jolla, CA (US); Scott Southwood, Santee, CA (US); Maria Vitiello, La Jolla, CA (US); Brian Livingston, San Diego, CA (US); Esteban Celis, Tampa, FL (US); Ralph Kubo, Carlsbad, CA (US); Howard Grey, La Jolla, CA (US); Robert Chesnut, Cardiff-by-the-sea, CA (US)

Correspondence Address: STERNE, KESSLER, GOLDSTEIN & FOX, P.L.L.C., 1100 NEW YORK AVE., WASHINGTON, DC, 20005, US

(73) Assignee: Pharmexa Inc., San Diego, CA (US)

(21) Appl. No.: 11/522,314

(22) Filed: Sep. 18, 2006

Related U.S. Application Data
(63) Continuation of application No. 10/363,990, filed on Sep. 3, 2003.
(63) Continuation-in-part of application No. 09/350,401, filed on Jul. 8, 1999.
Application No. 09/350,401 is a continuation-in-part of application No. 09/239,043, filed on Jan. 27, 1999, now Pat. No. 6,689,363.

(51) Int. Cl. C12Q 1/68; C12P 21/06

(52) U.S. Cl. 435/69.1; 435/6

(57) ABSTRACT

This invention uses our knowledge of the mechanisms by which antigen is recognized by T cells to develop epitope-based vaccines directed towards HBV. More specifically, this application communicates our discovery of pharmaceutical compositions and methods of use in the prevention and treatment of HBV infection.

### CROSS REFERENCE TO RELATED APPLICATIONS

The present application is a continuation of U.S. application Ser. No. 10/363,990, which is a national stage application of International Appl. No. PCT/US00/24802, filed Sep. 8, 2000, which published under PCT Article 21(2) in English, each of which is herein incorporated by reference; and is a continuation-in-part of U.S. application Ser. No. 09/350,401, filed Jul. 8, 1999, which is a continuation-in-part of U.S. application Ser. No. 09/239,043, filed Jan. 27, 1999, now U.S. Pat. No. 6,689,363 B1.

### STATEMENT REGARDING FEDERALLY-SPONSORED RESEARCH AND DEVELOPMENT

This invention was funded, in part, by the United States government under grants with the National Institutes of Health. The u.s. government has certain rights in this invention.

### REFERENCE TO A SEQUENCE LISTING SUBMITTED ON A COMPACT DISC

This application includes a “Sequence Listing,” which is provided as an electronic document on a compact disc (CD-R). This compact disc contains the file “Sequence Listing.txt” (808,960 bytes, created on Aug. 30, 2006), which is hereby incorporated by reference in its entirety.

### BACKGROUND OF THE INVENTION

### FIELD OF THE INVENTION

Chronic infection by hepatitis B virus (HBV) affects at least 5% of the world's population and is a major cause of cirrhosis and hepatocellular carcinoma (Hoofnagle, J., N. Engl. J. Med. 323:337, 1990; Fields, B. and Knipe, D., In: Fields Virology 2:2137, 1990). The World Health Organization lists hepatitis B as a leading cause of death worldwide, close behind chronic pulmonary disease, and more prevalent than AIDS. Chronic HBV infection can range from an asymptomatic carrier state to continuous hepatocellular necrosis and inflammation, and can lead to hepatocellular carcinoma.

The immune response to HBV is believed to play an important role in controlling hepatitis B infection. A variety of humoral and cellular responses to different regions of HBV including the nucleocapsid core, polymerase, and surface antigens have been identified. T cell-mediated immunity, particularly involving class I human leukocyte antigen-restricted cytotoxic T lymphocytes (CTL), is believed to be crucial in combatting established HBV infection.

Class I human leukocyte antigen (HLA) molecules are expressed on the surface of almost all nucleated cells. CTL recognize peptide fragments, derived from intracellular processing of various antigens, in the form of a complex with class I HLA molecules. This recognition event then results in the destruction of the cell bearing the HLA-peptide complex directly or the activation of non-destructive mechanisms e.g., the production of interferon, that inhibit viral replication.

Several studies have emphasized the association between self-limiting acute hepatitis and multispecific CTL responses (Penna, A. et al., J. Exp. Med. 174:1565, 1991; Nayersina, R. et al., J. Immunol. 150:4659, 1993). Spontaneous and interferon-related clearance of chronic HBV infection is also associated with the resurgence of a vigorous CTL response (Guidotti, L. G. et al., Proc. Natl. Acad. Sci. USA 91:3764, 1994). In all such cases the CTL responses are polyclonal, and specific for multiple viral proteins including the HBV envelope, core and polymerase antigens. By contrast, in patients with chronic hepatitis, the CTL activity is usually absent or weak, and antigenically restricted.

The crucial role of CTL in resolution of HBV infection has been further underscored by studies using HBV transgenic mice. Adoptive transfer of HBV-specific CTL into mice transgenic for the HBV genome resulted in suppression of virus replication. This effect was primarily mediated by a non-lytic, lymphokine-based mechanism (Guidotti, L. G. et al., Proc. Natl. Acad. Sci. USA 91:3764, 1994; Guidotti, L. G., Guilhot, S., and Chisari, F. V. J. Virol. 68:1265, 1994; Guidotti, L. G. et al., J. Virol. 69:6158, 1995; Gilles, P. N., Fey, G., and Chisari, F. V., J. Virol. 66:3955, 1992).

As is the case for HLA class I restricted responses, HLA class II restricted T cell responses are usually detected in patients with acute hepatitis, and are absent or weak in patients with chronic infection (Chisari, F. V. and Ferrari, C., Annu. Rev. Immunol. 13:29, 1995). HLA Class II responses are tied to activation of helper T cells (HTLs) Helper T lymphocytes, which recognize Class II HLA molecules, may directly contribute to the clearance of HBV infection through the secretion of cytokines which suppress viral replication (Franco, A. et al., J. Immunol. 159:2001, 1997). However, their primary role in disease resolution is believed to be mediated by inducing activation and expansion of virus-specific CTL and B cells.

In view of the heterogeneous immune response observed with HBV infection, induction of a multi-specific cellular immune response directed simultaneously against multiple epitopes appears to be important for the development of an efficacious vaccine against HBV. There is a need to establish vaccine embodiments that elicit immune responses that correspond to responses seen in patients that clear HBV infection. Epitope-based vaccines appear useful.

Upon development of appropriate technology, the use of epitope-based vaccines has several advantages over current vaccines. The epitopes for inclusion in such a vaccine are to be selected from conserved regions of viral or tumor-associated antigens, in order to reduce the likelihood of escape mutants. The advantage of an epitope-based approach over the use of whole antigens is that there is evidence that the immune response to whole antigens is directed largely toward variable regions of the antigen, allowing for immune escape due to mutations. Furthermore, immunosuppressive epitopes that may be present in whole antigens can be avoided with the use of epitope-based vaccines.

Additionally, with an epitope-based vaccine approach, there is an ability to combine selected epitopes (CTL and HTL) and additionally to modify the composition of the epitopes, achieving, for example, enhanced immunogenicity. Accordingly, the immune response can be modulated, as appropriate, for the target disease. Similar engineering of the response is not possible with traditional approaches.

Another major benefit of epitope-based immune-stimulating vaccines is their safety. The possible pathological side effects caused by infectious agents or whole protein antigens, which might have their own intrinsic biological activity, is eliminated.

An epitope-based vaccine also provides the ability to direct and focus an immune response to multiple selected antigens from the same pathogen. Thus, patient-by-patient variability in the immune response to a particular pathogen may be alleviated by inclusion of epitopes from multiple antigens from that pathogen in a vaccine composition. A “pathogen” may be an infectious agent or a tumor associated molecule.

However, one of the most formidable obstacles to the development of broadly efficacious epitope-based immunotherapeutics has been the extreme polymorphism of HLA molecules. To date, effective non-genetically biased coverage of a population has been a task of considerable complexity; such coverage has required that epitopes be used specific for HLA molecules corresponding to each individual HLA allele, therefore, impractically large numbers of epitopes would have to be used in order to cover ethnically diverse populations. There has existed a need to develop peptide epitopes that are bound by multiple HLA antigen molecules for use in epitope-based vaccines. The greater the number of HLA antigen molecules bound, the greater the breadth of population coverage by the vaccine.

Furthermore, as described herein in greater detail, a need has existed to modulate peptide binding properties, for example so that peptides that are able to bind to multiple HLA antigens do so with an affinity that will stimulate an immune response. Identification of epitopes restricted by more than one HLA allele at an affinity that correlates with immunogenicity is important to provide thorough population coverage, and to allow the elicitation of responses of sufficient vigor whereby the natural immune responses noted in self-limiting acute hepatitis, or of spontaneous clearance of chronic HBV infection is induced in a diverse segment of the population. Such a response can also target a broad array of epitopes. The technology disclosed herein provides for such favored immune responses.

The information provided in this section is intended to disclose the presently understood state of the art as of the filing date of the present application. Information is included in this section which was generated subsequent to the priority date of this application. Accordingly, background in this section is not intended, in any way, to delineate the priority date for the invention.

### BRIEF SUMMARY OF THE INVENTION

This invention applies our knowledge of the mechanisms by which antigen is recognized by T cells, for example, to develop epitope-based vaccines directed towards HBV. More specifically, this application communicates our discovery of specific epitope pharmaceutical compositions and methods of use in the prevention and treatment of HBV infection.

Upon development of appropriate technology, the use of epitope-based vaccines has several advantages over current vaccines, particularly when compared to the use of whole antigens in vaccine compositions. There is evidence that the immune response to whole antigens is directed largely toward variable regions of the antigen, allowing for immune escape due to mutations. The epitopes for inclusion in an epitope-based vaccine are selected from conserved regions of viral or tumor-associated antigens, which thereby reduces the likelihood of escape mutants. Furthermore, immunosuppressive epitopes that may be present in whole antigens can be avoided with the use of epitope-based vaccines.

An additional advantage of an epitope-based vaccine approach is the ability to combine selected epitopes (CTL and HTL), and further, to modify the composition of the epitopes, achieving, for example, enhanced immunogenicity. Accordingly, the immune response can be modulated, as appropriate, for the target disease. Similar engineering of the response is not possible with traditional approaches.

Another major benefit of epitope-based immune-stimulating vaccines is their safety. The possible pathological side effects caused by infectious agents or whole protein antigens, which might have their own intrinsic biological activity, is eliminated.

An epitope-based vaccine also provides the ability to direct and focus an immune response to multiple selected antigens from the same pathogen. Thus, patient-by-patient variability in the immune response to a particular pathogen may be alleviated by inclusion of epitopes from multiple antigens from that pathogen in a vaccine composition. A “pathogen” may be an infectious agent or a tumor associated molecule.

One of the most formidable obstacles to the development of broadly efficacious epitope-based immunotherapeutics, however, has been the extreme polymorphism of HLA molecules. To date, effective non-genetically biased coverage of a population has been a task of considerable complexity; such coverage has required that epitopes be used specific for HLA molecules corresponding to each individual HLA allele, therefore, impractically large numbers of epitopes would have to be used in order to cover ethnically diverse populations. Thus, there has existed a need to develop peptide epitopes that are bound by multiple HLA antigen molecules for use in epitope-based vaccines. The greater the number of HLA antigen molecules bound, the greater the breadth of population coverage by the vaccine.

Furthermore, as described herein in greater detail, a need has existed to modulate peptide binding properties, for example, so that peptides that are able to bind to multiple HLA antigens do so with an affinity that will stimulate an immune response. Identification of epitopes restricted by more than one HLA allele at an affinity that correlates with immunogenicity is important to provide thorough population coverage, and to allow the elicitation of responses of sufficient vigor to prevent or clear an infection in a diverse segment of the population. Such a response can also target a broad array of epitopes. The technology disclosed herein provides for such favored immune responses.

In a preferred embodiment, epitopes for inclusion in vaccine compositions of the invention are selected by a process whereby protein sequences of known antigens are evaluated for the presence of motif or supermotif-bearing epitopes. Peptides corresponding to a motif- or supermotif-bearing epitope are then synthesized and tested for the ability to bind to the HLA molecule that recognizes the selected motif. Those peptides that bind at an intermediate or high affinity i.e., an IC₅₀ (or a K_{D} value) of 500 nM or less for HLA class I molecules or 1000 nM or less for HLA class II molecules, are further evaluated for their ability to induce a CTL or HTL response. Immunogenic peptides are selected for inclusion in vaccine compositions.

Supermotif-bearing peptides may additionally be tested for the ability to bind to multiple alleles within the HLA supertype family. Moreover, peptide epitopes may be analogued to modify binding affinity and/or the ability to bind to multiple alleles within an HLA supertype.

The invention also includes an embodiment comprising a method for monitoring immunogenic activity of a vaccine for HBV in a patient having a known HLA-type, the method comprising incubating a T lymphocyte sample from the patient with a peptide composition comprising an HBV epitope consisting essentially of an amino acid sequence described in Tables VI to Table XX or Table XXII which binds the product of at least one HLA allele present in said patient, and detecting for the presence of a T lymphocyte that binds to the peptide. In a preferred embodiment, the peptide comprises a tetrameric complex.

An alternative modality for defining the peptides in accordance with the invention is to recite the physical properties, such as length; primary, potentially secondary and/or tertiary structure; or charge, which are correlated with binding to a particular allele-specific HLA molecule or group of allele-specific HLA molecules. A further modality for defining peptides is to recite the physical properties of an HLA binding pocket, or properties shared by several allele-specific HLA binding pockets (e.g. pocket configuration and charge distribution) and reciting that the peptide fits and binds to said pocket or pockets.

As will be apparent from the discussion below, other methods and embodiments are also contemplated. Further, novel synthetic peptides produced by any of the methods described herein are also part of the invention.

### BRIEF DESCRIPTION OF THE FIGURES

FIG. 1: FIG. 1 provides a graph of total frequency of genotypes as a function of the number of HBV candidate epitopes bound by HLA-A and B molecules, in an average population.

FIG. 2: FIG. 2 Illustrates the Position of Peptide Epitopes in Experimental Model Minigene Constructs

### DETAILED DESCRIPTION OF THE INVENTION

peptides and corresponding nucleic acid compositions of the present invention are useful for stimulating an immune response to HBV either by stimulating the production of CTL or HTL responses. The peptides, which are derived directly or indirectly from native HBV amino acid sequences, are able to bind to HLA molecules and stimulate an immune response to HBV. The complete polyprotein sequence from HBV and its variants can be obtained from Genbank. Peptides can also be readily determined from sequence information that may subsequently be discovered for heretofore unknown variants of HBV as will be clear from the disclosure provided below.

The peptides of the invention have been identified in a number of ways, as will be discussed below. Further, analog peptides have been derived and the binding activity for H HLA molecules modulated by modifying specific amino acid residues to create peptide analogs exhibiting altered immunogenicity. Further, the present invention provides compositions and combinations of compositions that enable epitope-based vaccines that are capable of interacting with multiple HLA antigens to provide broader population coverage than prior vaccines.

A. Definitions

The invention can be better understood with reference to the following definitions, which are listed alphabetically.

A “computer” or “computer system” generally includes: a processor; at least one information storage/retrieval apparatus such as, for example, a hard drive, a disk drive or a tape drive; at least one input apparatus such as, for example, a keyboard, a mouse, a touch screen, or a microphone; and display structure. Additionally, the computer may include a communication channel in communication with a network. Such a computer may include more or less than what is listed above.

A “construct” as used herein generally denotes a composition that does not occur in nature. A construct can be produced by synthetic technologies, e.g., recombinant DNA preparation and expression or chemical synthetic techniques for nucleic or amino acids. A construct can also be produced by the addition or affiliation of one material with another such that the result is not found in nature in that form.

“Cross-reactive binding” indicates that a peptide is bound by more than one HLA molecule; a synonym is degenerate binding.

A “cryptic epitope” elicits a response by immunization with an isolated peptide, but the response is not cross-reactive in vitro when intact whole protein which comprises the epitope is used as an antigen.

A “dominant epitope” is an epitope that induces an immune response upon immunization with a whole native antigen (see, e.g., Sercarz, et al., Annu. Rev. Immunol. 11:729-766, 1993). Such a response is cross-reactive in vitro with an isolated peptide epitope.

With regard to a particular amino acid sequence, an “epitope” is a set of amino acid residues which is involved in recognition by a particular immunoglobulin, or in the context of T cells, those residues necessary for recognition by T cell receptor (TCR) proteins and/or Major Histocompatibility Complex (MHC) receptors. In an immune system setting, in vivo or in vitro, an epitope is the collective features of a molecule, such as primary, secondary and tertiary peptide structure, and charge, that together form a site recognized by an immunoglobulin, TCR or HLA molecule. Throughout this disclosure epitope and peptide are often used interchangeably.

It is to be appreciated that protein or peptide molecules that comprise an epitope of the invention as well as additional amino acid(s) are still within the bounds of the invention. In certain embodiments, there is a limitation on the length of a peptide of the invention which is not otherwise a construct. An embodiment that is length-limited occurs when the protein/peptide comprising an epitope of the invention comprises a region (i.e., a contiguous series of amino acids) having 100% identity with a native sequence. In order to avoid the definition of epitope from reading, e.g., on whole natural molecules, there is a limitation on the length of any region that has 100% identity with a native peptide sequence. Thus, for a peptide comprising an epitope of the invention and a region with 100% identity with a native peptide sequence (and is not otherwise a construct), the region with 100% identity to a native sequence generally has a length of: less than or equal to 600 amino acids, often less than or equal to 500 amino acids, often less than or equal to 400 amino acids, often less than or equal to 250 amino acids, often less than or equal to 100 amino acids, often less than or equal to 85 amino acids, often less than or equal to 75 amino acids, often less than or equal to 65 amino acids, and often less than or equal to 50 amino acids. In certain embodiments, an “epitope” of the invention is comprised by a peptide having a region with less than 51 amino acids that has 100% identity to a native peptide sequence, in any increment of (49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) down to 5 amino acids.

Accordingly, peptide or protein sequences longer than 600 amino acids are within the scope of the invention, so long as they do not comprise any contiguous sequence of more than 600 amino acids that have 100% identity with a native peptide sequence, if they are not otherwise a construct. For any peptide that has five contiguous residues or less that correspond to a native sequence, there is no limitation on the maximal length of that peptide in order to fall within the scope of the invention. It is presently preferred that a CTL epitope be less than 600 residues long in any increment down to eight amino acid residues.

“Human Leukocyte Antigen” or “HLA” is a human class I or class II Major Histocompatibility Complex (MHC) protein (see, Stites, et al., Immunology, 8th Ed., Lange Publishing, Los Altos, Calif. (1994).

An “HLA supertype or family”, as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, and HLA xx-like supertype molecules (where xx denotes a particular HLA type) are synonyms.

Throughout this disclosure, results are expressed in terms of “IC₅₀'s.” IC₅₀ is the concentration of peptide in a binding assay at which 50% inhibition of binding of a reference peptide is observed. Given the conditions in which the assays are run (i.e., limiting HLA proteins and labeled peptide concentrations), these values approximate KD values. Assays for determining binding are described in detail, e.g., in PCT publications WO 94/20127 and WO 94/03205. It should be noted that IC₅₀ values can change, often dramatically, if the assay conditions are varied, and depending on the particular reagents used (e.g., HLA preparation, etc.). For example, excessive concentrations of HLA molecules will increase the apparent measured IC₅₀ of a given ligand.

Alternatively, binding is expressed relative to a reference peptide. Although as a particular assay becomes more, or less, sensitive, the IC₅₀'s of the peptides tested may change somewhat, the binding relative to the reference peptide will not significantly change. For example, in an assay run under conditions such that the IC₅₀ of the reference peptide increases 10-fold, the IC₅₀ values of the test peptides will also shift approximately 10-fold. Therefore, to avoid ambiguities, the assessment of whether a peptide is a good, intermediate, weak, or negative binder is generally based on its IC₅₀, relative to the IC₅₀ of a standard peptide.

Binding can also be determined using other assay systems including those using: live cells (e.g., Ceppellini et al., Nature 339:392, 1989; Christnick et al., Nature 352:67, 1991; Busch et al., Immunol. 2:443, 1990; Hill et al., J. Immunol. 147:189, 1991; del Guercio et al., J. Immunol. 154:685, 1995), cell free systems using detergent lysates (e.g., Cerundolo et al., J. Immunol. 21:2069, 1991), immobilized purified MHC (e.g., Hill et al., J. Immunol. 152, 2890, 1994; Marshall et al., J. Immunol. 152:4946, 1994), ELISA systems (e.g., Reay et al., EMBO J. 11:2829, 1992), surface plasmon resonance (e.g., Khilko et al., J. Biol. Chem. 268:15425, 1993); high flux soluble phase assays (Hammer et al., J. Exp. Med. 180:2353, 1994), and measurement of class I MHC stabilization or assembly (e.g., Ljunggren et al., Nature 346:476, 1990; Schumacher et al., Cell 62:563, 1990; Townsend et al., Cell 62:285, 1990; Parker et al., J. Immunol. 149:1896, 1992).

As used herein, “high affinity” with respect to HLA class I molecules is defined as binding with an IC₅₀, or K_{D} value, of 50 nM or less; “intermediate affinity” is binding with an IC₅₀ or K_{D} value of between about 50 and about 500 nM. “High affinity” with respect to binding to HLA class II molecules is defined as binding with an IC₅₀ or K_{D} value of 100 nM or less; “intermediate affinity” is binding with an IC₅₀ or K_{D} value of between about 100 and about 1000 nM.

The terms “identical” or percent “identity,” in the context of two or more peptide sequences, refer to two or more sequences or subsequences that are the same or have a specified percentage of amino acid residues that are the same, when compared and aligned for maximum correspondence over a comparison window, as measured using a sequence comparison algorithms or by manual alignment and visual inspection.

An “immunogenic peptide” or “peptide epitope” is a peptide that comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T cell response, or a helper T cell response, to the antigen from which the immunogenic peptide is derived.

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Link” or “join” refers to any method known in the art for functionally connecting peptides, including, without limitation, recombinant fusion, covalent bonding, disulfide bonding, ionic bonding, hydrogen bonding, and electrostatic bonding.

“Major Histocompatibility Complex” or “MHC” is a cluster of genes that plays a role in control of the cellular interactions responsible for physiologic immune responses. In humans, the MHC complex is also known as the HLA complex. For a detailed description of the MHC and HLA complexes, see, Paul, Fundamental Immunology, 3rd Ed., Raven Press, New York, 1993.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “negative binding residue” or “deleterious residue” is an amino acid which, if present at certain positions (typically not primary anchor positions) of a peptide epitope, results in decreased binding affinity of the peptide for the peptide's corresponding HLA molecule. Any residue that is not “deleterious” is a “non-deleterious” residue.

A “non-native” sequence or “construct” refers to a sequence that is not found in nature, i.e., is “non-naturally occurring”. Such sequences include, e.g., peptides that are lipidated or otherwise modified, and polyepitopic compositions that contain epitopes that are not contiguous in a native protein sequence.

The term “peptide” is used interchangeably with “oligopeptide” in the present specification to designate a series of residues, typically 1-amino acids, connected one to the other, typically by peptide bonds between the α-amino and carboxyl groups of adjacent amino acids. In some embodiments, the preferred CTL-inducing oligopeptides of the invention are 13 residues or less in length and usually consist of between about 8 and about 11 residues, preferably 9 or 10 residues. In some embodiments, the preferred HTL-inducing oligopeptides are less than about 50 residues in length and usually consist of between about 6 and about 30 residues, more usually between about 12 and 25, and often between about 15 and 20 residues.

“Pharmaceutically acceptable” refers to a generally non-toxic, inert, and physiologically compatible composition.

A “primary anchor residue” is an amino acid at a specific position along a peptide sequence which is understood to provide a contact point between the immunogenic peptide and the HLA molecule. One to three, usually two, primary anchor residues within a peptide of defined length generally defines a “motif” for an immunogenic peptide. These residues are understood to fit in close contact with peptide binding grooves of an HLA molecule, with their side chains buried in specific pockets of the binding grooves themselves. In one embodiment, the primary anchor residues are located at position 2 (from the amino terminal position) and at the carboxyl terminal position of a 9 residue peptide in accordance with the invention. The primary anchor positions for each motif and supermotif are set forth in Table I. For example, analog peptides can be created by altering the presence or absence of particular residues in these primary anchor positions. Such analogs are used to finely modulate the binding affinity of a peptide comprising a particular motif or supermotif.

“Promiscuous recognition” is where a distinct peptide is recognized by the same T cell clone in the context of multiple HLA molecules. Promiscuous binding is synonymous with cross-reactive binding.

A “protective immune response” or “therapeutic immune response” refers to a CTL and/or an HTL response to an antigen derived from an infectious agent or a tumor antigen, which prevents or at least partially arrests disease symptoms or progression. The immune response may also include an antibody response which has been facilitated by the stimulation of helper T cells.

The term “residue” refers to an amino acid or amino acid mimetic incorporated into an oligopeptide by an amide bond or amide bond mimetic.

A “secondary anchor residue” is an amino acid at a position other than a primary anchor position in a peptide which may influence peptide binding. A secondary anchor residue occurs at a significantly higher frequency amongst bound peptides than would be expected by random distribution of amino acids at one position. The secondary anchor residues are said to occur at “secondary anchor positions.” A secondary anchor residue can be identified as a residue which is present at a higher frequency among high affinity binding peptides, or a residue otherwise associated with high affinity binding. For example, analog peptides can be created by altering the presence or absence of particular residues in these secondary anchor positions. Such analogs are used to finely modulate the binding affinity of a peptide comprising a particular motif or supermotif.

A “subdominant epitope” is an epitope which evokes little or no response upon immunization with whole antigens which comprise the epitope, but for which a response can be obtained by immunization with an isolated peptide, and this response (unlike the case of cryptic epitopes) is detected when whole protein is used to recall the response in vitro or in vivo.

A “supermotif” is a peptide binding specificity shared by HLA molecules encoded by two or more HLA alleles. A supermotif-bearing epitope is preferably is recognized with high or intermediate affinity (as defined herein) by two or more HLA antigens.

“Synthetic peptide” refers to a peptide that is man-made using such methods as chemical synthesis or recombinant DNA technology.

As used herein, a “vaccine” is a composition that contains one or more peptides of the invention. There are numerous embodiments of vaccines in accordance with the invention, such as by a cocktail of one or more peptides; one or more epitopes of the invention comprised by a polyepitopic peptide; or nucleic acids that encode such peptides or polypeptides, e.g., a minigene that encodes a polyepitopic peptide. The “one or more peptides” can include any whole unit integer from 1-150, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, or 150 or more peptides of the invention. The peptides or polypeptides can optionally be modified, such as by lipidation, addition of targeting or other sequences. HLA class I-binding peptides of the invention can be admixed with, or linked to, HLA class II-binding peptides, to facilitate activation of both cytotoxic T lymphocytes and helper T lymphocytes. Vaccines can also comprise peptide-pulsed antigen presenting cells, e.g., dendritic cells.

The nomenclature used to describe peptide compounds follows the conventional practice wherein the amino group is presented to the left (the N-terminus) and the carboxyl group to the right (the C-terminus) of each amino acid residue. When amino acid residue positions are referred to in a peptide epitope they are numbered in an amino to carboxyl direction with position one being the position closest to the amino terminal. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The 1-form of an amino acid residue is represented by a capital single letter or a capital first letter of a three-letter symbol, and the d-form for those amino acids having d-forms is represented by a lower case single letter or a lower case three letter symbol. Glycine has no asymmetric carbon atom and is simply referred to as “Gly” or G. Symbols for the amino acids are shown below.

| Single Letter Symbol | Three Letter Symbol | Amino Acids |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

B. Stimulation of CTL and HTL Responses Against HBV

The mechanism by which T cells recognize antigens has been delineated during the past ten years. Based on our new understanding of the immune system we have generated efficacious peptide epitope vaccine compositions that can induce a therapeutic or prophylactic immune response to HBV infection in a broad population. For an understanding of the value and efficacy of the claimed compositions, a brief review of the technology is provided.

A complex of an HLA molecule and a peptidic antigen acts as the ligand recognized by HLA-restricted T cells (Buus, S. et al., Cell 47:1071, 1986; Babbitt, B. P. et al., Nature 317:359, 1985; Townsend, A. and Bodmer, H., Annu. Rev. Immunol. 7:601, 1989; Germain, R. N., Annu. Rev. Immunol. 11:403, 1993). Through the study of single amino acid substituted antigen analogs and the sequencing of endogenously bound, naturally processed peptides, critical residues that correspond to motifs required for specific binding to HLA antigen molecules have been identified and are described herein and are set forth in Tables I, II, and III (see also, e.g., Southwood, et al., J. Immunol. 160:3363, 1998; Rammensee, et al., Immunogenetics 41:178, 1995; Rammensee et al., SYFPEITHI, access via web at: http://134.2.96.221/scripts.hlaserver.dll/home.htm; Sette, A. and Sidney, J. Curr. Opin. Immunol. 10:478, 1998; Engelhard, V. H., Curr. Opin. Immunol. 6:13, 1994; Sette, A. and Grey, H. M., Curr. Opin. Immunol. 4:79, 1992; Sinigaglia, F. and Hammer, J. Curr. Biol. 6:52, 1994; Ruppert et al., Cell 74:929-937, 1993; Kondo et al., J. Immunol. 155:4307-4312, 1995; Sidney et al., J. Immunol. 157:3480-3490, 1996; Sidney et al., Human Immunol. 45:79-93, 1996; Sette, A. and Sidney, J. Immunogenetics, in press, 1999).

Furthermore, x-ray crystallographic analysis of HLA-peptide complexes has revealed pockets within the peptide binding cleft of HLA molecules which accommodate, in an allele-specific mode, residues borne by peptide ligands; these residues in turn determine the HLA binding capacity of the peptides in which they are present. (See, e.g., Madden, D. R. Annu. Rev. Immunol. 13:587, 1995; Smith, et al., Immunity 4:203, 1996; Fremont et al., Immunity 8:305, 1998; Stern et al., Structure 2:245, 1994; Jones, E. Y. Curr. Opin. Immunol. 9:75, 1997; Brown, J. H. et al., Nature 364:33, 1993; Guo, H. C. et al., Proc. Natl. Acad. Sci. USA 90:8053, 1993; Guo, H. C. et al., Nature 360:364, 1992; Silver, M. L. et al., Nature 360:367, 1992; Matsumura, M. et al., Science 257:927, 1992; Madden et al., Cell 70:1035, 1992; Fremont, D. H. et al., Science 257:919, 1992; Saper, M. A., Bjorkman, P. J. and Wiley, D. C., J. Mol. Biol. 219:277, 1991.)

Accordingly, the definition of class I and class II allele-specific HLA binding motifs or class I supermotifs allows identification of regions within a protein that have the potential of binding particular HLA antigens (see also e.g., Sette, A. and Grey, H. M., Curr. Opin. Immunol. 4:79, 1992; Sinigaglia, F. and Hammer, J., Curr. Biol. 6:52, 1994; Engelhard, V. H., Curr. Opin. Immunol. 6:13, 1994; Kast, W. M. et al., J. Immunol., 152:3904, 1994).

Furthermore, a variety of assays to quantify the affinity of interaction between peptide and HLA have also been established. Such assays include, for example, measures of IC₅₀ values, inhibition of antigen presentation (Sette et al., J. Immunol. 141:3893, 1991), in vitro assembly assays (Townsend et al., Cell 62:285, 1990), measures of dissociations rates (Parker et al., J. Immunol. 149:1896-1904, 1992), and FACS-based assays using mutated cells, such as RMA.S (Melief, et al., Eur. J. Immunol. 21:2963, 1991).

The present inventors have found that the correlation of binding affinity with immunogenicity is an important factor to be considered when evaluating candidate peptides. Thus, by a combination of motif searches and HLA-peptide binding assays, candidates for epitope-based vaccines have been identified. After determining their binding affinity, additional confirmatory work can be performed to select, amongst these vaccine candidates, epitopes with preferred characteristics in terms of antigenicity and immunogenicity. Various strategies can be utilized to evaluate immunogenicity, including:

1) Evaluation of primary T cell cultures from normal individuals (Wentworth, P. A. et al., Mol. Immunol. 32:603, 1995; Celis, E. et al., Proc. Natl. Acad. Sci. USA 91:2105, 1994; Tsai, V. et al., J. Immunol. 158:1796, 1997; Kawashima, I. et al., Human Immunol. 59:1, 1998); This procedure involves the stimulation of PBL from normal subjects with a test peptide in the presence of antigen presenting cells in vitro over a period of several weeks. T cells specific for the peptide become activated during this time and are detected using a ⁵¹Cr-release assay involving peptide sensitized target cells.

2) Immunization of HLA transgenic mice (Wentworth, P. A. et al., J. Immunol. 26:97, 1996; Wentworth, P. A. et al., Int. Immunol. 8:651, 1996; Alexander, J. et al., J. Immunol. 159:4753, 1997); In this method, peptides in incomplete Freund's adjuvant are administered subcutaneously to HLA transgenic mice. Several weeks following immunization, splenocytes are removed and cultured in vitro in the presence of test peptide for approximately one week. Peptide-specific T cells are detected using a ⁵¹Cr-release assay involving peptide sensitized target cells and target cells expressing endogenously generated antigen.

3) Demonstration of recall T cell responses from immune individuals who have recovered from infection, and/or from chronically infected patients (Rehermann, B. et al., J. Exp. Med. 181:1047, 1995; Doolan, D. L. et al., Immunity 7:97, 1997; Bertoni, R. et al., J. Clin. Invest. 100:503, 1997; Threlkeld, S. C. et al., J. Immunol. 159:1648, 1997; Diepolder, H. M. et al., J. Virol. 71:6011, 1997). In applying this strategy, recall responses were detected by culturing PBL from subjects that had been naturally exposed to the antigen, for instance through infection, and thus had generated an immune response “naturally”. PBL from subjects were cultured in vitro for 1-2 weeks in the presence of test peptide plus antigen presenting cells (APC) to allow activation of “memory” T cells, as compared to “naive” Tcells. At the end of the culture period, T cell activity is detected using assays for T cell activity including ⁵¹Cr release involving peptide-sensitized targets, T cell proliferation or lymphokine release.

The following describes the peptide epitopes and corresponding nucleic acids of the invention.

C. Binding Affinity of Peptide Epitopes for HLA Molecules

As indicated herein, the large degree of HLA polymorphism is an important factor to be taken into account with the epitope-based approach to vaccine development. To address this factor, epitope selection encompassing identification of peptides capable of binding at high or intermediate affinity to multiple HLA molecules is preferably utilized, most preferably these epitopes bind at high or intermediate affinity to two or more allele specific HLA molecules.

CTL-inducing peptides of interest for vaccine compositions preferably include those that have an IC₅₀ or binding affinity value for class I HLA molecules of 500 nM or less. HTL-inducing peptides preferably include those that have an IC₅₀ or binding affinity value for class II HLA molecules of 1000 nM or less. For example, peptide binding is assessed by testing the capacity of a candidate peptide to bind to a purified HLA molecule in vitro. Peptides exhibiting high or intermediate affinity are then considered for further analysis. Selected peptides are tested on other members of the supertype family. In preferred embodiments, peptides that exhibit cross-reactive binding are then used in vaccines or in cellular screening analyses.

As disclosed herein, high HLA binding affinity is correlated with greater immunogenicity. Greater immunogenicity can be manifested in several different ways. Immunogenicity corresponds to whether an immune response is elicited at all, and to the vigor of any particular response. For example, a peptide might elicit an immune response in a diverse array of the population, yet in no instance produce a vigorous response. In accordance with these principles, close to 90% of high binding peptides have been found to be immunogenic, as contrasted with about 50% of the peptides which bind with intermediate affinity. Moreover, higher binding affinity peptides leads to more vigorous immunogenic responses. As a result, less peptide is required to elicit a similar biological effect if a high affinity binding peptide is used. Thus, in preferred embodiments of the invention, high binding epitopes are particularly desired.

The relationship between binding affinity for HLA class I molecules and immunogenicity of discrete peptide epitopes on bound antigens has been determined for the first time in the art by the present inventors. The correlation between binding affinity and immunogenicity was analyzed in two different experimental approaches (Sette, et al., J. Immunol. 153:5586-5592, 1994). In the first approach, the immunogenicity of potential epitopes ranging in HLA binding affinity over a 10,000-fold range was analyzed in HLA-A*0201 transgenic mice. In the second approach, the antigenicity of approximately 100 different hepatitis B virus (HBV)-derived potential epitopes, all carrying A*0201 binding motifs, was assessed by using PBL (peripheral blood lymphocytes) of acute hepatitis patients. Pursuant to these approaches, it was determined that an affinity threshold of approximately 500 nM (preferably an IC₅₀ value of 500 nM or less) determines the capacity of a peptide epitope to elicit a CTL response. These data are true for class I binding affinity measurements for naturally processed peptides and for synthesized T cell epitopes. These data also indicate the important role of determinant selection in the shaping of T cell responses.

An affinity threshold associated with immunogenicity in the context of HLA class II DR molecules has also been delineated (Southwood et al. J. Immunology 160:3363-3373,1998, and U.S. Ser. No. 60/087192 filed May 29, 1998). In order to define a biologically significant threshold of DR binding affinity, a database of the binding affinities of 32 DR-restricted epitopes for their restricting element was compiled. In approximately half of the cases (15 of 32 epitopes), DR restriction was associated with high binding affinities, i.e., binding affinities of with an IC₅₀ value of 100 nM or less. In the other half of the cases (16 of 32), DR restriction was associated with intermediate affinity (binding affinities in the 100-1000 nM range). In only one of 32 cases was DR restriction associated with an IC₅₀ of 1000 nM or greater. Thus, 1000 nM can be defined as an affinity threshold associated with immunogenicity in the context of DR molecules.

The binding affinity of peptides for HLA molecules can be determined as described in Example 1, below.

D. Peptide Epitope Binding Motifs and Supermotifs

In the past few years evidence has accumulated to demonstrate that a large fraction of HLA class I, and possibly class II molecules can be classified into a relatively few supertypes characterized by largely overlapping peptide binding repertoires, and consensus structures of the main peptide binding pockets.

For HLA molecule pocket analyses, the residues comprising the B and F pockets of HLA class I molecules as described in crystallographic studies (Guo, H. C. et al., Nature 360:364, 1992; Saper, M. A. , Bjorkman, P. J. and Wiley, D. C., J. Mol. Biol. 219:277, 1991; Madden, D. R., Garboczi, D. N. and Wiley, D. C., Cell 75:693, 1993), have been compiled from the database of Parham, et al. (Parham, P., Adams, E. J., and Arnett, K. L., Immunol. Rev. 143:141, 1995). In these analyses, residues 9, 45, 63, 66, 67, 70, and 99 were considered to make up the B pocket, and to determine the specificity for the residue in the second position of peptide ligands. Similarly, residues 77, 80, 81, and 116 were considered to determine the specificity of the F pocket, and to determine the specificity for the C-terminal residue of a peptide ligand bound by the HLA molecule.

Through the study of single amino acid substituted antigen analogs and the sequencing of endogenously bound, naturally processed peptides, critical residues required for allele-specific binding to HLA molecules have been identified. The presence of these residues correlates with binding affinity for HLA molecules. The identification of motifs and/or supermotifs that correlate with high and intermediate affinity binding is an important issue with respect to the identification of immunogenic peptide epitopes for the inclusion in a vaccine. Kast et al. (J. Immunol. 152:3904-3912, 1994) have shown that motif-bearing peptides account for 90% of the epitopes that bind to allele-specific HLA class I molecules. In this study all possible peptides of 9 amino acids in length and overlapping by eight amino acids (240 peptides), which cover the entire sequence of the E6 and E7 proteins of human papillomavirus type 16, were evaluated for binding to five allele-specific HLA molecules that are expressed at high frequency among different ethnic groups. This unbiased set of peptides allowed an evaluation of the predictive value of HLA class I motifs. From the set of 240 peptides, 22 peptides were identified that bound to an allele-specific HLA molecules with high or intermediate affinity. Of these 22 peptides, 20, (i.e. 91%), were motif-bearing. Thus, this study demonstrates the value of motifs for the identification of peptide epitopes for inclusion in a vaccine: application of motif-based identification techniques eliminates screening of 90% of the potential epitopes.

Such peptide epitopes are identified in the Tables described below. The Tables for the HLA class I epitopes include over 90% of the peptides that will bind to an allele-specific HLA class I molecule with intermediate or high affinity.

Peptides of the present invention may also include epitopes that bind to MHC class II DR molecules. A significant difference between class I and class II HLA molecules is that, although a stringent size restriction exists for peptide binding to class I molecules, a greater degree of heterogeneity in both sizes and binding frame positions of the motif, relative to the N and C termini of the peptide, can be demonstrated for class II peptide ligands. This increased heterogeneity is due to the structure of the class II-binding groove which, unlike its class I counterpart, is open at both ends. Crystallographic analysis of DRB*0101-peptide complexes (see, e.g., Madden, D. R. Ann. Rev. Immunol. 13:587, 1995) showed that the residues occupying position 1 and position 6 of peptides complexed with DRB*0101 engage two complementary pockets on the DRBa*0101 molecules, with the P1 position corresponding to the most crucial anchor position as a crucial anchor residue for binding to various other DR molecules.

Thus, peptides of the present invention are identified by any one of several HLA-specific amino acid motifs(see, e.g., Tables I-III). If the presence of the motif corresponds to the ability to bind several allele-specific HLA antigens it is referred to as a supermotif. The allele-specific HLA molecules that bind to peptides that possess a particular amino acid supermotif are collectively referred to as an HLA “supertype.”

The peptide motifs and supermotifs described below provide guidance for the identification and use of peptides in accordance with the invention.

Examples of peptide epitopes bearing the respective supermotif or motif are included in Tables as designated in the description of each motif or supermotif. The Tables include a binding affinity ratio listing for some of the peptide epitopes. The ratio may be converted to IC₅₀ by using the following formula: IC₅₀ of the standard peptide/ratio=IC₅₀ of the test peptide (i.e. the peptide epitope). The IC₅₀ values of standard peptides used to determine binding affinities for Class I peptides are shown in Table IV. The IC₅₀ values of standard peptides used to determine binding affinities for Class II peptides are shown in Table V. The peptides used as standards for the binding assay are examples of standards; alternative standard peptides can also be used when performing such an analysis.

To obtain the peptide epitope sequences listed in each Table, protein sequence data from twenty HBV strains (HPBADR, HPBADR1CG, HPBADRA, HPBADRC, HPBADRCG, HPBCGADR, HPBVADRM, HPBADW, HPBADW1, HPBADW2, HPBADW3, HPBADWZ, HPBHEPB, HPBVADW2, HPBAYR, HPBV, HPBVAYWC, HPBVAYWCI, NAD HPBVAYWE) were evaluated for the presence of the designated supermotif or motif. Peptide epitopes were also selected on the basis of their conservancy. A criterion for conservancy requires that the entire sequence of a peptide be totally conserved in 75% of the sequences available for a specific protein. The percent conservancy of the selected peptide epitopes is indicated on the Tables. The frequency, i.e. the number of strains of the 20 strains in which the peptide sequence was identified, is also shown. The “1st position” column in the Tables designates the amino acid position of the HBV protein that corresponds to the first amino acid residue of the epitope. “Number of amino acids” indicates the number of residues in the epitope sequence.

HLA Class I Motifs Indicative of CTL Inducing Peptide Epitopes:

The primary anchor residues of the HLA class I peptide epitope supermotifs and motifs delineated below are summarized in Table I. The HLA class I motifs set out in Table I(a) are those most particularly relevant to the invention claimed here. Primary and secondary anchor positions are summarized in Table II. Allele-specific HLA molecules that comprise HLA class I supertype families are listed in Table VI.

1. HLA-A1 Supermotif

The HLA-A1 supermotif is characterized by the presence in peptide ligands of a small (T or S) or hydrophobic (L, I, V, or M) primary anchor residue in position 2, and an aromatic (Y, F, or W) primary anchor residue at the C-terminal position of the epitope. The corresponding family of HLA molecules that bind to the A1 supermotif (i.e., the HLA-A1 supertype) is comprised of at least A*0101, A*2601, A*2602, A*2501, and A*3201 (see, e.g., DiBrino, M. et al., J. Immunol. 151:5930, 1993; DiBrino, M. et al., J. Immunol. 152:620, 1994; Kondo, A. et al., Immunogenetics 45:249, 1997). Other allele-specific HLA molecules predicted to be members of the A1 superfamily are shown in Table VI. Peptides binding to each of the individual HLA proteins can be modulated by substitutions at primary and/or secondary anchor positions, preferably choosing respective residues specified for the supermotif.

Representative peptide epitopes that comprise the A1 supermotif are set forth on the attached Table VII.

2. HLA-A2 Supermotif

Primary anchor specificities for allele-specific HLA A2.1 molecules (Falk et al., Nature 351:290-296, 1991; Hunt et al., Science 255:1261-1263, 1992) and cross-reactive binding within the HLA A2 family (Fruci et al., Human Immunol. 38:187-192, 1993; Tanigaki et al., Human Immunol. 39:155-162, 1994) have been described. The present inventors have defined additional primary anchor residues that determine cross-reactive binding to multiple allele-specific HLA A2 molecules (Del Guercio et al., J. Immunol. 154:685-693, 1995). The HLA-A2 supermotif comprises peptide ligands with L, I, V, M, A, T, or Q as primary anchor residues at position 2 and L, I, V, M, A, or T as a primary anchor residue at the C-terminal position of the epitope.

The corresponding family of HLA molecules (i.e., the HLA-A2 supertype that binds these peptides) is comprised of at least: A*0201, A*0-202, A*0203, A*0204, A*0205, A*0206, A*0207, A*0209, a*0214, A*6802, and A*6901. Other allele-specific HLA molecules predicted to be members of the A2 superfamily are shown in Table VI. As explained in detail below, binding to each of the individual allele-specific HLA molecules can be modulated by substitutions at the primary anchor and/or secondary anchor positions, preferably choosing respective residues specified for the supermotif.

Representative peptide epitopes that comprise an A2 supermotif are set forth on the attached Table VIII. The motifs comprising the primary anchor residues V, A, T, or Q at position 2 and L, I, V, A, or T at the C-terminal position are those most particularly relevant to the invention claimed herein.

3. HLA-A3 Supermotif

The HLA-A3 supermotif is characterized by the presence in peptide ligands of A, L, I, V, M, S, or, T as a primary anchor at position 2, and a positively charged residue, R or K, at the C-terminal position of the epitope (e.g., in position 9 of 9-mers). Exemplary members of the corresponding family of HLA molecules (the HLA-A3 supertype) that bind the A3 supermotif include at least: A*0301, A*1101, A*3101, A*3301, and A*6801. Other allele-specific HLA molecules predicted to be members of the A3 superfamily are shown in Table VI. As explained in detail below, peptide binding to each of the individual allele-specific HLA proteins can be modulated by substitutions of amino acids at the primary and/or secondary anchor positions of the peptide, preferably choosing respective residues specified for the supermotif.

Representative peptide epitopes that comprise the A3 supermotif are set forth on the attached Table IX.

4. HLA-A24 Supermotif

The HLA-A24 supermotif is characterized by the presence in peptide ligands of an aromatic (F, W, or Y) residue as a primary anchor in position 2, and a hydrophobic (Y, F, L, I, V, or M) residue as primary anchor at the C-terminal position of the epitope. The corresponding family of HLA molecules that bind to the A24 supermotif (i.e., the A24 supertype) includes at least A*2402, A*3001, and A*2301. Other allele-specific HLA molecules predicted to be members of the A24 superfamily are shown in Table VI.Peptide binding to each of the allele-specific HLA molecules can be modulated by substitutions at primary anchor positions, preferably choosing respective residues specified for the supermotif.

Representative peptide epitopes that comprise the A24 supermotif are set forth on the attached Table X.

5. HLA-B7 Supermotif

The HLA-B7 supermotif is characterized by peptides bearing proline in position 2 as a primary anchor, and a hydrophobic or aliphatic amino acid (L, I, V, M, A, F, W, or Y) as the primary anchor at the C-terminal position of the epitope. The corresponding family of HLA molecules that bind the B7 supermotif (i.e., the HLA-B7 supertype) is comprised of at least twenty six HLA-B proteins including: B*0702, B*0703, B*0704, B*0705, B*1508, B*3501, B*3502, B*3503, B*3504, B*3505, B*3506, B*3507, B*3508, B*5101, B*5102, B*5103, B*5104, B*5105, B*5301, B*5401, B*5501, B*5502, B*5601, B*5602, B*6701, and B*7801 (see, e.g., Sidney, et al., J. Immunol. 154:247, 1995; Barber, et al., Curr. Biol. 5:179, 1995; Hill, et al., Nature 360:434, 1992; Rammensee, et al., Immunogenetics 41:178, 1995). Other allele-specific HLA molecules predicted to be members of the B7 superfamily are shown in Table VI. As explained in detail below, peptide binding to each of the individual allele-specific HLA proteins can be modulated by substitutions at the primary and/or secondary anchor positions of the peptide, preferably choosing respective residues specified for the supermotif.

Representative peptide epitopes that contain the B7 supermotif are set forth on the attached Table XI.

6. HLA-B27 Supermotif

The HLA-B27 supermotif is characterized by the presence in peptide ligands of a positively charged (R, H, or K) residue as a primary anchor at position 2, and a hydrophobic (F, Y, L, W, M, I, A, or V) residue as a primary anchor at the C-terminal position of the epitope. Exemplary members of the corresponding family of HLA molecules that bind to the B27 supermotif (i.e., the B27 supertype) include at least B*1401, B*1402, B*1509, B*2702, B*2703, B*2704, B*2705, B*2706, B*3801, B*3901, B*3902, and B*7301. Other allele-specific HLA molecules predicted to be members of the B27 superfamily are shown in Table VI. Peptide binding to each of the allele-specific HLA molecules can be modulated by substitutions at primary anchor positions, preferably choosing respective residues specified for the supermotif.

Representative peptide epitopes that comprise the B27 supermotif are set forth on the attached Table XII.

7. HLA-B44 Supermotif

The HLA-B44 supermotif is characterized by the presence in peptide ligands of negatively charged (D or E) residues as a primary anchor in position 2, and hydrophobic residues (F, W, Y, L, I, M, V, or A) as a primary anchor at the C-terminal position of the epitope. Exemplary members of the corresponding family of HLA molecules that bind to the B44 supermotif (i.e., the B44 supertype) include at least: B*1801, B*1802, B*3701, B*4001, B*4002, B*4006, B*4402, B*4403, and B*4006. Peptide binding to each of the allele-specific HLA molecules can be modulated by substitutions at primary anchor positions; preferably choosing respective residues specified for the supermotif.

8. HLA-B58 Supermotif

The HLA-B58 supermotif is characterized by the presence in peptide ligands of a small aliphatic residue (A, S, or T) as a primary anchor residue at position 2, and an aromatic or hydrophobic residue (F, W, Y, L, I, V, M, or A) as a primary anchor residue at the C-terminal position of the epitope. Exemplary members of the corresponding family of HLA molecules that bind to the B58 supermotif (i.e., the B58 supertype) include at least: B*1516, B*1517, B*5701, B*5702, and B*5801. Other allele-specific HLA molecules predicted to be members of the B58 superfamily are shown in Table VI. Peptide binding to each of the allele-specific HLA molecules can be modulated by substitutions at primary anchor positions, preferably choosing respective residues specified for the supermotif.

Representative peptide epitopes that comprise the B58 supermotif are set forth on the attached Table XIII.

9. HLA-B62 Supermotif

The HLA-B62 supermotif is characterized by the presence in peptide ligands of the polar aliphatic residue Q or a hydrophobic aliphatic residue (L, V, M, or I) as a primary anchor in position 2, and a hydrophobic residue (F, W, Y, M, I, V, L, or A) as a primary anchor at the C-terminal position of the epitope. Exemplary members of the corresponding family of HLA molecules that bind to the B62 supermotif (i.e., the B62 supertype) include at least: B*1501, B*1502, B*1513, and B5201. Other allele-specific HLA molecules predicted to be members of the B62 superfamily are shown in Table VI. Peptide binding to each of the allele-specific HLA molecules can be modulated by substitutions at primary anchor positions, preferably choosing respective residues specified for the supermotif.

Representative peptide epitopes that comprise the B62 supermotif are set forth on the attached Table XIV.

10. HLA-A1 Motif

The allele-specific HLA-A1 motif is characterized by the presence in peptide ligands of T, S, or M as a primary anchor residue at position 2 and the presence of Y as a primary anchor residue at the C-terminal position of the epitope. An alternative allele-specific A1 motif (i.e., a “submotif”) is characterized by a primary anchor residue at position 3 rather than position 2. This submotif is characterized by the presence of D, E, A, or S as a primary anchor residue in position 3, and a Y as a primary anchor residue at the C-terminal position of the epitope. An extended submotif is characterized by the presence of D in position 3 and A, I, L, or F at the C-terminus. Peptide binding to HLA A1 can be modulated by substitutions at primary and/or secondary anchor positions, preferably choosing respective residues specified for the motif.

Representative peptide epitopes that comprise either A1 motif are set forth on the attached Table XV. Those epitopes comprising T, S, or M at position 2 and Y at the C-terminal position are also included in the listing of HLA-A1 supermotif-bearing peptide epitopes listed in Table VII.

11. HLA-A2.1 Motif

An allele-specific HLA-A2.1 motif was first determined to be characterized by the presence in peptide ligands of L or M as a primary anchor residue in position 2, and L or V as a primary anchor residue at the C-terminal position of a 9 amino acid epitope (Falk et al., Nature 351:290-296, 1991). Furthermore, the A2.1 motif was determined to further comprise an I at position 2 and I or A at the C-terminal position of a nine amino acid peptide (Hunt et al., Science 255:1261-1263, Mar. 6, 1992). Additionally, the A2.1 allele-specific motif has been found to comprise a T at the C-terminal position (Kast et al., J. Immunol. 152:3904-3912, 1994). Subsequently, the A2.1 allele-specific motif has been defined by the present inventors to additionally comprise V, A, T, or Q as a primary anchor residue at position 2, and M as a primary anchor residue at the C-terminal position of the epitope. Thus, the HLA-A2.1 motif comprises peptide ligands with L, I, V, M, A, T, or Q as primary anchor residues at position 2 and L, I, V, M, A, or T as a primary anchor residue at the C-terminal position of the epitope. The preferred and tolerated residues that characterize the primary anchor positions of the HLA-A2.1 motif are identical to the preferred residues of the A2 supermotif. (for reviews of relevant data, see, e.g., Del Guercio et al., J. Immunol. 154:685-693, 1995; Sidney et al., Immunol. Today 17:261-266, 1996; Sette and Sidney, Curr. Opin. in Immunol. 10:478-482, 1998). Secondary anchor residues that characterize the A2.1 motif have additionally been defined as disclosed herein. These are disclosed in Table II. Peptide binding to HLA-A2.1 molecules can be modulated by substitutions at primary and/or secondary anchor positions, preferably choosing respective residues specified for the motif.

Representative peptide epitopes that comprise an A2.1 motif are set forth on the attached Table VII. The A2.1 motifs comprising the primary anchor residues V, A, T, or Q at position 2 and L, I, V, A, or T at the C-terminal position are those most particularly relevant to the invention claimed herein.

12. HLA-A3 Motif

The allele-specific HLA-A3 motif is characterized by the presence in peptide ligands of L, M, V, I, S, A, T, F, C, G, or D as a primary anchor residue at position 2, and the presence of K, Y, R, H, F, or A as a primary anchor residue at the C-terminal position of the epitope. Peptide binding to HLA-A3 can be modulated by substitutions at primary and/or secondary anchor positions, preferably choosing respective residues specified for the motif.

Representative peptide epitopes that comprise the A3 motif are set forth on the attached Table XVI. Those peptide epitopes that also comprise the A3 supermotif are also listed in Table IX.

13. HLA-A11 Motif

The allele-specific HLA-Al motif is characterized by the presence in peptide ligands of V, T, M, L, I, S, A, G, N, C, D, or F as a primary anchor residue in position 2, and K, R, Y, or H as a primary anchor residue at the C-terminal position of the epitope. Peptide binding to HLA-A11 can be modulated by substitutions at primary and/or secondary anchor positions, preferably choosing respective residues specified for the motif.

Representative peptide epitopes that comprise the A11 motif are set forth on the attached Table XVII; peptide epitopes comprising the A3 allele-specific motif are also present in this Table because of the extensive overlap between the A3 and A11 motif primary anchor specificities. Further, those peptide epitopes that comprise the A3 supermotif are also listed in Table IX.

14. HLA-A24 Motif

The allele-specific HLA-A24 motif is characterized by the presence in peptide ligands of Y, F, W, or M as a primary anchor residue in position 2, and F, L, I, or W as a primary anchor residue at the C-terminal position of the epitope. Peptide binding to HLA-A24 molecules can be modulated by substitutions at primary and/or secondary anchor positions; preferably choosing respective residues specified for the motif.

Representative epitopes that comprise the A24 motif are set forth on Table XVIII. These epitopes are also listed in Table X, HLA-A24-supermotif-bearing epitopes.

Motifs Indicative of HLA Class II HTL Epitopes

Primary and secondary anchor residues of the HLA class II supermotifs and motifs delineated below are summarized in Table III.

15. HLA DR-1-4-7 Supermotif

Motifs have also been identified for peptides that bind to three common HLA class II allele-specific HLA molecules: HLA DRB1*0401, DRB1*0101, and DRB1*0701. Collectively, the common residues from these motifs delineate the HLA DR-1-4-7 supermotif. Peptides that bind to these DR molecules carry a supermotif characterized by a large aromatic or hydrophobic residue (Y, F, W, L, I, V, or M) as a primary anchor residue in position 1, and a small, non-charged residue (S, T, C, A, P, V, I, L, or M) as a primary anchor residue in position 6 of the epitope. Allele-specific secondary effects and secondary anchors for each of these HLA types have also been identified. These are set forth in Table III. Peptide binding to HLA-DR4, DR1, and/or DR7 can be modulated by substitutions at primary and/or secondary anchor positions, preferably choosing respective residues specified for the supermotif.

Conserved peptide epitopes (i.e. 75% conservancy in the 20 HBV strains used for the analysis), corresponding to a nine residue core comprising the DR-1-4-7 supermotif (wherein position 1 of the motif is at position 1 of the nine residue core) are set forth in Table XIXa (see, e.g., Madden, Annu. Rev. Immunol. 13:587-622, 1995). Respective exemplary peptide epitopes of 15 amino acid residues in length, each of which comprise a conserved nine residue core, are also shown in section “a” of the Table. ° Cross-reactive binding data for the exemplary 15-residue supermotif-bearing peptides denoted by a peptide number are shown in Table XIXb.

16. HLA DR3 Motifs

Two alternative motifs (i.e., submotifs) characterize peptide epitopes that bind to HLA-DR3 molecules. In the first motif (submotif DR3A) a large, hydrophobic residue (L, I, V, M, F, or Y) is present in anchor position 1, and D is present as an anchor at position 4, towards the carboxyl terminus of the epitope.

The alternative DR3 submotif provides for lack of the large, hydrophobic residue at anchor position 1, and/or lack of the negatively charged or amide-like anchor residue at position 4, by the presence of a positive charge at position 6 towards the carboxyl terminus of the epitope. Thus, for the alternative allele-specific DR3 motif (submotif DR3B): L, I, V, M, F, Y, A, or Y is present at anchor position 1; D, N, Q, E, S, or T is present at anchor position 4; and K, R, or H is present at anchor position 6. Peptide binding to HLA-DR3 can be modulated by substitutions at primary and/or secondary anchor positions, preferably choosing respective residues specified for the motif.

Conserved peptide epitopes (i.e., sequences that are 75% conservaned in the 20 HBV strains used for the analysis), corresponding to a nine residue core comprising the DR3A submotif (wherein position 1 of the motif is at position 1 of the nine residue core) set forth in Table XXa. Respective exemplary peptide epitopes of 15 amino acid residues in length, each of which comprise a conserved nine residue core, are also shown in section “a” of the Table. Table XXb shows binding data of the exemplary DR3 submotif A-bearing peptides denoted by a peptide number.

Conserved peptide epitopes (i.e., 75% conservancy in the 20 HBV strains used for the analysis), corresponding to a nine residue core comprising the DR3B submotif and respective exemplary 15-mer peptides comprising the DR3 submotif-B epitope are set forth in Table XXc. Table XXd shows binding data of the exemplary DR3 submotif B-bearing peptides denoted by a peptide number.

Each of the HLA class I or class II peptide epitopes set out in the Tables herein are deemed singly to be an inventive aspect of this application. Further, it is also an inventive aspect of this application that each peptide epitope may be used in combination with any other peptide epitope.

E. Enhancing Population Coverage of the Vaccine

Vaccines that have broad population coverage are preferred because they are more commercially viable and generally applicable to the most people. Broad population coverage can be obtained using the peptides of the invention (and nucleic acid compositions that encode such peptides) through selecting peptide epitopes that bind to HLA alleles which, when considered in total, are present in most of the population. Table XXI lists the overall frequencies of the HLA class I supertypes in various ethnicities (Table XXIa) and the combined population coverage achieved by the A2-, A3-, and B7-supertypes (Table XXIb). The A2-, A3-, and B7 supertypes are each present on the average of over 40% in each of these five major ethnic groups. Coverage in excess of 80% is achieved with a combination of these supermotifs. These results suggest that effective and non-ethnically biased population coverage is achieved upon use of a limited number of cross-reactive peptides. Although the population coverage reached with these three main peptide specificities is high, coverage can be expanded to reach 95% population coverage and above, and more easily achieve truly multispecific responses upon use of additional supermotif or allele-specific motif bearing peptides.

The B44-, A1-, and A24-supertypes are present, on average, in a range from 25% to 40% of these major ethnic populations (Table XXIa). While less prevalent overall, the B27-, B58-, and B62 supertypes are each present with a frequency >25% in at least one major ethnic group (Table XXIa). Table XXIb summarizes the estimated combined prevalence in five major ethnic groups of HLA supertypes that have been identified. The incremental coverage obtained by the inclusion of A1,- A24-, and B44-supertypes to the A2, A3, and B7 coverage, or all of the supertypes described herein is shown. By including epitopes from the six most frequent supertypes, an average population coverage of 99% is obtained for five major ethnic groups.

The data presented herein, together with the previous definition of the A2-, A3-, and B7-supertypes, indicates that all antigens, with the possible exception of A29, B8, and B46, can be classified into a total of nine HLA supertypes. Focusing on the six most common supertypes affords population coverage greater than 98% for all major ethnic populations.

F. Immune Response Stimulating Peptide Analogs

Although peptides with suitable cross-reactivity among all alleles of a superfamily are identified by the screening procedures described above, cross-reactivity is not always complete and in such cases procedures to further increase cross-reactivity of peptides can be useful; such procedures can also be used to modify other properties of the peptides. Having established the general rules that govern cross-reactivity of peptides for HLA alleles within a given motif or supermotif, modification (i.e., analoging) of the structure of peptides of particular interest in order to achieve broader (or otherwise modified) HLA binding capacity can be performed. More specifically, peptides which exhibit the broadest cross-reactivity patterns, (both amongst the known T cell epitopes, as well as the more extended set of peptides that contain the appropriate supermotifs), can be produced in accordance with the teachings herein.

The strategy employed utilizes the motifs or supermotifs which correlate with binding to certain HLA molecules. The motifs or supermotifs are defined by having primary anchors, though secondary anchors can also be modified. Analog peptides can be created by substituting amino acids residues at primary anchor, secondary anchor, or at primary and secondary anchor positions. Generally, analogs are made for peptides that already bear a motif or supermotif. Preferred secondary anchor residues of supermotifs and motifs that have been defined for HLA class I and class II binding peptides are shown in Tables II and III, respectively.

For a number of the motifs or supermotifs in accordance with the invention, residues are defined which are deleterious to binding to allele-specific HLA molecules or members of HLA supertypes that bind to the respective motif or supermotif (Tables II and IIH). Accordingly, removal of residues that are detrimental to binding can be performed in accordance with the present invention. For example, in the case of the A3 supertype, when all peptides that have such deleterious residues are removed from the population of analyzed peptides, the incidence of cross-reactivity increases from 22% to 37% (see, e.g., Sidney, J. et al., Hu. Immunol. 45:79, 1996). Thus, one strategy to improve the cross-reactivity of peptides within a given supermotif is simply to delete one or more of the deleterious residues present within a peptide and substitute a small “neutral” residue such as Ala (that may not influence T cell recognition of the peptide). An enhanced likelihood of cross-reactivity is expected if, together with elimination of detrimental residues within a peptide, residues associated with high affinity binding to multiple alleles within a superfamily are inserted.

To ensure that an analog peptide, when used as a vaccine, actually elicits a CTL response to the native epitope in vivo (or, in the case of class II epitopes, elicits helper T cells that cross-react with the wild type peptides), the analog peptide may be used to immunize T cells in vitro from individuals of the appropriate HLA allele. Thereafter, the immunized cells' capacity to induce lysis of wild type peptide sensitized target cells is evaluated. It will be desirable to use as antigen presenting cells, cells that have been either infected, or transfected with the appropriate genes, or, in the cae of class II epitopes only, cells that have been pusled with whole protein antigens, to establish whether endogenously produced antigen is also recognized by the relevant T cells.

Another embodiment of the invention to ensure adequate numbers of cross-reactive cellular binders is to create analogs of weak binding peptides. Class I peptides exhibiting binding affinities of 500-50000 nM, and carrying an acceptable but suboptimal primary anchor residue at one or both positions can be “fixed” by substituting preferred anchor residues in accordance with the respective supertype. The analog peptides can then be tested for crossbinding activity.

Another embodiment for generating effective peptide analogs involves the substitution of residues that have an adverse impact on peptide stability or solubility in a liquid environment. This substitution may occur at any position of the peptide epitope. For example, a cysteine (C) can be substituted out in favor of ax-amino butyric acid. Due to its chemical nature, cysteine has the propensity to form disulfide bridges and sufficiently alter the peptide structurally so as to reduce binding capacity. Substituting α-amino butyric acid for C not only alleviates this problem, but actually improves binding and crossbinding capability in certain instances (Review: A. Sette et al., In: Persistent Viral Infections, Eds. R. Ahmed and I. Chen, John Wiley & Sons, England, 1999). Substitution of cysteine with α-amino butyric acid may occur at any residue of a peptide epitope, i.e. at either anchor or non-anchor positions.

In general, CTL and HTL responses are not directed against all possible epitopes. Rather, they are restricted to a few immunodominant determinants (Zinkemagel, et al., Adv. Immunol. 27:5159, 1979; Bennink, et al., J. Exp. Med. 168:19351939, 1988; Rawle, et al., J. Immunol. 146:3977-3984, 1991). It has been recognized that immunodominance (Benacerraf, et al., Science 175:273-279, 1972) could be explained by either the ability of a given epitope to selectively bind a particular HLA protein (determinant selection theory) (Vitiello, et al., J. Immunol. 131:1635, 1983); Rosenthal, et al., Nature 267:156-158, 1977), or being selectively recognized by the existing TCR (T cell receptor) specificities (repertoire theory) (Klein, J., Immunology, the Science of SelfNonself Discrimination, John Wiley & Sons, New York, pp. 270-310, 1982). It has been demonstrated that additional factors, mostly linked to processing events, can also play a key role in dictating, beyond strict immunogenicity, which of the many potential determinants will be presented as immunodominant (Sercarz, et al., Annu. Rev. Immunol. 11:729-766, 1993).

The concept of dominance and subdominance is relevant to immunotherapy of both infectious diseases and cancer. For example, in the course of chronic viral disease, recruitment of subdominant epitopes can be important for successful clearance of the infection, especially if dominant CTL or HTL specificities have been inactivated by functional tolerance, suppression, mutation of viruses and other mechanisms (Franco, et al., Curr. Opin. Immunol. 7:524-531, (1995)). In the case of cancer and tumor antigens, CTLs recognizing at least some of the highest binding affinity peptides might be functionally inactivated. Lower binding affinity peptides are preferentially recognized at these times.

In particular, it has been noted that a significant number of epitopes derived from known non-viral tumor associated antigens (TAA) bind HLA class I with intermediate affinity (IC₅₀ in the 50-500 nM range). For example, it has been found that 8 of 15 known TAA peptides recognized by tumor infiltrating lymphocytes (TIL) or CTL bound in the 50-500 nM range. (These data are in contrast with estimates that 90% of known viral antigens that were recognized as peptides bound HLA with IC₅₀ of 50 nM or less, while only approximately 10% bound in the 50-500 nM range (Sette, et al., J. Immunol., 153:558-5592 (1994)). In the cancer setting this phenomenon is probably due to elimination, or functional inhibition of the CTL recognizing several of the highest binding peptides, presumably because of T cell tolerization events.

Without intending to be bound by theory, it is believed that because T cells to dominant epitopes may have been clonally deleted, selecting subdominant epitopes may allow extant T cells to be recruited, which will then lead to a therapeutic response. However, the binding of HLA molecules to subdominant epitopes is often less vigorous than to dominant ones. Accordingly, there is a need to be able to modulate the binding affinity of particular immunogenic epitopes for one or more HLA molecules, and thereby to modulate the immune response elicited by the peptide. Thus, a need exists to prepare analog peptides which elicit a more vigorous response. This ability would greatly enhance the usefulness of peptide-based vaccines and therapeutic agents.

Representative analog peptides are set forth in Table XXII. The Table indicates the length and sequence of the analog peptide as well as the motif or supermotif, if appropriate. The information in the “Fixed Nomenclature” column indicates the residues substituted at the indicated position numbers for the respective analog.

G. Computer Screening of Protein Sequences from Disease-Related Antigens for Supermotif or Motif Containing Peptides

In order to identify supermotif- or motif-bearing epitopes in a target antigen, a native protein sequence, e.g., a tumor-associated antigen, or sequences from an infectious organism, or a donor tissue for transplantation, is screened using a means for computing, such as an intellectual calculation or a computer, to determine the presence of a supermotif or motif within the sequence. The information obtained from the analysis of native peptide can be used directly to evaluate the status of the native peptide or may be utilized subsequently to generate the peptide epitope.

Computer programs that allow the rapid screening of protein sequences for the occurrence of the subject supermotifs or motifs are encompassed by the present invention; as are programs that permit the generation of analog peptides. These programs are implemented to analyze any identified amino acid sequence or operate on an unknown sequence and simultaneously determine the sequence and identify motif-bearing epitopes thereof; analogs can be simultaneously determined as well. Generally, the identified sequences will be from a pathogenic organism or a tumor-associated peptide. For example, the target molecules considered herein include all of the HBV proteins (e.g. surface, core, polymerase, and X).

In cases where the sequence of multiple variants of the same target protein are available, peptides may also be selected on the basis of their conservancy. A presently preferred criterion for conservancy defines that the entire sequence of a peptide be totally conserved in 75% of the sequences evaluated for a specific protein; this definition of conservancy has been employed herein.

It is important that the selection criteria utilized for prediction of peptide binding are as accurate as possible, to correlate most efficiently with actual binding. Prediction of peptides that bind, for example, to HLA-A*0201, on the basis of the presence of the appropriate primary anchors, is positive at about a 30% rate (Ruppert, J. et al. Cell 74:929, 1993). However, by analyzing an extensive peptide-HLA binding database, the present inventors have developed a number of allele specific polynomial algorithms that dramatically increase the predictive value over identification on the basis of the presence of primary anchor residues alone. These algorithms take into account not only the presence or absence of the correct primary anchors, but also consider the positive or F deleterious presence of secondary anchor residues (to account for the impact of different amino acids at different positions). The algorithms are essentially based on the premise F that the overall affinity (or ΔG) of peptide-HLA interactions can be approximated as a linear polynomial function of the type:
ΔG=a1i×a2i×a3i . . . x ani
where aij is a coefficient that represents the effect of the presence of a given amino acid (i) at a given position (i) along the sequence of a peptide of n amino acids. An important assumption of this method is that the effects at each position are essentially independent of each other. This assumption is justified by studies that demonstrated that peptides are bound to HLA molecules and recognized by T cells in essentially an extended conformation. Derivation of specific algorithm coefficients has been described in Gulukota et al. (Gulukota, K. et al., J.Mol.Biol. 267:1258, 1997).

Additional methods to identify preferred peptide sequences, which also make use of specific motifs, include the use of neural networks and molecular modeling programs (see, e.g., Milik et al., Nature Biotechnology 16:753, 1998; Altuvia et al., Hum. Immunol. 58:1, 1997; Altuvia et al, J. Mol. Biol. 249:244, 1995; Buus, S. Curr. Opin. Immunol. 11:209-213, 1999; Brusic, V. et al., Bioinformatics 14:121-130, 1998; Parker et al., J. Immunol. 152:163, 1993; Meister et al., Vaccine 13:581, 1995; Hammer et al., J. Exp. Med. 180:2353, 1994; Sturniolo et al., Nature Biotechnol. 17:555 1999).

For example, it has been shown that in sets of A*0201 motif peptides, 69% of the peptides containing at least one preferred secondary anchor residue while avoiding the presence of any deleterious secondary anchor residues, will bind A*0201 with an IC₅₀ less than 500 nM (Ruppert, J. et al. Cell 74:929, 1993). These algorithms are also flexible in that cut-off scores may be adjusted to select sets of peptides with greater or lower predicted binding properties, as desired.

In utilizing computer screening to identify peptide epitopes, all protein sequence or translated sequence may be analyzed using software developed to search for motifs, for example the “FINDPATTERNS” program (Devereux, et al. Nucl. Acids Res. 12:387-395, 1984) or MotifSearch 1.4 software program (D. Brown, San Diego, Calif.) to identify potential peptide sequences containing appropriate HLA binding motifs. As appreciated by one of ordinary skill in the art a large array of software and hardware options are available which can be employed to implement the motifs of the invention relative to known or unknown peptide sequences. The identified peptides will then be scored using customized polynomial algorithms to predict their capacity to bind specific HLA class I or class II alleles.

In accordance with the procedures described above, HBV peptides and analogs thereof that are able to bind HLA supertype groups or allele-specific HLA molecules have been identified (Tables VII-XX; Table XXII).

H. Reparation of Peptide Epitopes

Peptides in accordance with the invention can be prepared synthetically, by recombinant DNA technology or chemical synthesis, or from natural sources such as native tumors or pathogenic organisms. Peptide epitopes may be synthesized individually or as polyepitopic peptides. Although the peptide will preferably be substantially free of other naturally occurring host cell proteins and fragments thereof, in some embodiments the peptides may be synthetically conjugated to native fragments or particles.

The peptides in accordance with the invention can be a variety of lengths, and either in their neutral (uncharged) forms or in forms which are salts. The peptides in accordance with the invention are either free of modifications such as glycosylation, side chain oxidation, or phosphorylation; or they contain these modifications, subject to the condition that modifications do not destroy the biological activity of the peptides as described herein.

When possible, it may be desirable to optimize HLA class I binding epitopes of the invention, such as can be used in a polyepitopic construct, to a length of about 8 to about 13 amino acid residues, often 8 to 11, preferably 9 to 10. HLA class II binding peptide epitopes of the invention may be optimized to a length of about 6 to about 30 amino acids in length, preferably to between about 13 and about 20 residues. Preferably, the peptide epitopes are commensurate in size with endogenously processed pathogen-derived peptides or tumor cell peptides that are bound to the relevant HLA molecules, however, the identification and preparation of peptides that comprise epitopes of the invention can also be carried out using the techniques described herein.

In alternative embodiments, epitopes of the invention can be linked as a polyepitopic peptide, or as a minigene that encodes a polyepitopic peptide.

In another embodiment, it is preferred to identify native peptide regions that contain a high concentration of class I and/or class II epitopes. Such a sequence is generally selected on the basis that it contains the greatest number of epitopes per amino acid length. It is to be appreciated that epitopes can be present in a nested or overlapping manner, e.g. a 10 amino acid long peptide could contain two 9 amino acid long epitopes and one 10 amino acid long epitope; upon intracellular processing, each epitope can be exposed and bound by an HLA molecule upon administration of such a peptide. This larger, preferably multi-epitopic, peptide can be generated synthetically, recombinantly, or via cleavage from the native source.

The peptides of the invention can be prepared in a wide variety of ways. For the preferred relatively short size, the peptides can be synthesized in solution or on a solid support in accordance with conventional techniques. Various automatic synthesizers are commercially available and can be used in accordance with known protocols. (See, for example, Stewart & Young, Solid Phase Peptide Synthesis, 2d. ed., Pierce Chemical Co., 1984). Further, individual peptide epitopes can be joined using chemical ligation to produce larger peptides that are still within the bounds of the invention.

Alternatively, recombinant DNA technology can be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook et al., Molecular Cloning, a Laboratory Manual, Cold Spring Harbor Press, Cold Spring Harbor, N.Y. (1989). Thus, recombinant polypeptides which comprise one or more peptide sequences of the invention can be used to present the appropriate T cell epitope.

The nucleotide coding sequence for peptide epitopes of the preferred lengths contemplated herein can be synthesized by chemical techniques, for example, the phosphotriester method of Matteucci, et al., J. Am. Chem. Soc. 103:3185 (1981). Peptide analogs can be made simply by substituting the appropriate and desired nucleic acid base(s) for those that encode the native peptide sequence; exemplary nucleic acid substitutions are those that encode an amino acid defined by the motifs/supermotifs herein. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast, insect or mammalian cell hosts may also be used, employing suitable vectors and control sequences.

I. Assays to Detect T-Cell Responses

Once HLA binding peptides are identified, they can be tested for the ability to elicit a T-cell response. The preparation and evaluation of motif-bearing peptides are described in PCT publications WO 94/20127 and WO 94/03205. Briefly, peptides comprising epitopes from a particular antigen are synthesized and tested for their ability to bind to the appropriate HLA proteins. These assays may involve evaluating the binding of a peptide of the invention to purified HLA class I molecules in relation to the binding of a radioiodinated reference peptide. Alternatively, cells expressing empty class I molecules (i.e. lacking peptide therein) may be evaluated for peptide binding by immunofluorescent staining and flow microfluorimetry. Other assays that may be used to evaluate peptide binding include peptide-dependent class I assembly assays and/or the inhibition of CTL recognition by peptide competition. Those peptides that bind to the class I molecule, typically with an affinity of 500 nM or less, are further evaluated for their ability to serve as targets for CTLs derived from infected or immunized individuals, as well as for their capacity to induce primary in vitro or in vivo CTL responses that can give rise to CTL populations capable of reacting with selected target cells associated with a disease.

Analogous assays are used for evaluation of HLA class II binding peptides. HLA class II motif-bearing peptides that are shown to bind, typically at an affinity of 1000 nM or less, are further evaluated for the ability to stimulate HTL responses.

Conventional assays utilized to detect T cell responses include proliferation assays, lymphokine secretion assays, direct cytotoxicity assays, and limiting dilution assays. For example, antigen-presenting cells that have been incubated with a peptide can be assayed for the ability to induce CTL responses in responder cell populations. Antigen-presenting cells can be normal cells such as peripheral blood mononuclear cells or dendritic cells. Alternatively, mutant non-human mammalian cell lines that are deficient in their ability to load class I molecules with internally processed peptides and that have been transfected with the appropriate human class I gene, may be used to test for the capacity of the peptide to induce in vitro primary CTL responses.

Peripheral blood mononuclear cells (PBMCs) may be used as the responder cell source of CTL precursors. The appropriate antigen-presenting cells are incubated with peptide, after which the peptide-loaded antigen-presenting cells are then incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the antigen from which the peptide sequence was derived.

Additionally, a method has been devised which allows direct quantification of antigen-specific T cells by staining with Fluorescein-labelled HLA tetrameric complexes (Altman, J. D. et al., Proc. Natl. Acad. Sci. USA 90:10330, 1993; Altman, J. D. et al., Science 274:94, 1996). Other relatively recent technical developments include staining for intracellular lymphokines, and interferon release assays or ELISPOT assays. Tetramer staining, intracellular lymphokine staining and ELISPOT assays all appear to be at least 10-fold more sensitive than more conventional assays (Lalvani, A. et al., J. Exp. Med. 186:859, 1997; Dunbar, P. R. et al., Curr. Biol. 8:413, 1998; Murali-Krishna, K. et al., Immunity 8:177, 1998).

HTL activation may also be assessed using such techniques known to those in the art such as T cell proliferation and secretion of lymphokines, e.g. IL-2 (see, e.g. Alexander et al., Immunity 1:751-761, 1994).

Alternatively, immunization of HLA transgenic mice can be used to determine immunogenicity of peptide epitopes. Several transgenic mouse models including mice with human A2.1, A11 (which can additionally be used to analyze HLA-A3 epitopes), and B7 alleles have been characterized and others (e.g., transgenic mice for HLA-A1 and A24) are being developed. HLA-DR1 and HLA-DR3 mouse models have also been developed. Additional transgenic mouse models with other HLA alleles may be generated as necessary. Mice may be immunized with peptides emulsified in Incomplete Freund's Adjuvant and the resulting T cells tested for their capacity to recognize peptide-pulsed target cells and target cells transfected with appropriate genes. CTL responses may be analyzed using cytotoxicity assays described above. Similarly, HTL responses may be analyzed using such assays as T cell proliferation or secretion of lymphokines.

Immunogenic peptide epitopes are set out in Table XXIII.

J. Use of Peptide Epitopes as Diagnostic Agents and for Evaluating Immune Responses

In one aspect of the invention, HLA class I and class II binding peptides as described herein can be used as reagents to evaluate an immune response. The immune response to be evaluated is induced by using as an immunogen any agent that may result in the production of antigen-specific CTLs or HTLs that recognize and bind to the peptide epitope(s) to be employed as the reagent. The peptide reagent need not be used as the immunogen. Assay systems that are used for such an analysis include relatively recent technical developments such as tetramers, staining for intracellular lymphokines and interferon release assays, or ELISPOT assays.

For example, a peptide of the invention is used in a tetramer staining assay to assess peripheral blood mononuclear cells for the presence of antigen-specific CTLs following exposure to a pathogen or immunogen. The HLA-tetrameric complex is used to directly visualize antigen-specific CTLs (see, e.g., Ogg et al., Science 279:2103-2106, 1998; and Altman et al., Science 174:94-96, 1996) and determine the frequency of the antigen-specific CTL population in a sample of peripheral blood mononuclear cells.

A tetramer reagent using a peptide of the invention is generated as follows: A peptide that binds to an HLA molecule is refolded in the presence of the corresponding HLA heavy chain and β2-microglobulin to generate a trimolecular complex. The complex is biotinylated at the carboxyl terminal end of the heavy chain at a site that was previously engineered into the protein. Tetramer formation is then induced by the addition of streptavidin. By means of fluorescently labeled streptavidin, the tetramer can be used to stain antigen-specific cells. The cells can then be readily identified, for example, by flow cytometry. Such procedures are used for diagnostic or prognostic purposes. Cells identified by the procedure can also be used for therapeutic purposes.

Peptides of the invention are also used as reagents to evaluate immune recall responses. (see, e.g., Bertoni et al., J. Clin. Invest. 100:503-513, 1997 and Penna et al., J. Exp. Med. 174:1565-1570, 1991.) For example, patient PBMC samples from individuals infected with HPV are analyzed for the presence of antigen-specific CTLs or HTLs using specific peptides. A blood sample containing mononuclear cells may be evaluated by cultivating the PBMCs and stimulating the cells with a peptide of the invention. After an appropriate cultivation period, the expanded cell population may be analyzed, for example, for CTL or for HTL activity.

The peptides are also used as reagents to evaluate the efficacy of a vaccine. PBMCs obtained from a patient vaccinated with an immunogen are analyzed using, for example, either of the methods described above. The patient is HLA typed, and peptide epitope reagents that recognize the allele-specific molecules present in that patient are selected for the analysis. The immunogenicity of the vaccine is indicated by the presence of HPV epitope-specific CTLs and/or HTLs in the PBMC sample.

The peptides of the invention are also be used to make antibodies, using techniques well known in the art (see, e.g. Current Protocols in Immunology, Wiley/Greene, N.Y.; and Antibodies A Laboratory Manual Harlow, Harlow and Lane, Cold Spring Harbor Laboratory Press, 1989), which may be useful as reagents to diagnose HPV infection. Such antibodies include those that recognize a peptide in the context of an HLA molecule, i.e., antibodies that bind to a peptide-MHC complex.

K. Vaccine Compositions

Vaccines and methods of preparing vaccines that contain an immunogenically effective amount of one or more peptides as described herein are further embodiments of the invention. Once appropriately immunogenic epitopes have been defined, they can be sorted and delivered by various means, herein referred to as “vaccine” compositions. Such vaccine compositions can include, for example, lipopeptides (e.g.,Vitiello, A. et al., J. Clin. Invest. 95:341, 1995), peptide compositions encapsulated in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995), peptide compositions contained in immune stimulating complexes (ISCOMS) (see, e.g., Takahashi et al., Nature 344:873-875, 1990; Hu et al., Clin Exp Immunol. 113:235-243, 1998), multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996), peptides formulated as multivalent peptides; peptides for use in ballistic delivery systems, typically crystallized peptides, viral delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990), particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995), adjuvants (Warren, H. S., Vogel, F. R., and Chedid, L. A. Annu. Rev. Immunol. 4:369, 1986; Gupta, R. K. et al., Vaccine 11:293, 1993), liposomes (Reddy, R. et al., J. Immunol. 148:1585, 1992; Rock, K. L., Immunol. Today 17:131, 1996), or, naked or particle absorbed cDNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993). Toxin-targeted delivery technologies, also known as receptor mediated targeting, such as those of Avant Immunotherapeutics, Inc. (Needham, Mass.) may also be used.

Vaccine compositions of the invention include nucleic acid-mediated modalities. DNA or RNA encoding one or more of the peptides of the invention can also be administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, for example, as a vector to express nucleotide sequences that encode the peptides of the invention. Upon introduction into an acutely or chronically infected host or into a non-infected host, the recombinant vaccinia virus expresses the immunogenic peptide, and thereby elicits a host CTL and/or HTL response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors e.g. adeno and adeno-associated virus vectors, retroviral vectors, Salmonella typhi vectors, detoxified anthrax toxin vectors, and the like, will be apparent to those skilled in the art from the description herein.

Furthermore, vaccines in accordance with the invention encompass compositions of one or more of the claimed peptides. A peptide can be present in a vaccine individually. Alternatively, the peptide can exist as a homopolymer comprising multiple copies of the same peptide, or as a heteropolymer of various peptides. Polymers have the advantage of increased immunological reaction and, where different peptide epitopes are used to make up the polymer, the additional ability to induce antibodies and/or CTLs that react with different antigenic determinants of the pathogenic organism or tumor-related peptide targeted for an immune response. The composition can be a naturally occurring region of an antigen or can be prepared, e.g., recombinantly or by chemical synthesis.

Carriers that can be used with vaccines of the invention are well known in the art, and include, e.g., thyroglobulin, albumins such as human serum albumin, tetanus toxoid, polyamino acids such as poly 1-lysine, poly 1-glutamic acid, influenza, hepatitis B virus core protein, and the like. The vaccines can contain a physiologically tolerable (i.e., acceptable) diluent such as water, or saline, preferably phosphate buffered saline. The vaccines also typically include an adjuvant. Adjuvants such as incomplete Freund's adjuvant, aluminum phosphate, aluminum hydroxide, or alum are examples of materials well known in the art. Additionally, as disclosed herein, CTL responses can be primed by conjugating peptides of the invention to lipids, such as tripalmitoyl-S-glycerylcysteinlyseryl-serine (P3CSS).

Upon immunization with a peptide composition in accordance with the invention, via injection, aerosol, oral, transdermal, transmucosal, intrapleural, intrathecal, or other suitable routes, the immune system of the host responds to the vaccine by producing large amounts of CTLs and/or HTLs specific for the desired antigen. Consequently, the host becomes at least partially immune to later infection, or at least partially resistant to developing an ongoing chronic infection, or derives at least some therapeutic benefit when the antigen was tumor-associated.

In some embodiments, it may be desirable to combine the class I peptide components with components that induce or facilitate neutralizing antibody and or helper T cell responses to the target antigen of interest. A preferred embodiment of such a composition comprises class I and class II epitopes in accordance with the invention. An alternative embodiment of such a composition comprises a class I and/or class II epitope in accordance with the invention, along with a PanDR molecule, e.g., PADRE® (Epimmune, San Diego, Calif.; described, e.g., in U.S. Pat. No. 5,736,142).

A vaccine of the invention can also include antigen-presenting cells (APC), such as dendritic cells (DC), as a vehicle to present peptides of the invention. Vaccine compositions can be created in vitro, following dendritic cell mobilization and harvesting, whereby loading of dendritic cells occurs in vitro. For example, dendritic cells are transfected, e.g., with a minigene in accordance with the invention, or are pulsed with peptides. The dendritic cell can then be administered to a patient to elicit immune responses in vivo.

Vaccine compositions, either DNA- or peptide-based, can also be administered in vivo in combination with dendritic cell mobilization whereby loading of dendritic cells occurs in vivo.

Antigenic peptides are used to elicit a CTL and/or HTL response ex vivo, as well. The resulting CTL or HTL cells, can be used to treat chronic infections, or tumors in patients that do not respond to other conventional forms of therapy, or will not respond to a therapeutic vaccine peptide or nucleic acid in accordance with the invention. Ex vivo CTL or HTL responses to a particular antigen (infectious or tumor-associated antigen) are induced by incubating in tissue culture the patient's, or genetically compatible, CTL or HTL precursor cells together with a source of APC, such as DC, and the appropriate immunogenic peptide. After an appropriate incubation time (typically about 7-28 days), in which the precursor cells are activated and expanded into effector cells, the cells are infused back into the patient, where they will destroy or facilitate destruction of their specific target cell (an infected cell or a tumor cell). Transfected dendritic cells may also be used as antigen presenting cells.

The vaccine compositions of the invention can also be used in combination with other treatments used for chronic viral infection, including use in combination with immune adjuvants such as IFN-γ and the like.

Preferably, the following principles are utilized when selecting an array of epitopes for inclusion in a polyepitopic composition for use in a vaccine, or for selecting discrete epitopes to be included in a vaccine and/or to be encoded by nucleic acids such as a minigene. It is preferred that each of the following principles are balanced in order to make the selection. The multiple epitopes to be incorporated in a given vaccine composition may be, but need not be, contiguous in sequence in the native antigen from which the epitopes are derived.

1.) Epitopes are selected which, upon administration, mimic immune responses that have been observed to be correlated with HBV clearance. For HLA Class I this includes 3-4 epitopes that come from at least one antigen of HBV. In other words, it has been observed that in patients who spontaneously clear HBV, that they had generated an immune response to at least 3 epitopes on at least one HBV antigen. For HLA Class II a similar rationale is employed; again 3-4 epitopes are selected from at least one HBV antigen (see e.g., Rosenberg et al. Science 278:1447-1450).

2.) Epitopes are selected that have the requisite binding affinity established to be correlated with immunogenicity: for HLA Class I an IC₅₀ of 500 nM or less, often 200 nM or less; and for Class II an IC₅₀ of 1000 nM or less.

3.) Sufficient supermotif bearing-peptides, or a sufficient array of allele-specific motif-bearing peptides, are selected to give broad population coverage. For example, it is preferable to have at least 80% population coverage. A Monte Carlo analysis, a statistical evaluation known in the art, can be employed to assess the breadth, or redundancy of, population coverage.

4.) When selecting epitopes from cancer-related antigens it is often useful to select analogs because the patient may have developed tolerance to the native epitope. When selecting epitopes for infectious disease-related antigens it is preferable to select either native or analoged epitopes.

5.) Of particular relevance are epitopes referred to as “nested epitopes.” Nested epitopes occur where at least two epitopes overlap in a given peptide sequence. A nested peptide sequence can comprise both HLA class I and HLA class II epitopes. When providing nested epitopes, a general objective is to provide the greatest number of epitopes per sequence. Thus, an aspect is to avoid providing a peptide that is any longer than the amino terminus of the amino terminal epitope and the carboxyl terminus of the carboxyl terminal epitope in the peptide. When providing a multi-epitopic sequence, such as a sequence comprising nested epitopes, it is generally important to screen the sequence in order to insure that it does not have pathological or other deleterious biological properties.

6.) If a polyepitopic protein is created, or when creating a minigene, an objective is to generate the smallest peptide that encompasses the epitopes of interest. This principle is similar, if not the same as that employed when selecting a peptide comprising nested epitopes. However, with an artificial polyepitopic peptide, the size minimization objective is balanced against the need to integrate any spacer sequences between epitopes in the polyepitopic protein. Spacer amino acid residues can, for example, be introduced to avoid junctional epitopes (an epitope recognized by the immune system, not present in the target antigen, and only created by the man-made juxtaposition of epitopes), or to facilitate cleavage between epitopes and thereby enhance epitope presentation. Junctional epitopes are generally to be avoided because the recipient may generate an immune response to that non-native epitope. Of particular concern is a junctional epitope that is a “dominant epitope.” A dominant epitope may lead to such a zealous response that immune responses to other epitopes are diminished or suppressed.

7.) In cases where the sequences of multiple variants of the same target protein are available, potential peptide epitopes can also be selected on the basis of their conservancy. For example, a criterion for conservancy may define that the entire sequence of an HLA class I binding peptide or the entire 9-mer core of a class II binding peptide be conserved in a designated percentage of the sequences evaluated for a specific protein antigen.

1. Minigene Vaccines

A number of different approaches are available which allow simultaneous delivery of multiple epitopes. Nucleic acids encoding the peptides of the invention are a particularly useful embodiment of the invention. Epitopes for inclusion in a minigene are preferably selected according to the guidelines set forth in the previous section. A preferred means of administering nucleic acids encoding the peptides of the invention uses minigene constructs encoding a peptide comprising one or multiple epitopes of the invention.

The use of multi-epitope minigenes is described below and in, e.g., co-pending application U.S. Ser. No. 09/311,784; Ishioka et al., J. Immunol. 162:3915-3925, 1999; An, L. and Whitton, J. L., J. Virol. 71:2292, 1997; Thomson, S. A. et al., J. Immunol. 157:822, 1996; Whitton, J. L. et al., J. Virol. 67:348, 1993; Hanke, R. et al., Vaccine 16:426, 1998. For example, a multi-epitope DNA plasmid encoding supermotif- and/or motif-bearing epitopes derived from multiple regions of one or more HBV antigens, a universal helper T cell epitope, e.g., PADRE®, (or multiple HTL epitopes from HBV antigens), and an endoplasmic reticulum-translocating signal sequence can be engineered. A vaccine may also comprise epitopes that are derived from other TAAs.

The immunogenicity of a multi-epitopic minigene can be tested in transgenic mice to evaluate the magnitude of CTL induction responses against the epitopes tested. Further, the immunogenicity of DNA-encoded epitopes in vivo can be correlated with the in vitro responses of specific CTL lines against target cells transfected with the DNA plasmid. Thus, these experiments can show that the minigene serves to both: 1.) generate a CTL response and 2.) that the induced CTLs recognized cells expressing the encoded epitopes.

For example, to create a DNA sequence encoding the selected epitopes (minigene) for expression in human cells, the amino acid sequences of the epitopes may be reverse translated. A human codon usage table can be used to guide the codon choice for each amino acid. These epitope-encoding DNA sequences may be directly adjoined, so that when translated, a continuous polypeptide sequence is created. To optimize expression and/or immunogenicity, additional elements can be incorporated into the minigene design. Examples of amino acid sequences that could be reverse translated and included in the minigene sequence include: HLA class I epitopes, HLA class II epitopes, a ubiquitination signal sequence, and/or an endoplasmic reticulum targeting signal. In addition, HLA presentation of CTL and HTL epitopes may be improved by including synthetic (e.g. poly-alanine) or naturally-occurring flanking sequences adjacent to the CTL or HTL epitopes; these larger peptides comprising the epitope(s) are within the scope of the invention.

The minigene sequence may be converted to DNA by assembling oligonucleotides that encode the plus and minus strands of the minigene. Overlapping oligonucleotides (30-100 bases long) may be synthesized, phosphorylated, purified and annealed under appropriate conditions using well known techniques. The ends of the oligonucleotides can be joined, for example, using T4 DNA ligase. This synthetic minigene, encoding the epitope polypeptide, can then be cloned into a desired expression vector.

Standard regulatory sequences well known to those of skill in the art are preferably included in the vector to ensure expression in the target cells. Several vector elements are desirable: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences and sequences for replication in mammalian cells may also be considered for increasing minigene expression.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

In addition, immunostimulatory sequences (ISSs or CpGs) appear to play a role in the immunogenicity of DNA vaccines. These sequences may be included in the vector, outside the minigene coding sequence, if desired to enhance immunogenicity.

In some embodiments, a bi-cistronic expression vector which allows production of both the minigene-encoded epitopes and a second protein (included to enhance or decrease immunogenicity) can be used. Examples of proteins or polypeptides that could beneficially enhance the immune response if co-expressed include cytokines (e.g., IL-2, IL-12, GM-CSF), cytokine-inducing molecules (e.g., LeIF) or costimulatory molecules. Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving CTL induction. In contrast to HTL or CTL induction, specifically decreasing the immune response by co-expression of immunosuppressive molecules (e.g. TGF-β) may be beneficial in certain diseases).

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins supplied by QIAGEN, Inc. (Valencia, Calif.). If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, known as “naked DNA,” is currently being used for intramuscular (IM) administration in clinical trials. To maximize the immunotherapeutic effects of minigene DNA vaccines, an alternative method for formulating purified plasmid DNA may be desirable. A variety of methods have been described, and new techniques may become available. Cationic lipids can also be used in the formulation (see, e.g., as described by WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In addition, glycolipids, fusogenic liposomes, peptides and compounds referred to collectively as protective, interactive, non-condensing compounds (PINC) could also be complexed to purified plasmid DNA to influence variables such as stability, intramuscular dispersion, or trafficking to specific organs or cell types.

Target cell sensitization can be used as a functional assay for expression and HLA class I presentation of minigene-encoded CTL epitopes. For example, the plasmid DNA is introduced into a mammalian cell line that is suitable as a target for standard CTL chromium release assays. The transfection method used will be dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 (⁵¹Cr) labeled and used as target cells for epitope-specific CTL lines; cytolysis, detected by ⁵¹Cr release, indicates both production of, and HLA presentation of, minigene-encoded CTL epitopes.

In vivo inmmunogenicity is a second approach for functional testing of minigene DNA formulations. Transgenic mice expressing appropriate human HLA proteins are immunized with the DNA product. The dose and route of administration are formulation dependent (e.g., IM for DNA in PBS, intraperitoneal (IP) for lipid-complexed DNA). Twenty-one days after immunization, splenocytes are harvested and restimulated for 1 week in the presence of peptides encoding each epitope being tested. Thereafter, for CTL effector cells, assays are conducted for cytolysis of peptide-loaded, ⁵¹Cr labeled target cells using standard techniques. Lysis of target cells sensitized by HLA loading of peptides corresponding to minigene-encoded epitopes demonstrates DNA vaccine function for in vivo induction of CTLs. Immunogenicity of HTL epitopes is evaluated in transgenic mice in an analogous manner.

Alternatively, the nucleic acids can be administered using ballistic delivery as described, for instance, in U.S. Pat. No. 5,204,253. Using this technique, particles comprised solely of DNA are administered. In a further alternative embodiment, DNA can be adhered to particles, such as gold particles.

Minigenes can also be delivered using other bacterial or viral delivery systems well known in the art, e.g., an expression construct encoding epitopes of the invention can be incorporated into a viral vector such as vaccinia.

2. Combinations of CTL Peptides with Helper Pepides

Vaccine compositions comprising CTL peptides of the invention can be modified to provide desired attributes, such as improved serum half life, broadened population coverage or enhanced immunogenicity.

For instance, the ability of a peptide to induce CTL activity can be enhanced by linking the peptide to a sequence which contains at least one epitope that is capable of inducing a T helper cell response. The use of T helper epitopes in conjunction with CTL epitopes to enhance immunogenicity is illustrated, for example, in the co-pending applications U.S. Ser. No. 08/820,360, U.S. Ser. No. 08/197,484, and U.S. Ser. No. 08/464,234.

Although a CTL peptide can be directly linked to a T helper peptide, often CTL epitope/HTL epitope conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues and sometimes 10 or more residues. The CTL peptide epitope can be linked to the T helper peptide epitope either directly or via a spacer either at the amino or carboxy terminus of the CTL peptide. The amino terminus of either the immunogenic peptide or the T helper peptide may be acylated.

In certain embodiments, the T helper peptide is one that is recognized by T helper cells present in the majority of the population. This can be accomplished by selecting peptides that bind to many, most, or all of the HLA class II molecules. These are known as “loosely HLA-restricted” or “promiscuous” T helper sequences. Examples of amino acid sequences that are promiscuous include sequences from antigens such as tetanus toxoid at positions 830-843 (QYIKANSKFIGITE; SEQ ID NO: 51484), Plasmodium falciparum circumsporozoite (CS) protein at positions 378-398 (DIEKKIAKMEKASSVFNVVNS; SEQ ID NO: 51485), and Streptococcus 18kD protein at positions 116 (GAVDSILGGVATYGAA; SEQ ID NO: 51486). Other examples include peptides bearing a DR 1-4-7 supermotif, or either of the DR3 motifs.

Alternatively, it is possible to prepare synthetic peptides capable of stimulating T helper lymphocytes, in a loosely HLA-restricted fashion, using amino acid sequences not found in nature (see, e.g., PCT publication WO 95/07707). These synthetic compounds called Pan-DR-binding epitopes (e.g., PADRE®, Epimmune, Inc., San Diego, Calif.) are designed to most preferrably bind most HLA-DR (human HLA class II) molecules. For instance, a pan-DR-binding epitope peptide having the formula: aKXVAAWTLKAAa, where “X” is either cyclohexylalanine, phenylalanine, or tyrosine, and a is either d-alanine or 1-alanine, has been found to bind to most HLA-DR alleles, and to stimulate the response of T helper lymphocytes from most individuals, regardless of their HLA type. An alternative of a pan-DR binding epitope comprises all “L” natural amino acids and can be provided in the form of nucleic acids that encode the epitope.

HTL peptide epitopes can also be modified to alter their biological properties. For example, they can be modified to include d-amino acids to increase their resistance to proteases and thus extend their serum half life, or they can be conjugated to other molecules such as lipids, proteins, carbohydrates, and the like to increase their biological activity. For example, a T helper peptide can be conjugated to one or more palmitic acid chains at either the amino or carboxyl termini.

3. Combinations of CTL Peptides with T Cell Priming Agents

In some embodiments it may be desirable to include in the pharmaceutical compositions of the invention at least one component which primes cytotoxic T lymphocytes. Lipids have been identified as agents capable of priming CTL in vivo against viral antigens. For example, palmitic acid residues can be attached to the ε- and α-amino groups of a lysine residue and then linked, e.g., via one or more linking residues such as Gly, Gly-Gly-, Ser, Ser-Ser, or the like, to an immunogenic peptide. The lipidated peptide can then be administered either directly in a micelle or particle, incorporated into a liposome, or emulsified in an adjuvant, e.g., incomplete Freund's adjuvant. In a preferred embodiment, a particularly effective immunogenic composition comprises palmitic acid attached to ε- and α-amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P3CSS) can be used to prime virus specific CTL when covalently attached to an appropriate peptide (see, e.g., Deres, et al., Nature 342:561, 1989). Peptides of the invention can be coupled to P3CSS, for example, and the lipopeptide administered to an individual to specifically prime a CTL response to the target antigen. Moreover, because the induction of neutralizing antibodies can also be primed with P3CSS-conjugated epitopes, two such compositions can be combined to more effectively elicit both humoral and cell-mediated responses.

CTL and/or HTL peptides can also be modified by the addition of amino acids to the termini of a peptide to provide for ease of linking peptides one to another, for coupling to a carrier support or larger peptide, for modifying the physical or chemical properties of the peptide or oligopeptide, or the like. Amino acids such as tyrosine, cysteine, lysine, glutamic or aspartic acid, or the like, can be introduced at the C— or N-terminus of the peptide or oligopeptide, particularly class I peptides. However, it is to be noted that modification at the carboxyl terminus of a CTL epitope may, in some cases, alter binding characteristics of the peptide. In addition, the peptide or oligopeptide sequences can differ from the natural sequence by being modified by terminal-NH2 acylation, e.g., by alkanoyl (C1-C20) or thioglycolyl acetylation, terminal-carboxyl amidation, e.g., ammonia, methylaamine, etc. In some instances these modifications may provide sites for linking to a support or other molecule.

4. Vaccine Compositions Comprising DC Pulsed with CTL and/or HTL Peptides

An embodiment of a vaccine composition in accordance with the invention comprises ex vivo administration of a cocktail of epitope-bearing peptides to PBMC, or isolated DC therefrom, from the patient's blood. A pharmaceutical to facilitate harvesting of DC can be used, such as Progenipoietin™ (Monsanto, St. Louis, Mo.) or GM-CSF/IL-4. After pulsing the DC with peptides and prior to reinfusion into patients, the DC are washed to remove unbound peptides. In this embodiment, a vaccine comprises peptide-pulsed DCs which present the pulsed peptide epitopes complexed with HLA molecules on their surfaces.

The DC can be pulsed ex vivo with a cocktail of peptides, some of which stimulate CTL responses to one or more HBV antigens of interest. Optionally, a helper T cell (HTL) peptide such as a PADRE family molecule, can be included to facilitate the CTL response. Thus, a vaccine in accordance with the invention, preferably comprising epitopes from multiple HBV antigens, is used to treat HBV infection.

L. Administration of Vaccines for Therapeutic or Prophylactic Purposes

The peptides of the present invention and pharmaceutical and vaccine compositions of the invention are typically used to treat and/or prevent HBV infection. Vaccine compositions containing the peptides of the invention are administered to a patient infected with HBV or to an individual susceptible to, or otherwise at risk for, HBV infection to elicit an immune response against HBV antigens and thus enhance the patient's own immune response capabilities.

As discussed herein, peptides comprising CTL and/or HTL epitopes of the invention induce immune responses when presented by HLA molecules and contacted with a CTL or HTL specific for an epitope comprised by the peptide. The peptides (or DNA encoding them) can be administered individually or as fusions of one or more peptide sequences. The manner in which the peptide is contacted with the CTL or HTL is not critical to the invention. For instance, the peptide can be contacted with the CTL or HTL either in vivo or in vitro. If the contacting occurs in vivo, the peptide itself can be administered to the patient, or other vehicles, e.g., DNA vectors encoding one or more peptides, viral vectors encoding the peptide(s), liposomes and the like, can be used, as described herein.

When the peptide is contacted in vitro, the vaccinating agent can comprise a population of cells, e.g., peptide-pulsed dendritic cells, or HPV-specific CTLs, which have been induced by pulsing antigen-presenting cells in vitro with the peptide or by transfecting antigen-presenting cells with a minigene of the invention. Such a cell population is subsequently administered to a patient in a therapeutically effective dose.

In therapeutic applications, peptide and/or nucleic acid compositions are administered to a patient in an amount sufficient to elicit an effective CTL and/or HTL response to the virus antigen and to cure or at least partially arrest or slow symptoms and/or complications. An amount adequate to accomplish this is defined as “therapeutically effective dose.” Amounts effective for this use will depend on, e.g., the particular composition administered, the manner of administration, the stage and severity of the disease being treated, the weight and general state of health of the patient, and the judgment of the prescribing physician.

For pharmaceutical compositions, the immunogenic peptides of the invention, or DNA encoding them, are generally administered to an individual already infected with HBV. The peptides or DNA encoding them can be administered individually or as fusions of one or more peptide sequences. HBV-infected patients can be treated with the immunogenic peptides separately or in conjunction with other treatments as appropriate.

For therapeutic use, administration should generally begin at the first diagnosis of HBV infection. This is followed by boosting doses until at least symptoms are substantially abated and for a period thereafter. The embodiment of the vaccine composition (i.e., including, but not limited to embodiments such as peptide cocktails, polyepitopic polypeptides, minigenes, or HBV antigen-specific CTLs or pulsed dendritic cells) delivered to the patient may vary according to the stage of the disease or the patient's health status. For example, in a patient with chronic HBV infection, a vaccine comprising HBV-specific CTL may be more efficacious in killing HBV-infected cells than alternative embodiments.

Where susceptible individuals are identified prior to or during infection, the composition can be targeted to them, thus minimizing the need for administration to a larger population.

The peptide or other compositions used for the treatment or prophylaxis of HBV infection can be used, e.g., in persons who have not manifested symptoms. In this context, it is generally important to provide an amount of the peptide epitope delivered by a mode of administration sufficient to effectively stimulate a cytotoxic T cell response; compositions which stimulate helper T cell responses can also be given in accordance with this embodiment of the invention.

The dosage for an initial therapeutic immunization generally occurs in a unit dosage range where the lower value is about 1, 5, 50, 500, or 1,000 μg and the higher value is about 10,000; 20,000; 30,000; or 50,000 μg. Dosage values for a human typically range from about 500 μg to about 50,000 μg per 70 kilogram patient. Boosting dosages of between about 1.0 μg to about 50,000 μg of peptide pursuant to a boosting regimen over weeks to months may be administered depending upon the patient's response and condition as determined by measuring the specific activity of CTL and HTL obtained from the patient's blood. Administration should continue until at least clinical symptoms or laboratory tests indicate that the viral infection has been eliminated or reduced and for a period thereafter. The dosages, routes of administration, and dose schedules are adjusted in accordance with methodologies known in the art.

In certain embodiments, the peptides and compositions of the present invention are employed in serious disease states, that is, life-threatening or potentially life threatening situations. In such cases, as a result of the minimal amounts of extraneous substances and the relative nontoxic nature of the peptides in preferred compositions of the invention, it is possible and may be felt desirable by the treating physician to administer substantial excesses of these peptide compositions relative to these stated dosage amounts.

The vaccine compositions of the invention can also be used purely as prophylactic agents. Generally the dosage for an initial prophylactic immunization generally occurs in a unit dosage range where the lower value is about 1, 5, 50, 500, or 1000 μg and the higher value is about 10,000; 20,000; 30,000; or 50,000 μg. Dosage values for a human typically range from about 500 μg to about 50,000 μg per 70 kilogram patient. This is followed by boosting dosages of between about 1.0 μg to about 50,000 μg of peptide administered at defined intervals from about four weeks to six months after the initial administration of vaccine. The immunogenicity of the vaccine can be assessed by measuring the specific activity of CTL and HTL obtained from a sample of the patient's blood.

The pharmaceutical compositions for therapeutic treatment are intended for parenteral, topical, oral, intrathecal, or local (e.g. as a cream or topical ointment) administration. Preferably, the pharmaceutical compositions are administered parentally, e.g., intravenously, subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH-adjusting and buffering agents, tonicity adjusting agents, wetting agents, preservatives, and the like, for example, sodium acetate, sodium lactate, sodium chloride, potassium chloride, calcium chloride, sorbitan monolaurate, triethanolamine oleate, etc.

The concentration of peptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

A human unit dose form of the peptide composition is typically included in a pharmaceutical composition that comprises a human unit dose of an acceptable carrier, preferably an aqueous carrier, and is administered in a volume of fluid that is known by those of skill in the art to be used for administration of such compositions to humans (see, e.g., Remington's Pharmaceutical Sciences, 17th Edition, A. Gennaro, Editor, Mack Publising Co., Easton, Pa., 1985).

The peptides of the invention, and/or nucleic acids encoding the peptides, can also be administered via liposomes, which may also serve to target the peptides to a particular tissue, such as lymphoid tissue, or to target selectively to infected cells, as well as to increase the half-life of the peptide composition. Liposomes include emulsions, foams, micelles, insoluble monolayers, liquid crystals, phospholipid dispersions, lamellar layers and the like. In these preparations, the peptide to be delivered is incorporated as part of a liposome, alone or in conjunction with a molecule which binds to a receptor prevalent among lymphoid cells, such as monoclonal antibodies which bind to the CD45 antigen, or with other therapeutic or immunogenic compositions. Thus, liposomes either filled or decorated with a desired peptide of the invention can be directed to the site of lymphoid cells, where the liposomes then deliver the peptide compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), and U.S. Pat. Nos. 4,235,871, 4,501,728, 4,837,028, and 5,019,369.

For targeting cells of the immune system, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

M. Kits

The peptide and nucleic acid compositions of this invention can be provided in kit form together with instructions for vaccine administration. Typically the kit would include desired peptide compositions in a container, preferably in unit dosage form and instructions for administration. An alternative kit would include a minigene construct with desired nucleic acids of the invention in a container, preferably in unit dosage form together with instructions for administration. Lymphokines such as IL-2 or IL-12 may also be included in the kit. Other kit components that may also be desirable include, for example, a sterile syringe, booster dosages, and other desired excipients.

Epitopes in accordance with the present invention were successfully used to induce an immune response. Immune responses with these epitopes have been induced by administering the epitopes in various forms. The epitopes have been administered as peptides, as nucleic acids, and as viral vectors comprising nucleic acids that encode the epitope(s) of the invention. Upon administration of peptide-based epitope forms, immune responses have been induced by direct loading of an epitope onto an empty HLA molecule that is expressed on a cell, and via internalization of the epitope and processing via the HLA class I pathway; in either event, the HLA molecule expressing the epitope was then able to interact with and induce a CTL response. Peptides can be delivered directly or using such agents as liposomes. They can additionally be delivered using ballistic delivery, in which the peptides are typically in a crystalline form. When DNA is used to induce an immune response, it is administered either as naked DNA, generally in a dose range of approximately 1-5 mg, or via the ballistic “gene gun” delivery, typically in a dose range of approximately 10-100 μg. The DNA can be delivered in a variety of conformations, e.g., linear, circular etc. Various viral vectors have also successfully been used that comprise nucleic acids which encode epitopes in accordance with the invention.

Accordingly compositions in accordance with the invention exist in several forms. Embodiments of each of these composition forms in accordance with the invention have been successfully used to induce an immune response.

One composition in accordance with the invention comprises a plurality of peptides. This plurality or cocktail of peptides is generally admixed with one or more pharmaceutically acceptable excipients. The peptide cocktail can comprise multiple copies of the same peptide or can comprise a mixture of peptides. The peptides can be analogs of naturally occurring epitopes. The peptides can comprise artificial amino acids and/or chemical modifications such as addition of a surface active molecule, e.g., lipidation; acetylation, glycosylation, biotinylation, phosphorylation etc. The peptides can be CTL or HTL epitopes. In a preferred embodiment the peptide cocktail comprises a plurality of different CTL epitopes and at least one HTL epitope. The HTL epitope can be naturally or non-naturally (e.g., PADRE®, Epimmune Inc., San Diego, Calif.). The number of distinct epitopes in an embodiment of the invention is generally a whole unit integer from one through one hundred fifty (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 150).

An additional embodiment of a composition in accordance with the invention comprises a polypeptide multi-epitope construct, i.e., a polyepitopic peptide. Polyepitopic peptides in accordance with the invention are prepared by use of technologies well-known in the art. By use of these known technologies, epitopes in accordance with the invention are connected one to another. The polyepitopic peptides can be linear or non-linear, e.g., multivalent. These polyepitopic constructs can comprise artificial amino acids, spacing or spacer amino acids, flanking amino acids, or chemical modifications between adjacent epitope units. The polyepitopic construct can be a heteropolymer or a homopolymer. The polyepitopic constructs generally comprise epitopes in a quantity of any whole unit integer between 2-150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 150). The polyepitopic construct can comprise CTL and/or HTL epitopes. One or more of the epitopes in the construct can be modified, e.g., by addition of a surface active material, e.g. a lipid, or chemically modified, e.g., acetylation, etc. Moreover, bonds in the multiepitopic construct can be other than peptide bonds, e.g., covalent bonds, ester or ether bonds, disulfide bonds, hydrogen bonds, ionic bonds etc.

Alternatively, a composition in accordance with the invention comprises construct which comprises a series, sequence, stretch, etc., of amino acids that have homology to (i.e., corresponds to or is contiguous with) to a native sequence. This stretch of amino acids comprises at least one subsequence of amino acids that, if cleaved or isolated from the longer series of amino acids, fictions as an HTA class I or HLA class II epitope in accordance with the invention. In this embodiment, the peptide sequence is modified, so as to become a construct as defined herein, by use of any number of techniques known or to be provided in the art. The polyepitopic constructs can contain homology to a native sequence in any whole unit integer increment from 70-100%, e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or, 100 percent.

A further embodiment of a composition in accordance with the invention is an antigen presenting cell that comprises one or more epitopes in accordance with the invention. The antigen presenting cell can be a “professional” antigen presenting cell, such as a dendritic cell. The antigen presenting cell can comprise the epitope of the invention by any means known or to be determined in the art. Such means include pulsing of dendritic cells with one or more individual epitopes or with one or more peptides that comprise multiple epitopes, by nucleic acid administration such as ballistic nucleic acid delivery or by other techniques in the art for administration of nucleic acids, including vector-based, e.g. viral vector, delivery of nucleic acids.

Further embodiments of compositions in accordance with the invention comprise nucleic acids that encode one or more peptides of the invention, or nucleic acids which encode a polyepitopic peptide in accordance with the invention. As appreciated by one of ordinary skill in the art, various nucleic acids compositions will encode the same peptide due to the redundancy of the genetic code. Each of these nucleic acid compositions falls within the scope of the present invention. This embodiment of the invention comprises DNA or RNA, and in certain embodiments a combination of DNA and RNA. It is to be appreciated that any composition comprising nucleic acids that will encode a peptide in accordance with the invention or any other peptide based composition in accordance with the invention, falls within the scope of this invention.

It is to be appreciated that peptide-based forms of the invention (as well as the nucleic acids that encode them) can comprise analogs of epitopes of the invention generated using principles already known, or to be known, in the art. Principles related to analoging are now known in the art, and are disclosed herein; moreover, analoging principles (heteroclitic analoging) are disclosed in co-pending application serial number U.S. Ser. No. 09/226,775 filed 6 Jan. 1999. Generally the compositions of the invention are isolated or purified.

### EXAMPLES

The invention will be described in greater detail by way of specific examples. The following examples are offered for illustrative purposes, and are not intended to limit the invention in any manner. Those of skill in the art will readily recognize a variety of non-critical parameters that can be changed or modified to yield alternative embodiments in accordance with the invention.

### Example 1

### HLA Class I and Class II Binding Assays

The following example of peptide binding to HLA molecules demonstrates quantification of binding affinities of HLA class I and class II peptides. Binding assays can be performed with peptides that are either motif-bearing or not motif-bearing.

Cell lysates were prepared and HLA molecules purified in accordance with disclosed protocols (Sidney et al., Current Protocols in Immunology 18.3.1 (1998); Sidney, et al., J. Immunol. 154:247 (1995); Sette, et al., Mol. Immunol. 31:813 (1994)). The cell lines used as sources of HLA molecules and the antibodies used for the extraction of the HLA molecules from the cell lysates are also described in these publications.

Epstein-Barr virus (EBV)-transformed homozygous cell lines, fibroblasts, CIR, or 721.221-transfectants were used as sources of HLA class I molecules. These cells were maintained in vitro by culture in RPMI 1640 medium supplemented with 2 mM L-glutamine (GIBCO, Grand Island, N.Y.), 50 μM 2-ME, 100 μg/ml of streptomycin, 100 U/ml of penicillin (Irvine Scientific) and 10% heat-inactivated FCS (Irvine Scientific, Santa Ana, Calif.). Cells were grown in 225-cm2 tissue culture flasks or, for large-scale cultures, in roller bottle apparatuses.

Cell lysates were prepared as follows. Briefly, cells were lysed at a concentration of 10⁸ cells/ml in 50 mM Tris-HCl, pH 8.5, containing 1% Nonidet P-40 (Fluka Biochemika, Buchs, Switzerland), 150 mM NaCl, 5 mM EDTA, and 2 mM PMSF. Lysates were cleared of debris and nuclei by centrifugation at 15,000×g for 30 min.

HLA molecules were purified from lysates by affinity chromatography. Lysates were passed twice through two pre-columns of inactivated Sepharose CL4-B and protein A-Sepharose. Next, the lysate was passed over a column of Sepharose CL-4B beads coupled to an appropriate antibody. The anti-HLA column was then washed with 10-column volumes of 10 mM Tris-HCL, pH 8.0, in 1% NP-40, PBS, 2-column volumes of PBS, and 2-column volumes of PBS containing 0.4% n-octylglucoside. Finally, MHC molecules were eluted with 50 mM diethylamine in 0.15M NaCl containing 0.4% n-octylglucoside, pH 11.5. A 1/25 volume of 2.0M Tris, pH 6.8, was added to the eluate to reduce the pH to ˜8.0. Eluates were then concentrated by centrifugation in Centriprep 30 concentrators at 2000 rpm (Amicon, Beverly, Mass.). Protein content was evaluated by a BCA protein assay (Pierce Chemical Co., Rockford, Ill.) and confirmed by SDS-PAGE.

A detailed description of the protocol utilized to measure the binding of peptides to Class I and Class II MHC has been published (Sette et al., Mol. Immunol. 31:813, 1994; Sidney et al., in Current Protocols in Immunology, Margulies, Ed., John Wiley & Sons, New York, Section 18.3, 1998). Briefly, purified MHC molecules (5 to 500 nM) were incubated with various unlabeled peptide inhibitors and 1-10 nM 125I-radiolabeled probe peptides for 48 h in PBS containing 0.05% Nonidet P-40 (NP40) (or 20% w/v digitonin for H-2 IA assays) in the presence of a protease inhibitor cocktail. The final concentrations of protease inhibitors (each from CalBioChem, La Jolla, Calif.) were 1 mM PMSF, 1.3 nM 1.10 phenanthroline, 73 μM pepstatin A, 8mM EDTA, 6 mM N-ethylmaleimide (for Class II assays), and 200 μM N alpha-p-tosyl-L-lysine chloromethyl ketone (TLCK). All assays were performed at pH 7.0 with the exception of DRB1*0301, which was performed at pH 4.5, and DRB1*1601 (DR2w21β1) and DRB4*0101 (DRw53), which were performed at pH 5.0. pH was adjusted as described elsewhere (see Sidney et al., in Current Protocols in Immunology, Margulies, Ed., John Wiley & Sons, New York, Section 18.3, 1998).

Following incubation, MHC-peptide complexes were separated from free peptide by gel filtration on 7.8 mm×15 cm TSK200 columns (TosoHaas 16215, Montgomeryville, Pa.), eluted at 1.2 mls/min with PBS pH 6.5 containing 0.5% NP40 and 0.1% NaN3. Because the large size of the radiolabeled peptide used for the DRB1*1501 (DR2w2β1) assay makes separation of bound from unbound peaks more difficult under these conditions, all DRB1*1501 (DR2w2β1) assays were performed using a 7.8 mm×30 cm TSK2000 column eluted at 0.6 mls/min. The eluate from the TSK columns was passed through a Beckman 170 radioisotope detector, and radioactivity was plotted and integrated using a Hewlett-Packard 3396A integrator, and the fraction of peptide bound was determined.

Radiolabeled peptides were iodinated using the chloramine-T method. Representative radiolabeled probe peptides utilized in each assay, and its assay specific IC₅₀ nM, are summarized in Tables IV and V. Typically, in preliminary experiments, each MHC preparation was titered in the presence of fixed amounts of radiolabeled peptides to determine the concentration of HLA molecules necessary to bind 10-20% of the total radioactivity. All subsequent inhibition and direct binding assays were performed using these HLA concentrations.

Since under these conditions [label]<[HLA] and IC₅₀≧[HLA], the measured IC₅₀ values are reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is calculated for each peptide by dividing the IC₅₀ of a positive control for inhibition by the IC₅₀ for each tested peptide (typically unlabeled versions of the radiolabeled probe peptide). For inter-experiment comparisons, relative binding values are compiled. These values can subsequently be converted back into IC₅₀ nM values by dividing the IC₅₀ nM of the positive controls for inhibition by the relative binding of the peptide of interest. This method of data compilation has proven to be the most accurate and consistent for comparing peptides that have been tested on different days, or with different lots of purified MHC.

Because the antibody used for HLA-DR purification (LB3.1) is α-chain specific, β1 molecules are not separated from β3 (and/or β4 and β5) molecules. The β1 specificity of the binding assay is obvious in the cases of DRB1*0101 (DR1), DRB1*0802 (DR8w2), and DRB1*0803 (DR8w3), where no β3 is expressed. It has also been demonstrated for DRB1*0301 (DR3) and DRB3*0101 (DR52a), DRB1*0401 (DR4w4), DRB1*0404 (DR4w14), DRB1*0405 (DR4w15), DRB1*1101 (DR5), DRB1*1201 (DR5w12), DRB1*1302 (DR6w19) and DRB1*0701 (DR7). The problem of β chain specificity for DRB1*1501 (DR2w2β1), DRB5*0101 (DR2w2β2), DRB1*1601 (DR2w21β1), DRB5*0201 (DR51Dw21), and DRB4*0101 (DRw53) assays is circumvented by the use of fibroblasts. Development and validation of assays with regard to DRP molecule specificity have been described previously (see, e.g., Southwood et al., J. Immunol. 160:3363-3373, 1998).

Binding assays as outlined above may be used to analyze supermotif and/or motif-bearing epitopes as, for example, described in Example 2.

### Example 2

### Identification of Conserved HLA Supermotif CTL Candidate Epitopes

Vaccine compositions of the invention can include multiple epitopes that comprise multiple HLA supermotifs or motifs to achieve broad population coverage. This example illustrates the identification of supermotif-bearing epitopes for the inclusion in such a vaccine composition. Calculation of population coverage was performed using the strategy described below. Epitopes were then selected to bear an HLA-A2, -A3, or -B7 supermotif or an HLA-A1 or -A24 motif.

Computer Searches and Algorthims for Identification of Supermotif and/or Motif-Bearing Epitopes

Computer searches for epitopes bearing HLA Class I or Class II supermotifs or motifs were performed as follows. All translated HBV isolate sequences were analyzed using a text string search software program, e.g., MotifSearch 1.4 (D. Brown, San Diego) to identify potential peptide sequences containing appropriate HLA binding motifs; alternative programs are readily produced in accordance with information in the art in view of the motif/supermotif disclosure herein. Furthermore, such calculations can be made mentally. Identified A2-, A3-, and DR-supermotif sequences were scored using polynomial algorithms to predict their capacity to bind to specific HLA-Class I or Class II molecules. These polynomial algorithms take into account both extended and refined motifs (that is, to account for the impact of different amino acids at different positions), and are essentially based on the premise that the overall affinity (or ΔG) of peptide-HLA molecule interactions can be approximated as a linear polynomial function of the type:
“ΔG”=a1i×a2i×a3i . . . x ani

where aji is a coefficient which represents the effect of the presence of a given amino acid (j) at a given position (i) along the sequence of a peptide of n amino acids. The crucial assumption of this method is that the effects at each position are essentially independent of each other (i.e., independent binding of individual side-chains). When residue j occurs at position i in the peptide, it is assumed to contribute a constant amount ji to the free energy of binding of the peptide irrespective of the sequence of the rest of the peptide. This assumption is justified by studies from our laboratories that demonstrated that peptides are bound to MHC and recognized by T cells in essentially an extended conformation (data omitted herein).

The method of derivation of specific algorithm coefficients has been described in Gulukota et al., i J. Mol. Biol. 267:1258-126, 1997; (see also Sidney et al., Human Immunol. 45:79-93, 1996; and Southwood et al., J. Immunol. 160:3363-3373, 1998). Briefly, for all i positions, anchor and non-anchor alike, the geometric mean of the average relative binding (ARB) of all peptides carrying j is calculated relative to the remainder of the group, and used as the estimate of ji. For Class II peptides, if multiple alignments are possible, only the highest scoring alignment is utilized, following an iterative procedure. To calculate an algorithm score of a given peptide in a test set, the ARB values corresponding to the sequence of the peptide are multiplied. If this product exceeds a chosen threshold, the peptide is predicted to bind. Appropriate thresholds are chosen as a function of the degree of stringency of prediction desired.

Selection of HLA-A2 Supertype Cross-Reactive Peptides

Complete sequences from 20 HBV isolates were aligned, then scanned, utilizing a customized computer program, to identify conserved 9- and 10-mer sequences containing the HLA-A2-supertype main anchor specificity.

A total of 150 conserved and motif-positive sequences were identified. These peptides were then evaluated for the presence of A*0201 preferred secondary anchor residues using an A*0201-specific polynomial algorithm. A total of 85 conserved, motif-positive sequences were selected and synthesized.

These 85 conserved, motif-containing 9- and 10-mer peptides were then tested for their capacity to bind purified HLA-A*0201 molecules in vitro. Thirty-four peptides were found to be good A*0201 binders (IC₅₀≦500 nM); 15 were high binders (IC₅₀≦50 nM) and 19 were intermediate binders (IC₅₀ of 50-500 nM) (Table XXVI).

In the course of independent analyses, 25 conserved, HBV-derived, 8 or 11-mer sequences with appropriate A2-supertype main anchors were also synthesized and tested for A*0201 binding. One peptide, HBV env 259 11-mer (peptide 1147.14), bound A*0201 with an IC₅₀ of 500 nM, or less, and has been included in Table XXVI. Also shown in Table XXVI is an analog peptide, representing a single substitution of the HBV pol 538 9-mer peptide, which binds A*0201 with an IC₅₀ of 5.1 nM (see below).

Thirty of the 36 A*0201 binders were subsequently tested for the capacity to bind to additional A2-supertype alleles (A*0202, A*0203, A*0206, and A*6802). As shown in Table XXVI, 15/30 (50%) peptides were found to be A2-supertype cross-reactive binders, binding at least 3 of the 5 A2-supertype alleles tested. These 15 peptides were selected for further analysis.

Selection of HLA-A3 Supermotif-Bearing Epitopes

The sequences from the same 20 isolates were also examined for the presence of conserved peptides with the HLA-A3-supermotif primary anchors. A total of 80 conserved 9- or 10-mer motif-containing sequences were identified. Further analysis using the A03 and A11 algorithms identified 40 sequences which scored high in either or both algorithms. Thirty-six of the corresponding peptides were synthesized and tested for binding to HLA-A*03 and HLA-A*11, the two most prevalent A3-supertype alleles. Twenty-three peptides were identified which bound A3 and/or A11 with affinities or IC₅₀ values of ≦500 nM (Table XXVII).

In the course of an independent series of studies 30 HBV-derived 8-mer, and 24 11-mer sequences, conserved in 75% or more of the isolates, were selected and tested for A*03 and A*11 binding. Four 8-mers and 9 11-mers were found to bind either or both alleles (Table XXVII). Finally, four 9-mer, and one 10-mer, conserved HBV-derived peptides not selected using the search criteria outlined above, but which have been shown to bind A*03 and/or A*11, have been identified, and are included in Table XXVII. Two of these peptides contain non-canonical anchors (peptides 20.0131, and 20.0130), and the other 3 are algorithm negative (peptides 1142.05, 1099.03, and 1090.15).

Thirty-eight of the 41 peptides binding A*03 and/or A*11 were subsequently tested for binding crossreactivity to the other common A3-supertype alleles (A*3101, A*3301, and A*6801). It was found that 17 of these peptides were A3-supertype cross-reactive, binding at least 3 of the 5 A3-supertype alleles tested (Table XXVI).

Selection of HLA-B7 Supermotif Bearing Epitopes

When the same 20 isolates were also analyzed for the presence of conserved 9- or 10-mer peptides with the HLA-B7-supermotif, 46 sequences were identified. Thirty-four of the corresponding peptides were synthesized and tested for binding to HLA-B*0702, the most common B7-supertype allele. Nine peptides bound B*0702 with an IC₅₀ value of ≦500 nM (Table XXVIII). These 9 peptides were then tested for binding to other common B7-supertype alleles (B*3501, B*51, B*5301, and B*5401). Five of the 9 B*0702 binders were capable of binding to 3 or more of the 5 B7-supertype alleles tested.

In separate studies investigating the secondary anchor requirements of B7-supertype alleles, all available peptides with the B7-supermotif were tested for binding to all B7 supertype alleles. As a result, all 34 peptides described above were also tested for binding to other B7-supertype alleles. These experiments identified an additional 10 peptides which bound at least one B7-supertype allele with an IC₅₀ value ≦500 nM, including 2 peptides which bound 3 or more alleles. These 10 peptides are also shown in Table XXVIII.

Because of the low numbers of conserved B7-supertype degenerate HBV-derived 9- and 10-mer peptides, compared to the A2- and A3-supertypes, the 20 isolates were again examined to identify conserved, motif-containing 8- and 11-mers. This re-scan identified 51 peptides. Thirty-one of these were synthesized and tested for binding to each of the 5 most common B7-supertype alleles. Nineteen 8- and 11-mer peptides bound with high or intermediate affinity to at least one B7-supertype allele (IC₅₀<500 nM) (Table XXVIII). Two peptides were degenerate binders, binding 3 of the 5 alleles tested.

In summary, a total of 9 HBV-derived peptides, conserved in 75% or more of the isolates analyzed, have been identified which are degenerate B7-supertype binders (Table XXVIII).

Selection of A1 and A24 Motif-Bearing Epitopes

To further increase population coverage, HLA-A1 and -A24 epitopes have been incorporated into the present analysis. A1 is, on average, present in 12%, and A24 is present in approximately 29%, of the population across five different major ethnic groups (Caucasian, North American Black, Chinese, Japanese, and Hispanic). Combined, these alleles would be represented with an average frequency of 39% in these same populations. The total coverage across the major ethnicities when A1 and A24 are combined with the coverage of the A2-, A3- and B7-supertype alleles is >95.4%; by comparison, coverage by combing the A2-, A3-, and B7-supertypes is 86.2%.

Systematic analyses of HBV for A1 and A24 binders have yet to be completed. However, in the course of independent studies, 15 conserved HBV-derived peptides have been identified that bind A*0101 with IC₅₀ less than 500 nM (Table XXIX); 7 of these bind with IC₅₀ less than 100 nM. In a similar context, 14 conserved A*2402 binding HBV-derived peptides have also been identified, 6 of which bind A*2402 with IC₅₀ less than 100 nM (Table XXIX).

### Example 3

### Confirmation of Immunogenicity

Evaluation of A*0201 Immunogenicity

The immunogenicity analysis of the 15 HBV-derived HLA-A2 supertype cross-reactive peptides identified above is summarized in Table XXX. Peptides were screened for immunogenicity in at least one of three systems. Peptides were screened for the induction of primary antigen-specific CTL in vitro using human PBMC (Wentworth et al., Molec. Immunol. 32:603, 1995); this data is indicated as “primary CTL” in Table XXX.

The protocol for in vitro induction of primary antigen-specific CTL from human PBMC has been described by Wentworth et al (Wentworth et al., Molec. Immunol. 32:603, 1995). PBMC from normal donors which had been enriched for CD8⁺ T cells were incubated with peptide loaded antigen-presenting cells (SAC-I activated PBMCs) in the presence of IL-7. After seven days cultures were restimulated using irradiated autologous adherent cells pulsed with peptide and then tested for cytotoxic activity seven days later.

In addition, HLA transgenic mice were used to evaluate peptide immunogenicity; this data is indicated as “transgenic CTL” in Table XXX. Previous studies have shown that CTL induced in A*0201/Kb transgenic mice exhibit specificity similar to CTL induced in humans (Vitiello et al., J. Exp. Med. 173:1007, 1991; Wentworth et al., Eur. J. Immunol. 26:97, 1996).

CTL induction in transgenic mice following peptide immunization has been described by Vitiello et al. (Vitiello et al., J. Exp. Med. 173:1007, 1991) and Alexander et al. (Alexander et al., J. Immunol. 159:4753, 1997). Briefly, synthetic peptides (50 μg/mouse) and the helper epitope HBV core 128 (140 μg/mouse) were emulsified in incomplete Freund's adjuvant (IFA) and injected subcutaneously at the base of the tail. Eleven days after injection, splenocytes were incubated in the presence of peptide-loaded syngenic LPS blasts. After six days cultures were assayed for cytotoxic activity using peptide-pulsed targets.

Peptides were also tested for the ability to stimulate recall CTL responses in acutely infected HBV patients (Bertoni et al., J. Clin. Invest. 100:503, 1997; Rehermann et al., J. Clin. Invest. 97:1655-1665, 1996; Nayersina et al., J. Immunol. 150:4659, 1993); these data are indicated as “patient CTL” in Table XXX. Patient immunogenicity data is particularly informative as it indicates that a peptide is recognized during the course of a natural infection. These data demonstrate that a peptide is processed and presented in human cells that would represent the targets for CTL. Moreover, this data is especially relevant for vaccine design as the induction of CTL responses in patients has been correlated to the resolution of infection.

For the evaluation of recall CTL responses, screening was carried out as described by Bertoni et al. (Bertoni et al., J. Clin. Invest. 100:503, 1997). Briefly, PBMC from patients acutely infected with HBV were cultured in the presence of 10 μg/ml of synthetic peptide. After seven days, the cultures were restimulated with peptide. The cultures were assayed for cytotoxic activity on day 14 using target cells pulsed with peptide.

Of the 15 A2 supertype binding peptides, 11 were found to be immunogenic in at least one of the systems utilized. Five of the 11 peptides had previously been identified in the patients with acute HBV (Bertoni et al., J. Clin. Invest. 100:503, 1997). Five additional degenerate peptides (1069.06, 1090.77, 1147.14, 927.42 and 927.46) induced CTL responses in HLA-A*0201 transgenic mice. The 11 immunogenic supertype cross-reactive peptides are encoded by three HBV antigens; core, envelope and polymerase.

This set of 11 immunogenic A2-supermotif-bearing epitopes includes one analog peptide, 1090.77. The wild type peptide, 1090.14, from which this analog is derived is A2-supertype non-cross-reactive, but has been shown to be recognized in recall CTL L responses from acute HBV patients, and to be immunogenic in HLA-A*0201 transgenic mice as well as primary human cultures (Table XXX). Further studies addressing the cross recognition of the wild type peptide 1090.14 and the 1090.77 analog are described in detail below.

In the course of independent analyses, 14 of the non-cross-reactive peptides shown in Table XXXb, including 1090.14, were found to be immunogenic in at least one system. Five peptides of these peptides were recognized in patients; 4 peptides induced CTL in transgenic mice.

In conclusion, 11 A2-supertype cross-reactive peptides have been identified that are capable of exhibiting immunogenicity in at least one of the three systems examined.

Seven of the 17 A3-supertype cross-reactive peptides have been evaluated for immunogenicity (Table XXXI). As described in the previous section, A3-supermotif-bearing peptides were screened using primary cultures, patient responses, or HLA-A11 transgenic mice (Alexander et al., J. Immunol. 159:4753, 1997). With the exception of peptide 1.0219, all of the conserved cross-reactive peptides listed in Table insert table XXXI were found to be immunogenic.

Additionally, a poorly conserved peptide (1150.51; 40% conserved) which exhibits cross-reactive supertype binding was found to be immunogenic in transgenic mice, and has been included in Table XXXI. Two other conserved, but non-cross-reactive, peptides have also been shown to be recognized in acutely infected HBV patients (Bertoni et al., J. Clin. Invest. 100:503, 1997). These epitiopes are shown in Table XXXI.

It is notable that for 7 of the 8 conserved immunogenic HBV-derived A3-supermotif-bearing epitopes, including all 6 of the cross-reactive peptides, positive data was obtained in patients. These epitopes are predominantly derived from the polymerase protein sequence, with only one epitope being derived from the core protein sequence. While a number of cross-reactive peptides have been identified in the X antigen (Table XXXI), to date these peptides have not been screened for immunogenicity.

In summary, 7 A3-supermotif-bearing, cross-reactive peptides have been identified that are recognized by CTL in acutely infected patients, or induce CTL in HLA-transgenic mict.

Evaluation of B7 Immunogenicity

The immunogenicity studies involving the HBV-derived HLA-B7-supermotif-bearing, cross-reactive peptides is summarized in Table XXXII. HLA-B7 peptides were screened exclusively in human systems measuring responses in either primary cultures or acutely infected HBV patients. Of the 7 degenerate peptides screened, 4 were shown to be immunogenic. One non-crossreactive peptide (XRN<3), 1147.04, was also shown to be recognized in acutely infected HBV patients (Bertoni et al., J. Clin. Invest. 100:503, 1997; see TableXXXII).

In summary, 5 conserved HBV-derived B7-supermotif-bearing epitopes that are recognized in acutely infected HBV patients have been identified. These epitopes afford coverage of 4 different HBV antigens (core, envelope, polymerase and X).

### Example 4

### Implementation of the Extended Supermotif to Improve the Binding Capacity of Native Peptides by Creating Analogs

HLA motifs and supernotifs (comprising primary and/or secondary residues) are useful in preparing highly cross-reactive native peptides, as demonstrated herein. Moreover, the definition of HLA motifs and supermotifs also allows one to engineer highly cross-reactive epitopes by identifying residues within a native peptide sequence which can be analoged, or “fixed”, to confer upon a peptide certain characteristics, e.g., greater cross-reactivity within the group of HLA molecules that make-up the supertype, and/or greater binding affinity for some or all of those HLA molecules Examples of analog peptides that exhibit modulated binding affinity are provided.

Analoging at Primary Anchor Residues

It has been shown that class I peptide ligands can be modified, or “fixed” to increase their binding affinity and/or degeneracy (Sidney et al., J. Immunol. 157:3480, 1996). These fixed peptides may also demonstrate increased immunogenicity and crossreactive recognition by T cells specific for the wild type epitope (Parkhurst et al., J. Immunol. 157:2539, 1996; Pogue et al., Proc. Natl. Acad. Sci. USA 92:8166, 1995). Specifically, the main anchors of A2 supertype peptides may be“fixed”, or analoged, to L or V (or M, if natural) at position 2, and V at the C-terminus. As indicated in Table XXVI, 9 of the 14 A2-supertype cross-reactive binding peptides are “fixable” by these criteria, as are 16 of the 21 non-cross-reactive binders. Ideal candidates for fixing would be peptides which bind at least 3 A2-supertype allele-specific molecules with IC₅₀≦5000 nM.

An example of the efficacy of this strategy to generate more broadly cross-reactive epitopes is provided by the case of peptide 1090.14 (Table XXVI). Previously, this peptide was shown to be highly immunogenic in each of the systems examined. However, it only exhibits binding to a single A2-supertype allele-specific molecule, A*0201. The non-crossreactive binding capacity of this epitope limits the population coverage and consequently the value of including this peptide in a candidate vaccine. In an effort to increase binding affinity and cross-reactivity the C-terminus of peptide 1090.14 was altered from ‘alanine’ to the A2-supermotif preferred residue ‘valine’. This change resulted in a dramatic (40-fold) increase in binding capacity for A*0201 (from 200 nM to 5.1 nM), but also produced a peptide capable of binding 3 other A2-supertype allele-specific molecules. (see peptide 1090.77, Table XXVI).

Studies with HLA-A*0201 transgenic mice have shown that the CTL response from mice immunized with the 1090.77 peptide recognize target cells loaded with either the naturally occurring peptide 1090.14 or the valine-substituted analog (i.e., 1090.77). In fact, the lysis effected by 1090.77 induced CTL was indistinguishable regardless whether the analog or the wild-type sequence was used to load the target cells (B. Livingston, unpublished data).

The relevance of these observations for the design of vaccine constructs is indicated by studies in which chronic HBV patients were treated with the potent viral replication inhibitor, lamivudine. Extended therapy with lamivudine resulted in the selection of drug-resistant strains of HBV that have a substitution of valine for methione at position 2 in the 1090.14 epitope, suggesting that epitope-based vaccines used in combination with lamivudine may need to have the ability to induce CTL responses that recognize both wild type and mutant sequences.

To demonstrate that cross-recognition is possible between the native peptide (1090.14), the analog peptide, and the lamivudine induced mutant M2 peptide, CTL were generated using the 1090.77 analog peptide. These CTL cultures were then stimulated with either the wild type peptide (1090.14), or the lamivudine induced mutant M2 peptide. The ability of these CTL to then lyse target cells loaded with either the wild type, or the lamivudine induced mutant peptide was then assayed. Target cells presenting either peptide were similarly lysed by either CTL culture (Table XXVI).

These studies demonstrate how analoging a peptide can result in dramatically increased HLA-A2 supertype degeneracy while still allowing cross-recognition between wildtype and mutant epitopes. More specifically, these results indicate that a vaccine utilizing the analog peptide 1090.77 should stimulate a response that will recognize both wild-type and lamivudine-resistant strains of HBV.

Similarly, analogs of HLA-A3 supermotif-bearing epitopes may also be generated. For example, peptides could be analogued to possess a preferred V at position 2, and R or K at the C-terminus. Twelve of the A3-supertype degenerate peptides identified in Table XXVII are candidates for main anchor fixing, as are 19 of the 24 non-cross-reactive binders.

Analog peptides are initially tested for binding to A*03 and A*11, and those that demonstrate equivalent, or improved, binding capacity relative to the parent peptide would then be tested for A3-supertype cross-reactivity. Analogs demonstrating improved cross-reactivity are then further evaluated for immunogenicity, as necessary.

Typically, it is more difficult to identify B7 supermotif-bearing epitopes. As in the cases of A2- and A3-supertype epitopes, a peptide analoguing strategy can be utilized to generate additional B7 supermotif-bearing epitopes with increased cross-reactive binding. In general, B7 supermotif-bearing peptides should be fixed to possess P in position 2, and I at their C-terminus.

Analogs representing primary anchor single amino acid residues substituted with I residues at the C-terminus of two different B7-like peptides (HBV env 313 and HBV pol 541) were synthesized and tested for their B7-supertype binding capacity. It was found that the I substitution had an overall positive effect on binding affinity and/or cross-reactivity in both cases. In the case of HBV env 313 the I9 (I at C-terminal position 9) replacement was effective in increasing cross-reactivity from 4 to 5 alleles bound by virtue of an almost 400-fold increase B*5401 binding affinity. In the case of HBV pol 541, increased cross-reactivity was similarly achieved by a substantial increase in B*5401 binding. Also, significant gains in binding affinity for B*0702, B51, and B*5301 were observed with the HBV pol 541 I9 analog.

Analoging at Secondary Anchor Residues

Moreover, HLA supermotifs are of value in engineering highly cross-reactive peptides by identifying particular residues at secondary anchor positions that are associated with such cross-reactive properties. Demonstrating this, the capacity of a second set of peptides representing discreet single amino acid substitutions at positions one and three of five different B7-supertype binding peptides were synthesized and tested for their B-7 supertype binding capacity. In 4/4 cases the effect of replacing the native residue at position 1 with the aromatic residue F (an “F1” substitution) resulted in an increase in cross-reactivity, compared to the parent peptide, and, in most instances, binding affinity was increased three-fold or better (Table XXVIII). More specifically, for HBV env 313, MAGE2 170, and HBV core 168 complete supertype cross-reactivity was achieved with the F1 substitution analogs. These gains were achieved by dramatically increasing B*5401 binding affinity. Also, gains in affinity were noted for other alleles in the cases of HBV core 168 (B*3501 and B*5301) and MAGE2 170 (B*3501, B51 and B*5301). Finally, in the case of MAGE3 196, the F1 replacement was effective in increasing cross-reactivity because of gains in B*0702 binding. An almost 70-fold increase in B51 binding capacity was also noted.

Two analogs were also made using the supernotif positive F substitution at position three (an “F3” substitution). In both instances increases in binding affinity and cross-reactivity were achieved. Specifically, in the case of HBV pol 541, the F3 substitution was effective in increasing cross-reactivity by virtue of its effect on B*5401 binding. In the case of MAGE3 196, complete supertype cross-reactivity was achieved by increasing B*0702 and B*3501 binding capacity. Also, in the case of MAGE3 196, it is notable that increases in binding capacity between 40- and 5000-fold were obtained for B*3501, B51, B*5301, and B*5401.

In conclusion, these data demonstrate that by the use of even single amino acid substitutions, it is possible to increase the binding affinity and/or cross-reactivity of peptide ligands for HLA supertype molecules.

### Example 5

### Identification of Conserved HBV-Derived Sequences with HLA-DR Binding Motifs

Peptide epitopes bearing an HLA class II supermotif or motif may also be identified as outlined below using methodology similar to that described in Examples 1-3.

Selection of HLA-DR-Supermotif-Bearing Epitopes

HLA-Class II molecules bind peptides typically between 12 and 20 residues in length. However, similar to HLA-Class I, the specificity and energy of interaction is usually contained within a short core region of about 9 residues. Most DR molecules share an overlapping specificity within this 9-mer core in which a hydrophobic residue in position 1 (P1) is the main anchor (O'Sullivan et al., J. Immunol. 147:2663, 1991; Southwood et al., J. Immunol. 160:3363, 1998). The presence of small or hydrophobic residues in position 6 (P6) is also important for most DR-peptide interactions. This overlapping P1-P6 specificity, within a 9-mer core region, has been defined as the DR-supermotif. Unlike Class I molecules, DR molecules are open at both ends of the binding groove, and can therefore accommodate longer peptides of varying length. Indeed, while most of the energy of peptide-DR interactions appears to be contributed by the core region, flanking residues appear to be important for high affinity interactions. Also, although not strictly necessary for MHC binding, flanking residues are clearly necessary in most instances for T cell recognition.

To identify HBV-derived DR cross-reactive HTL epitopes, the same 20 HBV polyproteins that were scanned for the identification of HLA Class I motif sequences were scanned for the presence of sequences with motifs for binding HLA-DR. Specifically, 15-mer sequences comprised of a DR-supermotif containing 9-mer core, and three residue N— and C-terminal flanking regions, were selected. It was also required that 100% of the 15-mer sequence be conserved in at least 85% (17/20) of the HBV strains scanned. Using these criteria, 36 non-redundant sequences were identified. Thirty-five of these peptides were subsequently synthesized.

Algorithms for predicting peptide binding to DR molecules have also been developed (Southwood et al., J. Immunol. 160:3363, 1998). These algorithms, specific for individual DR molecules, allow the scoring and ranking of 9-mer core regions. Using selection tables, it has been found that these algorithms efficiently select peptide sequences with a high probability of binding the appropriate DR molecule. Additionally, it has been found that running algorithms, specifically those for DR1, DR4w4, and DR7, sequentially can efficiently select DR cross-reactive peptides.

To see if these algorithms would identify additional peptides, the same HBV polyproteins used above were re-scanned for the presence of 15-mer peptides where 100% of the 9-mer core region was ³85% (17/20 strains) conserved. Next, the 9-mer core region of each of these peptides was scored using the DR1, DR4w4, and DR7 algorithms. As a result, 8 additional sequences were identified and synthesized.

In summary, 44 15-mer peptides in which a 9-mer core region contained the DRsupermotif, or was selected using an algorithm predicting DR-binding sequences, were identified. Forty-three of these peptides were synthesized (Table XXXII).

While performing the analyses of HBV-derived peptides described above, 9 peptides predicted on the basis of their DR1, DR4w4, and DR7 algorithm profiles to be DR-cross-reactive binding peptides, but which have 9-mer core regions that are only 80% conserved, were also identified. An additional peptide which contains a DR-supermotif core region that is 95% conserved, but is located only one residue removed from the N-terminus, was previously synthesized. These 10 peptides were also selected for further analysis, and are shown in Table XXXIII.

Finally, 2 peptides, CF-08 and 1186.25, which are redundant with a peptide selected above (27.0280), were considered for additional analysis. Peptide 1186.25 contains multiple DR-supermotif sequences. Peptide CF-08 is a 20-mer that nests both 27.0280 and 1186.25. These peptides are shown in Table XXXIII.

The 55 HBV-derived peptides identified above were tested for their capacity to bind common HLA-DR alleles. To maximize both population coverage, and the relationships between the binding repertoires of most DR alleles (see, e.g., Southwood et al., J. Immunol. 160:3363, 1998), peptides were screened for binding to sequential panels of DR assays. The composition of these screening panels, and the phenotypic frequency of associated antigens, are shown in Table XXXIV. All peptides were initially tested for binding to the alleles in the primary panel: DR1, DR4w4, and DR7. Only peptides binding at least 2 of these 3 alleles were then tested for binding in the secondary assays (DR2w2 β1, DR2w2 β2, DR6w19, and DR9). Finally, only peptides binding at least 2 of the 4 secondary panel alleles, and thus 4 of 7 alleles total, were screened for binding in the tertiary assays (DR4w15, DR5w11, and DR8w2).

Upon testing, it was found that 25 of the original 55 peptides (45%) bound two or more of the primary panel alleles. When these 25 peptides were subsequently tested in the secondary assays, 20 were found to bind at least 4 of the 7 DR alleles in the primary and secondary assay panels. Finally, 18 of the 20 peptides passing the secondary screening phase were tested for binding in the tertiary assays. As a result, 12 peptides were shown to bind at least 7 of 10 common HLA-DR alleles. The sequences of these 12 peptides, and their binding capacity for each assay in the primary through tertiary panels, are shown in Table XXXV. Also shown are peptides CF-08 and 857.02, which bound 5/5 and 5/6 of the alleles tested to date, respectively.

In summary, 14 peptides, derived from 12 independent regions of the HBV genome, have been identified that are capable of binding multiple HLA-DR alleles. This set of peptides includes at least 2 epitopes each from the Core (Nuc), Pol, and Env antigens.

Selection of Conserved DR3 Motif Peptides

Because HLA-DR3 is an allele that is prevalent in Caucasian, Black, and Hispanic populations, DR3 binding capacity is an important criterion in the selection of HTL epitopes. However, data generated previously indicated that DR3 only rarely cross-reacts with other DR alleles (Sidney et al., J. Immunol. 149:2634-2640, 1992; Geluk et al., J. Immunol. 152:5742-5748, 1994; Southwood et al., J. Immunol. 160:3363-3373, 1998). This is not entirely surprising in that the DR3 peptide-binding motif appears to be distinct from the specificity of most other DR alleles.

To efficiently identify peptides that bind DR3, target proteins were analyzed for conserved sequences carrying one of the two DR3 specific binding motifs reported by Geluk et al. (J. Immunol. 152:5742-5748, 1994). Eighteen sequences were identified. Eight of these sequences were largely redundant with peptides shown in Table XXXVI, and 3 with peptides that had previously been synthesized for other studies. The 7 unique sequences were synthesized.

Seventeen of the eighteen peptides containing a DR3 motif have been tested for their DR3 binding capacity. Four peptides were found to bind DR3 with an affinity of 1000 nM or better (Table XXXVI).

### Example 6

### Immunogenicity of HPV-Derived HTL Epitopes

This example determines immunogenic DR supermotif- and DR3 motif-bearing epitopes among those identified using the methodology in Example 5.

Immunogenicity of HTL epitopes are evaluated in a manner analogous to the determination of immunogenicity of CTL epitopes by assessing the ability to stimulate HTL responses and/or by using appropriate transgenic mouse models. Immunogenicity is determined by screening for: 1.) in vitro primary induction using normal PBMC or 2.) recall responses from cancer patient PBMCs.

### Example 7

### Calculation of Phenotypic Frequencies of HLA-Supertypes in Various Ethnic Backgrounds to Determine Breadth of Population Coverage

This example illustrates the assessment of the breadth of population coverage of a vaccine composition comprised of multiple epitopes comprising multiple supermotifs and/or motifs.

In order to analyze population coverage, gene frequencies of HLA alleles were determined. Gene frequencies for each HLA allele were calculated from antigen or allele frequencies utilizing the binomial distribution formulae gf=1−(SQRT(1−af)) (see, e.g., Sidney et al., Human Immunol. 45:79-93, 1996). To obtain overall phenotypic frequencies, cumulative gene frequencies were calculated, and the cumulative antigen frequencies derived by the use of the inverse formula [af=1−(1−Cgf)2].

Where frequency data was not available at the level of DNA typing, correspondence to the serologically defined antigen frequencies was assumed. To obtain total potential supertype population coverage no linkage disequilibrium was assumed, and only alleles confirmed to belong to each of the supertypes were included (minimal estimates). Estimates of total potential coverage achieved by inter-loci combinations were made by adding to the A coverage the proportion of the non-A covered population that could be expected to be covered by the B alleles considered (e.g., total=A+B*(1−A)). Confirmed members of the A3-like supertype are A3, A11, A31, A*3301, and A*6801. Although the A3-like supertype may potentially include A34, A66, and A*7401, these alleles were not included in overall frequency calculations. Likewise, confirmed members of the A2-like supertype family are A*0201, A*0202, A*0203, A*0204, A*0205, A*0206, A*0207, A*6802, and A*6901. Finally, the B7-like supertype-confirmed alleles are: B7, B*3501-03, B51, B*5301, B*5401, B*5501-2, B*5601, B*6701, and B*7801 (potentially also B*1401, B*3504-06, B*4201, and B*5602).

Population coverage achieved by combining the A2-, A3- and B7-supertypes is approximately 86% in five major ethnic groups (see Table XXI). Coverage may be extended by including peptides bearing the A1 and A24 motifs. On average, A1 is present in 12% and A24 in 29% of the population across five different major ethnic groups (Caucasian, North American Black, Chinese, Japanese, and Hispanic). Together, these alleles are represented with an average frequency of 39% in these same ethnic populations. The total coverage across the major ethnicities when A1 and A24 are combined with the coverage of the A2-, A3- and B7-supertype alleles is >95%.

Population coverage for HLA class II molecules can be developed analagously based on the present disclosure.

Summary of Candidate HLA Class I and Class II Epitopes

In summary, on the basis of the data presented above, 34 conserved CTL epitopes were selected as vaccine candidates (Table XXXVII). Of these 34 epitopes, 7 are derived from core, 18 from polymerase, and 9 from envelope. No epitopes from the X antigen were included in the package as this protein is expressed in low amounts and is, therefore, of less immunological interest.

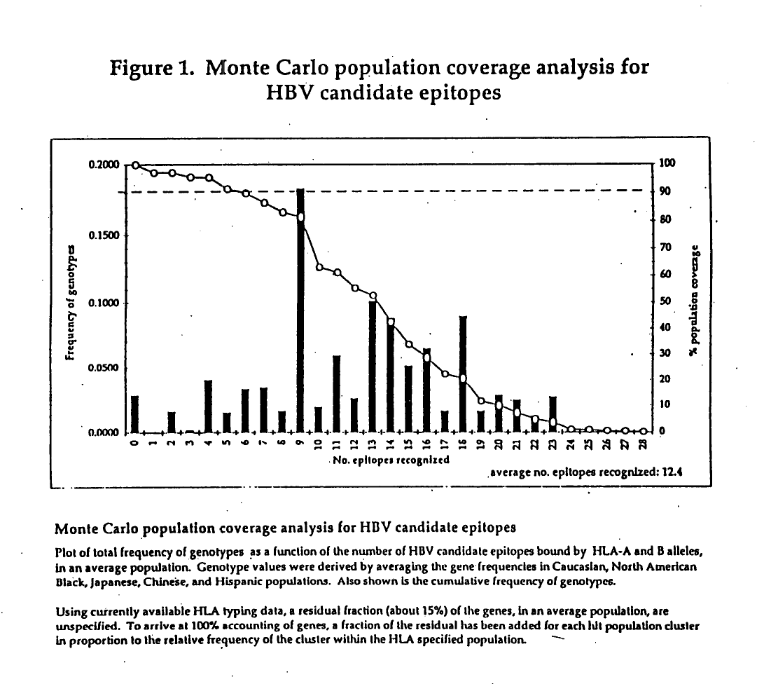

The population coverage afforded by this panel of CTL epitopes is estimated to exceed 95% in each of 5 major ethnic populations. Using a Monte Carlo analysis (FIG. 1), it is predicted that approximately 90% of the individuals in a population comprised of Caucasians, North American Blacks, Japanese, Chinese and Hispanics would recognize five or more of the vaccine candidate epitopes.

While preferred CTL epitopes includes 34 discrete peptides, two peptides are entirely nested within longer peptides, thus effectively reducing the numbers of peptides that would have to be included in a vaccine candidate. Specifically, the A2-restricted peptide 927.15 is nested within the B7-restricted peptide 26.0570 and the B7-restricted peptide 988.05 is nested within the A2-restricted peptide 924.07. Similarly, the A24-restricted peptide 20.0136 and the A2-restricted peptide 1013.01 contain the same core region, differing only at the first amino acid. On a related note, the A2-restricted peptide 1090.14 and the B7-restricted peptide 1147.05 overlap by two amino acids, raising the possibility of delivering these two epitopes as one contiguous peptide sequence.

The set of peptides includes 9 A2-restricted CTL epitopes; four polymerase-derived epitopes, four envelope-derived epitopes and a core epitope. Seven of these 9 peptides are recognized in recall CTL assays from acute patients. Of the 7 peptides recognized in patients, 2 are non-crossreactive binding peptides. The inclusion of these peptides as potential vaccine candidates stems from the observation that HLA-A*0201 is the predominantly expressed A2-supertype allele in all ethnicities examined. As such, inclusion of non-crossreactive A*0201 binding peptides increases the redundancy of antigen coverage and population coverage. The only two A2-restricted peptides that lack patient immunogenicity data are peptides 1090.77 and 1069.06. The 1090.77 peptide is an analog of a highly immunogenic peptide recognized in acute HBV patients. Although recall responses in patients have not been tested for the ability to recognize the analog peptide, immunogenicity studies conducted in HLA transgenic mice have shown that CTL induced with 1090.77 are capable of recognizing target cells loaded with the naturally occurring sequence. These data indicate that CTL raised to the 1090.77 peptide are cross-reactive and should recognize HBV-infected cells. The 1069.06 peptide was included as a potential vaccine epitope because its high binding affinity for A*6802 results in a greater population coverage. The peptide is immunogenic in HLA-A2 transgenic mice and primary human cultures.

Preferred CTL epitopes include 7 A3-supertype-resticted peptides; 6 derived from the polymerase antigen, and one from the core region. All of the A3-supertype vaccine candidate peptides are immunogenic in patients. Although peptide 1142.05 is a non-crossreactive A3-restricted peptide, it has been shown to be recognized in patients and is capable of binding HLA-A1.

Nine B7-restricted peptides are preferred CTL epitopes identified in the examples. Of this group, 3 epitopes have been shown to be recognized in patients. While one of these peptides, 1147.04, is a non-crossreactive binder, it binds 2 of the major B7 supertype alleles with an IC₅₀ or binding affinity value of less than 100 nM. Six B7-supertype epitopes were included as preferred epitopes based on supertype binding. Immunogenicity studies in humans (Bertoni et al., 1997; Doolan et al., 1997; Threlkeld et al., 1997) have demonstrated that highly cross-reactive peptides are almost always recognized as epitopes. Given these results, and in light of the limited immunogenicity data available, the use of B7-supertype binding affinity as a selection criterion was deemed appropriate.

Similarly, there is little immunogenicity data regarding A1- and A24-restricted peptides. One preferred CTL epitope, 1069.04, has been reported to be recognized in recall responses from acute HBV patients. As discussed in the preceding paragraph, a high percentage of the peptides with binding affinities <100 nM are found to be immunogenic. For this reason, all A1 and A24 peptides with binding affinities <100 nM were considered as preferred CTL epitopes. Using this selection criterion, 3 A1-restricted and 6 A24-restricted peptides are identified as candidate epitopes. Further analysis found that 3 core-derived peptides bound A24 with intermediate affinity. Since relatively few core epitopes were identified during the course of this study, the intermediate A24 binding core peptides were also included in the set of preferred epitopes to provide a greater degree of redundancy in antigen coverage.

The list of preferred HBV-derived HTL epitopes is summarized in Table XXXVII. The set of HTL epitopes includes 12 DR supermotif binding peptides and 4 DR3 binding peptides. The bulk of the HTL epitopes are derived from polymerase; 2 envelope and 2 core derived epitopes are also included in the set of preferred HTL epitopes. The total estimated population coverage represented by the panel of HTL epitopes is in excess of 91% in each of five major ethnic groups (Table XXXVIII).

### Example 9

### Recognition of Generation of Endogenous Processed Antigens after Priming

This example determines that CTL induced by native or analoged epitopes identified and selected as described in Examples 1-5 recognize endogenously synthesized, i.e., native antigens.

Effector cells isolated from transgenic mice immunized with peptide epitopes as in Example 3, for example, HLA-A2 supermotif-bearing epitopes, are re-stimulated in vitro using peptide-coated stimulator cells. Six days later, effector cells are assayed for cytotoxicity and the cell lines that contain peptide-specific cytotoxic activity are further re-stimulated. An additional six days later, these cell lines are tested for cytotoxic activity on ⁵¹Cr labeled Jurkat-A2.1/Kb target cells, in the absence or presence of peptide, and also tested on ⁵¹Cr labeled target cells bearing the endogenously synthesized antigen, e.g., cells that are stably transfected with HBV expression vectors.

The results show that CTL lines obtained from animals primed with peptide epitope recognize endogenously synthesized HBV antigen. The choice of transgenic mouse model to be used for such an analysis depends upon the epitope(s) that is being evaluated. In addition to HLA-A*0201/Kb transgenic mice, several other transgenic mouse models including mice with human A11, which may also be used to evaluate A3 epitopes, and B7 alleles have been characterized and others (e.g., transgenic mice for HLA-A1 and A24) are being developed. HLA-DR1 and HLA-DR3 mouse models have also been developed, which may be used to evaluate HTL epitopes.

### Example 9

### Activity of CTL-HTL Conjugated Epitopes in Transgenic Mice

This example illustrates the induction of CTLs and HTLs in transgenic mice by use of an HBV CTL/HTL peptide conjugate. An analogous study may be found in Oseroff et al. Vaccine 16:823-833 (1998).

The peptide composition can comprise multiple CTL and/or HTL epitopes and further, can comprise epitopes selected from multiple HPV target antigens. The epitopes are identified using methodology as described in Examples 1-6. For example, such a peptide composition can comprise an HTL epitope conjugated to a preferred CTL epitope containing, for example, at least one CTL epitope that binds to multiple HLA family members at an affinity of 500 nM or less, or analogs of that epitope. The peptides may be lipidated, if desired.

Immunization procedures: Immunization of transgenic mice is performed as described (Alexander et al., J. Immunol. 159:4753-4761, 1997). For example, A2/Kb mice, which are transgenic for the human HLA A2.1 allele and are useful for the assessment of the immunogenicity of HLA-A*0201 motif- or HLA-A2 supermotif-bearing epitopes, are primed subcutaneously (base of the tail) with a 0.1 ml of peptide in Incomplete Freund's Adjuvant, or if the peptide composition is a lipidated CTL/HTL conjugate, in DMSO/saline or if the peptide composition is a polypeptide, in PBS or Incomplete Freund's Adjuvant. Seven days after priming, splenocytes obtained from these animals are restimulated with syngenic irradiated LPS-activated lymphoblasts coated with peptide.

Cell lines: Target cells for peptide-specific cytotoxicity assays are Jurkat cells transfected with the HLA-A2.1/Kb chimeric gene (e.g., Vitiello et al., J. Exp. Med. 173:1007, 1991)

In vitro CTL activation: One week after priming, spleen cells (30×10⁶ cells/flask) are co-cultured at 37° C. with syngeneic, irradiated (3000 rads), peptide coated lymphoblasts (10×10⁶ cells/flask) in 10 ml of culture medium/T25 flask. After six days, effector cells are harvested and assayed for cytotoxic activity.

Assay for cytotoxic activity: Target cells (1.0 to 1.5×10⁶) are incubated at 37° C. in the presence of 200 μl of ⁵¹Cr. After 60 minutes, cells are washed three times and resuspended in R10 medium. Peptide is added where required at a concentration of 1 μg/ml. For the assay, 10^{4 51}Cr-labeled target cells are added to different concentrations of effector cells (final volume of 200 μl) in U-bottom 96-well plates. After a 6 hour incubation period at 37° C., a 0.1 ml aliquot of supernatant is removed from each well and radioactivity is determined in a Micromedic automatic gamma counter. The percent specific lysis is determined by the formula: percent specific release=100×(experimental release−spontaneous release)/(maximum release−spontaneous release). To facilitate comparison between separate CTL assays run under the same conditions, % ⁵¹Cr release data is expressed as lytic units/10⁶ cells. One lytic unit is arbitrarily defined as the number of effector cells required to achieve 30% lysis of 10,000 target cells in a 6 hour 51Cr release assay. To obtain specific lytic units/10⁶, the lytic units/10⁶ obtained in the absence of peptide is subtracted from the lytic units/10⁶ obtained in the presence of peptide. For example, if 30% ⁵¹Cr release is obtained at the effector (E): target (T) ratio of 50:1 (i.e., 5×10⁵ effector cells for 10,000 targets) in the absence of peptide and 5:1 (i.e., 5×10⁴ effector cells for 10,000 targets) in the presence of peptide, the specific lytic units would be: [( 1/50,000)−( 1/500,000)]×10⁶=18 LU.

The results are analyzed to assess the magnitude of the CTL responses of animals injected with the immunogenic CTL/HTL conjugate vaccine preparation and are compared to the magnitude of the CTL response achieved using the CTL epitope as outlined in Example 3. Analyses similar to this may be performed to evaluate the immunogenicity of peptide conjugates containing multiple CTL epitopes and/or multiple HTL epitopes. In accordance with these procedures it is found that a CTL response is induced, and concomitantly that an HTL response is induced upon administration of such compositions.

### Example 10

### Selection of CTL and HTL Epitopes for Inclusion in an HBV-Specific Vaccine

This example illustrates the procedure for the selection of peptide epitopes for vaccine compositions of the invention. The peptides in the composition can be in the form of a nucleic acid sequence, either single or one or more sequences (i.e., minigene) that encodes peptide(s), or can be single and/or polyepitopic peptides.

The following principles are utilized when selecting an array of epitopes for inclusion in a vaccine composition. Each of the following principles is balanced in order to make the selection.

Epitopes are selected which, upon administration, mimic immune responses that have been observed to be correlated with HBV clearance. The number of epitopes used depends on observations of patients who spontaneously clear HBV. For example, if it has been observed that patients who spontaneously clear HBV generate an immune response to at least 3 epitopes on at least one HPB antigen, then 3-4 epitopes should be included for HLA class I. A similar rationale is used to determine HLA class II epitopes.

Epitopes are often selected that have a binding affinity of an IC₅₀ of 500 nM or less for an HLA class I molecule, or for class II, an IC₅₀ of 1000 nM or less.

Sufficient supermotif bearing peptides, or a sufficient array of allele-specific motif bearing peptides, are selected to give broad population coverage. For example, epitopes are selected to provide at least 80% population coverage. A Monte Carlo analysis, a statistical evaluation known in the art, can be employed to assess breadth, or redundancy, of population coverage.

When creating a polyepitopic compositions, e.g. a minigene, it is typically desirable to generate the smallest peptide possible that encompasses the epitopes of interest. The principles employed are similar, if not the same, as those employed when selecting a peptide comprising nested epitopes.

In cases where the sequences of multiple variants of the same target protein are available, potential peptide epitopes can also be selected on the basis of their conservancy. For example, a criterion for conservancy may define that the entire sequence of an HLA class I binding peptide or the entire 9-mer core of a class 1L binding peptide be conserved in a designated percentage of the sequences evaluated for a specific protein antigen.

Epitopes for inclusion in vaccine compositions are, for example, selected from those listed in Table XXXVIIa and b. A vaccine composition comprised of selected peptides, when administered, is safe, efficacious, and elicits an immune response similar in magnitude of an immune response that clears an acute HBV infection.

### Example 11

### Construction of Minigene Multi-Epitope DNA Plasmids

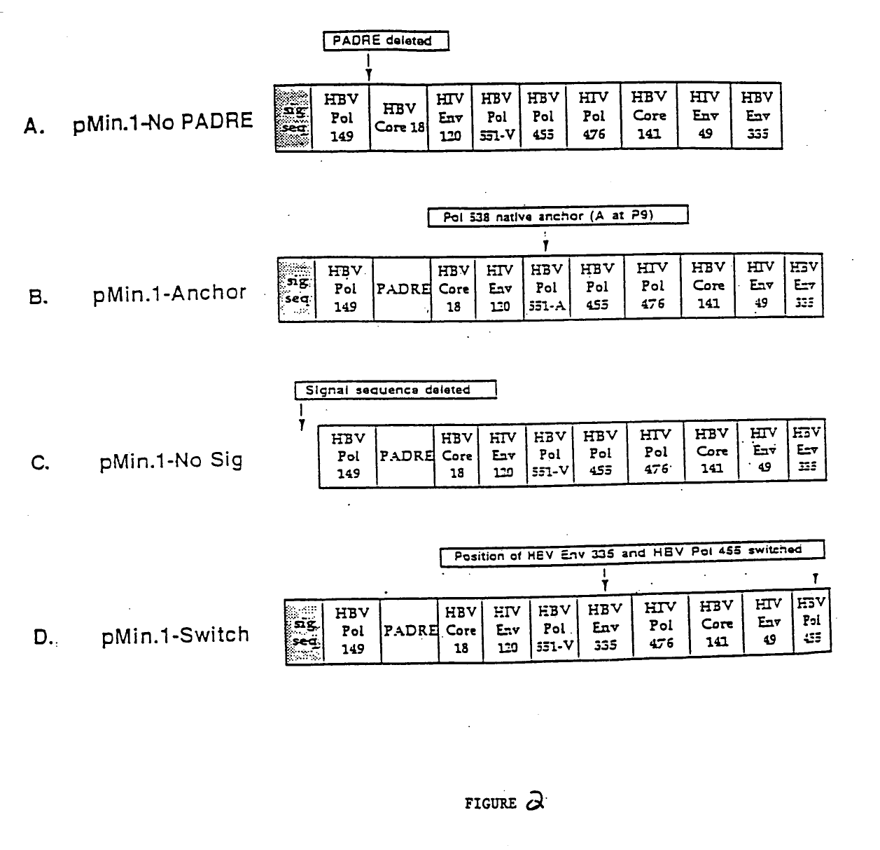

This example provides guidance for construction of a minigene expression plasmid. Minigene plasmids can, of course, contain various configurations of CTL and/or HTL epitopes or epitope analogs as described herein. Examples of the construction and evaluation of expression plasmids are described, for example, in co-pending U.S. Ser. No. 09/311,784 filed May 13, 1999. An example of such a plasmid is shown in FIG. 2, which illustrates the orientation of HBV epitopes in minigene constructs. Such a plasmid can, for example, also include multiple CTL and HTL peptide epitopes.

A minigene expression plasmid typically includes multiple CTL and HTL peptide epitopes. In the present example, HLA-A2, -A3, -B7 supermotif-bearing peptide epitopes and HLA-A1 and -A24 motif-bearing peptide epitopes are used in conjunction with DR supermotif-bearing epitopes and/or DR3 epitopes (FIG. 2). Preferred epitopes are identified, for example, in Tables XXVI-XXXIII, HLA class I supermotif or motif-bearing peptide epitopes derived from multiple HBV antigens, e.g., the core, polymerase, envelope and X proteins, are selected such that multiple supermotifs/motifs are represented to ensure broad population coverage. Similarly, HLA class II epitopes are selected from multiple HBV antigens to provide broad population coverage, i.e. both HLA DR-1-4-7 supermotif-bearing epitopes and HLA DR-3 motif-bearing epitopes are selected for inclusion in the minigene construct. The selected CTL and HTL epitopes are then incorporated into a minigene for expression in an expression vector.

This example illustrates the methods to be used for construction of such a minigene-bearing expression plasmid. Other expression vectors that may be used for minigene compositions are available and known to those of skill in the art.

The minigene DNA plasmid contains a consensus Kozak sequence and a consensus murine kappa Ig-light chain signal sequence followed by a string of CTL and/or HTL epitopes selected in accordance with principles disclosed herein.

Overlapping oligonucleotides, for example eight oligonucleotides, averaging approximately 70 nucleotides in length with 15 nucleotide overlaps, are synthesized and HPLC-purified. The oligonucleotides encode the selected peptide epitopes as well as appropriate linker nucleotides. The final multiepitope minigene is assembled by extending the overlapping oligonucleotides in three sets of reactions using PCR. A Perkin/Elmer 9600 PCR machine is used and a total of 30 cycles are performed using the following conditions: 95° C. for 15 sec, annealing temperature (50 below the lowest calculated Tm of each primer pair) for 30 sec, and 72° C. for 1 min.

For the first PCR reaction, 5 μg of each of two oligonucleotides are annealed and extended: Oligonucleotides 1+2, 3+4, 5+6, and 7+8 are combined in 100 μl reactions containing Pfu polymerase buffer (1×=10 mM KCL, 10 mM (NH4)2SO4, 20 mM Tris-chloride, pH 8.75, 2 mM MgSO4, 0.1% Triton X-100, 100 μg/ml BSA), 0.25 mM each dNTP, and 2.5 U of Pfu polymerase. The full-length dimer products are gel-purified, and two reactions containing the product of 1+2 and 3+4, and the product of 5+6 and 7+8 are mixed, annealed, and extended for 10 cycles. Half of the two reactions are then mixed, and 5 cycles of annealing and extension carried out before flanking primers are added to amplify the full length product for 25 additional cycles. The full-length product is gel-purified and cloned into pCR-blunt (Invitrogen) and individual clones are screened by sequencing.

### Example 12

### The Plasmid Construct and the Degree to which it Induces Immunogenicity

The degree to which a plasmid construct, for example a plasmid constructed in accordance with Example 11, is able to induce immunogenicity can be evaluated in vitro by testing for epitope presentation by APC following transduction or transfection of the APC with an epitope-expressing nucleic acid construct. Such a study determines “antigenicity” and allows the use of human APC. The assay determines the ability of the epitope to be presented by the APC in a context that is recognized by a T cell by quantifying the density of epitope-HLA class I complexes on the cell surface. Quantitation can be performed by directly measuring the amount of peptide eluted from the APC (see, e.g., Sijts et al., J. Immunol. 156:683-692, 1996; Demotz et al., Nature 342:682-684, 1989); or the number of peptide-HLA class I complexes can be estimated by measuring the amount of lysis or lymphokine release induced by infected or transfected target cells, and then determining the concentration of peptide necessary to obtained equivalent levels of lysis or lymphokine release (see, e.g., Kageyama et al., J. Immunol. 154:567-576, 1995).

Atlernatively, immunogenicity can be evaluated through in vivo injections into mice and subsequent in vitro assessment of CTL and HTL activity, which are analysed using cytotoxicity and proliferation assays, respectively, as detailed e.g., in copending U.S. Ser. No. 09/311,784 filed May 13, 1999 and Alexander et al., Immunity 1:751-761, 1994.

For example, to assess the capacity of a DNA minigene construct (e.g., a pMin minigene construct generated as decribed in U.S. Ser. No. 09/311,784) containing at least one HLA-A2 supermotif peptide to induce CTLs in vivo, HLA-A2.1/Kb transgenic mice, for example, are immunized intramuscularly with 100 μg of naked cDNA. As a means of comparing the level of CTLs induced by cDNA immunization, a control group of animals is also immunized with an actual peptide composition that comprises multiple epitopes synthesized as a single polypeptide as they would be encoded by the minigene.

Splenocytes from immunized animals are stimulated twice with each of the respective compositions (peptide epitopes encoded in the minigene or the polyepitopic peptide), then assayed for peptide-specific cytotoxic activity in a ⁵¹Cr release assay. The results indicate the magnitude of the CTL response directed against the A2-restricted epitope, thus indicating the in vivo immunogenicity of the minigene vaccine and polyepitopic vaccine. It is, therefore, found that the minigene elicits immune responses directed toward the HLA-A2 supermotif peptide epitopes as does the polyepitopic peptide vaccine. A similar analysis is also performed using other HLA-A3 and HLA-B7 transgenic mouse models to assess CTL induction by HLA-A3 and HLA-B7 motif or supermotif epitopes.

To assess the capacity of a class II epitope encoding minigene to induce HTLs in vivo, DR transgenic mice, or for those epitope that cross react with the appropriate mouse MHC molecule, I-Ab-restricted mice, for example, are immunized intramuscularly with 100 μg of plasmid DNA. As a means of comparing the level of HTLs induced by DNA immunization, a group of control animals is also immunized with an actual peptide composition emulsified in complete Freund's adjuvant. CD4⁺ T cells, i.e. HTLs, are purified from splenocytes of immunized animals and stimulated with each of the respective compositions (peptides encoded in the minigene). The HTL response is measured using a 3H-thymidine incorporation proliferation assay, (see, e.g., Alexander et al. Immunity 1:751-761, 1994). The results indicate the magnitude of the HTL response, thus demonstrating the in vivo immunogenicity of the minigene.

DNA minigenes, constructed as described in Example 11, may also be evaluated as a vaccine in combination with a boosting agent using a prime boost protocol. The boosting agent can consist of recombinant protein (e.g., Barnett et al., Aids Res. and Human Retroviruses 14, Supplement 3:S299-S309, 1998) or recombinant vaccinia, for example, expressing a minigene or DNA encoding the complete protein of interest (see, e.g., Hanke et al., Vaccine 16:439-445, 1998; Sedegah et al., Proc. Natl. Acad. Sci USA 95:7648-53, 1998; Hanke and McMichael, Immunol. Letters 66:177-181, 1999; and Robinson et al., Nature Med. 5:526-34, 1999).

For example, the efficacy of the DNA minigene used in a prime boost protocol is initially evaluated in transgenic mice. In this example, A2.1/Kb transgenic mice are immunized IM with 100 μg of a DNA minigene encoding the immunogenic peptides including at least one HLA-A2 supermotif-bearing peptide. After an incubation period (ranging from 3-9 weeks), the mice are boosted IP with 10⁷ pfu/mouse of a recombinant vaccinia virus expressing the same sequence encoded by the DNA minigene. Control mice are immunized with 100 μg of DNA or recombinant vaccinia without the minigene sequence, or with DNA encoding the minigene, but without the vaccinia boost. After an additional incubation period of two weeks, splenocytes from the mice are immediately assayed for peptide-specific activity in an ELISPOT assay. Additionally, splenocytes are stimulated in vitro with the A2-restricted peptide epitopes encoded in the minigene and recombinant vaccinia, then assayed for peptide-specific activity in an IFN-γ ELISA.

It is found that the minigene utilized in a prime-boost protocol elicits greater immune responses toward the HLA-A2 supermotif peptides than with DNA alone. Such an analysis can also be performed using HLA-A11 or HLA-B7 transgenic mouse models to assess CTL induction by HLA-A3 or HLA-B7 motif or supermotif epitopes.

The use of prime boost protocols in humans is described in Example 20.

### Example 13

### Peptide Composition for Prophylactic Uses

Vaccine compositions of the present invention can be used to prevent HJBV infection in persons who are at risk for such infection. For example, a polyepitopic peptide epitope composition (or a nucleic acid comprising the same) containing multiple CTL and HTL epitopes such as those selected in Examples 9 and/or 10, which are also selected to target greater than 80% of the population, is administered to individuals at risk for HBV infection.

For example, a peptide-based composition can be provided as a single polypeptide that encompasses multiple epitopes. The vaccine is typically administered in a physiological solution that comprises an adjuvant, such as Incomplete Freunds Adjuvant. The dose of peptide for the initial immunization is from about 1 to about 50,000 μg, generally 100-5,000 μg, for a 70 kg patient. The initial administration of vaccine is followed by booster dosages at 4 weeks followed by evaluation of the magnitude of the immune response in the patient, by techniques that determine the presence of epitope-specific CTL populations in a PBMC sample. Additional booster doses are administered as required. The composition is found to be both safe and efficacious as a prophylaxis against HPV infection.

Alternatively, a composition typically comprising transfecting agents can be used for the administration of a nucleic acid-based vaccine in accordance with methodologies known in the art and disclosed herein.

### Example 14

### Polyepitopic Vaccine Compositions Derived from Native HBV Sequences

A native HBV polyprotein sequence is screened, preferably using computer algorithms defined for each class I and/or class II supermotif or motif, to identify “relatively short” regions of the polyprotein that comprise multiple epitopes and is preferably less in length than an entire native antigen. This relatively short sequence that contains multiple distinct, even overlapping, epitopes is selected and used to generate a minigene construct. The construct is engineered to express the peptide, which corresponds to the native protein sequence. The “relatively short” peptide is generally less than 250 amino acids in length, often less than 100 amino acids in length, preferably less than 75 amino acids in length, and more preferably less than 50 amino acids in length. The protein sequence of the vaccine composition is selected because it has maximal number of epitopes contained within the sequence, i.e., it has a high concentration of epitopes. As noted herein, epitope motifs may be nested or overlapping (i.e., frame shifted relative to one another). For example, with f overlapping epitopes, two 9-mer epitopes and one 10-mer epitope can be present in a 10 amino acid peptide. Such a vaccine composition is administered for therapeutic or prophylactic purposes.

The vaccine composition will include, for example, three CTL epitopes from at least one HBV target antigen and at least one HTL epitope. This polyepitopic native sequence is administered either as a peptide or as a nucleic acid sequence which encodes the peptide. Alternatively, an analog can be made of this native sequence, whereby one or more of the epitopes comprise substitutions that alter the cross-reactivity and/or binding affinity properties of the polyepitopic peptide.

The embodiment of this example provides for the possibility that an as yet undiscovered aspect of immune system processing will apply to the native nested sequence and thereby facilitate the production of therapeutic or prophylactic immune response-inducing vaccine compositions. Additionally such an embodiment provides for the possibility of motif-bearing epitopes for an HLA makeup that is presently unknown. Furthermore, this embodiment (absent analogs) directs the immune response to multiple peptide sequences that are actually present in native HBV antigens thus avoiding the need to evaluate any junctional epitopes. Lastly, the embodiment provides an economy of scale when producing nucleic acid vaccine compositions.

Related to this embodiment, computer programs can be derived in accordance with principles in the art, which identify in a target sequence, the greatest number of epitopes per sequence length.

### Example 15

### Polyepitopic Vaccine Compositions Directed to Multiple Diseases

The HBV peptide epitopes of the present invention are used in conjunction with peptide epitopes from target antigens related to one or more other diseases, to create a vaccine composition that is useful for the prevention or treatment of HBV as well as another disease. Examples of other diseases include, but are not limited to, HIV, HCV, and HPV.

For example, a polyepitopic peptide composition comprising multiple CTL and HTL epitopes that target greater than 98% of the population may be created for administration to individuals at risk for both HBV and HIV infection. The composition can be provided as a single polypeptide that incorporates the multiple epitopes from the various disease-associated sources, or can be administered as a composition comprising one or more discrete epitopes.

### Example 16

### Use of Peptides to Evaluate an Immune Response

Peptides of the invention may be used to analyze an immune response for the presence of specific CTL or HTL populations directed to HBV. Such an analysis may be performed in a manner as that described by Ogg et al., Science 279:2103-2106, 1998. In the following example, peptides in accordance with the invention are used as a reagent for diagnostic or prognostic purposes, not as an immunogen.

In this example highly sensitive human leukocyte antigen tetrameric complexes (“tetramers”) are used for a cross-sectional analysis of, for example, HBV HLA-A*0201-specific CTL frequencies from HLA A*0201-positive individuals at different stages of infection or following immunization using an HBV peptide containing an A*0201 motif. Tetrameric complexes are synthesized as described (Musey et al., N. Engl. J. Med. 337:1267, 1997). Briefly, purified HLA heavy chain (A*0201 in this example) and P2-microglobulin are synthesized by means of a prokaryotic expression system. The heavy chain is modified by deletion of the transmembrane-cytosolic tail and COOH-terminal addition of a sequence containing a BirA enzymatic biotinylation site. The heavy chain, β2-microglobulin, and peptide are refolded by dilution. The 45-kD refolded product is isolated by fast protein liquid chromatography and then biotinylated by BirA in the presence of biotin (Sigma, St. Louis, Mo.), adenosine 5′triphosphate and magnesium. Streptavidin-phycoerythrin conjugate is added in a 1:4 molar ratio, and the tetrameric product is concentrated to 1 mg/ml. The resulting product is referred to as tetramer-phycoerythrin.

For the analysis of patient blood samples, approximately one million PBMCs are centrifuged at 300 g for 5 minutes and resuspended in 50 μl of cold phosphate-buffered saline. Tri-color analysis is performed with the tetramer-phycoerythrin, along with anti-CD8-Tricolor, and anti-CD38. The PBMCs are incubated with tetramer and antibodies on ice for 30 to 60 min and then washed twice before formaldehyde fixation. Gates are applied to contain >99.98% of control samples. Controls for the tetramers include both A*0201-negative individuals and A*0201-positive uninfected donors. The percentage of cells stained with the tetramer is then determined by flow cytometry. The results indicate the number of cells in the. PBMC sample that contain epitope-restricted CTLs, thereby readily indicating the extent of immune response to the HBV epitope, and thus the stage of infection with HBV, the status of exposure to HBV, or exposure to a vaccine that elicits a protective or therapeutic response.

### Example 17

### Use of Peptide Epitopes to Evaluate Recall Responses

The peptide epitopes of the invention are used as reagents to evaluate T cell responses, such as acute or recall responses, in patients. Such an analysis may be performed on patients who have recovered from infection, who are chronically infected with HBV, or who have been vaccinated with an HBV vaccine.

For example, the class I restricted CTL response of persons who have been vaccinated may be analyzed. The vaccine may be any HBV vaccine. PBMC are collected from vaccinated individuals and HLA-typed. Appropriate peptide epitopes of the invention that, optimally, bear supermotifs to provide cross-reactivity with multiple HLA supertype family members, are then used for analysis of samples derived from individuals who bear that HLA type.

PBMC from vaccinated individuals are separated on Ficoll-Histopaque density gradients (Sigma Chemical Co., St. Louis, Mo.), washed three times in HBSS (GIBCO Laboratories), resuspended in RPMI-1640 (GIBCO Laboratories) supplemented with L-glutamine (2 mM), penicillin (50 U/ml), streptomycin (50 μg/ml), and Hepes (10 mM) containing 10% heat-inactivated human AB serum (complete RPMI) and plated using microculture formats. A synthetic peptide comprising an epitope of the invention is added at 10 μg/ml to each well and HBV core 128-140 epitope is added at 1 μg/ml to each well as a source of T cell help during the first week of stimulation.

In the microculture format, 4×10⁵ PBMC are stimulated with peptide in 8 replicate cultures in 96-well round bottom plate in 100 μl/well of complete RPMI. On days 3 and 10, 100 ml of complete RPMI and 20 U/ml final concentration of rIL-2 are added to each well. On day 7 the cultures are transferred into a 96-well flat-bottom plate and restimulated with peptide, rIL-2 and 10⁵ irradiated (3,000 rad) autologous feeder cells. The cultures are tested for cytotoxic activity on day 14. A positive CTL response requires two or more of the eight replicate cultures to display greater than 10% specific ⁵¹Cr release, based on comparison with uninfected control subjects as previously described (Rehermann, et al., Nature Med. 2:1104,1108, 1996; Rehermann et al., J. Clin. Invest. 97:1655-1665, 1996; and Rehermann et al. J. Clin. Invest. 98:1432-1440, 1996).

Target cell lines are autologous and allogeneic EBV-transformed B-LCL that are either purchased from the American Society for Histocompatibility and Immunogenetics (ASHI, Boston, Mass.) or established from the pool of patients as described (Guilhot, et al. J. Virol. 66:2670-2678, 1992).

Cytotoxicity assays are performed in the following manner. Target cells consist of either allogeneic HLA-matched or autologous EBV-transformed B lymphoblastoid cell line that are incubated overnight with the synthetic peptide epitope of the invention at 10 μM, and labeled with 100 μCi of ⁵¹Cr (Amersham Corp., Arlington Heights, Ill.) for 1 hour after which they are washed four times with HBSS.

Cytolytic activity is determined in a standard 4-h, split well ⁵¹Cr release assay using U-bottomed 96 well plates containing 3,000 targets/well. Stimulated PBMC are tested at effector/target (E/T) ratios of 20-50:1 on day 14. Percent cytotoxicity is determined from the formula: 100×[(experimental release-spontaneous release)/maximum release-spontaneous release)]. Maximum release is determined by lysis of targets by detergent (2% Triton X-100; Sigma Chemical Co., St. Louis, Mo.). Spontaneous release is <25% of maximum release for all experiments.

The results of such an analysis indicate the extent to which HLA-restricted CTL populations have been stimulated by previous exposure to HBV or an HBV vaccine.

Class II restricted HTL responses an also be analyzed. Purified PBMC are cultured in a 96-well flat bottom plate at a density of 1.5×10⁵ cells/well and are stimulated with 10 μg/ml synthetic peptide, whole antigen, or PHA. Cells are routinely plated in replicates of 4-6 wells for each condition. After seven days of culture, the medium is removed and replaced with fresh medium containing 10 U/ml IL-2. Two days later, 1 μCi 3H-thymidine is added to each well and incubation is continued for an additional 18 hours. Cellular DNA is then harvested on glass fiber mats and analyzed for 3H-thymidine incorporation. Antigen-specific T cell proliferation is calculated as the ratio of 3H-thymidine incorporation in the presence of antigen divided by the 3H-thymidine incorporation in the absence of antigen.

### Example 18

### Induction of Specific CTL Response in Humans

A human clinical trial for an immunogenic composition comprising HBV CTL and HTL epitopes of the invention is set up as an SD Phase I, dose escalation study (5, 50 and 500 μg) and carried out as a randomized, double-blind, placebo-controlled trial. Such a trial is designed, for example, as follows:

A total of about 27 subjects are enrolled and divided into 3 groups:

Group I: 3 subjects are injected with placebo and 6 subjects are injected with 5 μg of peptide composition;

Group II: 3 subjects are injected with placebo and 6 subjects are injected with 50 μg peptide composition;

Group III: 3 subjects are injected with placebo and 6 subjects are injected with 500 μg of peptide composition.

After 4 weeks following the first injection, all subjects receive a booster inoculation at the same dosage.

The endpoints measured in this study relate to the safety and tolerability of the peptide composition as well as its immunogenicity. Cellular immune responses to the peptide composition are an index of the intrinsic activity of this the peptide composition, and can therefore be viewed as a measure of biological efficacy. The following summarize the clinical and laboratory data that relate to safety and efficacy endpoints.

Safety: The incidence of adverse events is monitored in the placebo and drug treatment group and assessed in terms of degree and reversibility.

Evaluation of Vaccine Efficacy: For evaluation of vaccine efficacy, subjects are bled before and after injection. Peripheral blood mononuclear cells are isolated from fresh heparinized blood by Ficoll-Hypaque density gradient centrifugation, aliquoted in freezing media and stored frozen. Samples are assayed for CTL and HTL activity.

Thus, the vaccine is found to be both safe and efficacious.

### Example 19

### Phase II Trials in Patients Infected with HBV

Phase II trials are performed to study the effect of administering the CTL-HTL peptide compositions to patients (male and female ) having chronic HBV infection. A main objective of the trials is to determine an effective dose and regimen for inducing CTLs in chronically infected HBV patients, to establish the safety of inducing a CTL response in these patients, and to see to what extent activation of CTLs improves the clinical picture of chronically infected CTL patients, as manifested by a transient flare in alanine aminotransferase (ALT), normalization of ALT, and reduction in HBV DNA. Such a study is designed, for example, as follows:

The studies are performed in multiple centers in the U.S. and Canada. The trial design is an open-label, uncontrolled, dose escalation protocol wherein the peptide composition is administered as a single dose followed six weeks later by a single booster shot of the same dose. The dosages are 50, 500 and 5,000 micrograms per injection. Drug-associated adverse effects are recorded.

There are three patient groupings. The first group is injected with 50 micrograms of the peptide composition and the second and third groups with 500 and 5,000 micrograms of peptide composition, respectively. The patients within each group range in age from 21-65 and include both males and females. The patients represent diverse ethnic backgrounds. All of them are infected with HBV for over five years and are HIV, HCV and HDV negative, but have positive levels of HBe antigen and HBs antigen.

The magnitude and incidence of ALT flares and the levels of HBV DNA in the blood are monitored to assess the effects of administering the peptide compositions. The levels of HBV DNA in the blood are an indirect indication of the progress of treatment. The vaccine composition is found to be both safe and efficacious in the treatment of chronic HBV infection.

### Example 20

### Induction of CTL Responses Using a Prime Boost Protocol

A prime boost protocol can also be used for the administration of the vaccine to humans. Such a vaccine regimen can include an initial administration of, for example, naked DNA followed by a boost using recombinant virus encoding the vaccine, or recombinant protein/polypeptide or a peptide mixture administered in an adjuvant.

For example, the initial immunization may be performed using an expression vector, such as that constructed in Example 11, in the form of naked nucleic acid administered IM (or SC or ID) in the amounts of 0.5-5 mg at multiple sites. The nucleic acid (0.1 to 1000 μg) can also be administered using a gene gun. Following an incubation period of 3-4 weeks, a booster dose is then administered. The booster can be recombinant fowlpox virus administered at a dose of 5×10⁷ to 5×10⁹ pfu. An alternative recombinant virus, such as an MVA, canarypox, adenovirus, or adeno-associated virus, can also be used for the booster, or the polyepitopic protein or a mixture of the peptides can be administered. For evaluation of vaccine efficacy, patient blood samples will be obtained before immunization as well as at intervals following administration of the initial vaccine and booster doses of the vaccine. Peripheral blood mononuclear cells are isolated from fresh heparinized blood by Ficoll-Hypaque density gradient centrifugation, aliquoted in freezing media and stored frozen. Samples are assayed for CTL and HTL activity.

The results indicate that a magnitude of response sufficient to achieve protective immunity against HBV or to treat HBV infection is generated.

### Example 21

### Administration of Vaccine Compositions Using Dendritic Cells (DC)

Vaccines comprising peptide epitopes of the invention can be administered using APCs, such as DC. In this example, the peptide-pulsed DC are administered to a patient to stimulate a CTL response in vivo. In this method, dendritic cells are isolated, expanded, and pulsed with a vaccine comprising peptide CTL and HTL epitopes of the invention. The dendritic cells are infused back into the patient to elicit CTL and HTL responses in vivo. The induced CTL and HTL then destroy or facilitate destruction of the specific target cells that bear the proteins from which the epitopes in the vaccine are derived.

For example, a cocktail of epitope-bearing peptides is administered ex vivo to PBMC, or isolated DC therefrom. A pharmaceutical to facilitate harvesting of DC can be used, such as Progenipoietin™ (Monsanto, St. Louis, Mo.) or GM-CSF/IL-4. After pulsing the DC with peptides and prior to reinfusion into patients, the DC are washed to remove unbound peptides.

As appreciated clinically, and readily determined by one of skill based on clinical outcomes, the number of DC reinfused into the patient can vary (see, e.g., Nature Med. 4:328, 1998; Nature Med. 2:52, 1996 and Prostate 32:272, 1997). Although 2-50×10⁶ DC per patient are typically administered, larger number of DC, such as 10⁷ or 10⁸ can also be provided. Such cell populations typically contain between 50-90% DC.

In some embodiments, peptide-loaded PBMC are injected into patients without purification of the DC. For example, PBMC containing DC generated after treatment with an agent such as Progenipoietin™ are injected into patients without purification of the DC. The total number of PBMC that are administered often ranges from 10⁸ to 10¹⁰. Generally, the cell doses injected into patients is based on the percentage of DC in the blood of each patient, as determined, for example, by immunofluorescence analysis with specific anti-DC antibodies. Thus, for example, if Progenipoietin™ mobilizes 2% DC in the peripheral blood of a given patient, and that patient is to receive 5×10⁶ DC, then the patient will be injected with a total of 2.5×10⁸ peptide-loaded PBMC. The percent DC mobilized by an agent such as Progenipoietin™ is typically estimated to be between 2-10%, but can vary as appreciated by one of skill in the art.

Ex vivo Activation of CTL/HTL Responses

Alternatively, ex vivo CTL or HTL responses to HPV antigens can be induced by incubating in tissue culture the patient's, or genetically compatible, CTL or HTL precursor cells together with a source of APC, such as DC, and the appropriate immunogenic peptides. After an appropriate incubation time (typically about 7-28 days), in which the precursor cells are activated and expanded into effector cells, the cells are infused back into the patient, where they will destroy (CTL) or facilitate destruction (HTL) of their specific target cells.

### Example 22

### Alternative Method of Identifying Motif-Bearing Peptides

Another method of identifying motif-bearing peptides is to elute them from cells bearing defined MHC molecules. For example, EBV transformed B cell lines used for tissue typing have been extensively characterized to determine which HLA molecules they express. In certain cases these cells express only a single type of HLA molecule. These cells can be infected with a pathogenic organism or transfected with nucleic acids that express the antigen of interest, e.g. HBV proteins. Peptides produced by endogenous antigen processing of peptides produced consequent to infection (or as a result of transfection) will then bind to HLA molecules within the cell and be transported and displayed on the cell surface. Peptides are then eluted from the HLA molecules by exposure to mild acid conditions and their amino acid sequence determined, e.g., by mass spectral analysis (e.g., Kubo et al., J. Immunol. 152:3913, 1994). Because the majority of peptides that bind a particular HLA molecule are motif-bearing, this is an alternative modality for obtaining the motif-bearing peptides correlated with the particular HLA molecule expressed on the cell.

Alternatively, cell lines that do not express endogenous HLA molecules can be transfected with an expression construct encoding a single HLA allele. These cells can then be used as described, i.e., they can be infected with a pathogen or transfected with nucleic acid encoding an antigen of interest to isolate peptides corresponding to the pathogen or antigen of interest that have been presented on the cell surface. Peptides obtained from such an analysis will bear motif(s) that correspond to-binding to the single HLA allele that is expressed in the cell.

As appreciated by one in the art, one can perform a similar analysis on a cell bearing more than one HLA allele and subsequently determine peptides specific for each HLA allele expressed. Moreover, one of skill would also recognize that means other than infection or transfection, such as loading with a protein antigen, can be used to provide a source of antigen to the cell.

The examples herein are provided to illustrate the invention but not to limit its scope. Other variants of the invention will be readily apparent to one of ordinary skill in the art and are encompassed by the appended claims. All publications, patents, and patent application cited herein are hereby incorporated by reference for all purposes.

TABLE IV

HLA Class I Standard Peptide Binding Affinity.

| | | | | |
|---|---|---|---|---|
| | | | | BINDING |
| | STANDARD | | | AFFINITY |
| ALLELE | PEPTIDE | SEQUENCE | SEQ ID NO: | (nM) |

| | | | | |
|---|---|---|---|---|
| A*0101 | 944.02 | YLEPAIAKY | 3475 | 25 |
| A*0201 | 941.01 | FLPSDYFPSV | 3476 | 5.0 |
| A*0202 | 941.01 | FLPSDYFPSV | 3476 | 4.3 |
| A*0203 | 941.01 | FLPSDYFPSV | 3476 | 10 |
| A*0206 | 941.01 | FLPSDYFPSV | 3476 | 3.7 |
| A*0207 | 941.01 | FLPSDYFPSV | 3476 | 23 |
| A*6802 | 1141.02 | FTQAGYPAL | 3477 | 40 |
| A*0301 | 941.12 | KVFPYALINK | 3478 | 11 |
| A*1101 | 940.06 | AVDLYHFLK | 3479 | 6.0 |
| A*3101 | 941.12 | KVFPYALINK | 3478 | 18 |
| A*3301 | 1083.02 | STLPETYVVRR | 3480 | 29 |
| A*6801 | 941.12 | KVFPYALINK | 3479 | 8.0 |
| A*2402 | 979.02 | AYIDNYNKF | 3481 | 12 |
| B*0702 | 1075.23 | APRTLVYLL | 3482 | 5.5 |
| B*3501 | 1021.05 | FPFKYAAAF | 3483 | 7.2 |
| B51 | 1021.05 | FPFKYAAAF | 3483 | 5.5 |
| B*5301 | 1021.05 | PPFKYAAAF | 3483 | 93 |
| B*5401 | 1021.05 | FPFKYAAAF | 3483 | 10 |

TABLE V

HLA Class II Standard Peptide Binding Affinity.

| | | | | | |
|---|---|---|---|---|---|
| | | | | | Binding |
| | | Standard | | SEQ ID | Affinity |
| Allele | Nomenclature | Peptide | Sequence | NO: | (nM) |

| | | | | | |
|---|---|---|---|---|---|
| DRB1*0101 | DR1 | 515.01 | PKYVKQNTLKLAT | 3484 | 5.0 |
| DRB1*0301 | DR3 | 829.02 | YKTIAFDEEARR | 3485 | 300 |
| DRB1*0401 | DR4w4 | 515.01 | PKYVKQNTLKLAT | 3484 | 45 |
| DRB1*0404 | DR4w14 | 717.01 | YARFQSQTTLKQKT | 3486 | 50 |
| DRB1*0405 | DR4w15 | 717.01 | YARFQSQTTLKQKT | 3486 | 38 |
| DRB1*0701 | DR7 | 553.01 | QYIKANSKFIGITE | 3487 | 25 |
| DRB1*0802 | DR8w2 | 553.01 | QYIKANSKFIGITE | 3487 | 49 |
| DRB1*0803 | DR8w3 | 553.01 | QYIKANSKFIGITE | 3487 | 1600 |
| DRB1*0901 | DR9 | 553.01 | QYIKANSKFIGITE | 3487 | 75 |
| DRB1*1101 | DR5w11 | 553.01 | QYIKANSKFIGITE | 3487 | 20 |
| DRB1*1201 | DR5w12 | 1200.05 | EALIHQLKINPYVLS | 3488 | 298 |
| DRB1*1302 | DR6w19 | 650.22 | QYIKANAKFIGITE | 3489 | 3.5 |
| DRB1*1501 | DR2w2β1 | 507.02 | GRTQDENPVVHFFKNI | 3490 | 9.1 |
| | | | VTPRTPPP | | |
| DRB3*0101 | DR52a | 511 | NGQIGNDPNRDIL | 3491 | 470 |
| DRB4*0101 | DRw53 | 717.01 | YARFQSQTTLKQKT | 3486 | 58 |
| DRB5*0101 | DR2w2β1 | 553.01 | QYIKANSKFIGITE | 3487 | 20 |

The “Nomenclature” column lists the allelic designations used in Tables XIX and XX.

TABLE VI

| HLA- | Allele-specific HLA-supertype members | |
|---|---|---|
| supertype | Verified^{a} | Predicted^{b} |
| A1 | A*0101, A*2501, A*2601, | A*0102, A*2604, A*3601, |
| | A*2602, A*3201 | A*4301, A*8001 |
| A2 | A*0201, A*0202, A*0203, | A*0208, A*0210, A*0211, |
| | A*0204, A*0205, A*0206, | A*0212, A*0213 |
| | A*0207, A*0209, A*0214, | |
| | A*6802, A*6901 | |
| A3 | A*0301, A*1101, A*3101, | A*0302, A*1102, A*2603, |
| | A*3301, A*6801 | A*3302, A*3303, A*3401, |
| | | A*3402, A*6601, A*6602, |
| | | A*7401 |
| A24 | A*2301, A*2402, A*3001 | A*2403, A*2404, A*3002, |
| | | A*3003 |
| B7 | B*0702, B*0703, B*0704, | B*1511, B*4201, B*5901 |
| | B*0705, B*1508, B*3501, | |
| | B*3502, B*3503, B*3504, | |
| | B*3505, B*3506, B*3507, | |
| | B*3508, B*5101, B*5102, | |
| | B*5103, B*5104, B*5105, | |
| | B*5301, B*5401, B*5501, | |
| | B*5502, B*5601, B*5602, | |
| | B*6701, B*7801 | |
| B27 | B*1401, B*1402, B*1509, | B*2701, B*2707, B*2708, |
| | B*2702, B*2703, B*2704, | B*3802, B*3903, B*3904, |
| | B*2705, B*2706, B*3801, | B*3905, B*4801, B*4802, |
| | B*3901, B*3902, B*7301 | B*1510, B*1518, B*1503 |
| B44 | B*1801, B*1802, B*3701, | B*4101, B*4501, B*4701, |
| | B*4402, B*4403, B*4404, | B*4901, B*5001 |
| | B*4001, B*4002, B*4006 | |
| B58 | B*5701, B*5702, B*5801, | |
| | B*5802, B*1516, B*1517 | |
| B62 | B*1501, B*1502, B*1513, | B*1301, B*1302, B*1504, |
| | B*5201 | B*1505, B*1506, B*1507, |
| | | B*1515, B*1520, B*1521, |
| | | B*1512, B*1514, B*1519 |

^{a}Verified alleles includes alleles whose specificity has been determined by pool sequencing analysis, peptide binding assays, or by analysis of the sequences of CTL epitopes.
^{b}Predicted alleles are alleles whose specificity is predicted on the basis of B and F pocket structure to overlap with the supertype specificity.

TABLE VII

HBV A01 SUPER MOTIF (With binding information)

| | | | | | | | |
|---|---|---|---|---|---|---|---|
| Conservancy | Freq. | Protein | Position | Sequence | SEQ ID NO: | String | A*0101 |

| | | | | | | | |
|---|---|---|---|---|---|---|---|
| 95 | 19 | POL | 521 | AJCSVVRRAF | 1 | XIXXXXXXXF | |
| 95 | 19 | NUC | 54 | ALRQAILCW | 2 | XLXXXXXXXW | |
| 80 | 16 | ENV | 108 | AMOWNSTTF | 3 | XMXXXXXXXF | |
| 100 | 20 | POL | 166 | ASFCGSPY | 4 | XSXXXXXY | |
| 100 | 20 | POL | 166 | ASFCGSPYSW | 5 | XSXXXXXXXW | |
| 90 | 18 | NUC | 19 | ASKLCLGW | 6 | XSXXXXXW | |
| 85 | 17 | NUC | 19 | ASKLCLGWLW | 7 | XSXXXXXXXW | |
| 80 | 16 | POL | 822 | ASPLHVAW | 8 | XSXXXXXW | |
| 100 | 20 | ENV | 312 | CIPIPSSW | 9 | XIXXXXXW | |
| 100 | 20 | ENV | 312 | CIPIPSSWAF | 10 | XIXXXXXXXF | |
| 95 | 19 | ENV | 253 | CLIFLLVLLDY | 11 | XLXXXXXXXXY | |
| 95 | 19 | ENV | 239 | CLRRFIIF | 12 | XLXXXXXF | |
| 75 | 15 | ENV | 239 | CLRRFIFLF | 13 | XLXXXXXXXF | |
| 95 | 19 | POL | 523 | CSVVRRAF | 14 | XSXXXXXF | |
| 100 | 20 | ENV | 310 | CTCIPIPSSW | 15 | XTXXXXXXXW | |
| 90 | 18 | NUC | 31 | DIDPYKEF | 16 | XIXXXXXF | |
| 85 | 17 | NUC | 29 | DLLDTASALY | 17 | XLXXXXXXXY | 11.1000 |
| 95 | 19 | ENV | 196 | DSWWTSLNF | 18 | XSXXXXXXF | |
| 95 | 19 | NUC | 43 | ELLSFLPSDF | 19 | XLXXXXXXXF | |
| 95 | 19 | NUC | 43 | ELLSFLPSDFF | 20 | XLXXXXXXXXF | |
| 95 | 19 | POL | 374 | ESRLVVDF | 21 | XSXXXXXF | |
| 95 | 19 | POL | 374 | ESRLVVDFSOF | 22 | XSXXXXXXXXF | |
| 80 | 16 | ENV | 248 | FILLLCLIF | 23 | XIXXXXXXF | |
| 80 | 16 | ENV | 246 | FLFILLLCLIF | 24 | XLXXXXXXXXF | |
| 95 | 19 | ENV | 256 | FLLVLLDY | 25 | XIXXXXXY | |
| 95 | 19 | POL | 658 | FSPTYKAF | 26 | XSXXXXXF | |
| 90 | 18 | X | 63 | FSSAGPCALRF | 27 | XSXXXXXXXXF | |
| 100 | 20 | ENV | 333 | FSWLSLLVPF | 28 | XSXXXXXXXF | |
| 95 | 19 | POL | 656 | FTFSPTYKAF | 29 | XTXXXXXXXF | |
| 95 | 19 | ENV | 346 | FVGLSPTVW | 30 | XVXXXXXXW | |
| 95 | 19 | POL | 627 | GLLGFAAPF | 31 | XLXXXXXXF | |
| 95 | 19 | POL | 509 | GLSPFLLAOF | 32 | XLXXXXXXXF | |
| 85 | 17 | NUC | 29 | GMDIDPYKEF | 33 | XMXXXXXXXF | |
| 95 | 19 | NUC | 123 | GVWIRTPPAY | 34 | XVXXXXXXXY | 0.0017 |
| 75 | 15 | POL | 569 | HLNPNKTKRW | 35 | XLXXXXXXXW | |
| 80 | 16 | POL | 491 | HLYSHPILGF | 36 | XLXXXXXXXXF | |
| 85 | 17 | POL | 715 | HTAELLAACF | 37 | XTXXXXXXXF | |
| 95 | 19 | NUC | 52 | HTALROAILCW | 38 | XTXXXXXXXXW | |
| 100 | 20 | POL | 149 | HTLWKAGILY | 39 | XTXXXXXXXY | 0.0300 |
| 100 | 20 | ENV | 249 | ILLLCUF | 40 | XLXXXXXF | |
| 80 | 16 | POL | 760 | ILRGTSFVY | 41 | XLXXXXXXY | 0.0017 |
| 90 | 18 | ENV | 188 | ILTIPOSLDSW | 42 | XLXXXXXXXXW | |
| 90 | 18 | POL | 625 | IVGLLGFAAPF | 43 | XVXXXXXXXXF | |
| 80 | 16 | POL | 503 | KIPMGVGLSPF | 44 | XIXXXXXXXXF | |
| 85 | 17 | NUC | 21 | KLCLGWLW | 45 | XLXXXXXW | |
| 75 | 15 | POL | 108 | KLIMPARF | 46 | XLXXXXXF | |
| 75 | 15 | POL | 108 | KLIMPARFY | 47 | XLXXXXXXXY | 0.0017 |
| 80 | 16 | POL | 610 | KLPVNRPIDW | 48 | XLXXXXXXXW | |
| 85 | 17 | POL | 574 | KTKRWGYSLNF | 49 | XTXXXXXXXXF | |
| 95 | 19 | POL | 55 | KVGNFTGLY | 50 | XVXXXXXXXY | 0.0680 |
| 95 | 19 | ENV | 254 | LIFLLVLLDY | 51 | XIXXXXXXXY | 0.0084 |
| 100 | 20 | POL | 109 | LIMPARFY | 52 | XIXXXXXY | |
| 85 | 17 | NUC | 30 | LLOTASALY | 53 | XLXXXXXXY | 25.0000 |
| 80 | 16 | POL | 752 | LLGCAANW | 54 | XLXXXXXXW | |
| 95 | 19 | POL | 628 | LLGFAAPF | 55 | XLXXXXXF | |
| 100 | 20 | ENV | 378 | LLPIFFCLW | 56 | XLXXXXXXXW | |
| 100 | 20 | ENV | 378 | LLPIFFCLWVY | 57 | XLXXXXXXXXY | |
| 95 | 19 | NUC | 44 | LLSFLPSDP | 58 | XLXXXXXXF | |
| 95 | 19 | NUC | 44 | LLSFLPSDFF | 59 | XLXXXXXXXF | |
| 90 | 18 | POL | 407 | LLSSNLSW | 60 | XLXXXXXXW | |
| 95 | 19 | ENV | 175 | LLVLQAGF | 61 | XLXXXXXF | |
| 95 | 19 | ENV | 175 | LLVLQAGFF | 62 | XLXXXXXXF | |
| 100 | 20 | ENV | 338 | LLVPFVQW | 63 | XLXXXXXW | |
| 100 | 20 | ENV | 338 | LLVPFVQWF | 64 | XLXXXXXXF | |
| 85 | 17 | NUC | 100 | LLWFHISCLTF | 65 | XLXXXXXXXXF | |
| 95 | 19 | NUC | 45 | LSFLPSDF | 66 | XSXXXXXF | |
| 95 | 19 | NUC | 45 | LSFLPSDFF | 67 | XSXXXXXXF | |
| 95 | 19 | POL | 415 | LSLDVSAAF | 68 | XSXXXXXXF | |
| 95 | 19 | POL | 415 | LSLDVSAAFY | 69 | XSXXXXXXXY | 4.2000 |
| 100 | 20 | ENV | 336 | LSLLVPFVQW | 70 | XSXXXXXXXW | |
| 100 | 20 | ENV | 336 | LSLLVPFQWF | 71 | XSXXXXXXXXF | |
| 95 | 19 | X | 53 | LSLRGLPVCAF | 72 | XSXXXXXXXXF | |
| 95 | 19 | POL | 510 | LSPFLLAQF | 73 | XSXXXXXXF | |
| 75 | 15 | ENV | 349 | LSPTVWLSVTW | 74 | XSXXXXXXXXW | |
| 85 | 17 | POL | 742 | LSRKYTSF | 75 | XSXXXXXF | |
| 85 | 17 | POL | 742 | LSRKYTSFPW | 76 | XSXXXXXXXW | |
| 75 | 15 | ENV | 16 | LSVPNPLGF | 77 | XSXXXXXXF | |
| 75 | 15 | NUC | 137 | LTFGRETVLEY | 78 | XTXXXXXXXXY | |
| 90 | 18 | ENV | 189 | LTIPQSLDSW | 79 | XTXXXXXXXW | |
| 90 | 18 | ENV | 189 | LTIPQSLDSWW | 80 | XTXXXXXXXXW | |
| 90 | 18 | POL | 404 | LTNLLSSNLSW | 81 | XTXXXXXXXXW | |
| 95 | 19 | ENV | 176 | LVLQAGFF | 82 | XVXXXXXF | |
| 100 | 20 | ENV | 339 | LVPFVQWF | 83 | XVXXXXXF | |
| 100 | 20 | POL | 377 | LVVDFSQF | 84 | XVXXXXXF | |
| 85 | 17 | ENV | 360 | MMWYWGPSLY | 85 | XMXXXXXXXY | 0.0810 |
| 75 | 15 | X | 103 | MSTTDLEAY | 86 | XSXXXXXXY | 0.8500 |
| 75 | 15 | X | 103 | MSTTDLEAYF | 87 | XSXXXXXXXXF | |
| 95 | 19 | POL | 42 | NLGNLNVSIPW | 88 | XLXXXXXXXXW | |
| 90 | 18 | POL | 406 | NLLSSNLSW | 89 | XLXXXXXXW | |
| 95 | 19 | POL | 45 | NLNSIPW | 90 | XLXXXXXW | |
| 75 | 15 | ENV8W | 15 | NLSVPNPLGF | 91 | XLXXXXXXXF | |
| 90 | 18 | POL | 738 | NSVVLSRKY | 92 | XSXXXXXXY | 0.0005 |
| 100 | 20 | ENV | 380 | PIFFCLWVY | 93 | XIXXXXXXY | 0.0078 |
| 100 | 20 | ENV | 314 | PIPSSWAF | 94 | XIXXXXXF | |
| 100 | 20 | POL | 124 | PLDKFIKPY | 95 | XLXXXXXXY | 0.0190 |
| 100 | 20 | POL | 124 | PLDKGIKPYY | 96 | XLXXXXXXXY | 0.1600 |
| 100 | 20 | ENV | 377 | PLLPIFFCLW | 97 | XXXXXXXXW | |
| 95 | 19 | ENV | 174 | PLLVLQAGF | 98 | XLXXXXXXF | |
| 95 | 19 | ENV | 174 | PLLVLQAGFF | 99 | XLXXXXXXXF | |
| 80 | 16 | POL | 505 | PMGVGLSPF | 100 | XMXXXXXXF | |
| 85 | 17 | POL | 797 | PTTGRTSLY | 101 | XTXXXXXXY | 0.7700 |
| 75 | 15 | ENV | 351 | PTVWSVTW | 102 | XTXXXXXXW | |
| 85 | 17 | POL | 612 | PNRPIDW | 103 | XVXXXXXW | |
| 95 | 19 | POL | 685 | QVFADATPTG | 104 | XVXXXXXXXXW | |
| 90 | 18 | POL | 624 | RIVGLLGF | 105 | XIXXXXXF | |
| 75 | 15 | POL | 106 | RLKIMPARF | 106 | XLXXXXXXXF | |
| 75 | 15 | POL | 106 | RLKLIMPARFY | 107 | XLXXXXXXXXY | |
| 95 | 19 | POL | 376 | RLVVDFSCF | 108 | XLXXXXXXF | |
| 90 | 18 | POL | 353 | RTPARVTGGVF | 109 | XTXXXXXXXXF | |
| 100 | 20 | POL | 49 | SIPWTHKVGNF | 110 | XIXXXXXXXXXF | |
| 95 | 19 | ENV | 194 | SLDSWWTSLNF | 111 | XLXXXXXXXXF | |
| 95 | 19 | POL | 416 | SLDVSAAF | 112 | XLXXXXXF | |
| 95 | 19 | POL | 416 | SLDVSAAFY | 113 | XLXXXXXXY | 17.2000 |
| 100 | 20 | ENV | 337 | SLLVPFQW | 114 | XLXXXXXXW | |
| 100 | 20 | ENV | 337 | SLLVPFQWF | 115 | XWXXXXXXXF | |
| 95 | 19 | X | 54 | SLRGLPVCAF | 116 | XLXXXXXXXF | |
| 90 | 18 | X | 64 | SSAGPCALRF | 117 | XSXXXXXXXF | |
| 75 | 15 | X | 104 | STTDLEAY | 118 | XTXXXXXY | |
| 75 | 15 | X | 104 | STTDLEAYF | 119 | XTXXXXXXF | |
| 75 | 15 | ENV | 17 | SVPNPLGF | 120 | XVXXXXXF | |
| 90 | 18 | POL | 739 | SVVLSRKY | 121 | XVXXXXXY | |
| 85 | 17 | POL | 739 | SVVLSRKYTSF | 122 | XVXXXXXXXXF | |
| 90 | 18 | ENV | 190 | TIPQSLDSW | 123 | XIXXXXXXW | |
| 90 | 18 | ENV | 190 | TIPQSLDSWW | 124 | XIXXXXXXXW | |
| 100 | 20 | POL | 150 | TLWKAGILY | 125 | XLXXXXXXXY | 0.0017 |
| 75 | 15 | X | 105 | TTDLEAYF | 126 | XTXXXXXF | |
| 85 | 17 | POL | 798 | TTGRTSLY | 127 | XTXXXXXY | |
| 80 | 16 | NUC | 16 | TVQASKLCLGW | 128 | XVXXXXXXXXW | |
| 75 | 15 | ENV | 352 | TVWLSVIW | 129 | XVXXXXXW | |
| 85 | 17 | POL | 741 | VLSRKYTSF | 130 | XLXXXXXXF | |
| 85 | 17 | POL | 741 | VLSRKYTSFPW | 131 | XLXXXXXXXXW | |
| 85 | 17 | POL | 740 | VVLSRKYTSF | 132 | XVXXXXXXXF | |
| 80 | 16 | POL | 759 | WILRGTSF | 133 | XIXXXXXF | |
| 80 | 18 | POL | 759 | WILRGTSFVY | 134 | XIXXXXXXXY | 0.0023 |
| 95 | 19 | NUC | 125 | WIRTPPAY | 135 | XIXXXXXY | |
| 80 | 16 | POL | 751 | WLLGCAANW | 136 | XLXXXXXXW | |
| 95 | 19 | POL | 414 | WLSLDVSAAF | 137 | XLXXXXXXXF | |
| 95 | 19 | POL | 414 | WLSLDVSAAFY | 138 | XLXXXXXXXXY | |
| 100 | 20 | ENV | 335 | WLSLLVPF | 139 | XLXXXXXF | |
| 100 | 20 | ENV | 335 | WLSLLVPFVQW | 140 | XLXXXXXXXXW | |
| 85 | 17 | NUC | 26 | WLWGMDIDPY | 141 | XLXXXXXXXY | 0.0810 |
| 95 | 19 | ENV | 237 | WMCLRRFIF | 142 | XMXXXXXXXF | |
| 85 | 17 | ENV | 359 | WMMWYWGPS | 143 | XMXXXXXXXXY | |
| 100 | 20 | POL | 52 | WTHKVGNF | 144 | XTXXXXXF | |
| 100 | 20 | POL | 122 | YLPLDKGIKPY | 145 | XLXXXXXXXXY | |
| 90 | 18 | NUC | 118 | YLVSFGVW | 146 | XLXXXXXW | |
| 80 | 16 | POL | 493 | YSHPIILGF | 147 | XSXXXXXXF | |
| 85 | 17 | POL | 580 | YSLNFMGY | 148 | XSXXXXXY | |

TABLE VIII

HBV A02 SUPER MOTIF (With binding information)

| | | | | | | | | | | | |
|---|---|---|---|---|---|---|---|---|---|---|---|
| | | | | | SEQ ID | | | | | | |
| Conservancy | Frequency | Protein | Position | Sequence | NO: | AA | A*0201 | A*0202 | A*0203 | A*0206 | A*6602 |

| | | | | | | | | | | | |
|---|---|---|---|---|---|---|---|---|---|---|---|
| 85 | 17 | POL | 721 | AACFARSRSGA | 149 | 11 | | | | | |
| 85 | 17 | POL | 431 | AAMPHLLV | 150 | 8 | | | | | |
| 80 | 16 | POL | 758 | AANWILRGT | 151 | 9 | | | | | |
| 95 | 19 | POL | 632 | AAPFTQCGYPA | 152 | 11 | | | | | |
| 95 | 19 | POL | 521 | AICSVVRRA | 153 | 9 | 0.0001 | | | | |
| 90 | 18 | NUC | 58 | AILCWGEL | 154 | 8 | | | | | |
| 90 | 18 | NUC | 58 | AILCWGELM | 155 | 9 | | | | | |
| 95 | 19 | POL | 642 | ALMPLYACI | 1 56 | 9 | 0.5000 | 0.0340 | 3.3000 | 0.2500 | 0.0470 |
| 80 | 16 | ENV | 108 | AMQWNSTT | 157 | 8 | | | | | |
| 75 | 15 | X | 102 | AMSTTDLEA | 158 | 9 | 0.0013 | | | | |
| 95 | 19 | POL | 516 | AQFTSAICSV | 159 | 10 | | | | | |
| 95 | 19 | POL | 516 | AQFTSAICSVV | 160 | 11 | | | | | |
| 95 | 19 | POL | 690 | ATPTGWGL | 161 | 8 | | | | | |
| 80 | 16 | POL | 690 | ATPTGWGLA | 162 | 9 | | | | | |
| 75 | 15 | POL | 690 | ATPTGWGLAI | 163 | 10 | | | | | |
| 95 | 19 | POL | 397 | AVPNLQSL | 164 | 8 | | | | | |
| 95 | 19 | POL | 397 | AVPNLQSLT | 165 | 9 | 0.0001 | | | | |
| 95 | 19 | POL | 397 | AVPNLQSLTNL | 166 | 11 | | | | | |
| 80 | 16 | POL | 755 | CAANWILRGT | 167 | 10 | | | | | |
| 95 | 19 | X | 61 | CAFSSAGPCA | 168 | 10 | 0.0001 | | | | |
| 95 | 19 | X | 61 | CAFSSAGPCAL | 169 | 11 | | | | | |
| 90 | 18 | X | 69 | CALRFTSA | 170 | 8 | | | | | |
| 100 | 20 | ENV | 312 | CIPIPSSWA | 171 | 9 | 0.0010 | | | | |
| 80 | 16 | ENV | 312 | CIPIPSSWAFA | 172 | 11 | | | | | |
| 90 | 18 | POL | 533 | CLAFSYMDDV | 173 | 10 | 0.0008 | | | | |
| 90 | 18 | POL | 533 | CLAFSYMDDVV | 174 | 11 | | | | | |
| 85 | 17 | NUC | 23 | CLGWLWGM | 175 | 8 | | | | | |
| 85 | 17 | NUC | 23 | CLGWLWGMDI | 176 | 10 | 0.0093 | | | | |
| 100 | 20 | ENV | 253 | CLIFLLVL | 177 | 8 | 0.0002 | | | | |
| 100 | 20 | ENV | 253 | CLIFLLVLL | 178 | 9 | 0.0006 | | | | |
| 95 | 19 | ENV | 239 | CLRRFIIFL | 179 | 9 | 0.0002 | | | | |
| 75 | 15 | ENV | 239 | CLRRFIIFLFI | 180 | 11 | 0.0004 | | | | |
| 90 | 18 | NUC | 107 | CLTFGRET | 181 | 8 | | | | | |
| 90 | 18 | NUC | 107 | CLTFGRETV | 182 | 9 | 0.0001 | | | | |
| 80 | 16 | X | 7 | CQLDPARDV | 183 | 9 | | | | | |
| 80 | 16 | X | 7 | CQLDPARDVL | 184 | 10 | | | | | |
| 85 | 17 | POL | 622 | CQRIVGLL | 185 | 8 | | | | | |
| 85 | 17 | POL | 622 | CQRIVGLLGFA | 186 | 11 | | | | | |
| 95 | 19 | POL | 684 | CQVFADAT | 187 | 8 | | | | | |
| 95 | 19 | POL | 684 | CQVFADATPT | 188 | 10 | | | | | |
| 100 | 20 | ENV | 310 | CTCIPIPSSWA | 189 | 11 | | | | | |
| 95 | 19 | POL | 689 | DATPTGWGL | 190 | 9 | 0.0001 | | | | |
| 80 | 16 | POL | 689 | DATPTGWGLA | 191 | 10 | | | | | |
| 75 | 15 | POL | 689 | DATPTGWGLAI | 192 | 11 | | | | | |
| 90 | 18 | NUC | 31 | DIDPYKEFGA | 193 | 10 | | | | | |
| 85 | 17 | NUC | 29 | DLLDTASA | 194 | 8 | | | | | |
| 85 | 17 | NUC | 29 | DLLDTASAL | 195 | 9 | 0.0001 | | | | |
| 95 | 19 | POL | 40 | DLNLGNLNV | 196 | 9 | 0.0004 | | | | |
| 95 | 19 | POL | 40 | DLNLGNLNVSI | 197 | 11 | | | | | |
| 80 | 16 | NUC | 32 | DTASALYREA | 198 | 10 | | | | | |
| 80 | 16 | NUC | 32 | DTASALYREAL | 199 | 11 | | | | | |
| 95 | 19 | X | 14 | DVLCLRPV | 200 | 8 | | | | | |
| 95 | 19 | X | 14 | DVLCLRPVGA | 201 | 10 | 0.0001 | | | | |
| 90 | 18 | POL | 541 | DVVLGAKSV | 202 | 9 | 0.0003 | | | | |
| 100 | 20 | POL | 17 | EAGPLEEEL | 203 | 9 | 0.0001 | | | | |
| 80 | 16 | X | 122 | ELGEERL | 204 | 8 | | | | | |
| 90 | 18 | POL | 718 | ELLAACFA | 205 | 8 | | | | | |
| 75 | 15 | NUC | 142 | ETVLEYLV | 206 | 8 | | | | | |
| 95 | 19 | POL | 687 | FADATPTGWGL | 207 | 11 | | | | | |
| 85 | 17 | POL | 724 | FARSRSGA | 208 | 8 | | | | | |
| 80 | 16 | POL | 821 | FASPLHVA | 209 | 8 | | | | | |
| 95 | 19 | POL | 396 | FAVPNLQSL | 210 | 9 | | | | | |
| 95 | 19 | POL | 396 | FAVPNLQSLT | 211 | 10 | 0.0003 | | | | |
| 80 | 16 | ENV | 243 | FIIFLFIL | 212 | 8 | 0.0006 | | | | |
| 80 | 16 | ENV | 243 | FIIFLFILL | 213 | 9 | 0.0002 | | | | |
| 80 | 18 | ENV | 243 | FIIFLFILLL | 214 | 10 | 0.0012 | | | | |
| 80 | 16 | ENV | 248 | FILLLCLI | 215 | 8 | 0.0003 | | | | |
| 80 | 16 | ENV | 248 | FILLLCLIFL | 216 | 10 | 0.0280 | | | | |
| 80 | 16 | ENV | 248 | FILLLCLIFLL | 217 | 11 | 0.0010 | | | | |
| 80 | 16 | ENV | 246 | FLFILLLCL | 218 | 9 | 0.0002 | | | | |
| 80 | 16 | ENV | 246 | FLFILLLCLI | 219 | 10 | 0.0013 | | | | |
| 75 | 15 | ENV | 171 | FLGPLLVL | 220 | 8 | | | | | |
| 75 | 15 | ENV | 171 | FLGPLLVLQA | 221 | 10 | 0.0190 | | | | |
| 95 | 19 | POL | 513 | FLLAQFTSA | 222 | 9 | 0.2400 | | | | |
| 95 | 19 | POL | 513 | FLLAQFTSAI | 223 | 10 | 0.2100 | 0.0320 | 7.0000 | 0.1100 | 0.0880 |
| 95 | 19 | POL | 562 | FLLSLGIHL | 224 | 9 | 0.6500 | 0.0010 | 0.0100 | 0.1100 | 0.0035 |
| 80 | 16 | ENV | 183 | FLLTRILT | 225 | 8 | | | | | |
| 80 | 16 | ENV | 183 | FLLTRILTI | 226 | 9 | 0.5100 | 0.0430 | 8.0000 | 0.2000 | 0.0010 |
| 95 | 19 | ENV | 256 | FLLVLLDYQGM | 227 | 11 | | | | | |
| 100 | 20 | POL | 363 | FLVDKNPHNT | 228 | 10 | 0.0012 | | | | |
| 95 | 19 | POL | 656 | FTFSPTYKA | 229 | 9 | 0.0056 | 0.0150 | 0.0031 | 0.8000 | 7.3000 |
| 95 | 19 | POL | 656 | FTFSPTYKAFL | 230 | 11 | | | | | |
| 95 | 19 | POL | 59 | FTGLYSST | 231 | 8 | | | | | |
| 90 | 18 | POL | 59 | FTGLYSSTV | 232 | 9 | 0.0005 | | | | |
| 95 | 19 | POL | 635 | FTQCGYPA | 233 | 8 | | | | | |
| 95 | 19 | POL | 835 | FTQCGYPAL | 234 | 9 | 0.0009 | | | | |
| 95 | 19 | POL | 635 | FTQCGYPALM | 235 | 10 | 0.0024 | | | | |
| 95 | 19 | POL | 518 | FTSAICSV | 236 | 8 | | | | | |
| 95 | 19 | POL | 518 | FTSAICSVV | 237 | 9 | 0.0090 | | | | |
| 95 | 19 | ENV | 346 | FVGLSPTV | 238 | 8 | | | | | |
| 95 | 19 | ENV | 346 | FVGLSPTVWL | 239 | 10 | 0.0008 | | | | |
| 90 | 18 | X | 132 | FVLGGCRHKL | 240 | 10 | 0.0030 | | | | |
| 90 | 18 | X | 132 | FVLGGCRHKLV | 241 | 11 | | | | | |
| 95 | 19 | ENV | 342 | FVQWFVGL | 242 | 8 | | | | | |
| 95 | 19 | ENV | 342 | FVQWFVGLSPT | 243 | 11 | | | | | |
| 90 | 18 | POL | 768 | FVYVPSAL | 244 | 8 | | | | | |
| 90 | 18 | POL | 766 | FVYVPSALNPA | 245 | 11 | | | | | |
| 95 | 19 | X | 50 | GAHLSLRGL | 246 | 9 | 0.0001 | | | | |
| 90 | 18 | X | 50 | GAHLSLRGLPV | 247 | 11 | | | | | |
| 85 | 17 | POL | 545 | GAKSVQHL | 248 | 8 | | | | | |
| 85 | 17 | POL | 545 | GAKSVQHLESL | 249 | 11 | | | | | |
| 75 | 15 | POL | 567 | GIHLNPNKT | 250 | 9 | | | | | |
| 90 | 18 | POL | 155 | GILYKRET | 251 | 8 | | | | | |
| 90 | 18 | POL | 155 | GILYKRETT | 252 | 9 | | | | | |
| 85 | 17 | POL | 682 | GLCQVFADA | 253 | 9 | 0.0024 | | | | |
| 85 | 17 | POL | 682 | GLCQVFADAT | 254 | 10 | | | | | |
| 95 | 19 | POL | 627 | GLLGFAAPFT | 255 | 10 | 0.0049 | | | | |
| 85 | 17 | ENV | 62 | GLLGWSPQA | 256 | 9 | 0.4000 | 0.0003 | 0.0350 | 0.2800 | 0.0005 |
| 95 | 19 | X | 57 | GLPVCAFSSA | 257 | 10 | 0.0008 | | | | |
| 95 | 19 | POL | 509 | GLSPFLLA | 258 | 8 | | | | | |
| 95 | 19 | POL | 509 | GLSPFLLAQFT | 259 | 11 | | | | | |
| 100 | 20 | ENV | 348 | GLSPTVWL | 260 | 8 | 0.0036 | | | | |
| 75 | 15 | ENV | 348 | GLSPTVWLSV | 261 | 10 | 0.2800 | | | | |
| 75 | 15 | ENV | 348 | GLSPTVWLSVI | 262 | 11 | 0.0036 | | | | |
| 90 | 18 | ENV | 265 | GMLPVCPL | 263 | 8 | | | | | |
| 90 | 18 | POL | 735 | GTDNSVVL | 264 | 8 | | | | | |
| 75 | 15 | ENV | 13 | GTNLSVPNPL | 265 | 10 | | | | | |
| 80 | 16 | POL | 763 | GTSFVYVPSA | 266 | 10 | | | | | |
| 80 | 16 | POL | 763 | GTSFVYVPSAL | 267 | 11 | | | | | |
| 80 | 16 | POL | 507 | GVGLSPFL | 268 | 8 | | | | | |
| 80 | 16 | POL | 507 | GVGLSPFLL | 269 | 9 | 0.0002 | | | | |
| 80 | 18 | POL | 507 | GVGLSPFLLA | 270 | 10 | | | | | |
| 95 | 19 | NUC | 123 | GVWIRTPPA | 271 | 9 | 0.0030 | | | | |
| 90 | 18 | NUC | 104 | HISCLTFGRET | 272 | 11 | | | | | |
| 80 | 16 | POL | 435 | HLLVGSSGL | 273 | 9 | 0.0031 | | | | |
| 90 | 18 | X | 52 | HLSLRGLPV | 274 | 9 | 0.0014 | | | | |
| 90 | 18 | X | 52 | HLSLRGLPVCA | 275 | 11 | | | | | |
| 80 | 16 | POL | 491 | HLYSHPII | 276 | 8 | | | | | |
| 80 | 16 | POL | 491 | HLYSHPIIL | 277 | 9 | 0.2200 | 0.0003 | 0.9300 | 0.1700 | 0.0530 |
| 85 | 17 | POL | 715 | HTAELLAA | 278 | 8 | | | | | |
| 85 | 17 | POL | 715 | HTAELLAACFA | 279 | 11 | | | | | |
| 100 | 20 | NUC | 52 | HTALRQAI | 280 | 8 | | | | | |
| 95 | 19 | NUC | 52 | HTALRQAIL | 281 | 9 | 0.0001 | | | | |
| 100 | 20 | POL | 149 | HTLWKAGI | 282 | 8 | | | | | |
| 100 | 20 | POL | 149 | HTLWKAGIL | 283 | 9 | 0.0001 | | | | |
| 80 | 16 | ENV | 244 | IIFIFILL | 284 | 8 | 0.0004 | | | | |
| 80 | 16 | ENV | 244 | IIFIFILIL | 285 | 9 | 0.0002 | | | | |
| 80 | 16 | ENV | 244 | IIFLFILILCL | 286 | 11 | 0.0002 | | | | |
| 80 | 16 | POL | 497 | IILGFRKI | 287 | 8 | | | | | |
| 80 | 18 | POL | 497 | IILGFRKIPM | 288 | 10 | | | | | |
| 90 | 18 | NUC | 59 | ILCWGELM | 289 | 8 | | | | | |
| 80 | 16 | POL | 498 | ILGFRKIPM | 290 | 9 | 0.0002 | | | | |
| 100 | 20 | ENV | 249 | ILLLCLIFI | 291 | 9 | 0.0015 | | | | |
| 100 | 20 | ENV | 249 | ILLLCLIFIL | 292 | 10 | 0.0190 | 0.0001 | 0.0002 | 0.1300 | 0.0015 |
| 100 | 20 | ENV | 249 | ILLLCLIFLLV | 293 | 11 | 0.0056 | | | | |
| 80 | 16 | POL | 760 | ILRGTSFV | 294 | 8 | | | | | |
| 80 | 16 | POL | 760 | ILRGTSFVYV | 295 | 10 | 0.0160 | | | | |
| 100 | 20 | NUC | 139 | ILSTLPET | 296 | 8 | | | | | |
| 100 | 20 | NUC | 139 | ILSTLPETT | 297 | 9 | 0.0001 | | | | |
| 100 | 20 | NUC | 139 | ILSTLPETTV | 298 | 10 | 0.0210 | 0.0085 | 0.0770 | 0.3100 | 0.0067 |
| 100 | 20 | NUC | 139 | ILSTLPETTVV | 299 | 11 | | | | | |
| 95 | 19 | ENV | 188 | ILTIPQSL | 300 | 8 | | | | | |
| 90 | 18 | POL | 156 | ILYKRETT | 301 | 8 | | | | | |
| 90 | 18 | POL | 625 | IVGLLGFA | 302 | 8 | | | | | |
| 90 | 18 | POL | 625 | IVGLLGFAA | 303 | 9 | 0.0009 | | | | |
| 90 | 18 | POL | 153 | KAGILYKRET | 304 | 10 | | | | | |
| 90 | 18 | POL | 153 | KAGILYKRETT | 305 | 11 | | | | | |
| 80 | 16 | POL | 503 | KIPMGVGL | 306 | 8 | | | | | |
| 85 | 17 | NUC | 21 | KLCLGWLWGM | 307 | 10 | 0.0001 | | | | |
| 95 | 19 | POL | 489 | KLHLYSHPI | 308 | 9 | 0.0690 | 0.0340 | 2.7000 | 0.5900 | 0.0015 |
| 80 | 16 | POL | 489 | KLHLYSHPII | 309 | 10 | | | | | |
| 80 | 16 | POL | 489 | KLHLYSHPIIL | 310 | 11 | | | | | |
| 80 | 16 | POL | 610 | KLPVNRPI | 311 | 8 | | | | | |
| 95 | 19 | POL | 653 | KQAFTFSPT | 312 | 9 | | | | | |
| 95 | 19 | POL | 574 | KTKRWGYSL | 313 | 9 | 0.0001 | | | | |
| 85 | 17 | POL | 620 | KVCQRIVGL | 314 | 9 | 0.0003 | | | | |
| 85 | 17 | POL | 620 | KVCQRIVGLL | 315 | 10 | 0.0001 | | | | |
| 95 | 19 | POL | 55 | KVGNFTGL | 316 | 8 | | | | | |
| 85 | 17 | X | 91 | KVLHKRTL | 317 | 8 | | | | | |
| 85 | 17 | X | 91 | KVLHKRTLGL | 318 | 10 | 0.0004 | | | | |
| 90 | 18 | POL | 534 | LAFSYMDDV | 319 | 9 | 0.0002 | | | | |
| 90 | 18 | POL | 534 | LAFSYMDDVV | 320 | 10 | 0.0003 | | | | |
| 90 | 18 | POL | 534 | LAFSYMDDVVL | 321 | 11 | | | | | |
| 95 | 19 | POL | 515 | LAQFTSAI | 322 | 8 | | | | | |
| 95 | 19 | POL | 515 | LAQFTSAICSV | 323 | 11 | | | | | |
| 100 | 20 | ENV | 254 | LIFLLVLL | 324 | 8 | 0.0025 | | | | |
| 95 | 19 | POL | 514 | LLAQFTSA | 325 | 8 | | | | | |
| 95 | 19 | POL | 514 | LLAQFTSAI | 326 | 9 | 0.1000 | 0.2700 | 3.7000 | 0.2600 | 0.7900 |
| 100 | 20 | ENV | 251 | LLCLIFLL | 327 | 8 | 0.0004 | | | | |
| 100 | 20 | ENV | 251 | LLCLIFLLV | 328 | 9 | 0.0048 | | | | |
| 100 | 20 | ENV | 251 | LLCLIFLLVL | 329 | 10 | 0.0075 | | | | |
| 100 | 20 | ENV | 251 | LLCLIFLLVLL | 330 | 11 | 0.0013 | | | | |
| 85 | 17 | NUC | 30 | LLDTASAL | 331 | 8 | | | | | |
| 95 | 19 | ENV | 260 | LLDYQGML | 332 | 8 | 0.0004 | | | | |
| 90 | 18 | ENV | 260 | LLDYQGMLPV | 333 | 10 | 0.0980 | 0.0001 | 0.0200 | 0.6700 | 0.0009 |
| 80 | 16 | POL | 752 | LLGCAANWI | 334 | 9 | 0.0011 | | | | |
| 80 | 16 | POL | 752 | LLGCAANWIL | 335 | 10 | 0.0140 | | | | |
| 95 | 19 | POL | 628 | LLGFAAPFT | 336 | 9 | 0.0008 | | | | |
| 85 | 17 | ENV | 63 | LLGWSPQA | 337 | 8 | | | | | |
| 75 | 15 | ENV | 63 | LLGWSPQAQGI | 338 | 11 | | | | | |
| 100 | 20 | ENV | 250 | LLLCLIFL | 339 | 8 | 0.0006 | | | | |
| 100 | 20 | ENV | 250 | LLLCLIFLL | 340 | 9 | 0.0065 | | | | |
| 100 | 20 | ENV | 250 | LLLCLIFLLV | 341 | 10 | 0.0036 | | | | |
| 100 | 20 | ENV | 250 | LLLCLIFLLVL | 342 | 11 | 0.0005 | | | | |
| 100 | 20 | ENV | 378 | LLPIFFCL | 343 | 8 | 0.0055 | | | | |
| 100 | 20 | ENV | 378 | LLPIFFCLWV | 344 | 10 | 0.0320 | 0.0008 | 0.0150 | 0.8000 | 0.0005 |
| 95 | 19 | POL | 563 | LLSLGIHL | 345 | 8 | | | | | |
| 90 | 18 | POL | 407 | LLSSNLSWL | 346 | 9 | 0.0110 | 0.0780 | 3.9000 | 0.2700 | 0.0100 |
| 90 | 18 | POL | 407 | LLSSNLSWLSL | 347 | 11 | | | | | |
| 80 | 16 | ENV | 184 | LLTRILTI | 348 | 8 | 0.0026 | | | | |
| 80 | 16 | POL | 436 | LLVGSSGL | 349 | 8 | | | | | |
| 95 | 19 | ENV | 257 | LLVLLDYQGM | 350 | 10 | 0.0050 | | | | |
| 95 | 19 | ENV | 257 | LLVLLDYQGML | 351 | 11 | | | | | |
| 90 | 18 | ENV | 175 | LLVLQAGFFL | 352 | 10 | 0.0310 | 0.0037 | 0.0045 | 0.1500 | 0.0110 |
| 90 | 18 | ENV | 175 | LLVLQAGFFLL | 353 | 11 | 0.0074 | | | | |
| 95 | 19 | ENV | 338 | LLVPFVQWFV | 354 | 10 | 0.6700 | 0.3800 | 1.7000 | 0.2900 | 0.1400 |
| 90 | 18 | NUC | 100 | LLWFHISCL | 355 | 9 | 0.0130 | 0.0002 | 0.0420 | 0.3100 | 0.0098 |
| 85 | 17 | NUC | 100 | LLWFHISCLT | 356 | 10 | | | | | |
| 95 | 19 | POL | 643 | LMPLYACI | 357 | 8 | | | | | |
| 95 | 19 | ENV | 178 | LQAGFFLL | 358 | 8 | | | | | |
| 95 | 19 | ENV | 178 | LQAGFFLLT | 359 | 9 | | | | | |
| 80 | 16 | ENV | 178 | LQAGFFLLTRI | 360 | 11 | | | | | |
| 100 | 20 | POL | 401 | LQSLTNLL | 361 | 8 | | | | | |
| 95 | 19 | NUC | 108 | LTFGRETV | 362 | 8 | | | | | |
| 75 | 15 | NUC | 137 | LTFGRETVL | 363 | 9 | | | | | |
| 90 | 18 | POL | 404 | LTNLLSSNL | 364 | 9 | | | | | |
| 80 | 18 | ENV | 185 | LTRILTIPQSL | 365 | 11 | | | | | |
| 85 | 17 | POL | 99 | LTVNEKRRL | 366 | 9 | | | | | |
| 100 | 20 | POL | 364 | LVDKNPHNT | 367 | 9 | 0.0001 | | | | |
| 95 | 19 | ENV | 258 | LVLLDYQGM | 368 | 9 | 0.0001 | | | | |
| 95 | 19 | ENV | 258 | LVLLDYQGML | 369 | 10 | 0.0001 | | | | |
| 90 | 18 | ENV | 176 | LVLQAGFFL | 370 | 9 | 0.0096 | | | | |
| 90 | 18 | ENV | 176 | LVLQAGFFLL | 371 | 10 | 0.0022 | | | | |
| 90 | 18 | ENV | 176 | LVLQAGFFLLT | 372 | 11 | | | | | |
| 95 | 19 | ENV | 339 | LVPFVQWFV | 373 | 9 | 0.0420 | 0.0150 | 0.0048 | 0.7900 | 2.8000 |
| 95 | 19 | ENV | 339 | LVPFVQWFVGL | 374 | 11 | | | | | |
| 90 | 18 | NUC | 119 | LVSFGVWI | 375 | 8 | 0.0004 | | | | |
| 90 | 18 | NUC | 119 | LVSFGVWIRT | 376 | 10 | | | | | |
| 85 | 17 | ENV | 360 | MMWYWGPSL | 377 | 9 | 0.6400 | | | | |
| 75 | 15 | NUC | 1 | MQLFHLCL | 378 | 8 | | | | | |
| 100 | 20 | NUC | 136 | NAPILSTL | 379 | 8 | | | | | |
| 100 | 20 | NUC | 136 | NAPILSTLPET | 380 | 11 | | | | | |
| 95 | 19 | POL | 42 | NLGNLNVSI | 381 | 9 | 0.0047 | | | | |
| 90 | 18 | POL | 406 | NLLSSNLSWL | 382 | 10 | 0.0016 | | | | |
| 95 | 19 | POL | 45 | NLNVSIPWT | 383 | 9 | 0.0005 | | | | |
| 100 | 20 | POL | 400 | NLQSLTNL | 384 | 8 | | | | | |
| 100 | 20 | POL | 400 | NLQSLTNLL | 385 | 9 | 0.0047 | | | | |
| 75 | 15 | ENV | 15 | NLSVPNPL | 386 | 8 | | | | | |
| 90 | 18 | POL | 411 | NLSWLSLDV | 387 | 9 | 0.0650 | 0.0051 | 0.6400 | 0.1600 | 0.0990 |
| 90 | 18 | POL | 411 | NLSWLSLDVSA | 388 | 11 | | | | | |
| 100 | 20 | POL | 47 | NVSIPWTHKV | 389 | 10 | 0.0001 | | | | |
| 100 | 20 | POL | 430 | PAAMPHLL | 390 | 8 | | | | | |
| 85 | 17 | POL | 430 | PAAMPHLLV | 391 | 9 | | | | | |
| 90 | 18 | POL | 775 | PADDPSRGRL | 392 | 10 | | | | | |
| 90 | 18 | ENV | 131 | PAGGSSSGT | 393 | 9 | | | | | |
| 90 | 18 | ENV | 131 | PAGGSSSGTV | 394 | 10 | | | | | |
| 95 | 19 | POL | 641 | PALMPLYA | 395 | 8 | | | | | |
| 95 | 19 | POL | 641 | PALMPLYACI | 396 | 10 | 0.0001 | | | | |
| 75 | 15 | X | 145 | PAPCNFFT | 397 | 8 | | | | | |
| 75 | 15 | X | 145 | PAPCNFFTSA | 398 | 10 | | | | | |
| 80 | 16 | X | 11 | PARDVLCL | 399 | 8 | | | | | |
| 75 | 15 | X | 11 | PARDVLCLRPV | 400 | 11 | | | | | |
| 90 | 18 | POL | 355 | PARVTGGV | 401 | 8 | | | | | |
| 90 | 18 | POL | 355 | PARVTGGVFL | 402 | 10 | | | | | |
| 90 | 18 | POL | 355 | PARVTGGVFLV | 403 | 11 | | | | | |
| 95 | 19 | NUC | 130 | PAYRPPNA | 404 | 8 | | | | | |
| 95 | 19 | NUC | 130 | PAYRPPNAPI | 405 | 10 | 0.0001 | | | | |
| 95 | 19 | NUC | 130 | PAYRPPNAPIL | 406 | 11 | | | | | |
| 85 | 17 | POL | 616 | PIDWKVCQRI | 407 | 10 | 0.0001 | | | | |
| 85 | 17 | POL | 616 | PIDWKVCQRIV | 408 | 11 | | | | | |
| 100 | 20 | ENV | 380 | PIFFCLWV | 409 | 8 | | | | | |
| 100 | 20 | ENV | 380 | PIFFCLWVYI | 410 | 10 | 0.0004 | | | | |
| 85 | 17 | POL | 713 | PIHTAELL | 411 | 8 | | | | | |
| 85 | 17 | POL | 713 | PIHTAELLA | 412 | 9 | | | | | |
| 85 | 17 | POL | 713 | PIHTAELLAA | 413 | 10 | | | | | |
| 80 | 16 | POL | 496 | PIILGFRKI | 414 | 9 | 0.0001 | | | | |
| 80 | 18 | POL | 496 | PIILGFRKIPM | 415 | 11 | | | | | |
| 100 | 20 | NUC | 138 | PILSTIPET | 416 | 9 | 0.0001 | | | | |
| 100 | 20 | NUC | 138 | PILSTLPETT | 417 | 10 | 0.0001 | | | | |
| 100 | 20 | NUC | 138 | PILSTLPETTV | 418 | 11 | 0.0001 | | | | |
| 80 | 16 | ENV | 314 | PIPSSWAFA | 419 | 9 | | | | | |
| 95 | 19 | POL | 20 | PLEEELPRL | 420 | 9 | 0.0003 | | | | |
| 90 | 18 | POL | 20 | PLEEELPRLA | 421 | 10 | 0.0001 | | | | |
| 95 | 19 | ENV | 10 | PLGFFPDHQL | 422 | 10 | 0.0002 | | | | |
| 100 | 20 | POL | 427 | PLHPAAMPHL | 423 | 10 | 0.0001 | | | | |
| 100 | 20 | POL | 427 | PLHPAAMPHLL | 424 | 11 | | | | | |
| 100 | 20 | ENV | 377 | PLLPIFFCL | 425 | 9 | 0.0650 | 0.0001 | 0.0018 | 0.1100 | 0.0047 |
| 100 | 20 | ENV | 377 | PLLPIFFCLWV | 426 | 11 | | | | | |
| 90 | 18 | ENV | 174 | PLLVLQAGFFL | 427 | 11 | 0.0008 | | | | |
| 80 | 16 | POL | 711 | PLPIHTAEL | 428 | 9 | 0.0004 | | | | |
| 80 | 16 | POL | 711 | PLPIHTAELL | 429 | 10 | 0.0001 | | | | |
| 80 | 16 | POL | 711 | PLPIHTAELLA | 430 | 11 | | | | | |
| 75 | 15 | POL | 2 | PLSYQHFRKL | 431 | 10 | 0.0001 | | | | |
| 75 | 15 | POL | 2 | PLSYQHFRKLL | 432 | 11 | | | | | |
| 85 | 17 | POL | 98 | PLTVNEKRRL | 433 | 10 | 0.0001 | | | | |
| 80 | 16 | POL | 505 | PMGVGLSPFL | 434 | 10 | 0.0001 | | | | |
| 80 | 16 | POL | 505 | PMGVGLSPFLL | 435 | 11 | | | | | |
| 95 | 19 | ENV | 106 | PQAMQWNST | 436 | 9 | | | | | |
| 80 | 16 | ENV | 106 | PQAMQWNSTT | 437 | 10 | | | | | |
| 90 | 18 | ENV | 192 | PQSLDSWWT | 438 | 9 | | | | | |
| 90 | 18 | ENV | 192 | PQSLDSWWTSL | 439 | 11 | | | | | |
| 75 | 15 | POL | 692 | PTGWGLAI | 440 | 8 | | | | | |
| 80 | 16 | ENV | 219 | PTSNHSPT | 441 | 8 | | | | | |
| 85 | 17 | POL | 797 | PTTGRTSL | 442 | 8 | | | | | |
| 85 | 17 | POL | 797 | PTTGRTSLYA | 443 | 10 | | | | | |
| 80 | 16 | NUC | 15 | PTVQASKL | 444 | 8 | | | | | |
| 80 | 16 | NUC | 15 | PTVQASKLCL | 445 | 10 | | | | | |
| 75 | 15 | ENV | 351 | PTVWLSVI | 446 | 8 | | | | | |
| 75 | 15 | ENV | 351 | PTVWLSVIWM | 447 | 10 | | | | | |
| 95 | 19 | X | 59 | PVCAFSSA | 448 | 8 | | | | | |
| 85 | 17 | POL | 612 | PVNRPIDWKV | 449 | 10 | 0.0002 | | | | |
| 95 | 19 | POL | 654 | QAFTFSPT | 450 | 8 | | | | | |
| 95 | 19 | POL | 654 | QAFTFSPTYKA | 451 | 11 | | | | | |
| 95 | 19 | ENV | 179 | QAGFFLLT | 452 | 8 | | | | | |
| 80 | 16 | ENV | 179 | QAGFFLLTRI | 453 | 10 | | | | | |
| 80 | 16 | ENV | 179 | QAGFFLLTRIL | 454 | 11 | | | | | |
| 90 | 18 | NUC | 57 | QAILCWGEL | 455 | 9 | | | | | |
| 90 | 18 | NUC | 57 | QAILCWGELM | 456 | 10 | | | | | |
| 95 | 19 | ENV | 107 | QAMQWNST | 457 | 8 | | | | | |
| 80 | 16 | ENV | 107 | QAMQWNSTT | 458 | 9 | | | | | |
| 80 | 16 | NUC | 18 | QASKLCLGWL | 459 | 10 | | | | | |
| 80 | 16 | X | 8 | QLDPARDV | 460 | 8 | 0.0001 | | | | |
| 80 | 16 | X | 8 | QLDPARDVL | 461 | 9 | 0.0001 | | | | |
| 80 | 16 | X | 8 | QLDPARDVLCL | 462 | 11 | 0.0001 | | | | |
| 90 | 18 | NUC | 99 | QLLWFHISCL | 463 | 10 | 0.0060 | | | | |
| 85 | 17 | NUC | 99 | QLLWFHISCLT | 464 | 11 | | | | | |
| 95 | 19 | POL | 685 | QVFADATPT | 465 | 9 | 0.0001 | | | | |
| 95 | 19 | POL | 528 | RAFPHCLA | 466 | 8 | | | | | |
| 80 | 16 | ENV | 187 | RILTIPQSL | 467 | 9 | 0.0010 | | | | |
| 90 | 18 | POL | 624 | RIVGLLGFA | 468 | 9 | | | | | |
| 90 | 18 | POL | 624 | RIVGLLGFAA | 469 | 10 | | | | | |
| 75 | 15 | POL | 106 | RLKLIMPA | 470 | 8 | | | | | |
| 90 | 18 | NUC | 56 | RQAILCWGEL | 471 | 10 | | | | | |
| 90 | 18 | NUC | 56 | RQAILCWGELM | 472 | 11 | | | | | |
| 90 | 18 | NUC | 98 | RQLLWFHI | 473 | 8 | | | | | |
| 90 | 18 | NUC | 98 | RQLLWFHISCL | 474 | 11 | | | | | |
| 85 | 17 | ENV | 88 | RQSGRQPT | 475 | 8 | | | | | |
| 90 | 18 | POL | 353 | RTPARVTGGV | 476 | 10 | | | | | |
| 95 | 19 | NUC | 127 | RTPPAYRPPNA | 477 | 11 | | | | | |
| 95 | 19 | POL | 36 | RVAEDLNL | 478 | 8 | | | | | |
| 90 | 18 | POL | 36 | RVAEDLNLGNL | 479 | 11 | | | | | |
| 80 | 16 | POL | 818 | RVHFASPL | 480 | 8 | | | | | |
| 75 | 15 | POL | 818 | RVHFASPLHV | 481 | 10 | 0.0001 | | | | |
| 75 | 15 | POL | 818 | RVHFASPLHVA | 482 | 11 | | | | | |
| 100 | 20 | POL | 357 | RVTGGVFL | 483 | 8 | | | | | |
| 100 | 20 | POL | 357 | RVTGGVFLV | 484 | 9 | 0.0041 | | | | |
| 90 | 18 | X | 65 | SAGPCALRFT | 485 | 10 | | | | | |
| 95 | 19 | POL | 520 | SAICSVVRRA | 486 | 10 | 0.0001 | | | | |
| 90 | 18 | NUC | 35 | SALYREAL | 487 | 8 | | | | | |
| 100 | 20 | POL | 49 | SIPWTHKV | 488 | 8 | | | | | |
| 95 | 19 | ENV | 194 | SLDSWWTSL | 489 | 9 | | | | | |
| 75 | 15 | POL | 565 | SLGIHLNPNKT | 490 | 11 | | | | | |
| 95 | 19 | ENV | 337 | SLLVPFVQWFV | 491 | 11 | | | | | |
| 75 | 15 | POL | 581 | SLNFMGYV | 492 | 8 | | | | | |
| 75 | 15 | POL | 581 | SLNFMGYVI | 493 | 9 | 0.0038 | | | | |
| 95 | 19 | X | 54 | SLRGLPVCA | 494 | 9 | 0.0007 | | | | |
| 90 | 18 | POL | 403 | SLTNLLSSNL | 495 | 10 | 0.0014 | | | | |
| 75 | 15 | ENV | 216 | SQSPTSNHSPT | 496 | 11 | | | | | |
| 75 | 15 | ENV | 280 | STGPCKTCT | 497 | 9 | | | | | |
| 100 | 20 | NUC | 141 | STLPETTV | 498 | 8 | | | | | |
| 100 | 20 | NUC | 141 | STLPETTVV | 499 | 9 | 0.0019 | | | | |
| 80 | 16 | ENV | 85 | STNRQSGRQPT | 500 | 11 | | | | | |
| 85 | 17 | POL | 548 | SVQHLESL | 501 | 8 | | | | | |
| 80 | 16 | ENV | 330 | SVRFSWLSL | 502 | 9 | 0.0001 | | | | |
| 80 | 16 | ENV | 330 | SVRFSWLSLL | 503 | 10 | 0.0004 | | | | |
| 80 | 16 | ENV | 330 | SVRFSWLSLLV | 504 | 11 | | | | | |
| 90 | 18 | POL | 739 | SVVLSRKYT | 505 | 9 | | | | | |
| 95 | 19 | POL | 524 | SVVRRAFPHCL | 506 | 11 | | | | | |
| 85 | 17 | POL | 716 | TAELLAACFA | 507 | 10 | | | | | |
| 95 | 19 | NUC | 53 | TALRQAIL | 508 | 8 | | | | | |
| 80 | 16 | NUC | 33 | TASALYREA | 509 | 9 | | | | | |
| 80 | 16 | NUC | 33 | TASALYREAL | 510 | 10 | | | | | |
| 90 | 18 | ENV | 190 | TIPQSLDSWWT | 511 | 11 | | | | | |
| 100 | 20 | NUC | 142 | TLPETTVV | 512 | 8 | | | | | |
| 100 | 20 | POL | 150 | TLWKAGIL | 513 | 8 | | | | | |
| 95 | 19 | POL | 636 | TQCGYPAL | 514 | 8 | | | | | |
| 95 | 19 | POL | 636 | TQCGYPALM | 515 | 9 | | | | | |
| 95 | 19 | POL | 836 | TQCGYPALMPL | 516 | 11 | | | | | |
| 85 | 17 | POL | 798 | TTGRTSLYA | 517 | 9 | | | | | |
| 75 | 15 | ENV | 278 | TTSTGPCKT | 518 | 9 | | | | | |
| 75 | 15 | ENV | 278 | TTSTGPCKTCT | 519 | 11 | | | | | |
| 85 | 17 | POL | 100 | TVNEKRRL | 520 | 8 | | | | | |
| 80 | 16 | NUC | 16 | TVQASKLCL | 521 | 9 | 0.0002 | | | | |
| 75 | 15 | ENV | 352 | TVWLSVIWM | 522 | 9 | 0.0002 | | | | |
| 95 | 19 | POL | 37 | VAEDLNLGNL | 523 | 10 | 0.0001 | | | | |
| 95 | 19 | X | 15 | VLCLRPVGA | 524 | 9 | 0.0014 | | | | |
| 85 | 17 | POL | 543 | VLGAKSVQHL | 525 | 10 | 0.0001 | | | | |
| 90 | 18 | X | 133 | VLGGCRHKL | 526 | 9 | 0.0009 | | | | |
| 90 | 18 | X | 133 | VLGGCRHKLV | 527 | 10 | 0.0001 | | | | |
| 85 | 17 | X | 92 | VLHKRTLGL | 528 | 9 | 0.0012 | | | | |
| 95 | 19 | ENV | 259 | VLLDYQGM | 529 | 8 | | | | | |
| 95 | 19 | ENV | 259 | VLLDYQGML | 530 | 9 | 0.0440 | 0.0001 | 0.0210 | 0.9000 | 0.0002 |
| 90 | 18 | ENV | 259 | VLLDYQGMLPV | 531 | 11 | 0.5800 | 0.2200 | 4.9000 | 0.3400 | 0.0170 |
| 95 | 19 | ENV | 177 | VLQAGFFL | 532 | 8 | 0.0019 | | | | |
| 95 | 19 | ENV | 177 | VLQAGFFLL | 533 | 9 | 0.0660 | | | | |
| 95 | 19 | ENV | 177 | VLQAGFFLLT | 534 | 10 | 0.0011 | | | | |
| 80 | 16 | NUC | 17 | VQASKLCL | 535 | 8 | | | | | |
| 80 | 16 | NUC | 17 | VQASKLCLGWL | 536 | 11 | | | | | |
| 95 | 19 | ENV | 343 | VQWFVGLSPT | 537 | | | | | | |
| 95 | 19 | ENV | 343 | VQWFVGLSPTV | 538 | 11 | | | | | |
| 100 | 20 | POL | 358 | VTGGVFLV | 539 | 8 | | | | | |
| 90 | 18 | POL | 542 | VVLGAKSV | 540 | 8 | | | | | |
| 80 | 16 | POL | 542 | VVLGAKSVQHL | 541 | 11 | | | | | |
| 90 | 18 | POL | 740 | VVLSRKYT | 542 | 8 | | | | | |
| 95 | 19 | POL | 525 | VVARAFPHCL | 543 | 10 | 0.0003 | | | | |
| 95 | 19 | POL | 525 | VVRRAFPHCIA | 544 | 11 | | | | | |
| 80 | 16 | POL | 759 | WILRGTSFV | 545 | 9 | 0.0270 | | | | |
| 80 | 18 | POL | 759 | WILRGTSFVYV | 546 | 11 | | | | | |
| 80 | 16 | POL | 751 | WLLGCAANWI | 547 | 10 | 0.0053 | | | | |
| 80 | 16 | POL | 751 | WLLGCAANWIL | 548 | 11 | | | | | |
| 100 | 20 | POL | 414 | WLSLDVSA | 549 | 8 | | | | | |
| 95 | 19 | POL | 414 | WLSLDVSAA | 550 | 9 | 0.0059 | | | | |
| 100 | 20 | ENV | 335 | WLSLLVPFV | 551 | 9 | 1.1000 | 0.0380 | 7.2000 | 0.3600 | 0.0310 |
| 95 | 19 | ENV | 237 | WMCLRRFI | 552 | 8 | | | | | |
| 95 | 19 | ENV | 237 | WMCLRRFII | 553 | 9 | 0.0005 | | | | |
| 95 | 19 | ENV | 237 | WMCLRRFIIFL | 554 | 11 | 0.0019 | | | | |
| 85 | 17 | ENV | 359 | WMMWYWGPSL | 555 | 10 | 0.0009 | | | | |
| 100 | 20 | POL | 52 | WTHKVGNFT | 556 | 9 | 0.0001 | | | | |
| 95 | 19 | POL | 52 | WTHKVGNFTGL | 557 | 11 | | | | | |
| 100 | 20 | POL | 147 | YLHTLWKA | 558 | 8 | | | | | |
| 100 | 20 | POL | 147 | YLHTLWKAGI | 559 | 10 | 0.0160 | 0.0005 | 0.5600 | 0.1000 | 0.0320 |
| 100 | 20 | POL | 147 | YLHTLWKAGIL | 560 | 11 | | | | | |
| 100 | 20 | POL | 122 | YLPDKGI | 561 | 8 | | | | | |
| 90 | 18 | NUC | 118 | YLVSFGVWI | 562 | 9 | 0.3800 | | | | |
| 90 | 18 | NUC | 118 | YLVSFGVWIRT | 563 | 11 | | | | | |
| 90 | 18 | POL | 538 | YMDDVVLGA | 564 | 9 | 0.0250 | 0.0001 | 0.0024 | 0.1000 | 0.0002 |
| 90 | 18 | ENV | 263 | YQGMLPVCPL | 565 | 10 | | | | | |
| 75 | 15 | POL | 5 | YQHFRKLL | 566 | 8 | | | | | |
| 75 | 15 | POL | 5 | YQHFRKLLL | 567 | 9 | | | | | |
| 75 | 15 | POL | 5 | YQHFRKLLLL | 568 | 10 | | | | | |
| 85 | 17 | POL | 746 | YTSFPWLL | 569 | 8 | | | | | |
| 75 | 15 | POL | 746 | YTSFPWLLGCA | 570 | 11 | | | | | |
| 90 | 18 | POL | 768 | YVPSALNPA | 571 | 9 | 0.0039 | | | | |

TABLE IX

HBV A03 SUPER MOTIF (With binding information)

| | | | | | | | | | | | | | |
|---|---|---|---|---|---|---|---|---|---|---|---|---|---|
| | | Pro- | | | | C- | | | | | | | SEQ ID |
| Conservancy | Frequency | tein | Position | Sequence | P2 | term | AA | A*0301 | A*1101 | A*3101 | A*3301 | A*6801 | NO: |

| | | | | | | | | | | | | | |
|---|---|---|---|---|---|---|---|---|---|---|---|---|---|
| 85 | 17 | POL | 721 | AACFARSR | A | R | 8 | 0.0004 | 0.0003 | 0.0058 | 0.0035 | 0.0014 | 572 |
| 95 | 19 | POL | 521 | AICSVVRA | I | R | 8 | −0.0002 | 0.0003 | 0.0014 | −0.0009 | 0.0006 | 573 |
| 90 | 18 | POL | 772 | ALNPADDPSR | L | R | 10 | 0.0003 | 0.0001 | | | | 574 |
| 85 | 17 | X | 70 | ALRFTSAR | L | R | 8 | 0.0047 | 0.0009 | 0.0450 | 0.0230 | 0.0004 | 575 |
| 80 | 16 | POL | 822 | ASPLHVAWR | S | R | 9 | | | | | | 576 |
| 75 | 15 | ENV | 84 | ASTNRQSGR | S | R | 9 | 0.0009 | 0.0002 | 0.0088 | 0.0008 | 0.0001 | 577 |
| 80 | 16 | POL | 755 | CAANWILR | A | R | 8 | | | | | | 578 |
| 85 | 17 | X | 69 | CALRFTSAR | A | R | 9 | 0.0034 | 0.0230 | 1.5000 | 8.0000 | 0.7300 | 579 |
| 90 | 18 | X | 17 | CLRPVGAESR | L | R | 10 | 0.0011 | 0.0001 | | | | 580 |
| 100 | 20 | NUC | 48 | CSPHHTALR | S | R | 9 | 0.0029 | 0.0001 | 0.0520 | 0.0250 | 0.0440 | 581 |
| 85 | 17 | NUC | 29 | DLLDTASALYR | L | R | 11 | 0.0042 | −0.0003 | −0.0012 | 3.7000 | 0.0410 | 582 |
| 85 | 17 | NUC | 32 | DTASALYR | T | R | 8 | 0.0004 | −0.0002 | −0.0009 | 0.0018 | 0.0009 | 583 |
| 95 | 19 | POL | 17 | EAGPLEEELPR | A | R | 11 | −0.0009 | −0.0003 | −0.0012 | 0.0015 | 0.0110 | 584 |
| 90 | 18 | POL | 718 | ELLAACFAR | L | R | 9 | 0.0002 | 0.0004 | | | | 585 |
| 85 | 17 | POL | 718 | ELLAACFARSR | L | R | 11 | 0.0062 | 0.0018 | 0.0200 | 0.2000 | 0.1600 | 586 |
| 95 | 19 | NUC | 174 | ETTVVRRR | T | R | 8 | 0.0003 | −0.0002 | −0.0009 | 0.1400 | 0.0027 | 587 |
| 80 | 16 | NUC | 174 | ETTVVRRRGR | T | R | 10 | 0.0003 | 0.0001 | | | | 588 |
| 80 | 16 | POL | 821 | FASPLHVAWR | A | R | 10 | | | | | | 589 |
| 90 | 18 | X | 63 | FSSAGPCALR | S | R | 10 | | | | | | 590 |
| 95 | 19 | POL | 656 | FTFSPTYK | T | K | 8 | 0.0100 | 0.0100 | 0.0023 | 0.2100 | 0.0590 | 591 |
| 95 | 19 | POL | 518 | FTSAICSVVR | T | R | 10 | 0.0003 | 0.0003 | | | | 592 |
| 95 | 19 | POL | 518 | FTSAICSVVRR | T | R | 11 | 0.0065 | 0.0092 | 0.0170 | 0.0350 | 1.5000 | 593 |
| 90 | 18 | X | 132 | FVLGGCRHK | V | K | 9 | 0.0430 | 0.0090 | | | | 594 |
| 75 | 15 | POL | 567 | GHLNPNK | I | K | 8 | | | | | | 595 |
| 75 | 15 | POL | 567 | GIHLNPNKTK | I | K | 10 | 0.0025 | 0.0011 | 0.0009 | 0.0009 | 0.0003 | 596 |
| 75 | 15 | POL | 567 | GIHLNPNKTKR | I | R | 11 | | | | | | 597 |
| 85 | 17 | NUC | 29 | GMDIDPYK | M | K | 8 | 0.0008 | 0.0004 | −0.0009 | −0.0009 | 0.0001 | 598 |
| 90 | 18- | POL | 735 | GTDNSVVLSR | T | R | 10 | 0.0010 | 0.0420 | 0.0030 | 0.0019 | 0.0008 | 599 |
| 90 | 18 | POL | 735 | GTDNSVVLSRK | T | K | 11 | 0.0140 | 0.5600 | −0.0002 | −0.0006 | 0.0001 | 600 |
| 95 | 19 | NUC | 123 | GVWIRTPPAYR | V | R | 11 | 0.1900 | 0.1700 | 6.8000 | 0.7300 | 0.6600 | 601 |
| 90 | 18 | NUC | 104 | HISCLTFGR | I | R | 9 | 0.0160 | 0.0065 | | | | 602 |
| 75 | 15 | POL | 569 | HLNPNKTK | L | K | 8 | | | | | | 603 |
| 75 | 15 | POL | 569 | HLNPNKTKR | L | R | 9 | 0.0025 | 0.0001 | | | | 604 |
| 100 | 20 | POL | 149 | HTLWKAGILYK | T | K | 11 | 0.5400 | 0.4400 | 0.0370 | 0.0720 | 0.1900 | 605 |
| 90 | 18 | NUC | 105 | ISCLTFGR | S | R | 8 | 0.0004 | 0.0002 | 0.0017 | −0.0009 | 0.0017 | 606 |
| 100 | 20 | POL | 153 | KAGILYKA | A | R | 8 | 0.0002 | −0.0002 | 0.0015 | −0.0009 | 0.0001 | 607 |
| 80 | 16 | POL | 810 | KLPVNRPIDWK | L | K | 11 | | | | | | 608 |
| 75 | 15 | X | 130 | KVFVLGGCR | V | R | 9 | 0.0420 | 0.0820 | 0.6000 | 0.0710 | 0.0030 | 609 |
| 85 | 17 | POL | 720 | LAACFARSR | A | R | 9 | 0.0058 | 0.0065 | | | | 610 |
| 90 | 18 | POL | 719 | LLAACFAR | L | R | 8 | 0.0024 | 0.0003 | 0.0015 | 0.0029 | 0.0064 | 611 |
| 85 | 17 | POL | 719 | LLAACFARSR | L | R | 10 | | | | | | 612 |
| 85 | 17 | NUC | 30 | LLDTASALYR | L | R | 10 | 0.0050 | 0.0002 | | | | 613 |
| 80 | 16 | POL | 752 | LLGCAANWILR | L | R | 11 | | | | | | 614 |
| 75 | 15 | POL | 564 | LSLGIHLNPNK | S | K | 11 | | | | | | 615 |
| 95 | 19 | NUC | 169 | LSTLPETTVVR | S | R | 11 | −0.0009 | 0.0008 | −0.0012 | −0.0023 | 0.0078 | 616 |
| 75 | 15 | POL | 3 | LSYQHFRK | S | K | 8 | | | | | | 617 |
| 85 | 17 | POL | 99 | LTVNEKRR | T | R | 8 | −0.0002 | −0.0002 | −0.0009 | −0.0009 | 0.0001 | 618 |
| 90 | 18 | NUC | 119 | LVSFGVWIR | V | R | 9 | 0.0028 | 0.0120 | | | | 619 |
| 100 | 20 | POL | 377 | LVVDFSQFSR | V | R | 10 | 0.0016 | 0.3600 | 0.0260 | 0.2300 | 0.4900 | 620 |
| 75 | 15 | X | 103 | MSTTDLEAYFK | S | K | 11 | | | | | | 621 |
| 90 | 18 | NUC | 75 | NLEDPASR | L | A | 8 | −0.0002 | −0.0002 | −0.0009 | −0.0009 | 0.0001 | 622 |
| 95 | 19 | POL | 45 | NLNVSIPWTHK | L | K | 11 | −0.0009 | 0.0005 | −0.0012 | −0.0023 | 0.0019 | 623 |
| 90 | 18 | POL | 738 | NSVVLSRK | S | K | 8 | 0.0006 | 0.0010 | −0.0009 | −0.0009 | 0.0007 | 624 |
| 100 | 20 | POL | 47 | NVSIPWTHK | V | K | 9 | 0.0820 | 0.0570 | 0.0002 | 0.0100 | 0.0320 | 625 |
| 90 | 18 | POL | 775 | PADDPSRGR | A | R | 9 | 0.0008 | 0.0002 | 0.0004 | 0.0015 | 0.0002 | 626 |
| 80 | 16 | X | 11 | PARDVLCLR | A | R | 9 | 0.0002 | 0.0002 | 0.0100 | 0.0180 | 0.0002 | 627 |
| 75 | 15 | ENV | 83 | PASTNRQSGR | A | R | 10 | | | | | | 628 |
| 90 | 18 | POL | 616 | PIDWKCQR | I | R | 9 | 0.0002 | 0.0005 | | | | 629 |
| 80 | 18 | POL | 496 | PILGFRK | I | K | 8 | | | | | | 630 |
| 95 | 19 | POL | 20 | PLEEELPR | L | R | 8 | 0.0002 | −0.0002 | −0.0009 | −0.0009 | 0.0001 | 631 |
| 100 | 20 | POL | 2 | PLSYQHFR | L | R | 8 | −0.0002 | −0.0002 | −0.0009 | −0.0009 | 0.0001 | 632 |
| 75 | 15 | POL | 2 | PLSYQHFRK | L | K | 9 | 0.0011 | 0.0031 | 0.0006 | 0.0008 | 0.0002 | 633 |
| 85 | 17 | POL | 98 | PLTVNEKR | L | R | 8 | 0.0002 | −0.0002 | −0.0009 | −0.0009 | 0.0001 | 634 |
| 85 | 17 | POL | 98 | PLTVNEKRR | L | R | 9 | 0.0008 | 0.0005 | 0.0004 | 0.0027 | 0.0002 | 635 |
| 90 | 18 | X | 20 | PVGAESRGR | V | R | 9 | 0.0002 | 0.0005 | 0.0004 | 0.0043 | 0.0002 | 636 |
| 85 | 17 | POL | 612 | PVNRPIDWK | V | K | 9 | 0.0310 | 0.1400 | 0.0002 | 0.0006 | 0.0009 | 637 |
| 95 | 19 | POL | 654 | QAFTFSPTYK | A | K | 10 | 0.0450 | 0.5400 | 0.0010 | 0.0057 | 1.2000 | 638 |
| 80 | 16 | ENV | 179 | QAGFFLLTR | A | R | 9 | | | | | | 639 |
| 75 | 15 | NUC | 169 | QSPRRRRSQSR | S | R | 11 | | | | | | 640 |
| 80 | 16 | POL | 189 | QSSGILSR | S | R | 8 | | | | | | 641 |
| 75 | 15 | POL | 106 | FILKLIMPAR | L | R | 9 | 0.0950 | 0.0002 | 3.1000 | 0.0490 | 0.0002 | 642 |
| 75 | 15 | X | 128 | RLKVFVLGGCR | L | R | 11 | | | | | | 643 |
| 95 | 19 | POL | 376 | RLVVDFSQFSR | L | R | 11 | 0.2800 | 3.8000 | 2.6000 | 1.2000 | 8.1000 | 644 |
| 95 | 19 | NUC | 183 | RSPRRRTPSPR | S | R | 11 | −0.0007 | −0.0003 | 0.0190 | −0.0023 | 0.0003 | 645 |
| 75 | 15 | NUC | 167 | RSQSPRRR | S | R | 8 | | | | | | 646 |
| 75 | 15 | NUC | 167 | RSQSPRRRR | S | R | 9 | | | | | | 647 |
| 95 | 19 | NUC | 188 | ATPSPRRR | T | R | 8 | −0.0002 | −0.0002 | 0.0033 | 0.0014 | 0.0002 | 648 |
| 95 | 19 | NUC | 188 | RTPSPRRRR | T | R | 9 | 0.0054 | 0.0005 | 0.2000 | 0.0016 | 0.0003 | 649 |
| 100 | 20 | POL | 357 | RVTGGVFLVDK | V | K | 11 | 0.0190 | 0.0290 | −0.0002 | −0.0003 | 0.0001 | 650 |
| 90 | 18 | X | 65 | SAGPCALR | A | R | 8 | −0.0002 | 0.0020 | 0.0029 | 0.0024 | 0.0360 | 651 |
| 95 | 19 | POL | 520 | SAICSVVR | A | R | 8 | .0.0002 | 0.0071 | 0.0280 | 0.0081 | 0.0690 | 652 |
| 95 | 19 | POL | 520 | SAICSVVRR | A | R | 9 | 0.0058 | 0.2100 | 0.1500 | 0.0650 | 0.3800 | 653 |
| 90 | 18 | POL | 771 | SALNPADDPSR | A | R | 11 | −0.0004 | −0.0003 | −0.0012 | −0.0023 | 0.0003 | 654 |
| 75 | 15 | POL | 565 | SLGIHLNPNK | L | K | 10 | | | | | | 655 |
| 90 | 18 | X | 64 | SSAGPCALR | S | R | 9 | 0.0080 | 0.1400 | 0.3300 | 0.1600 | 0.7500 | 656 |
| 95 | 19 | NUC | 170 | STLPETTVVR | T | R | 10 | 0.0007 | 0.0600 | 0.0080 | 0.0240 | 0.0250 | 657 |
| 95 | 19 | NUC | 170 | STLPETTVVRR | T | R | 11 | 0.0150 | 1.4000 | 0.1000 | 0.1600 | 0.3100 | 658 |
| 80 | 16 | ENV | 85 | STNQSGR | T | R | 8 | | | | | | 659 |
| 75 | 15 | X | 104 | STTDLEAYFK | T | K | 10 | 0.0066 | 2.7000 | | | | 660 |
| 85 | 17 | POL | 716 | TAELLAACFAR | A | R | 11 | 0.0006 | 0.0023 | 0.0066 | 0.1600 | 0.0590 | 661 |
| 95 | 19 | NUC | 171 | TLPETTVVR | L | R | 9 | 0.0008 | 0.0002 | 0.0009 | 0.0024 | 0.0180 | 662 |
| 95 | 19 | NUC | 171 | TLPETTVVRR | L | R | 10 | 0.0007 | 0.0230 | 0.0006 | 0.0120 | 0.0440 | 663 |
| 95 | 19 | NUC | 171 | TLPETTVVRRR | L | R | 11 | 0.0005 | 0.0160 | 0.0061 | 0.0710 | 0.6400 | 664 |
| 100 | 20 | POL | 150 | TLWKAGILYK | L | K | 10 | 5.3000 | 0.3800 | 0.0051 | 0.0010 | 0.0130 | 665 |
| 100 | 20 | POL | 150 | TLWKAGILYKR | L | R | 11 | 0.0082 | 0.0095 | 0.1000 | 0.1100 | 0.0640 | 666 |
| 95 | 19 | POL | 519 | TSAICSVVR | S | R | 9 | 0.0005 | 0.0008 | 0.0600 | 0.0200 | 0.0820 | 667 |
| 95 | 19 | POL | 519 | TSAICSVVRR | S | R | 10 | 0.0018 | 0.0008 | 0.0030 | 0.0066 | 0.0048 | 668 |
| 75 | 15 | X | 105 | TTDLEAYFK | T | K | 9 | 0.0006 | 0.9200 | 0.0006 | 0.0012 | 0.0170 | 669 |
| 75 | 15 | ENV | 278 | TTSTGPCK | T | K | 8 | | | | | | 670 |
| 80 | 16 | NUC | 175 | TTVVRRRGR | T | R | 9 | 0.0008 | 0.0005 | 0.2500 | 0.1400 | 0.0095 | 671 |
| 80 | 16 | NUC | 176 | TVVRRRGR | V | R | 8 | 0.0003 | 0.0001 | | | | 672 |
| 80 | 16 | NUC | 176 | TVVRRRGRSPR | V | R | 11 | | | | | | 673 |
| 90 | 18 | X | 133 | VLGGCRHK | L | K | 8 | 0.0150 | 0.0002 | −0.0005 | −0.0009 | 0.0001 | 674 |
| 80 | 16 | ENV | 177 | VLQAGFFLLTR | I | R | 11 | | | | | | 675 |
| 90 | 18 | NUC | 120 | VSFGVWIR | S | R | 8 | 0.0040 | 0.0290 | 0.0750 | 0.0270 | 0.0360 | 676 |
| 100 | 20 | POL | 48 | VSIPWTHK | S | K | 8 | 0.0130 | 0.0170 | 0.0031 | 0.0013 | 0.0004 | 677 |
| 100 | 20 | POL | 358 | VTGGVFLVDK | T | K | 10 | 0.0390 | 0.0920 | 0.0002 | 0.0006 | 0.0022 | 678 |
| 100 | 20 | POL | 378 | WDFSQFSR | V | R | 9 | 0.0015 | 0.0750 | 0.0013 | 0.0170 | 0.0330 | 679 |
| 80 | 16 | NUC | 177 | WRRRGRSPR | V | R | 10 | 0.0027 | 0.0001 | | | | 680 |
| 80 | 16 | NUC | 177 | WRRRGRSPRR | V | R | 11 | | | | | | 681 |
| 95 | 19 | NUC | 125 | WIRTPPAYR | I | R | 9 | 0.0008 | 0.0005 | | | | 682 |
| 90 | 18 | POL | 314 | WLQFRNSK | L | K | 8 | −0.0002 | 0.0005 | 0.0020 | 0.0052 | 0.0001 | 683 |
| 85 | 17 | NUC | 28 | WLWGMDIDPYK | L | K | 11 | 0.0030 | 0.0013 | −0.0003 | 0.0039 | 0.0490 | 684 |
| 100 | 20 | POL | 122 | YLPLDKGIK | L | K | 9 | 0.0001 | 0.0001 | 0.0006 | 0.0006 | 0.0002 | 685 |
| 90 | 18 | NUC | 118 | YLVSFGVWIR | L | R | 10 | 0.0005 | 0.0002 | | | | 686 |
| 90 | 18 | POL | 538 | YMDDVVLGAK | M | K | 10 | 0.0330 | 0.0043 | 0.0002 | 0.0006 | 0.0001 | 687 |
| 80 | 16 | POL | 493 | YSHPIILGFR | S | R | 10 | | | | | | 688 |
| 80 | 16 | POL | 493 | YSHPIILGFRK | S | K | 11 | | | | | | 689 |

TABLE X

HBV A24 SUPER MOTIF (With binding information)

| | | | | | | | |
|---|---|---|---|---|---|---|---|
| Conservancy | Freq | Protein | Position | Sequence | String | A*2401 | SEQ ID NO: |

| | | | | | | | |
|---|---|---|---|---|---|---|---|
| 95 | 19 | POL | 529 | AFPHCLAF | XFXXXXXF | | 690 |
| 95 | 19 | POL | 529 | AFPHCLAFSY | XFXXXXXXXY | | 691 |
| 95 | 19 | POL | 529 | AFPHCLAFSYM | XFXXXXXXXM | | 692 |
| 95 | 19 | X | 62 | AFSSAGPCAL | XFXXXXXXXL | 0.0012 | 693 |
| 90 | 18 | POL | 535 | AFSYMDDVVL | XFXXXXXXXL | 0.0009 | 694 |
| 95 | 19 | POL | 655 | AFTFSPTY | XFXXXXXY | | 695 |
| 95 | 19 | POL | 655 | AFTFSPTYKAF | XFXXXXXXXXF | | 696 |
| 95 | 19 | POL | 521 | AICSVVRRAF | XIXXXXXXXF | | 697 |
| 90 | 18 | NUC | 58 | AILCWGEL | XIXXXXXL | | 698 |
| 90 | 18 | NUC | 58 | AILCWGELM | XIXXXXXXM | | 699 |
| 95 | 19 | POL | 642 | ALMPLYACI | XLXXXXXXI | | 700 |
| 95 | 19 | NUC | 54 | ALRQAILCW | XLXXXXXXW | | 701 |
| 80 | 16 | ENV | 108 | AMQWNSTTF | XMXXXXXXF | | 702 |
| 95 | 19 | POL | 690 | ATPTGWGL | XTXXXXXL | | 703 |
| 75 | 15 | POL | 690 | ATPTGWGLAI | XTXXXXXXXI | | 704 |
| 95 | 19 | POL | 397 | AVPNLQSL | XVXXXXXL | | 705 |
| 95 | 19 | POL | 397 | AVPNLQSLTNL | XVXXXXXXXXL | | 706 |
| 100 | 20 | NUC | 131 | AYRPPNAPI | XYXXXXXXI | 0.0260 | 707 |
| 100 | 20 | NUC | 131 | AYRPPNAPIL | XYXXXXXXXL | 0.0220 | 708 |
| 75 | 15 | POL | 607 | CFRKLPVNRPI | XFXXXXXXXXI | | 709 |
| 100 | 20 | ENV | 312 | CIPIPSSW | XIXXXXXW | | 710 |
| 100 | 20 | ENV | 312 | CIPIPSSWAF | XIXXXXXXXF | | 711 |
| 85 | 17 | NUC | 23 | CLGWLWGM | XLXXXXXM | | 712 |
| 85 | 17 | NUC | 23 | CLGWLWGMDI | XLXXXXXXI | | 713 |
| 100 | 20 | ENV | 253 | CLIFLLVL | XLXXXXXL | | 714 |
| 100 | 20 | ENV | 253 | CLIFLLVLL | XLXXXXXXL | | 715 |
| 95 | 19 | ENV | 253 | CLIFLLVLLDY | XLXXXXXXXXY | | 716 |
| 95 | 19 | ENV | 239 | CLRRFIIF | XLXXXXXF | | 717 |
| 95 | 19 | ENV | 239 | CLRRFIIFL | XLXXXXXXL | | 718 |
| 75 | 15 | ENV | 239 | CLRRFIIFLF | XLXXXXXXXF | | 719 |
| 75 | 15 | ENV | 239 | CLRRFIIFLFI | XLXXXXXXXI | | 720 |
| 100 | 20 | ENV | 310 | CTCIPIPSSW | XTXXXXXXXW | | 721 |
| 90 | 18 | NUC | 31 | DIDPYKEF | XIXXXXXF | | 722 |
| 85 | 17 | NUC | 29 | DLLDTASAL | XLXXXXXXL | | 723 |
| 85 | 17 | NUC | 29 | DLLDTASALY | XLXXXXXXXY | | 724 |
| 95 | 19 | POL | 40 | DLNLGNLNVSI | XLXXXXXXXXI | | 725 |
| 80 | 16 | NUC | 32 | DTASALYREAL | XTXXXXXXXXL | | 726 |
| 85 | 17 | POL | 618 | DWKCQRI | XWXXXXXI | | 727 |
| 85 | 17 | POL | 618 | DWKVCQRIVGL | XWXXXXXXXXL | | 728 |
| 90 | 18 | ENV | 262 | DYQGMLPVCPL | XYXXXXXXXXL | 0.0002 | 729 |
| 80 | 16 | X | 122 | ELGEEIRL | XLXXXXXL | | 730 |
| 95 | 19 | NUC | 43 | ELLSFLPSDF | XIXXXXXXXF | | 731 |
| 95 | 19 | NUC | 43 | ELLSFLPSDPP | XLXXXXXXXXF | | 732 |
| 90 | 18 | NUC | 117 | EYLVSRGVW | XYXXXXXXW | | 733 |
| 90 | 18 | NUC | 117 | EYLVSFGVWI | XYXXXXXXXI | 0.0340 | 734 |
| 100 | 20 | ENV | 382 | FFCLWVYI | XFXXXXXI | | 735 |
| 80 | 16 | ENV | 182 | FFLLTRIL | XFXXXXXL | | 736 |
| 80 | 16 | ENV | 182 | FFLLTRILTI | XFXXXXXXXI | | 737 |
| 85 | 17 | ENV | 13 | FFPDHQLDPAF | XFXXXXXXXXF | | 738 |
| 80 | 16 | ENV | 243 | FIIFLFIL | XIXXXXXL | | 739 |
| 80 | 16 | ENV | 243 | FIIFLFILL | XIXXXXXXL | | 740 |
| 80 | 16 | ENV | 243 | FIIFLFILLL | XIXXXXXXXL | | 741 |
| 80 | 16 | ENV | 248 | FILLLCLI | XIXXXXXI | | 742 |
| 60 | 16 | ENV | 248 | FILLLCLIF | XIXXXXXXF | | 743 |
| 80 | 16 | ENV | 248 | FILLLCLIFL | XIXXXXXXXL | | 744 |
| 80 | 16 | ENV | 248 | FILLLCLIFLL | XIXXXXXXXXL | | 745 |
| 80 | 16 | ENV | 246 | FLFILLLCL | XLXXXXXXL | | 746 |
| 80 | 16 | ENV | 246 | FLFILLLCLI | XLXXXXXXXI | | 747 |
| 80 | 16 | ENV | 246 | FLFILLLCLIF | XLXXXXXXXXF | | 748 |
| 75 | 15 | ENV | 171 | FLGPLLVL | XLXXXXXL | | 749 |
| 95 | 19 | POL | 513 | FLLAQFTSAI | XLXXXXXXXI | | 750 |
| 95 | 19 | POL | 562 | FLLSLGIHL | XLXXXXXXL | | 751 |
| 80 | 16 | ENV | 183 | FLLTRILTI | XLXXXXXXI | | 752 |
| 95 | 19 | ENV | 256 | FLLVLLDY | XLXXXXXY | | 753 |
| 95 | 19 | ENV | 256 | FLLVLLDYQGM | XLXXXXXXXXM | | 754 |
| 95 | 19 | POL | 656 | FTFSPTYKAF | XTXXXXXXXF | | 755 |
| 95 | 19 | POL | 656 | FTFSPTYKAFL | XTXXXXXXXXL | | 756 |
| 95 | 19 | POL | 635 | FTQCGYPAL | XTXXXXXXL | | 757 |
| 95 | 19 | POL | 635 | FTQCGYPALM | XTXXXXXXXM | | 758 |
| 95 | 19 | ENV | 346 | FVGLSPTVW | XVXXXXXXW | | 759 |
| 95 | 19 | ENV | 346 | FVGLSPTVWL | XVXXXXXXXL | | 760 |
| 90 | 18 | X | 132 | FVLGGCRHKL | XVXXXXXXXL | | 761 |
| 95 | 19 | ENV | 342 | FVQWFVGL | XVXXXXXL | | 762 |
| 90 | 18 | POL | 766 | FVYVPSAL | XVXXXXXL | | 763 |
| 95 | 19 | POL | 630 | GFAAPFTQCGY | XFXXXXXXXXY | | 764 |
| 60 | 16 | ENV | 181 | GFFLLTRI | XFXXXXXI | | 765 |
| 80 | 16 | ENV | 181 | GFFLLTRIL | XFXXXXXXL | | 766 |
| 80 | 16 | ENV | 181 | GFFLLTRILTI | XFXXXXXXXXI | | 767 |
| 95 | 19 | ENV | 12 | GFFPCHCL | XFXXXXXL | | 768 |
| 75 | 15 | ENV | 170 | GFLGPLLVL | XFXXXXXXL | | 769 |
| 80 | 16 | POL | 500 | GFRKIPMGVGL | XFXXXXXXXXL | | 770 |
| 95 | 19 | POL | 627 | GLLGFAAPF | XLXXXXXXF | | 771 |
| 95 | 19 | POL | 509 | GLSPFLLAQF | XLXXXXXXXF | | 772 |
| 100 | 20 | ENV | 348 | GLSPTVWL | XLXXXXXL | | 773 |
| 75 | 15 | ENV | 348 | GLSPTVWLSVI | XLXXXXXXXXI | | 774 |
| 85 | 17 | PLC | 29 | GMDIDPYKEF | XMXXXXXXF | | 775 |
| 90 | 18 | ENV | 265 | GMLPVCPL | XMXXXXXL | | 776 |
| 90 | 18 | POL | 735 | GTDNSVVL | XTXXXXXL | | 777 |
| 75 | 15 | ENV | 13 | GTNLSVPNPL | XTXXXXXXXL | | 778 |
| 80 | 16 | POL | 763 | GTSFVYVPSAL | XTXXXXXXXXXL | | 779 |
| 80 | 16 | POL | 507 | GVGLSPFL | XVXXXXXL | | 780 |
| 80 | 16 | POL | 507 | GVGLSPFLL | XVXXXXXXL | | 781 |
| 95 | 19 | NUC | 123 | GVWIRTPPAY | XVXXXXXXXY | | 782 |
| 85 | 17 | NUC | 25 | GWLWGMDI | XWXXXXXI | | 783 |
| 85 | 17 | NUC | 25 | GWLWGMDIDPY | XWXXXXXXXXY | | 784 |
| 85 | 17 | ENV | 85 | GWSPQPQGI | XWXXXXXXI | 0.0024 | 785 |
| 85 | 17 | ENV | 65 | GWSPQAQGIL | XWXXXXXXXL | 0.0003 | 786 |
| 95 | 19 | POL | 639 | GYPALMPL | XYXXXXXL | | 787 |
| 95 | 19 | POL | 639 | GYPALMPL | XYXXXXXL | 0.0490 | 788 |
| 95 | 19 | ENV | 234 | GYRWMCLRRF | XYXXXXXXXF | 0.0110 | 789 |
| 95 | 19 | ENV | 234 | GYRWMCLRRFI | XYXXXXXXXXI | | 790 |
| 85 | 17 | POL | 579 | GYSLNFMGY | XYXXXXXXY | 0.0002 | 791 |
| 75 | 15 | POL | 579 | GYSLNFMGYVI | XYXXXXXXXXI | | 792 |
| 80 | 16 | POL | 820 | HFASPLHVAW | XFXXXXXXXW | | 793 |
| 75 | 15 | POL | 7 | HFRKLLLL | XFXXXXXL | | 794 |
| 80 | 16 | POL | 435 | HLLVGSSGL | XLXXXXXXL | | 795 |
| 75 | 15 | POL | 569 | HLNPNKTKRW | XLXXXXXXXW | | 796 |
| 80 | 16 | POL | 491 | HLYSHPII | XLXXXXXI | | 797 |
| 80 | 16 | POL | 491 | HLYSHPIIL | XLXXXXXXL | | 798 |
| 80 | 16 | POL | 491 | HLYSHPIILGF | XLXXXXXXXXXF | | 799 |
| 85 | 17 | POL | 715 | HTAELLAACF | XTXXXXXXXF | | 800 |
| 100 | 20 | NUC | 52 | HTALRQAI | XTXXXXXI | | 801 |
| 95 | 19 | NUC | 52 | HTALRQAIL | XTXXXXXXL | | 802 |
| 95 | 19 | NUC | 52 | HTALRQAILCW | XTXXXXXXXXW | | 803 |
| 100 | 20 | POL | 149 | HTLWKAGI | XTXXXXXI | | 804 |
| 100 | 20 | POL | 149 | HTLWKAGIL | XTXXXXXXL | | 805 |
| 100 | 20 | POL | 149 | HTLWKAGILY | XTXXXXXXXY | | 806 |
| 100 | 20 | POL | 146 | HYLHTLWKAGI | XYXXXXXXXXI | | 807 |
| 100 | 20 | ENV | 381 | IFFCLWVY | XFXXXXXY | | 808 |
| 100 | 20 | ENV | 381 | IFFCLWVYI | XFXXXXXXI | 0.0087 | 809 |
| 80 | 16 | ENV | 245 | IFLFILLL | XFXXXXXL | | 810 |
| 80 | 16 | ENV | 245 | IFLFILLLCL | XFXXXXXXXL | | 811 |
| 80 | 16 | ENV | 245 | IFLFILLLCLI | XFXXXXXXXXI | | 812 |
| 95 | 19 | ENV | 255 | IFLLVLLDY | XFXXXXXXY | | 813 |
| 80 | 16 | ENV | 244 | IIFLFILL | XIXXXXXL | | 814 |
| 80 | 16 | ENV | 244 | IIFLFILLL | XIXXXXXXL | | 815 |
| 80 | 16 | ENV | 244 | IIFLFILLLCL | XIXXXXXXXXL | | 816 |
| 80 | 16 | POL | 497 | IILGFRKI | XIXXXXXI | | 817 |
| 80 | 16 | POL | 497 | IILGFRKIPM | XIXXXXXXXM | | 818 |
| 90 | 18 | NUC | 59 | ILCWGELM | XLXXXXXM | | 819 |
| 80 | 16 | POL | 498 | ILGFRKIPM | XLXXXXXXXM | | 820 |
| 100 | 20 | ENV | 249 | ILLLCLIF | XLXXXXXF | | 821 |
| 100 | 20 | ENV | 249 | ILLLCLIFL | XLXXXXXXL | | 822 |
| 100 | 20 | ENV | 249 | ILLLCLIFLL | XLXXXXXXXL | | 823 |
| 80 | 16 | POL | 760 | ILRGTSFVY | XLXXXXXXY | | 824 |
| 95 | 19 | ENV | 188 | ILTIPQSL | XLXXXXXL | | 825 |
| 90 | 18 | ENV | 188 | ILTIPQSLDSW | XLXXXXXXXXW | | 826 |
| 90 | 18 | POL | 625 | IVGLLGFAAPF | XVXXXXXXXXF | | 827 |
| 8S | 17 | ENV | 358 | IWMMWYWGPS | XWXXXXXXXXL | 0.0004 | 828 |
| 95 | 19 | POL | 395 | KFAVPNLQSL | XFXXXXXXXL | 0.0020 | 829 |
| 80 | 16 | POL | 503 | KIPMGVGL | XIXXXXXL | | 830 |
| 80 | 16 | POL | 503 | KIPMGVGLSPF | XIXXXXXXXXF | | 831 |
| 85 | 17 | NUC | 21 | KLCLGWLW | XLXXXXXW | | 832 |
| 85 | 17 | NUC | 21 | KLCLGWLWGM | XLXXXXXXXM | | 833 |
| 95 | 19 | POL | 489 | KLHLYSHPI | XLXXXXXXI | | 834 |
| 80 | 16 | POL | 489 | KLHLYSHPII | XLXXXXXXXI | | 835 |
| 80 | 16 | POL | 489 | KLHLYSHPIIL | XLXXXXXXXXL | | 836 |
| 75 | 15 | POL | 108 | KLIMPARF | XLXXXXXF | | 837 |
| 75 | 15 | POL | 108 | KLIMPARFY | XLXXXXXXY | | 838 |
| 80 | 16 | POL | 610 | KLPVNRPI | XLXXXXXI | | 839 |
| 80 | 16 | POL | 610 | KLPVNRPIDW | XLXXXXXXXW | | 840 |
| 95 | 19 | POL | 574 | KTKRWGYSL | XTXXXXXXL | | 841 |
| 85 | 17 | POL | 574 | KTKRWGYSLNF | XTXXXXXXXF | | 842 |
| 85 | 17 | POL | 620 | KVCQRIVGL | XVXXXXXXL | | 843 |
| 85 | 17 | POL | 620 | KVCQRVGLL | XVXXXXXXXL | | 844 |
| 95 | 19 | POL | 55 | KVGNFTGL | XVXXXXXL | | 845 |
| 95 | 19 | POL | 55 | KVGNFTGLY | XVXXXXXXY | | 846 |
| 85 | 17 | X | 91 | KVLHKRTLGL | XVXXXXXXXL | | 847 |
| 85 | 17 | X | 91 | KVLHKRTLGL | XVXXXXXXXL | | 848 |
| 100 | 20 | POL | 121 | KYLPLDKGI | XYXXXXXXI | 0.0028 | 849 |
| 85 | 17 | POL | 745 | KYTSFPWL | XYXXXXXXL | | 850 |
| 8S | 17 | POL | 745 | KYTSFPWLL | XYXXXXXXL | 3.6000 | 851 |
| 80 | 16 | ENV | 247 | LFILLLCL | XFXXXXXL | | 852 |
| 80 | 16 | ENV | 247 | LFILLLCLI | XFXXXXXXI | | 853 |
| 80 | 16 | ENV | 247 | LFILLLCLIF | XFXXXXXXXF | | 854 |
| 80 | 16 | ENV | 247 | LFILLLCLIFL | XFXXXXXXXXL | | 855 |
| 100 | 20 | ENV | 254 | LIFLLVL | XIXXXXXXL | | 856 |
| 95 | 19 | ENV | 254 | LIFLLVLLDY | XIXXXXXXXY | | 857 |
| 100 | 20 | POL | 109 | LIMPARFY | XIXXXXXY | | 858 |
| 95 | 19 | POL | 514 | LLAQFTSAI | XLXXXXXXI | | 859 |
| 100 | 20 | ENV | 251 | LLCLIFLL | XLXXXXXL | | 860 |
| 100 | 20 | ENV | 251 | LLCLIFLLVL | XLXXXXXXXL | | 861 |
| 100 | 20 | ENV | 251 | LLCIFLLVLL | XLXXXXXXXXL | | 862 |
| 85 | 17 | NUC | 30 | LLDTASAL | XLXXXXXL | | 863 |
| 85 | 17 | NUC | 30 | LLDTASALY | XLXXXXXXY | | 864 |
| 95 | 19 | ENV | 260 | LLDYQGML | XLXXXXXL | | 865 |
| 80 | 16 | POL | 752 | LLGCAANW | XLXXXXXW | | 866 |
| 80 | 16 | POL | 752 | LLGCAANWI | XLXXXXXXI | | 867 |
| 80 | 16 | POL | 752 | LLGCAANWIL | XLXXXXXXXL | | 868 |
| 95 | 19 | POL | 628 | LLGFAAPF | XLXXXXXF | | 869 |
| 75 | 15 | ENV | 63 | LLGWSPQAQGI | XLXXXXXXXXI | | 870 |
| 100 | 20 | ENV | 250 | LLLCLIFL | XLXXXXXL | | 871 |
| 100 | 20 | ENV | 250 | LLLCLIFLL | XLXXXXXXL | | 872 |
| 100 | 20 | ENV | 250 | LLLCLIFLLVL | XLXXXXXXXXL | | 873 |
| 100 | 20 | ENV | 378 | LLPIFFCL | XLXXXXXL | | 874 |
| 100 | 20 | ENV | 378 | LLPIFFCLW | XLXXXXXXW | | 875 |
| 100 | 20 | ENV | 378 | LLPIFFCLWVY | XLXXXXXXXXY | | 876 |
| 95 | 19 | NUC | 44 | LLSFLPSDF | XLXXXXXXF | | 877 |
| 95 | 19 | NUC | 44 | LLSFLPSDFF | XLXXXXXXXF | | 878 |
| 95 | 19 | POL | 563 | LLSLGIHL | XLXXXXXL | | 879 |
| 90 | 18 | POL | 407 | LLSSNLSW | XLXXXXXL | | 880 |
| 90 | 18 | POL | 407 | LLSSNLSWLSL | XLXXXXXXL | | 881 |
| 90 | 18 | POL | 407 | LLSSNSWLSL | XLXXXXXXXXL | | 882 |
| 80 | 16 | ENV | 184 | LLTRILTI | XLXXXXXI | | 883 |
| 80 | 16 | POL | 436 | LLVGSSGL | XLXXXXXL | | 884 |
| 95 | 19 | ENV | 257 | LLVLLDYQGM | XLXXXXXXXM | | 885 |
| 95 | 19 | ENV | 257 | LLVLLDTQGML | XLXXXXXXXXL | | 886 |
| 95 | 19 | ENV | 175 | LLVLQAGF | XLXXXXXF | | 887 |
| 95 | 19 | ENV | 175 | LLVLQAGFF | XLXXXXXXF | | 888 |
| 90 | 18 | ENV | 175 | LLVLQAGFFL | XLXXXXXXXL | | 889 |
| 90 | 18 | ENV | 175 | LLVLQAGFFLL | XLXXXXXXXXL | | 890 |
| 100 | 20 | ENV | 338 | LLVPFVQW | XLXXXXXW | | 891 |
| 100 | 20 | ENV | 338 | LLVPFVQWF | XLXXXXXXF | | 892 |
| 90 | 18 | NUC | 100 | LLWFHISCL | XLXXXXXXL | | 893 |
| 85 | 17 | NUC | 100 | LLWFHISCLTF | XLXXXXXXXXF | | 894 |
| 95 | 19 | POL | 643 | LMPLYACI | XMXXXXXI | | 895 |
| 75 | 15 | NUC | 137 | LTFGRETVL | XTXXXXXXL | | 896 |
| 75 | 15 | NUC | 137 | LTFGRETVLEY | XTXXXXXXXXY | | 897 |
| 90 | 18 | ENV | 189 | LTIPQSLDSW | XTXXXXXXXW | | 898 |
| 90 | 18 | ENV | 189 | LTIPQSLDSWW | XTXXXXXXXXW | | 899 |
| 90 | 18 | POL | 404 | LTNLLSSNL | XTXXXXXXL | | 900 |
| 90 | 18 | POL | 404 | LTNLLSSNLSW | XTXXXXXXXXW | | 901 |
| 80 | 16 | ENV | 185 | LTRILTIPQSL | XTXXXXXXXXL | | 902 |
| 85 | 17 | POL | 99 | LTVNEKRRL | XTXXXXXXL | | 903 |
| 95 | 19 | ENV | 258 | LVLLDYQGM | XVXXXXXXM | | 904 |
| 95 | 19 | ENV | 258 | LVLLDYQGML | XVXXXXXXXL | | 905 |
| 95 | 19 | ENV | 176 | LVLQAGFF | XVXXXXXF | | 906 |
| 90 | 18 | ENV | 176 | LVLQAGFFL | XVXXXXXXL | | 907 |
| 90 | 18 | ENV | 176 | LVLQAGFFLL | XVXXXXXXXL | | 908 |
| 100 | 20 | ENV | 339 | LVPFVQWF | XVXXXXXXF | | 909 |
| 95 | 19 | ENV | 339 | LVPFVQWFVGL | XVXXXXXXXXL | | 910 |
| 90 | 18 | NUC | 119 | LVSFGVWI | XVXXXXXI | | 911 |
| 100 | 20 | POL | 377 | LVVDFSQF | XVXXXXXF | | 810 |
| 90 | 18 | NUC | 101 | LWFHISCL | XWXXXXXL | | 913 |
| 85 | 17 | NUC | 101 | LWFHISCLTF | XWXXXXXXXF | | 914 |
| 85 | 17 | NUC | 27 | LWGMDIDPY | XWXXXXXXY | | 915 |
| 100 | 20 | POL | 151 | LWKAGILY | XWXXXXXY | | 916 |
| 80 | 16 | POL | 492 | LYSHPIIL | XYXXXXXL | | 917 |
| 80 | 16 | POL | 492 | LYSHPIILGF | XYXXXXXXXF | 1.1000 | 918 |
| 85 | 17 | ENV | 360 | MMWYWGPSL | XMXXXXXXL | 0.0012 | 919 |
| 85 | 17 | ENV | 360 | MMWYWGPSLY | XMXXXXXXXY | 0.0001 | 920 |
| 85 | 17 | ENV | 361 | MWYWGPSL | XWXXXXXL | | 921 |
| 85 | 17 | ENV | 361 | MWYWGPSLY | XWXXXXXXY | 0.0027 | 922 |
| 95 | 19 | POL | 561 | NFLLSLGI | XFXXXXXI | | 923 |
| 95 | 19 | POL | 561 | NFLLSLGIHL | XFXXXXXXXL | 0.0099 | 924 |
| 95 | 19 | POL | 42 | NLGNLNVSI | XLXXXXXXI | | 925 |
| 95 | 19 | POL | 42 | NLGNLNVSIPW | XLXXXXXXXXW | | 926 |
| 90 | 18 | POL | 406 | NLLSSNLSW | XLXXXXXXW | | 927 |
| 90 | 18 | POL | 406 | NLLSSNLSWL | XLXXXXXXXL | | 928 |
| 95 | 19 | POL | 45 | NLNVSIPW | XLXXXXXXW | | 929 |
| 100 | 20 | POL | 400 | NLQSLTNL | XLXXXXXL | | 930 |
| 100 | 20 | POL | 400 | NLQSLTNLL | XLXXXXXXL | | 931 |
| 75 | 15 | ENV | 15 | NLSVPNPL | XLXXXXXL | | 932 |
| 75 | 15 | ENV | 15 | NLSVPNPLGF | XLXXXXXXXF | | 933 |
| 80 | 16 | POL | 758 | NWILRGTSF | XWXXXXXXF | | 934 |
| 80 | 16 | POL | 758 | NWILRGTSFVY | XWXXXXXXXXY | | 935 |
| 95 | 19 | POL | 512 | PFLLAQFTSAI | XFXXXXXXXXI | | 936 |
| 95 | 19 | POL | 634 | PFTQCGYPAL | XFXXXXXXXL | 0.0002 | 937 |
| 95 | 19 | POL | 634 | PFTQCGYPALM | XFXXXXXXXXM | | 938 |
| 95 | 19 | ENV | 341 | PFVQWFVGL | XFXXXXXXL | 0.0003 | 939 |
| 85 | 17 | POL | 616 | PIDWKVCQRI | XIXXXXXXXI | | 940 |
| 100 | 20 | ENV | 380 | PIFFCLWVY | XIXXXXXXY | | 941 |
| 100 | 20 | ENV | 380 | PIFFCLWVYI | XIXXXXXXXI | | 942 |
| 85 | 17 | POL | 713 | PIHTAELL | XIXXXXXL | | 943 |
| 80 | 16 | POL | 496 | PIILGFRKI | XIXXXXXXI | | 944 |
| 80 | 16 | POL | 496 | PIILGFRKIPM | XIXXXXXXXXM | | 945 |
| 100 | 20 | ENV | 314 | PIPSSWAF | XIXXXXXF | | 946 |
| 100 | 20 | POL | 124 | PLDKGIKPY | XLXXXXXXY | | 947 |
| 100 | 20 | POL | 124 | PLDKGIKPY | XLXXXXXXY | | 948 |
| 95 | 19 | POL | 20 | PLEEELPRL | XLXXXXXXL | | 949 |
| 95 | 19 | ENV | 10 | PLGFFPDHQL | XLXXXXXXXXL | | 950 |
| 100 | 20 | POL | 427 | PLHPAAMPHL | XLXXXXXXXL | | 951 |
| 100 | 20 | POL | 427 | PLHPAAMPHLL | XLXXXXXXXXL | | 952 |
| 100 | 20 | ENV | 377 | PLLPIFFCL | XLXXXXXXL | | 953 |
| 100 | 20 | ENV | 377 | PLLPIFFCLW | XLXXXXXXXXW | | 954 |
| 95 | 19 | ENV | 174 | PLLVLQAGF | XLXXXXXXF | | 955 |
| 95 | 19 | ENV | 174 | PLLVLQAGFF | XLXXXXXXXF | | 956 |
| 90 | 18 | ENV | 174 | PLLVLQAGFFL | XLXXXXXXXXL | | 957 |
| 80 | 16 | POL | 711 | PLPIHTAEL | XLXXXXXXL | | 958 |
| 80 | 16 | POL | 711 | PLPIHTAELL | XLXXXXXXXL | | 959 |
| 75 | 15 | POL | 2 | PLSYQHFRKL | XLXXXXXXXL | | 960 |
| 75 | 15 | POL | 2 | PLSYQHFRKLL | XLXXXXXXXXL | | 961 |
| 85 | 17 | POL | 98 | PLTVNEKRRL | XLXXXXXXXL | | 962 |
| 80 | 16 | POL | 505 | PMGVGLSPF | XMXXXXXXF | | 963 |
| 80 | 16 | POL | 505 | PMGVGLSPFL | XMXXXXXXXL | | 964 |
| 80 | 16 | POL | 505 | PMGVGLSPFLL | XMXXXXXXXXL | | 965 |
| 75 | 15 | POL | 692 | PTGWGLAI | XTXXXXXI | | 966 |
| 85 | 17 | POL | 797 | PTTGRTSL | XTXXXXXL | | 967 |
| 85 | 17 | POL | 797 | PTTGRTSLY | XTXXXXXXY | | 968 |
| 80 | 16 | NUC | 15 | PTVQASKL | XTXXXXXL | | 969 |
| 80 | 16 | NUC | 15 | PTVQASKLCL | XTXXXXXXXL | | 970 |
| 75 | 15 | ENV | 351 | PTVWLSVI | XTXXXXXI | | 971 |
| 75 | 15 | ENV | 351 | PTVWLSVIW | XTXXXXXXW | | 972 |
| 75 | 15 | ENV | 351 | PTVWLSVIWM | XTXXXXXXXM | | 973 |
| 85 | 17 | POL | 612 | PVNRPIDW | XVXXXXXW | | 974 |
| 80 | 16 | POL | 750 | PWLLGCAANW | XWXXXXXXXW | | 975 |
| 80 | 16 | POL | 750 | PWLLGCAANWI | XWXXXXXXXXI | | 976 |
| 100 | 20 | POL | 51 | PWTHKVGNF | XWXXXXXXF | 0.0290 | 977 |
| 80 | 16 | X | 8 | QLDPARDVL | XLXXXXXXL | | 978 |
| 80 | 16 | X | 8 | QLDPARDVLCL | XLXXXXXXXXL | | 979 |
| 90 | 18 | NUC | 99 | QLLWFHISCL | XLXXXXXXXL | | 980 |
| 95 | 19 | POL | 685 | QVFADATPTGW | XVXXXXXXXXW | | 981 |
| 95 | 19 | ENV | 344 | QWFVGLSPTVW | XWXXXXXXXX | | 982 |
| 75 | 15 | ENV | 242 | RFIIFLFI | XFXXXXXI | | 983 |
| 75 | 15 | ENV | 242 | RFIIFLFIL | XFXXXXXXL | | 984 |
| 75 | 15 | ENV | 242 | RFIIFLFILL | XFXXXXXXXL | | 985 |
| 75 | 15 | ENV | 242 | RFIIFLFILLL | XFXXXXXXXXL | | 986 |
| 100 | 20 | ENV | 332 | RFSWLSLL | XFXXXXXL | | 987 |
| 100 | 20 | ENV | 332 | RFSWLSLLVPF | XFXXXXXXXXF | | 988 |
| 80 | 16 | ENV | 187 | RILTIPQSL | XIXXXXXXL | | 989 |
| 90 | 18 | POL | 624 | RIGLLGF | XIXXXXXF | | 990 |
| 75 | 15 | POL | 106 | RLKLIMPARF | XLXXXXXXXF | | 991 |
| 75 | 15 | POL | 106 | RLKLIMPARFY | XLXXXXXXXXY | | 992 |
| 95 | 19 | POL | 376 | RLVVDVSQF | XLXXXXXXF | | 993 |
| 90 | 18 | POL | 353 | RTPARVTGGVF | XTXXXXXXXXF | | 994 |
| 95 | 19 | POL | 36 | RVAEDLNL | XVXXXXXL | | 995 |
| 90 | 18 | POL | 36 | RVAEDLNLGNL | XVXXXXXXXXL | | 996 |
| 80 | 16 | POL | 818 | RVHFASPL | XVXXXXXL | | 997 |
| 100 | 20 | POL | 357 | RVTGGVFL | XVXXXXXL | | 998 |
| 85 | 17 | POL | 577 | RWGYSLNF | XWXXXXXF | | 999 |
| 85 | 17 | POL | 577 | RWGYSLNFM | XWXXXXXXM | | 1000 |
| 85 | 17 | POL | 577 | RWGYSLNFMGY | XWXXXXXXXXY | | 1001 |
| 95 | 19 | ENV | 236 | RWMCLRRF | XWXXXXXF | | 1002 |
| 95 | 19 | ENV | 236 | RWMCLRRFI | XWXXXXXXI | 0.0710 | 1003 |
| 95 | 19 | ENV | 236 | RWMCLRRFII | XWXXXXXXXI | 1.1000 | 1004 |
| 95 | 19 | ENV | 236 | RWMCLRRFIIF | XWXXXXXXXXF | | 1005 |
| 100 | 20 | POL | 167 | SFCGSPYSW | XFXXXXXXW | 0.0710 | 1006 |
| 95 | 19 | NUC | 46 | SFLPSDFF | XFXXXXXF | | 1007 |
| 80 | 16 | POL | 765 | SFVYVPSAL | XFXXXXXXL | | 1008 |
| 100 | 20 | POL | 49 | SIPWTHKVGNF | XIXXXXXXXXF | | 1009 |
| 95 | 19 | ENV | 194 | SLDSWWTSL | XLXXXXXXL | | 1010 |
| 95 | 19 | ENV | 194 | SLDSWWTSLNF | XLXXXXXXXXF | | 1011 |
| 95 | 19 | POL | 416 | SLDVSAAF | XLXXXXXF | | 1012 |
| 95 | 19 | POL | 416 | SLDVSAAFY | XLXXXXXXY | | 1013 |
| 100 | 20 | ENV | 337 | SLLVPFVQW | XLXXXXXXW | | 1014 |
| 100 | 20 | ENV | 337 | SLLVPFVQWF | XLXXXXXXXF | | 1015 |
| 75 | 15 | POL | 581 | SLNFMGYVI | XLXXXXXXI | | 1016 |
| 95 | 19 | X | 54 | SLRGLPVCAF | XLXXXXXXXF | | 1017 |
| 90 | 18 | POL | 403 | SLTNLLSSNL | XLXXXXXXXL | | 1018 |
| 75 | 15 | X | 104 | STTDLEAY | XTXXXXXY | | 1019 |
| 75 | 15 | X | 104 | STTDLEAYF | XTXXXXXXF | | 1020 |
| 75 | 15 | ENV | 17 | SVPNPLGF | XVXXXXXF | | 1021 |
| 85 | 17 | POL | 548 | SVQHLESL | XVXXXXXL | | 1022 |
| 80 | 16 | ENV | 330 | SVRFSNWLSL | XVXXXXXXL | | 1023 |
| 80 | 16 | ENV | 330 | SVRFSWLSLL | XVXXXXXXXL | | 1024 |
| 90 | 18 | POL | 739 | SVVLSRKY | XVXXXXXY | | 1025 |
| 85 | 17 | POL | 739 | SVVLSRKYTSF | XVXXXXXXXXXF | | 1026 |
| 95 | 19 | POL | 524 | SVVRRAFPHCL | XVXXXXXXXXL | | 1027 |
| 95 | 19 | POL | 413 | SWLSLDVSAAF | XWXXXXXXXXF | | 1028 |
| 100 | 20 | ENV | 334 | SWLSLLVPF | XWXXXXXXF | 0.3900 | 1029 |
| 95 | 19 | POL | 392 | SWPKFAVPNL | XWXXXXXXXL | 5.6000 | 1030 |
| 100 | 20 | ENV | 197 | SWWTSLNF | XWXXXXXF | | 1031 |
| 95 | 19 | ENV | 197 | SWWTSLNFL | XWXXXXXXL | 0.3800 | 1032 |
| 90 | 18 | POL | 537 | SWMDDVVL | XYXXXXXL | | 1033 |
| 75 | 15 | POL | 4 | SYQHFRKL | XYXXXXXL | | 1034 |
| 75 | 15 | POL | 4 | SYQHFRKLL | XYXXXXXXL | 0.0051 | 1035 |
| 75 | 15 | POL | 4 | SYQHFRKLLL | XYXXXXXXXL | 0.0660 | 1036 |
| 75 | 15 | POL | 4 | SYQHFRKLLLL | XYXXXXXXXXL | | 1037 |
| 75 | 15 | NUC | 138 | TFGRETVL | XFXXXXXL | | 1038 |
| 75 | 15 | NUC | 138 | TFGRETVLEY | XFXXXXXXXY | | 1039 |
| 75 | 15 | NUC | 138 | TFGRETVLEYL | XFXXXXXXXXXL | | 1040 |
| 95 | 19 | POL | 657 | TFSPTYKAF | XFXXXXXXF | 0.0060 | 1041 |
| 95 | 19 | POL | 657 | TFSPTYKAFL | XFXXXXXXXL | 0.0043 | 1042 |
| 90 | 18 | ENV | 190 | TIPQSLDSW | XIXXXXXXW | | 1043 |
| 90 | 18 | ENV | 190 | TIPQSLDSWW | XIXXXXXXXW | | 1044 |
| 100 | 20 | POL | 150 | TLWKAGIL | XLXXXXXL | | 1045 |
| 100 | 20 | POL | 150 | TLWKAGILY | XLXXXXXXY | | 1046 |
| 75 | 15 | X | 105 | TTDLEAYF | XTXXXXXF | | 1047 |
| 85 | 17 | POL | 798 | TTGRTSLY | XTXXXXXY | | 1048 |
| 85 | 17 | POL | 100 | TVNEKRRL | XVXXXXXL | | 1049 |
| 80 | 16 | NUC | 16 | TVQASKLCL | XVXXXXXXL | | 1050 |
| 80 | 16 | NUC | 16 | TVQASKLCLGW | XVXXXXXXXXW | | 1051 |
| 75 | 15 | ENV | 352 | TVWLSVIW | XVXXXXXW | | 1052 |
| 75 | 15 | ENV | 352 | TVWLSVIWM | XVXXXXXXM | | 1053 |
| 95 | 19 | POL | 686 | VFADATPTGW | XFXXXXXXXW | 0.0180 | 1054 |
| 75 | 15 | X | 131 | VFVLGGCRHKL | XFXXXXXXXXL | | 1055 |
| 85 | 17 | POL | 543 | VLGAKSVQHL | XLXXXXXXXL | | 1056 |
| 90 | 18 | X | 133 | VLGGCRHKL | XLXXXXXXL | | 1057 |
| 85 | 17 | X | 92 | VLHKRTLGL | XLXXXXXXL | | 1058 |
| 95 | 19 | ENV | 259 | VLLDYQGM | XLXXXXXM | | 1059 |
| 95 | 19 | ENV | 259 | VLLDYQGML | XLXXXXXXL | | 1060 |
| 95 | 19 | ENV | 177 | VLQAGFFL | XLXXXXXL | | 1061 |
| 95 | 19 | ENV | 177 | VLQAGFFLL | XLXXXXXXL | | 1062 |
| 85 | 17 | POL | 741 | VLSRKYTSF | XLXXXXXXF | | 1063 |
| 85 | 17 | POL | 741 | VLSRKYTSFPW | XLXXXXXXXXXW | | 1064 |
| 80 | 16 | POL | 542 | VVLGAKSVQHL | XVXXXXXXXXL | | 1065 |
| 85 | 17 | POL | 740 | VVLSRKYTSF | XVXXXXXXXF | | 1066 |
| 95 | 19 | POL | 525 | VVRRAFPHCL | XVXXXXXXXL | | 1067 |
| 95 | 19 | NUC | 124 | VWIRTPPAY | XWXXXXXXY | | 1068 |
| 75 | 15 | ENV | 353 | VWLSVIWM | XWXXXXXM | | 1069 |
| 90 | 18 | NUC | 102 | WFHISCLTF | XFXXXXXXF | 0.0300 | 1070 |
| 95 | 19 | ENV | 345 | WFVGLSPTVW | XFXXXXXXXW | 0.0120 | 1071 |
| 95 | 19 | ENV | 345 | WFVGLSPTVWL | XFXXXXXXXXL | | 1072 |
| 80 | 16 | POL | 759 | WLRGTSF | XIXXXXXF | | 1073 |
| 80 | 16 | POL | 759 | WILRGTSFVY | XIXXXXXXXY | | 1074 |
| 95 | 19 | NUC | 125 | WIRTPPAY | XIXXXXXY | | 1075 |
| 80 | 16 | POL | 751 | WLLGCAANW | XLXXXXXXW | | 1076 |
| 80 | 16 | POL | 751 | WLLGCAANWI | XLXXXXXXXI | | 1077 |
| 80 | 16 | POL | 751 | WLLGCAANWIL | XLXXXXXXXXL | | 1078 |
| 95 | 19 | POL | 414 | WLSLDVSAAF | XLXXXXXXF | | 1079 |
| 95 | 19 | POL | 414 | WLSLDVSAAFY | XLXXXXXXXXY | | 1080 |
| 100 | 20 | ENV | 335 | WLSLLVPF | XIXXXXXF | | 1081 |
| 100 | 20 | ENV | 335 | WLSLLVPRVQW | XLXXXXXXXXW | | 1082 |
| 85 | 17 | NUC | 26 | WLWGMDIDPY | XLXXXXXXXY | | 1083 |
| 95 | 19 | ENV | 237 | WMCLRRFI | XMXXXXXI | | 1084 |
| 95 | 19 | ENV | 237 | WMCLRRFII | XMXXXXXXI | 0.0230 | 1085 |
| 95 | 19 | ENV | 237 | WMCLRRFIIF | XMXXXXXXXF | 0.0013 | 1086 |
| 95 | 19 | ENV | 237 | WMCLRRFIIFL | XMXXXXXXXXL | | 1087 |
| 85 | 17 | ENV | 359 | WMMWYWGPSL | XMXXXXXXXXL | 0.0005 | 1088 |
| 85 | 17 | ENV | 359 | WMMWYWGPSL | XMXXXXXXXXY | | 1089 |
| 100 | 20 | POL | 52 | WTHKVGNF | XTXXXXXF | | 1090 |
| 95 | 19 | POL | 52 | WTHKVGNFTGL | XTXXXXXXXXL | | 1091 |
| 95 | 19 | ENV | 198 | WWTSLNFL | XWXXXXXL | | 1092 |
| 85 | 17 | ENV | 362 | WYWGPSLY | XYXXXXXY | 0.0001 | 1093 |
| 100 | 20 | POL | 147 | YLHTLWKAGI | XLXXXXXXXI | | 1094 |
| 100 | 20 | POL | 147 | YLHTLWKAGIL | XLXXXXXXXXL | | 1095 |
| 100 | 20 | POL | 122 | YLPLDKGI | XLXXXXXI | | 1096 |
| 100 | 20 | POL | 122 | YLPLDKGIKPY | XLXXXXXXXXY | | 1097 |
| 90 | 18 | PLC | 118 | YLVSFGVW | XLXXXXXW | | 1098 |
| 90 | 18 | PLC | 118 | YLVSFGVWI | XLXXXXXXI | | 1099 |
| 85 | 17 | POL | 746 | YTSFPWLL | XTXXXXXL | | 1100 |

TABLE XI

HBV B07 SUPER MOTIF (With binding information)

| | | | | | | | | | | | | | |
|---|---|---|---|---|---|---|---|---|---|---|---|---|---|
| | | Pro- | | | | C- | | | | | | | SEQ |
| Conservancy | Frequency | tein | Position | Sequence | P2 | term | AA | B*0702 | B*3501 | B*5101 | B*5301 | B*5401 | ID NO |

| | | | | | | | | | | | | | |
|---|---|---|---|---|---|---|---|---|---|---|---|---|---|
| 75 | 15 | X | 148 | APCNFFTSA | P | A | 9 | | | | | | 1101 |
| 95 | 19 | POL | 833 | APFTQCGY | P | Y | 8 | 0.0001 | 0.0012 | 0.0019 | 0.0002 | 0.0002 | 1102 |
| 95 | 19 | POL | 633 | APFTQCGYPA | P | A | 10 | 0.0029 | 0.0001 | 0.0002 | 1.4000 | 1103 | |
| 95 | 19 | POL | 633 | APFTQCGYPAL | P | L | 11 | 0.2300 | 0.0010 | 0.0004 | −0.0003 | 0.0093 | 1104 |
| 100 | 20 | ENV | 232 | CPGYRWMCL | P | L | 9 | | | | | | 1105 |
| 80 | 16 | NUC | 14 | CPTVQASKL | P | L | 9 | | | | | | 1106 |
| 80 | 16 | NUC | 14 | CPTVQASKLCL | P | L | 11 | | | | | | 1107 |
| 80 | 16 | X | 10 | DPARDVLCL | P | L | 9 | | | | | | 1108 |
| 80 | 16 | ENV | 122 | DPRVRGLY | P | Y | 8 | | | | | | 1109 |
| 90 | 18 | POL | 778 | DPSRGRLGL | P | L | 9 | 0.0120 | 0.0001 | 0.0001 | 0.0001 | 0.0001 | 1110 |
| 90 | 18 | NUC | 33 | DPYKEFGA | P | A | 8 | 0.0001 | 0.0001 | 0.0019 | 0.0002 | 0.0019 | 1111 |
| 75 | 15 | ENV | 130 | FPAGGSSSGTV | P | V | 11 | | | | | | 1112 |
| 90 | 18 | ENV | 14 | FPDHQLDPA | P | A | 9 | | | | | | 1113 |
| 85 | 17 | ENV | 14 | FPDHQLDPAF | P | F | 10 | 0.0002 | 0.0016 | 0.0003 | 0.0011 | 0.0021 | 1114 |
| 95 | 19 | POL | 530 | FPHCLAFSY | P | Y | 9 | 0.0001 | 0.5250 | 0.0665 | 0.5400 | 0.0199 | 1115 |
| 95 | 19 | POL | 530 | FPHCLAFSYM | P | M | 10 | 0.0990 | 0.2200 | 0.0900 | 0.0790 | 0.0480 | 1116 |
| 75 | 15 | POL | 749 | FPWLLGCA | P | A | 8 | | | | | | 1117 |
| 75 | 15 | POL | 749 | FPWLLGCAA | P | A | 9 | | | | | | 1118 |
| 75 | 15 | POL | 749 | FPWLLGCAANW | P | W | 11 | | | | | | 1119 |
| 90 | 18 | X | 67 | GPCALRFTSA | P | A | 10 | 0.0900 | 0.0001 | 0.0001 | 0.0002 | 0.0035 | 1120 |
| 95 | 19 | POL | 19 | GPLEEELPRL | P | L | 10 | 0.0001 | 0.0001 | 0.0002 | 0.0001 | 0.0002 | 1121 |
| 90 | 18 | POL | 19 | GPLEEELPRLA | P | A | 11 | −0.0002 | 0.0001 | 0.0001 | −0.0003 | 0.0001 | 1122 |
| 95 | 19 | ENV | 173 | GPLLVLQA | P | A | 8 | 0.0003 | 0.0001 | 0.0110 | 0.0002 | 0.0065 | 1123 |
| 95 | 19 | ENV | 173 | GPLLVLQAGF | P | F | 10 | 0.0001 | 0.0001 | 0.0002 | 0.0001 | 0.0002 | 1124 |
| 95 | 19 | ENV | 173 | GPLLVLQAGFF | P | F | 11 | 0.0011 | 0.0001 | 0.0001 | 0.0008 | 0.0009 | 1125 |
| 85 | 17 | POL | 97 | GPLTVNEKRRL | P | 1 | 11 | 0.0031 | 0.0001 | 0.0001 | −0.0003 | 0.0001 | 1126 |
| 100 | 20 | POL | 429 | HPAAMPHL | P | L | 8 | 0.0650 | 0.0004 | 0.3100 | 0.0037 | 0.0160 | 1127 |
| 100 | 20 | POL | 429 | HPAAMPHLL | P | L | 9 | 0.0980 | 0.0270 | 0.0110 | 0.0500 | 0.0120 | 1128 |
| 85 | 17 | POL | 429 | HPAAMPHLLV | P | V | 10 | 0.0160 | 0.0020 | 0.0078 | 0.0140 | 0.0170 | 1129 |
| 80 | 16 | POL | 495 | HPIILGFRKI | P | I | 10 | | | | | | 1130 |
| 100 | 20 | ENV | 313 | IPIPSSWA | P | A | 8 | 0.0004 | 0.0004 | 0.0019 | 0.0002 | 0.0600 | 1131 |
| 100 | 20 | ENV | 313 | IPIPSSWAF | P | F | 9 | 0.1300 | 2.7679 | 2.3500 | 0.7450 | 0.0034 | 1132 |
| 80 | 16 | ENV | 313 | IPIPSSWAFA | P | A | 10 | 0.0013 | 0.0024 | | 0.0014 | 0.4500 | 1133 |
| 80 | 16 | POL | 504 | IPMGVGLSPF | P | F | 10 | | | | | | 1134 |
| 80 | 16 | POL | 504 | IPMGVGLSPFL | P | L | 11 | | | | | | 1135 |
| 90 | 18 | ENV | 191 | IPQSLDSW | P | W | 8 | | | | | | 1136 |
| 90 | 18 | ENV | 191 | IPQSLDSWW | P | W | 9 | | | | | | 1137 |
| 80 | 16 | ENV | 315 | IPSSWAFA | P | A | 8 | | | | | | 1138 |
| 100 | 20 | POL | 50 | IPWTHKVGNF | P | F | 10 | 0.0013 | 0.0001 | 0.0007 | 0.0001 | 0.0002 | 1139 |
| 100 | 20 | ENV | 379 | LPIFFCLW | P | W | 8 | 0.0001 | 0.0001 | 0.0360 | 0.1400 | 0.0035 | 1140 |
| 100 | 20 | ENV | 379 | LPIFFCLWV | P | V | 9 | | | | | | 1141 |
| 100 | 20 | ENV | 379 | LPIFFCLWVY | P | V | 10 | 0.0002 | 0.0079 | 0.0002 | 0.0006 | 0.0002 | 1142 |
| 100 | 20 | ENV | 379 | LPIFFCLWVYI | P | I | 11 | 0.0002 | 0.0001 | 0.0043 | 0.0139 | 0.0021 | 1143 |
| 85 | 17 | POL | 712 | LPIHTAEL | P | L | 8 | | | | | | 1144 |
| 85 | 17 | POL | 712 | LPIHTAELL | P | L | 9 | 0.0040 | 0.0630 | 0.0052 | 0.3100 | 0.0005 | 1145 |
| 85 | 17 | POL | 712 | LPIHTAELLA | P | A | 10 | 0.0018 | 0.0011 | | 0.0016 | 0.3300 | 1146 |
| 85 | 17 | POL | 712 | LPIHTAELLAA | P | A | 11 | 0.0090 | 0.0027 | −0.0003 | 0.0120 | 2.7500 | 1147 |
| 80 | 16 | X | 89 | LPKVLHKRTL | P | L | 10 | | | | | | 1148 |
| 100 | 20 | POL | 123 | LPLDKGIKPY | P | Y | 10 | 0.0001 | 0.0290 | 0.0002 | 0.0003 | 0.0002 | 1149 |
| 100 | 20 | POL | 123 | LPLDKGIKPYY | P | Y | 11 | −0.0002 | 0.0009 | 0.0001 | 0.0007 | 0.0001 | 1150 |
| 95 | 19 | X | 58 | LPVCAFSSA | P | A | 9 | 0.0480 | 0.0710 | 0.0110 | 0.0009 | 19.0000 | 1151 |
| 80 | 16 | POL | 611 | LPVNRPIDW | P | W | 9 | | | | | | 1152 |
| 80 | 16 | POL | 611 | LPVNRPIDWKV | P | V | 11 | | | | | | 1153 |
| 80 | 16 | POL | 433 | MPHLLVGSSGL | P | L | 11 | | | | | | 1154 |
| 100 | 20 | POL | 1 | MPLSYQHF | P | F | 8 | 0.0001 | 0.0097 | 0.0120 | 0.0370 | 0.0190 | 1155 |
| 75 | 15 | POL | 1 | MPLSYQHFRKL | P | L | 11 | | | | | | 1156 |
| 90 | 18 | POL | 774 | NPADDPSRGRL | P | L | 11 | 0.0120 | 0.0001 | 0.0001 | −0.0003 | 0.0001 | 1157 |
| 95 | 19 | ENV | 9 | NPLGFFPDQL | P | L | 11 | 0.0012 | 0.0021 | 0.0001 | 0.0028 | 0.0001 | 1158 |
| 75 | 15 | POL | 571 | NPNKTKRW | P | W | 8 | | | | | | 1159 |
| 75 | 15 | POL | 571 | NPNKTKRWGY | P | Y | 10 | | | | | | 1160 |
| 95 | 19 | NUC | 129 | PPAYRPPNA | P | A | 9 | 0.0001 | 0.0001 | 0.0001 | 0.0002 | 0.0003 | 1161 |
| 95 | 19 | NUC | 129 | PPAYRPPNAPI | P | I | 11 | 0.0003 | 0.0001 | 0.0001 | −0.0003 | 0.0001 | 1162 |
| 85 | 17 | ENV | 58 | PPHGGLLGW | P | W | 9 | 0.0001 | 0.0002 | 0.0001 | 0.0003 | 0.0002 | 1163 |
| 100 | 20 | NUC | 134 | PPNAPILSTL | P | L | 10 | 0.0001 | 0.0001 | 0.0035 | 0.0001 | 0.0002 | 1164 |
| 80 | 18 | POL | 615 | RPIDWKVCQRI | P | I | 11 | | | | | | 1165 |
| 100 | 20 | NUC | 133 | RPPNAPIL | P | L | 8 | 0.0076 | 0.0001 | 0.0280 | 0.0002 | 0.0002 | 1166 |
| 100 | 20 | NUC | 133 | RPPNAPILSTL | P | L | 11 | 0.1300 | 0.0001 | 0.0018 | −0.0003 | 0.0001 | 1167 |
| 100 | 20 | NUC | 44 | SPEHCSPHTTA | P | A | 11 | −0.0002 | 0.0001 | 0.0001 | −0.0003 | 0.0011 | 1168 |
| 95 | 19 | POL | 511 | SPFLLAQF | P | F | 8 | 0.5500 | 0.0009 | 0.0180 | 0.0009 | 0.0093 | 1169 |
| 95 | 19 | POL | 511 | SPFLLAQFTSA | P | A | 11 | 0.0820 | 0.0001 | 0.0001 | −0.0003 | 12.0500 | 1170 |
| 100 | 20 | NUC | 49 | SPHHTALRQA | P | A | 10 | 0.0012 | 0.0001 | | 0.0002 | 0.0035 | 1171 |
| 100 | 20 | NUC | 49 | SPHHTALRQAI | P | I | 11 | 0.5800 | 0.0001 | 0.0004 | 0.0005 | 0.0002 | 1172 |
| 85 | 17 | ENV | 67 | SPQAQGIL | P | L | 8 | | | | | | 1173 |
| 85 | 17 | POL | 808 | SPSVPSHL | P | L | 8 | | | | | | 1174 |
| 75 | 15 | ENV | 350 | SPTVWLSV | P | V | 8 | | | | | | 1175 |
| 75 | 15 | ENV | 350 | SPTVWLSVI | P | I | 9 | | | | | | 1178 |
| 75 | 15 | ENV | 350 | SPTVWLSVIW | P | W | 10 | | | | | | 1177 |
| 75 | 15 | ENV | 350 | SPTVWLSVIWM | P | M | 11 | | | | | | 1178 |
| 95 | 19 | POL | 659 | SPTYKAFL | P | L | 8 | 0.3900 | 0.0001 | 0.0019 | 0.0002 | 0.0002 | 1179 |
| 90 | 18 | POL | 354 | TPARVTGGV | P | V | 9 | 0.0078 | 0.0001 | 0.0013 | 0.0001 | 0.0015 | 1180 |
| 90 | 18 | POL | 354 | TPARVTGGVF | P | F | 10 | 0.3200 | 0.1000 | 0.0001 | 0.0099 | 0.0006 | 1181 |
| 90 | 18 | POL | 354 | TPARVTGGVFL | P | L | 11 | 0.0950 | 0.0001 | 0.0001 | 0.0005 | 0.0005 | 1182 |
| 95 | 19 | NUC | 128 | TPPAYRPPNA | P | A | 10 | 0.0001 | 0.0001 | | 0.0002 | 0.0100 | 1183 |
| 75 | 15 | ENV | 57 | TPPHGGLL | P | L | 8 | | | | | | 1184 |
| 75 | 15 | ENV | 57 | TPPHGGLLGW | P | W | 10 | | | | | | 1185 |
| 80 | 18 | POL | 691 | TPTGWGLA | P | A | 8 | | | | | | 1188 |
| 75 | 15 | POL | 691 | TPTGWGLAI | P | I | 9 | | | | | | 1187 |
| 95 | 19 | ENV | 340 | VPFVQWFV | P | V | 8 | 0.0010 | 0.0001 | 19.0000 | 0.0002 | 0.1100 | 1188 |
| 95 | 19 | ENV | 340 | VPFVQWFVGL | P | L | 10 | 0.0011 | 0.0001 | 0.0100 | 0.0001 | 0.0025 | 1189 |
| 95 | 19 | POL | 398 | VPNLQSLTNL | P | L | 10 | 0.0008 | 0.0001 | 0.0004 | 0.0001 | 0.0002 | 1190 |
| 95 | 19 | POL | 398 | VPNLQSLTNLL | P | L | 11 | 0.0004 | 0.0001 | 0.0001 | −0.0003 | 0.0002 | 1191 |
| 90 | 18 | POL | 769 | VPSALNPA | P | A | 8 | 0.0011 | 0.0001 | 0.0070 | 0.0002 | 1.0000 | 1192 |
| 95 | 19 | POL | 393 | WPKFAVPNL | P | L | 9 | 0.0054 | 0.0002 | 0.0015 | 0.0001 | 0.0015 | 1193 |
| 95 | 19 | POL | 640 | YPALMPLY | P | V | 8 | 0.0004 | 0.2600 | 0.4100 | 0.0450 | 0.0056 | 1194 |
| 95 | 19 | POL | 640 | YPALMPLYA | P | A | 9 | 0.0180 | 0.0480 | 0.0340 | 0.0140 | 16.0000 | 1195 |
| 95 | 19 | POL | 640 | YPALMPLYACI | P | I | 11 | 0.0040 | 0.0001 | 0.0470 | 0.0320 | 0.0700 | 1196 |

TABLE XII

HBV B27 Super Motif (No binding data available)

| | | | | | | |
|---|---|---|---|---|---|---|
| | | Position in | No. of | Sequence | Conservancy | |
| Protein | Sequence | HBV | Amino Acids | Frequency | (%) | Seq ID Num |

| | | | | | | |
|---|---|---|---|---|---|---|
| | | | | | | 1197 |
| AYW | AHLSLRGL | 51 | 8 | 19 | 95 | 1198 |
| AYW | ARVTGGVF | 356 | 8 | 18 | 90 | 1199 |
| AYW | DHGAHLSL | 48 | 8 | 19 | 95 | 1200 |
| AYW | DHQLDPAF | 16 | 8 | 18 | 90 | 1201 |
| AYW | DKGIKPYY | 128 | 8 | 20 | 100 | 1202 |
| AWY | FHISCLTF | 103 | 8 | 18 | 90 | 1203 |
| AYW | FRKIPMGV | 501 | 8 | 16 | 80 | 1204 |
| AYR | GRETVLEY | 140 | 8 | 15 | 75 | 1205 |
| AYW | HHTALRQA | 51 | 8 | 20 | 100 | 1206 |
| AYW | IHTAELLA | 714 | 8 | 17 | 85 | 1207 |
| AYW | LHKRTLGL | 93 | 8 | 18 | 90 | 1208 |
| AYW | LHLYSHPI | 490 | 8 | 19 | 95 | 1209 |
| AYW | LRGLPVCA | 55 | 8 | 19 | 95 | 1210 |
| AYW | LRGTSFVY | 761 | 8 | 16 | 80 | 1211 |
| AYW | LRQAILCW | 55 | 8 | 19 | 95 | 1212 |
| AYW | LRRFIIFL | 240 | 8 | 19 | 95 | 1213 |
| AYW | NKTKRWGY | 573 | 8 | 15 | 75 | 1214 |
| AYW | NRPIDWKV | 614 | 8 | 18 | 90 | 1215 |
| AYW | NRRVAEDL | 34 | 8 | 17 | 85 | 1218 |
| AYW | PHCLAFSY | 531 | 8 | 19 | 95 | 1217 |
| AYW | PHGGLLGW | 59 | 8 | 17 | 85 | 1218 |
| AYW | PKFAVPNL | 394 | 8 | 19 | 95 | 1219 |
| AYR | QHFRKLLL | 8 | 8 | 15 | 75 | 1220 |
| AYW | RHYLHTLW | 145 | 8 | 20 | 100 | 1221 |
| AYW | RKYTSFPW | 744 | 8 | 17 | 85 | 1222 |
| AYW | RRAFPHCL | 527 | 8 | 19 | 95 | 1223 |
| AYW | RRFIIFLF | 241 | 8 | 15 | 75 | 1224 |
| AYW | SHPIILGF | 494 | 8 | 16 | 80 | 1225 |
| AYW | SKLCLGWL | 20 | 8 | 18 | 90 | 1226 |
| AYW | SRNLYVSL | 472 | 8 | 16 | 80 | 1227 |
| AYW | TKRWGVSL | 575 | 8 | 19 | 95 | 1228 |
| AYW | TRHYLHTL | 144 | 8 | 20 | 100 | 1229 |
| AYW | VRFSWLSL | 331 | 8 | 18 | 80 | 1230 |
| AYW | WKVCQRIV | 619 | 8 | 17 | 85 | 1231 |
| AYW | YRPPNAPI | 132 | 8 | 20 | 100 | 1232 |
| AYW | ARVTGGVFL | 356 | 9 | 18 | 90 | 1233 |
| AYW | EHCSPHHTA | 46 | 9 | 20 | 100 | 1234 |
| AYR | GRETVLEYL | 140 | 9 | 15 | 75 | 1235 |
| AYW | HHTALRQAI | 51 | 9 | 20 | 100 | 1238 |
| AYW | HKVGNFTGL | 54 | 9 | 19 | 95 | 1237 |
| AYW | IHTAELLAA | 714 | 9 | 17 | 85 | 1238 |
| AYW | KRWGYSLNF | 576 | 9 | 17 | 85 | 1239 |
| AYW | LHLYSHPII | 490 | 9 | 18 | 80 | 1240 |
| AYW | LHPAAMPHL | 428 | 9 | 20 | 100 | 1241 |
| AYW | LHTLWKAGI | 148 | 9 | 20 | 100 | 1242 |
| AYR | LKLIMPARF | 107 | 9 | 15 | 75 | 1243 |
| AYW | LRGLPVCAF | 55 | 9 | 19 | 95 | 1244 |
| AYW | LRGTSFVYV | 761 | 9 | 16 | 60 | 1245 |
| AYW | LRRFIIFLF | 240 | 9 | 15 | 75 | 1246 |
| AYW | PHCLAFSYM | 531 | 9 | 19 | 95 | 1247 |
| AYW | PHHTALRQA | 50 | 9 | 20 | 100 | 1248 |
| AYW | PKVLHKRTL | 90 | 9 | 17 | 85 | 1249 |
| AYR | QHFRKLLLL | 6 | 9 | 15 | 75 | 1250 |
| AYW | QRIVGLLGF | 623 | 9 | 18 | 90 | 1251 |
| AYW | RKIPMGVGL | 502 | 9 | 16 | 80 | 1252 |
| AYW | RKLPVNRPI | 609 | 9 | 16 | 80 | 1253 |
| AYW | RKYTSFPWL | 744 | 9 | 17 | 85 | 1254 |
| AYW | RRAFPHCLA | 527 | 9 | 19 | 95 | 1255 |
| AYW | RRFIIFLFI | 241 | 9 | 15 | 75 | 1256 |
| AYR | RRLKLIMPA | 105 | 9 | 15 | 75 | 1257 |
| AYW | RRVAEDLNL | 35 | 9 | 18 | 90 | 1258 |
| AYW | SKLCLGWLW | 20 | 9 | 17 | 85 | 1259 |
| AYW | SRKYTSFPW | 743 | 9 | 17 | 85 | 1260 |
| AYW | TRHYLHTLW | 144 | 9 | 20 | 100 | 1261 |
| AYW | VHFASPLHV | 819 | 9 | 16 | 80 | 1262 |
| AYW | VRFSWLSLL | 331 | 9 | 16 | 80 | 1263 |
| AYW | VRRAFPHCL | 526 | 9 | 19 | 95 | 1264 |
| AYW | YRPPNAPIL | 132 | 9 | 20 | 100 | 1265 |
| AYW | YRWMCLRRF | 235 | 9 | 19 | 95 | 1266 |
| AYW | AHLSLRGLPV | 51 | 10 | 18 | 90 | 1267 |
| AYW | AKSVQHLESL | 546 | 10 | 17 | 85 | 1268 |
| AYW | ARDVLCLRPV | 12 | 10 | 15 | 75 | 1289 |
| AYW | ARVTGGVFLV | 356 | 10 | 18 | 90 | 1270 |
| AYW | EHCSPHHTAL | 46 | 10 | 20 | 100 | 1271 |
| AYW | FRKIPMGVGL | 501 | 10 | 16 | 80 | 1272 |
| AYW | FRKLPVNRPI | 608 | 10 | 16 | 80 | 1273 |
| AYR | GRETVLEYL | 140 | 10 | 15 | 75 | 1274 |
| AYW | HHTALRQAIL | 51 | 10 | 19 | 95 | 1275 |
| AYW | HKVGNFTGLY | 54 | 10 | 19 | 95 | 1276 |
| AYW | KRWGYSLNFM | 576 | 10 | 17 | 85 | 1277 |
| AYW | LHLYSHPIIL | 490 | 10 | 16 | 80 | 1278 |
| AYW | LHPAAMPHLL | 428 | 10 | 20 | 100 | 1279 |
| AYW | LHTLWKAGIL | 148 | 10 | 20 | 100 | 1280 |
| AYR | LKLIMPARFY | 107 | 10 | 15 | 75 | 1281 |
| AYW | LRRFIIFLFI | 240 | 10 | 15 | 75 | 1282 |
| AYW | NKTKRWGYSL | 573 | 10 | 15 | 75 | 1283 |
| AYW | NRRVAEDLNL | 34 | 10 | 17 | 85 | 1284 |
| AYW | PHHTALRQAI | 50 | 10 | 20 | 100 | 1285 |
| AYW | PHLLVGSSGL | 434 | 10 | 16 | 80 | 1286 |
| AYW | QRIVGLLGFA | 623 | 10 | 18 | 90 | 1287 |
| AYW | RHYLHTLWKA | 145 | 10 | 20 | 100 | 1288 |
| AYW | RKYTSFPWLL | 744 | 10 | 17 | 85 | 1289 |
| AYW | RRAFPHCLAF | 527 | 10 | 19 | 95 | 1290 |
| AYW | RRFIIFLFIL | 241 | 10 | 15 | 75 | 1291 |
| AYW | SRKYTSFPWL | 743 | 10 | 17 | 85 | 1292 |
| AYW | SRLVVDFSQF | 375 | 10 | 19 | 95 | 1293 |
| AYW | THKVGNFTGL | 53 | 10 | 19 | 95 | 1294 |
| AYW | TKRWGYSLNF | 575 | 10 | 17 | 85 | 1295 |
| AYW | TKYLPLDKGI | 120 | 10 | 20 | 100 | 1296 |
| AYW | TRILTIPQSL | 186 | 10 | 18 | 80 | 1297 |
| AYW | VHFASPLHVA | 819 | 10 | 16 | 80 | 1298 |
| AYW | VRFSWLSLLV | 331 | 10 | 16 | 80 | 1299 |
| AYW | VRRAFPHCLA | 526 | 10 | 19 | 95 | 1300 |
| AYW | WKVCQRIVGL | 619 | 10 | 17 | 85 | 1301 |
| AYW | YRWMCLRRFI | 235 | 10 | 19 | 95 | 1302 |
| AYW | DHGAHLSLRGL | 48 | 11 | 19 | 95 | 1303 |
| AYW | IHLNPNKTKRW | 568 | 11 | 15 | 75 | 1304 |
| AYW | IHTAELLAACF | 714 | 11 | 17 | 85 | 1305 |
| AYW | LHPAAMPHLLV | 428 | 11 | 17 | 85 | 1306 |
| AYW | LHTLWKAGILY | 148 | 11 | 20 | 100 | 1307 |
| AYW | LRQAILCWGEL | 55 | 11 | 18 | 90 | 1308 |
| AYW | LRRFIIFLFIL | 240 | 11 | 15 | 75 | 1309 |
| AYW | PHHTALRQAIL | 50 | 11 | 19 | 95 | 1310 |
| AYW | PKFAVPNLQSL | 394 | 11 | 19 | 95 | 1311 |
| AYW | PKVLHKRTLGL | 90 | 11 | 17 | 85 | 1312 |
| AYW | PRTPARVTGGV | 352 | 11 | 18 | 90 | 1313 |
| AYW | QRIVGLLGFAA | 623 | 11 | 18 | 90 | 1314 |
| AYW | RKLPVNRPIDW | 809 | 11 | 16 | 80 | 1315 |
| AYW | RRFIIFLFILL | 241 | 11 | 15 | 75 | 1316 |
| AYR | RRLKLIMPARF | 105 | 11 | 15 | 75 | 1317 |
| AYW | SHPIILGFRKI | 494 | 11 | 16 | 80 | 1318 |
| AYW | SKLCLGWLWGM | 20 | 11 | 17 | 85 | 1319 |
| AYW | SRKYTSFPWLL | 743 | 11 | 17 | 85 | 1320 |
| AYW | THKVGNFTGLY | 53 | 11 | 19 | 95 | 1321 |
| AYW | TKRWGYSLNFM | 575 | 11 | 17 | 85 | 1322 |
| AYW | TRHYLHTLWKA | 144 | 11 | 20 | 100 | 1323 |
| AYW | VHFASPLHVAW | 819 | 11 | 16 | 80 | 1324 |
| AYW | VRRAFPHCLAF | 526 | 11 | 19 | 95 | 1325 |
| AYW | WKVCQRIVGLL | 619 | 11 | 17 | 85 | 1326 |
| AYW | YRWMCLRRFII | 235 | 11 | 19 | 95 | 1327 |
| POL | AAMPHLLV | 431 | 8 | 17 | 85 | 1328 |
| NUC | ASALYREA | 34 | 8 | 17 | 85 | 1329 |
| POL | ASFCGSPY | 166 | 8 | 20 | 100 | 1330 |
| NUC | ASKLCLGW | 19 | 8 | 18 | 90 | 1331 |
| POL | ASPLHVAW | 822 | 8 | 16 | 80 | 1332 |
| ENV | ASVRFSWL | 329 | 8 | 16 | 80 | 1333 |
| POL | ATPTGWGL | 690 | 8 | 19 | 95 | 1334 |
| X | CALRFTSA | 69 | 8 | 18 | 90 | 1335 |
| NUC | CSPHHTAL | 48 | 8 | 20 | 100 | 1336 |
| POL | CSVVRRAF | 523 | 8 | 19 | 95 | 1337 |
| POL | ESRLVVDF | 374 | 8 | 19 | 95 | 1338 |
| NUC | ETVLEYLV | 142 | 8 | 15 | 75 | 1339 |
| POL | FARSRSGA | 724 | 8 | 17 | 85 | 1340 |
| POL | FASPLHVA | 821 | 8 | 16 | 80 | 1341 |
| POL | FSPTYKAF | 658 | 8 | 19 | 95 | 1342 |
| X | FSSAGPCA | 63 | 8 | 19 | 95 | 1343 |
| ENV | FSWLSLLV | 333 | 8 | 20 | 100 | 1344 |
| POL | FSYMDDW | 536 | 8 | 18 | 90 | 1345 |
| POL | FTQCGYPA | 635 | 8 | 19 | 95 | 1346 |
| POL | FTSAICSV | 518 | 8 | 19 | 95 | 1347 |
| POL | GAKSVQHL | 545 | 8 | 17 | 85 | 1348 |
| POL | GTDNSVVL | 735 | 8 | 18 | 90 | 1349 |
| POL | HTAELLAA | 715 | 8 | 17 | 85 | 1350 |
| NUC | HTALRQAI | 52 | 8 | 20 | 100 | 1351 |
| POL | HTLWKAGI | 149 | 8 | 20 | 100 | 1352 |
| POL | LAQFTSAI | 515 | 8 | 19 | 95 | 1353 |
| NUC | LSFLPSDF | 45 | 8 | 19 | 95 | 1354 |
| POL | LSLDVSAA | 415 | 8 | 19 | 95 | 1355 |
| ENV | LSLLVPFV | 338 | 8 | 20 | 100 | 1356 |
| X | LSLRGLPV | 53 | 8 | 19 | 95 | 1357 |
| POL | LSRKYTSF | 742 | 8 | 17 | 85 | 1358 |
| POL | LSSNLSWL | 408 | 8 | 18 | 90 | 1359 |
| POL | LSWLSLDV | 412 | 8 | 20 | 100 | 1360 |
| NUC | LTFGRETV | 108 | 8 | 19 | 95 | 1361 |
| X | MSTTDLEA | 103 | 8 | 18 | 80 | 1362 |
| NUC | NAPILSTL | 138 | 8 | 20 | 100 | 1363 |
| POL | PAAMPHLL | 430 | 8 | 20 | 100 | 1364 |
| POL | PALMPLYA | 641 | 8 | 19 | 95 | 1365 |
| X | PARDVLCL | 11 | 8 | 16 | 80 | 1366 |
| POL | PARVTGGV | 355 | 8 | 18 | 90 | 1367 |
| NUC | PAYRPPNA | 130 | 8 | 19 | 95 | 1368 |
| POL | PSRGRLGL | 779 | 8 | 18 | 90 | 1369 |
| POL | PTGWGLAI | 692 | 8 | 15 | 75 | 1370 |
| POL | PTTGRTSL | 797 | 8 | 17 | 85 | 1371 |
| NUC | PTVQASKL | 15 | 8 | 18 | 80 | 1372 |
| ENV | PTVWLSVI | 351 | 8 | 15 | 75 | 1373 |
| POL | RAFPHCLA | 528 | 8 | 19 | 95 | 1374 |
| X | RTLGLSAM | 96 | 8 | 24 | 120 | 1375 |
| NUC | SALYREAL | 35 | 8 | 18 | 90 | 1376 |
| X | SSAGPCAL | 64 | 8 | 19 | 95 | 1377 |
| ENV | SSGTVNPV | 136 | 8 | 15 | 75 | 1378 |
| ENV | SSKPRQGM | 5 | 8 | 18 | 90 | 1379 |
| ENV | STLPETTV | 141 | 8 | 20 | 100 | 1380 |
| X | STTDLEAY | 104 | 8 | 15 | 75 | 1381 |
| NUC | TALRQAIL | 53 | 8 | 19 | 95 | 1382 |
| POL | TSAICSVV | 519 | 8 | 19 | 95 | 1383 |
| ENV | TSGFLGPL | 168 | 8 | 16 | 80 | 1384 |
| X | TTDLEAYF | 105 | 8 | 15 | 75 | 1385 |
| POL | TTGRTSLY | 798 | 8 | 17 | 85 | 1386 |
| POL | VSWPKFAV | 391 | 8 | 19 | 95 | 1387 |
| NUC | VSYVNVNM | 115 | 8 | 20 | 100 | 1388 |
| POL | VTGGVFLV | 358 | 8 | 20 | 100 | 1389 |
| ENV | WSPQAQGI | 66 | 8 | 17 | 85 | 1390 |
| POL | WTHKVGNF | 52 | 8 | 20 | 100 | 1391 |
| POL | YSLNFMGY | 580 | 8 | 17 | 85 | 1392 |
| POL | YTSFPWLL | 748 | 8 | 17 | 85 | 1393 |
| POL | AAPFTQCGY | 632 | 9 | 19 | 95 | 1394 |
| NUC | ASALYREAL | 34 | 9 | 17 | 85 | 1395 |
| NUC | ASKLCLGWL | 19 | 9 | 18 | 90 | 1396 |
| POL | ATPTGWGLA | 690 | 9 | 16 | 80 | 1397 |
| POL | CSRNLYVSL | 471 | 9 | 18 | 80 | 1398 |
| POL | DATPTGWGL | 689 | 9 | 19 | 95 | 1399 |
| ENV | DSWWTSLNF | 196 | 9 | 19 | 95 | 1400 |
| POL | EAGPLEEEL | 17 | 9 | 20 | 100 | 1401 |
| POL | FADATPTGW | 687 | 9 | 19 | 95 | 1402 |
| POL | FASPLHVAW | 821 | 9 | 16 | 80 | 1403 |
| POL | FAVPNLQSL | 396 | 9 | 19 | 95 | 1404 |
| POL | FSPTYKAFL | 658 | 9 | 19 | 95 | 1405 |
| X | FSSAGPCAL | 63 | 9 | 19 | 95 | 1406 |
| POL | FSYMDDVVL | 536 | 9 | 18 | 90 | 1407 |
| POL | FTFSPTYKA | 656 | 9 | 19 | 95 | 1408 |
| POL | FTGLYSSTV | 59 | 9 | 18 | 90 | 1409 |
| POL | FTQCGYPAL | 635 | 9 | 19 | 95 | 1410 |
| POL | FTSAICSVV | 518 | 9 | 19 | 95 | 1411 |
| X | GAHLSLRGL | 50 | 9 | 19 | 95 | 1412 |
| NUC | HTALRQAIL | 52 | 9 | 19 | 95 | 1413 |
| POL | HTLWKAGIL | 149 | 9 | 20 | 100 | 1414 |
| POL | KSVQHLESL | 547 | 9 | 17 | 85 | 1415 |
| POL | KTKRWGYSL | 574 | 9 | 19 | 95 | 1416 |
| POL | LAFSYMDDV | 534 | 9 | 18 | 90 | 1417 |
| NUC | LSFLPSDFF | 45 | 9 | 19 | 95 | 1418 |
| POL | LSLDVSAAF | 415 | 9 | 19 | 95 | 1419 |
| POL | LSPFLLAQF | 510 | 9 | 19 | 95 | 1420 |
| ENV | LSPTVWLSV | 349 | 9 | 15 | 75 | 1421 |
| NUC | LSTLPETTV | 140 | 9 | 20 | 100 | 1422 |
| ENV | LSVPNPLGF | 16 | 9 | 15 | 75 | 1423 |
| POL | LSYQHFRKL | 3 | 9 | 15 | 75 | 1424 |
| NUC | LTFGRETVL | 137 | 9 | 15 | 75 | 1425 |
| POL | LTNLLSSNL | 404 | 9 | 18 | 90 | 1426 |
| POL | LTVNEKRRL | 99 | 9 | 17 | 85 | 1427 |
| X | MSTTDLEAY | 103 | 9 | 15 | 75 | 1428 |
| POL | NSVVLSRKY | 738 | 9 | 18 | 90 | 1429 |
| POL | PAAMPHLLV | 430 | 9 | 17 | 85 | 1430 |
| POL | PARVTGGVF | 355 | 9 | 18 | 90 | 1431 |
| POL | PTTGRTSLY | 797 | 9 | 17 | 85 | 1432 |
| ENV | PTVWLSVIW | 351 | 9 | 15 | 75 | 1433 |
| POL | QAFTFSPTY | 654 | 9 | 19 | 95 | 1434 |
| NUC | QALCWGEL | 57 | 9 | 18 | 90 | 1435 |
| NUC | QASKLCLGW | 18 | 9 | 18 | 80 | 1436 |
| POL | RAFPHCLAF | 528 | 9 | 19 | 95 | 1437 |
| ENV | RTGDPAPNM | 167 | 9 | 16 | 80 | 1438 |
| X | SAGPCALRF | 65 | 9 | 18 | 90 | 1439 |
| POL | SASFCGSPY | 165 | 9 | 20 | 100 | 1440 |
| POL | SSNLSWLSL | 409 | 9 | 18 | 90 | 1441 |
| ENV | SSSGTVNPV | 135 | 9 | 15 | 75 | 1442 |
| NUC | STLPETTVV | 141 | 9 | 20 | 100 | 1443 |
| X | STTDLEAYF | 104 | 9 | 15 | 75 | 1444 |
| POL | TAELLAACF | 716 | 9 | 17 | 85 | 1445 |
| NUC | TASALYREA | 33 | 9 | 16 | 80 | 1446 |
| POL | TSFVYVPSA | 764 | 9 | 16 | 80 | 1447 |
| ENV | TSGFLGPLL | 168 | 9 | 15 | 75 | 1448 |
| POL | TTGRTSLYA | 798 | 9 | 17 | 85 | 1449 |
| POL | VSIPWTHKV | 48 | 9 | 20 | 100 | 1450 |
| ENV | WSPQAQGIL | 66 | 9 | 17 | 85 | 1451 |
| ENV | WSSKPRQGM | 4 | 9 | 18 | 90 | 1452 |
| POL | YSHPIILGF | 493 | 9 | 16 | 80 | 1453 |
| POL | YSLNFMGYV | 580 | 9 | 15 | 75 | 1454 |
| POL | ASFCGSPYSW | 166 | 10 | 20 | 100 | 1455 |
| NUC | ASKLCLGWLW | 19 | 10 | 17 | 65 | 1456 |
| ENV | ASVRFSWLSL | 329 | 10 | 16 | 80 | 1457 |
| POL | ATPTGWGLAI | 690 | 10 | 15 | 75 | 1458 |
| X | CAFSSAGPCA | 61 | 10 | 19 | 95 | 1459 |
| ENV | CTCIPIPSSW | 310 | 10 | 20 | 100 | 1460 |
| ENV | CTIPAQGTSM | 298 | 10 | 16 | 80 | 1461 |
| POL | DATPTGWGLA | 689 | 10 | 16 | 80 | 1462 |
| ENV | DSWWTSLNFL | 196 | 10 | 18 | 90 | 1463 |
| NUC | DTASALYREA | 32 | 10 | 16 | 80 | 1464 |
| POL | FAAPFTQCGY | 6311 | 10 | 19 | 95 | 1465 |
| ENV | FSWLSLLVPF | 333 | 10 | 20 | 100 | 1468 |
| POL | FTFSPTYKAF | 658 | 10 | 19 | 95 | 1467 |
| POL | FTQCGYPALM | 635 | 10 | 38 | 190 | 1468 |
| ENV | GSSSGTVNPV | 134 | 10 | 15 | 75 | 1469 |
| ENV | GTNLSVPNPL | 13 | 10 | 15 | 75 | 1470 |
| POL | GTSFVYVPSA | 763 | 10 | 16 | 80 | 1471 |
| POL | HTAELLAACF | 715 | 10 | 17 | 85 | 1472 |
| POL | HTLWKAGILY | 149 | 10 | 20 | 100 | 1473 |
| POL | LAFSYMDDVV | 534 | 10 | 18 | 90 | 1474 |
| POL | LSLDVSAAFY | 415 | 10 | 19 | 95 | 1475 |
| ENV | LSLLVPFVQW | 336 | 10 | 20 | 100 | 1476 |
| X | LSLRGLPVCA | 53 | 10 | 19 | 95 | 1477 |
| ENV | LSPTVWLSVI | 349 | 10 | 15 | 75 | 1478 |
| POL | LSRKYTSFPW | 742 | 10 | 17 | 85 | 1479 |
| POL | LSSNLSWLSL | 408 | 10 | 18 | 90 | 1480 |
| NUC | LSTLPETTVV | 140 | 10 | 20 | 100 | 1481 |
| POL | LSWLSLDVSA | 412 | 10 | 20 | 100 | 1482 |
| POL | LSYQHFRKLL | 3 | 10 | 15 | 75 | 1483 |
| ENV | LTIPQSLDSW | 189 | 10 | 18 | 90 | 1484 |
| X | MSTTDLEAYF | 103 | 10 | 15 | 75 | 1485 |
| POL | PADDPSRGRL | 775 | 10 | 18 | 90 | 1486 |
| ENV | PAGGSSSGTV | 131 | 10 | 18 | 90 | 1487 |
| POL | PALMPLYACI | 641 | 10 | 19 | 95 | 1488 |
| X | PAPCNFFTSA | 145 | 10 | 15 | 75 | 1489 |
| POL | PARVTGGVFL | 355 | 10 | 18 | 90 | 1490 |
| NUC | PAYRPPNAPI | 130 | 10 | 19 | 95 | 1491 |
| POL | PTTGRTSLYA | 797 | 10 | 17 | 85 | 1492 |
| NUC | PTVQASKLCL | 15 | 10 | 16 | 80 | 1493 |
| ENV | PTVWLSVIWM | 351 | 10 | 30 | 150 | 1494 |
| ENV | QAGFFLLTRI | 179 | 10 | 16 | 80 | 1495 |
| NUC | QAILCWGELM | 57 | 10 | 36 | 180 | 1496 |
| ENV | QAMQWNSTTF | 107 | 10 | 16 | 80 | 1497 |
| NUC | QASKLCLGWL | 18 | 10 | 16 | 80 | 1498 |
| ENV | QSLDSWWTSL | 193 | 10 | 18 | 90 | 1499 |
| POL | RTPARVTGGV | 353 | 10 | 18 | 90 | 1500 |
| POL | SAICSVVRRA | 520 | 10 | 19 | 95 | 1501 |
| X | SSAGPCALRF | 64 | 10 | 18 | 90 | 1502 |
| POL | TAELLAACFA | 716 | 10 | 17 | 85 | 1503 |
| NUC | TALRQAILCW | 53 | 10 | 19 | 95 | 1504 |
| NUC | TASALYREAL | 33 | 10 | 16 | 80 | 1505 |
| POL | TSFPWLLGCA | 747 | 10 | 15 | 75 | 1506 |
| POL | TSFVYVPSAL | 764 | 10 | 16 | 80 | 1507 |
| ENV | TSGFLGPLLV | 168 | 10 | 15 | 75 | 1508 |
| POL | VAEDLNLGNL | 37 | 10 | 19 | 95 | 1509 |
| POL | YSLNFMGYVI | 580 | 10 | 15 | 75 | 1510 |
| POL | AACFARSRSGA | 721 | 11 | 17 | 85 | 1511 |
| POL | AAPFTQCGYPA | 632 | 11 | 19 | 95 | 1512 |
| ENV | ASVRFSWLSLL | 329 | 11 | 16 | 60 | 1513 |
| X | CAFSSAGPCAL | 61 | 11 | 19 | 95 | 1514 |
| X | CALRFTSARRM | 69 | 11 | 26 | 130 | 1515 |
| NUC | CSPHHTALRQA | 48 | 11 | 20 | 100 | 1516 |
| ENV | CTCIPIPSSWA | 310 | 11 | 20 | 100 | 1517 |
| POL | DATPTGWGLAI | 689 | 11 | 15 | 75 | 1518 |
| NUC | DTASALYREAL | 32 | 11 | 16 | 80 | 1519 |
| POL | ESRLVVDFSQF | 374 | 11 | 19 | 95 | 1520 |
| POL | FADATPTGWGL | 687 | 11 | 19 | 95 | 1521 |
| X | FSSAGPCALRF | 63 | 11 | 18 | 90 | 1522 |
| ENV | FSWLSLLVPFV | 333 | 11 | 20 | 100 | 1523 |
| POL | FSYMDDVVLGA | 536 | 11 | 18 | 90 | 1524 |
| POL | FTFSPTYKAFL | 656 | 11 | 19 | 95 | 1525 |
| X | GAHLSLRGLPV | 50 | 11 | 18 | 90 | 1526 |
| POL | GAKSVQHLESL | 545 | 11 | 17 | 85 | 1527 |
| POL | GTSFVYVPSAL | 763 | 11 | 16 | 80 | 1528 |
| POL | HTAELLAACFA | 715 | 11 | 17 | 85 | 1529 |
| NUC | HTALRQAILCW | 52 | 11 | 19 | 95 | 1530 |
| NUC | ISCLTFGRETV | 105 | 11 | 18 | 90 | 1531 |
| POL | KTKRWGYSLNF | 574 | 11 | 17 | 85 | 1532 |
| POL | LAFSYMDDVVL | 534 | 11 | 18 | 90 | 1533 |
| POL | LAQFTSAICSV | 515 | 11 | 19 | 95 | 1534 |
| ENV | LSLLVPFVQWF | 336 | 11 | 20 | 100 | 1535 |
| X | LSLRGLPVCAF | 53 | 11 | 19 | 95 | 1536 |
| ENV | LSPTVWLSVIW | 349 | 11 | 15 | 75 | 1537 |
| POL | LSRKYTSFPWL | 742 | 11 | 17 | 85 | 1538 |
| POL | LSWLSLDVSAA | 412 | 11 | 19 | 95 | 1539 |
| POL | LSYQHFRKLLL | 3 | 11 | 15 | 75 | 1540 |
| NUC | LTFGRETVLEY | 137 | 11 | 15 | 75 | 1541 |
| ENV | LTIPQSLDSWW | 189 | 11 | 18 | 90 | 1542 |
| POL | LTNLLSSNLSW | 404 | 11 | 18 | 90 | 1543 |
| ENV | LTRILTIPQSL | 185 | 11 | 16 | 80 | 1544 |
| X | PARDVLCLRPV | 11 | 11 | 15 | 75 | 1545 |
| POL | PARVTGGVFLV | 355 | 11 | 18 | 90 | 1546 |
| NUC | PAYRPPNAPIL | 139 | 11 | 19 | 95 | 1547 |
| ENV | PTVWLSVIWMM | 351 | 11 | 28 | 140 | 548 |
| POL | QAFTFSPTYKA | 654 | 11 | 19 | 95 | 1549 |
| ENV | QAGFFLLTRIL | 179 | 11 | 16 | 80 | 1550 |
| NUC | QASLCLGWLW | 18 | 11 | 15 | 75 | 1551 |
| POL | QSLTNLLSSNL | 402 | 11 | 18 | 90 | 1552 |
| POL | RAFPHCLAFSY | 528 | 11 | 19 | 95 | 1553 |
| POL | RTPARVTGGVF | 353 | 11 | 18 | 90 | 1554 |
| NUC | RTPPAYRPPNA | 127 | 11 | 19 | 95 | 1555 |
| POL | SAICSVVRRAF | 520 | 11 | 19 | 95 | 1556 |
| POL | SASFCGSPYSW | 165 | 11 | 20 | 100 | 1557 |
| POL | SSNLSWLSLDV | 409 | 11 | 18 | 90 | 1558 |
| POL | TSAICSWRRA | 519 | 11 | 19 | 95 | 1559 |
| POL | TSFPWLLGCAA | 747 | 11 | 15 | 75 | 1560 |
| ENV | TSGFLGPLLVL | 168 | 11 | 15 | 75 | 1561 |
| POL | VSWPKFAVPNL | 391 | 11 | 19 | 95 | 1562 |
| POL | WTHKVGNFTGL | 52 | 11 | 19 | 95 | 1563 |
| POL | YTSFPWLLGCA | 746 | 11 | 15 | 75 | 1564 |

TABLE XIV

HBV B62 Super Motif

| | | | | | | |
|---|---|---|---|---|---|---|
| | | | No. of | Sequence | Conservancy | |
| Protein | Sequence | Position | Amino Acids | Frequency | (%) | SEQ IN NO: |

| | | | | | | |
|---|---|---|---|---|---|---|
| NUC | AILCWGEL | 58 | 8 | 18 | 90 | 1585 |
| POL | APFTQCGY | 633 | 8 | 19 | 95 | 1566 |
| POL | AVPNLQStL | 397 | 8 | 19 | 95 | 1567 |
| ENV | CIPIPSSW | 312 | 8 | 20 | 100 | 1568 |
| NUC | CLGWLWGM | 23 | 8 | 17 | 85 | 1569 |
| ENV | CLIFLLVL | 253 | 8 | 20 | 100 | 1570 |
| ENV | CLRRFIIF | 239 | 8 | 19 | 95 | 1571 |
| POL | CORVGLL | 622 | 8 | 17 | 85 | 1572 |
| NUC | DIDPYKEF | 31 | 8 | 18 | 90 | 1573 |
| NUC | DLLDTASA | 29 | 8 | 17 | 85 | 1574 |
| ENV | DPRVRGLY | 122 | 8 | 16 | 80 | 1575 |
| NUC | DPYKEFGA | 33 | 8 | 18 | 90 | 1576 |
| X | DVLCLRPV | 14 | 8 | 19 | 95 | 1577 |
| X | ELGEERL | 122 | 8 | 16 | 80 | 1578 |
| POL | ELLAACFA | 718 | 8 | 18 | 90 | 1579 |
| ENV | FIIFLFIL | 243 | 8 | 16 | 80 | 1560 |
| ENV | FILLLCLI | 248 | 8 | 16 | 80 | 1581 |
| ENV | FLGPLLVL | 171 | 8 | 15 | 75 | 1582 |
| ENV | FLLVLLDY | 256 | 8 | 19 | 95 | 1583 |
| POL | FPWLLGCA | 749 | 8 | 15 | 75 | 1584 |
| ENV | FVGLSPTV | 348 | 8 | 19 | 95 | 1585 |
| ENV | FVQWFVGL | 342 | 8 | 19 | 95 | 1586 |
| POL | FVYVPSAL | 768 | 8 | 18 | 90 | 1587 |
| POL | GLSPFLLA | 509 | 8 | 19 | 95 | 1588 |
| ENV | GLSPTVWL | 348 | 8 | 20 | 100 | 1589 |
| ENV | GMLPVCPL | 265 | 8 | 18 | 90 | 1590 |
| ENV | GPLLVLQA | 173 | 8 | 19 | 95 | 1591 |
| POL | GVGLSPFL | 507 | 8 | 16 | 80 | 1592 |
| POL | HLYSHPII | 491 | 8 | 16 | 80 | 1593 |
| POL | HPAAMPHL | 429 | 8 | 20 | 100 | 1594 |
| ENV | IIFLFILL | 244 | 8 | 16 | 80 | 1595 |
| POL | IILGFRKI | 497 | 8 | 16 | 80 | 1596 |
| NUC | LCWGELM | 59 | 8 | 18 | 90 | 1597 |
| ENV | ILLLCLIF | 249 | 8 | 20 | 100 | 1598 |
| POL | ILRGTSFV | 760 | 8 | 16 | 80 | 1599 |
| ENV | ILTIPQSL | 188 | 8 | 19 | 95 | 1600 |
| ENV | IPIPSSWA | 313 | 8 | 20 | 100 | 1601 |
| ENV | IPQSLDSW | 191 | 8 | 18 | 90 | 1602 |
| ENV | IPSSWAFA | 315 | 8 | 16 | 80 | 1603 |
| POL | IVGLLGFA | 625 | 8 | 18 | 90 | 1604 |
| POL | KIPMGVGL | 503 | 8 | 16 | 80 | 1805 |
| NUC | KLCLGWLW | 21 | 8 | 17 | 85 | 1806 |
| POL | KUMPARF | 108 | 8 | 15 | 75 | 1607 |
| POL | KLPVNRPI | 610 | 8 | 16 | 80 | 1608 |
| POL | KVGNFTGL | 55 | 8 | 19 | 95 | 1609 |
| X | KVLHKRTL | 91 | 8 | 17 | 85 | 1610 |
| ENV | LIFLLVLL | 254 | 8 | 20 | 100 | 1611 |
| POL | LIMPARFY | 109 | 8 | 20 | 100 | 1612 |
| POL | LLAQFTSA | 514 | 8 | 19 | 95 | 1813 |
| ENV | LLCLFLL | 251 | 8 | 20 | 100 | 1614 |
| NUC | LLDTASAL | 30 | 8 | 17 | 85 | 1615 |
| ENV | LLDYQGML | 260 | 8 | 19 | 95 | 1616 |
| POL | LLGCAANW | 752 | 8 | 16 | 80 | 1617 |
| POL | LLGFAAPF | 628 | 8 | 19 | 95 | 1618 |
| ENV | LLGWSPQA | 63 | 8 | 17 | 85 | 1619 |
| ENV | LLLCLIFL | 250 | 8 | 20 | 100 | 1620 |
| ENV | LLPIFFCL | 378 | 8 | 20 | 100 | 1621 |
| POL | LLSLGIHL | 563 | 8 | 19 | 95 | 1622 |
| POL | LLSSNLSW | 407 | 8 | 18 | 90 | 1623 |
| ENV | LLTRILTI | 184 | 8 | 16 | 80 | 1624 |
| POL | LLVGSSGL | 436 | 8 | 18 | 80 | 1625 |
| ENV | LLVLQAGF | 175 | 8 | 19 | 95 | 1626 |
| ENV | LLVPFVQW | 338 | 8 | 20 | 100 | 1627 |
| POL | LMPLYACI | 643 | 8 | 19 | 95 | 1628 |
| ENV | LPIFFCLW | 379 | 8 | 20 | 100 | 1629 |
| POL | LPIHTAEL | 712 | 8 | 17 | 85 | 1630 |
| ENV | LQAGFFLL | 178 | 8 | 19 | 95 | 1631 |
| POL | LQSLTNLL | 401 | 8 | 20 | 100 | 1632 |
| ENV | LVLQAGFF | 176 | 8 | 19 | 95 | 1633 |
| ENV | LVPFVQWF | 339 | 8 | 20 | 100 | 1634 |
| NUC | LVSFGVWI | 119 | 8 | 18 | 90 | 1635 |
| POL | LVVDFSQF | 377 | 8 | 20 | 100 | 1636 |
| POL | MPLSYQHF | 1 | 8 | 20 | 100 | 1637 |
| NUC | MQLGHLCL | 1 | 8 | 15 | 75 | 1638 |
| ENV | MQWNSTTF | 109 | 8 | 16 | 80 | 1639 |
| POL | NLNVSIPW | 45 | 8 | 19 | 95 | 1640 |
| POL | NLQSLTNL | 400 | 8 | 20 | 100 | 1641 |
| ENV | NLSVPNPL | 15 | 8 | 15 | 75 | 1642 |
| POL | NPNKTKRW | 571 | 8 | 15 | 75 | 1643 |
| ENV | PIFFCLWV | 380 | 8 | 20 | 100 | 1644 |
| POL | PIHTAELL | 713 | 8 | 17 | 85 | 1645 |
| ENV | PIPSSWAF | 314 | 8 | 20 | 100 | 1646 |
| ENV | PQSLDWW | 192 | 8 | 18 | 90 | 1647 |
| X | PVCAFSSA | 59 | 8 | 19 | 95 | 1648 |
| POL | PVNRPIDW | 612 | 8 | 17 | 85 | 1649 |
| X | QLDPARDV | 8 | 8 | 16 | 80 | 1650 |
| POL | RIVGLLGF | 624 | 8 | 18 | 90 | 1651 |
| POL | RLKLIMPA | 106 | 8 | 15 | 75 | 1652 |
| NUC | RPPNAPIL | 133 | 8 | 20 | 100 | 1653 |
| NUC | RQLWFHI | 98 | 8 | 18 | 90 | 1654 |
| POL | RVAEDLNL | 36 | 8 | 19 | 95 | 1655 |
| POL | RVHFASPL | 818 | 8 | 16 | 80 | 1656 |
| POL | RVTGGVFL | 357 | 8 | 20 | 100 | 1657 |
| POL | SIPWTHKV | 49 | 8 | 20 | 100 | 1658 |
| POL | SLDVSAAF | 416 | 8 | 19 | 95 | 1659 |
| POL | SLNFMGYV | 581 | 8 | 15 | 75 | 1660 |
| POL | SPFLLAQF | 511 | 8 | 19 | 95 | 1661 |
| ENV | SPQAQGIL | 67 | 8 | 17 | 85 | 1662 |
| POL | SPSVPSHL | 808 | 8 | 17 | 85 | 1663 |
| ENV | SPTVWLSV | 350 | 8 | 15 | 75 | 1664 |
| POL | SPTYKAFL | 659 | 8 | 19 | 95 | 1665 |
| ENV | SVPNPLGF | 17 | 8 | 15 | 75 | 1666 |
| POL | SVQHLESL | 548 | 8 | 17 | 85 | 1667 |
| POL | SVVLSRKY | 739 | 8 | 18 | 90 | 1668 |
| NUC | TLPETTVV | 142 | 8 | 20 | 100 | 1669 |
| POL | TLWKAGIL | 150 | 8 | 20 | 100 | 1670 |
| ENV | TPPHGGL | 57 | 8 | 15 | 75 | 1671 |
| POL | TPTGWGLA | 691 | 8 | 16 | 80 | 1672 |
| POL | TQCGYPAL | 638 | 8 | 19 | 95 | 1673 |
| POL | TVNEKRRL | 100 | 8 | 17 | 85 | 1674 |
| ENV | TVWLSVTW | 352 | 8 | 15 | 75 | 1675 |
| ENV | VLLDYQGM | 259 | 8 | 19 | 95 | 1676 |
| ENV | VLQAGFFL | 177 | 8 | 19 | 95 | 1677 |
| ENV | VPFVQMFV | 340 | 8 | 19 | 95 | 1678 |
| POL | VPSALNPA | 769 | 8 | 18 | 90 | 1679 |
| NUC | VQASKLCL | 17 | 8 | 16 | 80 | 1680 |
| POL | WLGAKSV | 542 | 8 | 18 | 90 | 1681 |
| POL | WILRGTSF | 759 | 8 | 16 | 80 | 1682 |
| NUC | WIRTPPAY | 125 | 8 | 19 | 95 | 1683 |
| POL | WLSLDVSA | 414 | 8 | 20 | 100 | 1664 |
| ENV | WLSLLVPF | 335 | 8 | 20 | 100 | 1685 |
| ENV | WMCLRRFI | 237 | 8 | 19 | 95 | 1686 |
| POL | YLHTLWKA | 147 | 8 | 20 | 100 | 1687 |
| POL | YLPLDKGI | 122 | 8 | 20 | 100 | 1688 |
| NUC | YLVSFGVW | 118 | 8 | 18 | 90 | 1689 |
| POL | YPALMPLY | 640 | 8 | 19 | 95 | 1690 |
| POL | YQHFRKLL | 5 | 8 | 15 | 75 | 1691 |
| POL | AICSVVRRA | 521 | 9 | 19 | 95 | 1692 |
| NUC | AILCWGELM | 58 | 9 | 18 | 90 | 1693 |
| POL | ALMPLYACI | 642 | 9 | 19 | 95 | 1694 |
| NUC | ALRQAILCW | 54 | 9 | 19 | 95 | 1695 |
| ENV | AMQWNSTTF | 108 | 9 | 16 | 80 | 1696 |
| X | AMSTTDLEA | 102 | 9 | 15 | 75 | 1697 |
| X | APCNFFTSA | 146 | 9 | 15 | 75 | 1698 |
| ENV | CIPIPSSWA | 312 | 9 | 20 | 100 | 1699 |
| ENV | CLIFLLVLL | 253 | 9 | 20 | 100 | 1700 |
| ENV | CLRRFIIFL | 239 | 9 | 19 | 95 | 1701 |
| NUC | CLTGRETV | 107 | 9 | 18 | 90 | 1702 |
| ENV | CPGYRWMCL | 232 | 9 | 20 | 100 | 1703 |
| NUC | CPTVQASKL | 14 | 9 | 16 | 80 | 1704 |
| X | CQLDPARDV | 7 | 9 | 16 | 80 | 1705 |
| NUC | DLLDTASAL | 29 | 9 | 17 | 85 | 1708 |
| POL | DLNLGNLNV | 40 | 9 | 19 | 95 | 1707 |
| X | DPARDVLCL | 10 | 9 | 16 | 80 | 1708 |
| POL | DPSRGRLGL | 778 | 9 | 18 | 90 | 1709 |
| POL | DVVLGAKSV | 541 | 9 | 18 | 90 | 1710 |
| ENV | FIIFLFILL | 243 | 9 | 16 | 80 | 1711 |
| ENV | FILLLCLIF | 248 | 9 | 16 | 80 | 1712 |
| ENV | FLFILLLCL | 248 | 9 | 16 | 80 | 1713 |
| POL | FLLAQFTSA | 513 | 9 | 19 | 95 | 1714 |
| POL | FLLSLGIHL | 562 | 9 | 19 | 95 | 1715 |
| ENV | FLLTRILTI | 183 | 9 | 16 | 80 | 1716 |
| ENV | FPDHQLDPA | 14 | 9 | 18 | 90 | 1717 |
| POL | FPHCLAFSY | 530 | 9 | 19 | 95 | 1718 |
| POL | FPWLLGCAA | 749 | 9 | 15 | 75 | 1719 |
| ENV | FVGLSPTVW | 346 | 9 | 19 | 95 | 1720 |
| POL | GLCQVFADA | 682 | 9 | 17 | 85 | 1721 |
| POL | GLLGFAAPF | 627 | 9 | 19 | 95 | 1722 |
| ENV | GLLGWSPQA | 62 | 9 | 17 | 85 | 1723 |
| POL | GVGLSPFLL | 507 | 9 | 16 | 80 | 1724 |
| NUC | GVWIRTPPA | 123 | 9 | 19 | 95 | 1725 |
| POL | HLLVGSSGL | 435 | 9 | 16 | 80 | 1726 |
| X | HLSLRGLPV | 52 | 9 | 18 | 90 | 1727 |
| POL | HLYSHPIIL | 491 | 9 | 16 | 80 | 1728 |
| POL | HPAAMPHLL | 429 | 9 | 20 | 100 | 1729 |
| ENV | IIFLFILLL | 244 | 9 | 16 | 80 | 1730 |
| POL | ILGFRKIPM | 498 | 9 | 16 | 80 | 1731 |
| ENV | ILLLCLIFL | 249 | 9 | 20 | 100 | 1732 |
| POL | ILRGTSFVY | 760 | 9 | 16 | 80 | 1733 |
| ENV | IPIPSSWAF | 313 | 9 | 20 | 100 | 1734 |
| ENV | IPQSLDSWW | 191 | 9 | 18 | 90 | 1735 |
| POL | IVGLLGFAA | 625 | 9 | 18 | 90 | 1736 |
| POL | KLHLYSHPI | 489 | 9 | 19 | 95 | 1737 |
| POL | KLIMPARFY | 108 | 9 | 15 | 75 | 1738 |
| POL | KVCQRIVGL | 620 | 9 | 17 | 85 | 1739 |
| POL | KVGNFTGLY | 55 | 9 | 19 | 95 | 1740 |
| POL | LLAQFTSAI | 514 | 9 | 19 | 95 | 1741 |
| ENV | LLCLIFLLV | 251 | 9 | 20 | 100 | 1742 |
| NUC | LLDTASALY | 30 | 9 | 17 | 85 | 1743 |
| POL | LLGCAANWI | 752 | 9 | 16 | 80 | 1744 |
| ENV | LLLCLIFLL | 250 | 9 | 20 | 100 | 1745 |
| ENV | LLPIFFCLW | 378 | 9 | 20 | 100 | 1746 |
| NUC | LLSFLPSDF | 44 | 9 | 19 | 95 | 1747 |
| POL | LLSSNLSWL | 407 | 9 | 18 | 90 | 1748 |
| ENV | LLVQAGFF | 175 | 9 | 19 | 95 | 1749 |
| ENV | LLVPFVQWF | 338 | 9 | 20 | 100 | 1750 |
| NUC | LLWFHISCL | 100 | 9 | 18 | 90 | 1751 |
| ENV | LPIFFCLWV | 379 | 9 | 20 | 100 | 1752 |
| POL | LPIHTAELL | 712 | 9 | 17 | 85 | 1753 |
| X | LPVCAFSSA | 58 | 9 | 19 | 95 | 1754 |
| POL | LPVNRPIDW | 611 | 9 | 16 | 50 | 1755 |
| ENV | LVLLDYQGM | 255 | 9 | 19 | 95 | 1758 |
| ENV | LVLQAGFFL | 176 | 9 | 18 | 90 | 1757 |
| ENV | LVPFVQWFV | 339 | 9 | 19 | 95 | 1758 |
| ENV | MMWYWGPSL | 360 | 9 | 17 | 85 | 1759 |
| POL | NLGNLNSI | 42 | 9 | 19 | 95 | 1760 |
| POL | NLLSSNLSW | 406 | 9 | 18 | 90 | 1761 |
| POL | NLQSLTNLL | 400 | 9 | 20 | 100 | 1762 |
| POL | NLSWLSLDV | 411 | 9 | 18 | 90 | 1763 |
| ENV | PIFFCLWVY | 380 | 9 | 20 | 100 | 1764 |
| POL | PIHTAELLA | 713 | 9 | 17 | 85 | 1765 |
| POL | PIILGFRKI | 496 | 9 | 16 | 80 | 1766 |
| ENV | PIPSSWAFA | 314 | 9 | 16 | 80 | 1767 |
| POL | PLDKGIKPY | 124 | 9 | 20 | 100 | 1768 |
| POL | PLEEELPRL | 20 | 9 | 19 | 95 | 1769 |
| ENV | PLLPIFFCL | 377 | 9 | 20 | 100 | 1770 |
| ENV | PLLVLQAGF | 174 | 9 | 19 | 95 | 1771 |
| POL | PLPIHTAEL | 711 | 9 | 16 | 80 | 1772 |
| POL | PMGVGLSPF | 505 | 9 | 16 | 80 | 1773 |
| NUC | PPAYRPPNA | 129 | 9 | 19 | 95 | 1774 |
| ENV | PPHGGLLGW | 58 | 9 | 17 | 65 | 1775 |
| X | QLDPARDVL | 8 | 9 | 16 | 80 | 1776 |
| ENV | RILTIPQSL | 187 | 9 | 16 | 80 | 1777 |
| POL | RIVGLLGFA | 624 | 9 | 18 | 90 | 1778 |
| POL | RLWDFSQF | 376 | 9 | 19 | 95 | 1779 |
| POL | RVTGGVFLV | 357 | 9 | 20 | 100 | 1780 |
| ENV | SLDSWWTSL | 194 | 9 | 19 | 95 | 1781 |
| POL | SLDVSAAFY | 418 | 9 | 19 | 95 | 1782 |
| ENV | SLLVPFVQW | 337 | 9 | 20 | 100 | 1783 |
| POL | SLNFMGYVI | 581 | 9 | 15 | 75 | 1784 |
| X | SLRGLPVCA | 54 | 9 | 19 | 95 | 1785 |
| ENV | SPTVWLSVI | 350 | 9 | 15 | 75 | 1786 |
| ENV | SVRFSWLSL | 330 | 9 | 16 | 80 | 1767 |
| ENV | TIPQSLDSW | 190 | 9 | 18 | 90 | 1788 |
| POL | TLWKAGILY | 150 | 9 | 20 | 100 | 1789 |
| POL | TPARVTGGV | 354 | 9 | 18 | 90 | 1790 |
| POL | TPTGWGLAI | 691 | 9 | 15 | 75 | 1791 |
| POL | TQCGYPALM | 636 | 9 | 19 | 95 | 1792 |
| NUC | TVQASKLCL | 16 | 9 | 16 | 80 | 1793 |
| ENV | TVWLSVIWM | 352 | 9 | 15 | 75 | 1794 |
| X | VLCLRPVGA | 15 | 9 | 19 | 95 | 1795 |
| X | VLGGCRHKL | 133 | 9 | 18 | 90 | 1796 |
| X | VLHKRTLGL | 92 | 9 | 17 | 85 | 1797 |
| ENV | VLLDYQGML | 259 | 9 | 19 | 95 | 1798 |
| ENV | VLQAGFFLL | 177 | 9 | 19 | 95 | 1799 |
| POL | VLSRKYTSF | 741 | 9 | 17 | 85 | 1600 |
| POL | WILRGTSFV | 759 | 9 | 16 | 80 | 1801 |
| POL | WLLGCAANW | 751 | 9 | 16 | 80 | 1802 |
| POL | WLSLDVSAA | 414 | 9 | 19 | 95 | 1803 |
| ENV | WLSLLVPFV | 335 | 9 | 20 | 100 | 1804 |
| ENV | WMCLRRFII | 237 | 9 | 19 | 95 | 1805 |
| POL | WPKFAVPNL | 393 | 9 | 19 | 95 | 1806 |
| NUC | YLVSFGVWI | 118 | 9 | 18 | 90 | 1807 |
| POL | YMDDVVLGA | 538 | 9 | 18 | 90 | 1808 |
| POL | YPALMPLYA | 640 | 9 | 19 | 95 | 1809 |
| POL | YQHFRKLLL | 5 | 9 | 15 | 75 | 1810 |
| POL | YVPSALNPA | 788 | 9 | 18 | 90 | 1811 |
| POL | AICSVVRRAF | 521 | 10 | 19 | 95 | 1812 |
| POL | APFTQCGYPA | 633 | 10 | 19 | 95 | 1813 |
| POL | AQFTSAICSV | 518 | 10 | 19 | 95 | 1814 |
| ENV | CIPIPSSWAF | 312 | 10 | 20 | 100 | 1815 |
| POL | CLAFSYMDDV | 533 | 10 | 18 | 90 | 1816 |
| NUC | CLGWLWGMDI | 23 | 10 | 17 | 85 | 1817 |
| ENV | CLRRFIIFLF | 239 | 10 | 15 | 75 | 1818 |
| X | CQLDPARDVL | 7 | 10 | 16 | 80 | 1819 |
| POL | CQRVGLLGF | 622 | 10 | 17 | 85 | 1820 |
| NUC | DIDPYKEFGA | 31 | 10 | 18 | 90 | 1821 |
| NUC | DILDTASALY | 29 | 10 | 17 | 85 | 1822 |
| X | DVLCLRPVGA | 14 | 10 | 19 | 95 | 1823 |
| NUC | ELLSFLPSDF | 43 | 10 | 19 | 95 | 1824 |
| ENV | FIIFLFILLL | 243 | 10 | 16 | 80 | 1825 |
| ENV | FILLLCLIFL | 248 | 10 | 16 | 80 | 1826 |
| ENV | FLFILLLCLI | 246 | 10 | 16 | 80 | 1827 |
| ENV | FLGPLLVLQA | 171 | 10 | 15 | 75 | 1828 |
| POL | FLLAQFTSAI | 513 | 10 | 19 | 95 | 1829 |
| ENV | FPDHQLDPAF | 14 | 10 | 17 | 85 | 1830 |
| POL | FPHCLAFSYM | 530 | 10 | 19 | 95 | 1831 |
| ENV | FVGLSPTVWL | 346 | 10 | 19 | 95 | 1832 |
| X | FVLGGCRHKL | 132 | 10 | 18 | 90 | 1833 |
| X | GLPVCAFSSA | 57 | 10 | 19 | 95 | 1834 |
| POL | GLSPFLLAQF | 509 | 10 | 19 | 95 | 1835 |
| ENV | GLSPTVWLSV | 348 | 10 | 15 | 75 | 1836 |
| NUC | GMDIDPYKEF | 29 | 10 | 17 | 85 | 1837 |
| X | GPCALRFTSA | 67 | 10 | 18 | 90 | 1838 |
| POL | GPLEEELPRL | 19 | 10 | 19 | 95 | 1839 |
| ENV | GPLLVLQAGF | 173 | 10 | 19 | 95 | 1840 |
| POL | GVGLSPFLLA | 507 | 10 | 16 | 80 | 1841 |
| NUC | GVWIRTPPAY | 123 | 10 | 19 | 95 | 1842 |
| POL | HLNPNKTKRW | 569 | 10 | 15 | 75 | 1843 |
| POL | HPAAMPHLLV | 429 | 10 | 17 | 85 | 1844 |
| POL | HPIILGFRKI | 495 | 10 | 16 | 80 | 1845 |
| POL | IILGFRKIPM | 497 | 10 | 16 | 80 | 1846 |
| ENV | ILLLCLIFLL | 249 | 10 | 20 | 100 | 1847 |
| POL | ILRGTSFVYV | 780 | 10 | 16 | 80 | 1848 |
| NUC | ILSTLPETTV | 139 | 10 | 20 | 100 | 1849 |
| ENV | IPIPSSWAFA | 313 | 10 | 16 | 80 | 1850 |
| POL | IPMGVGLSPF | 504 | 10 | 16 | 80 | 1851 |
| POL | IPWTHKVGNF | 50 | 10 | 20 | 100 | 1852 |
| NUC | KLCLGWLWGM | 21 | 10 | 17 | 85 | 1853 |
| POL | KLHLYSHPII | 489 | 10 | 16 | 80 | 1854 |
| POL | KLPVNRPIDW | 610 | 10 | 16 | 80 | 1855 |
| POL | KQAFTFSPTY | 653 | 10 | 19 | 95 | 1856 |
| POL | KVCQRVGLL | 620 | 10 | 17 | 85 | 1857 |
| X | KVLHKRTLGL | 91 | 10 | 17 | 85 | 1858 |
| ENV | LIFLLVLLDY | 254 | 10 | 19 | 95 | 1859 |
| ENV | LLCLIFLLVL | 251 | 10 | 20 | 100 | 1860 |
| ENV | LLDYQGMLPV | 260 | 10 | 18 | 90 | 1861 |
| POL | LLGCAANWIL | 752 | 10 | 16 | 80 | 1862 |
| ENV | LLLCLIFLLV | 250 | 10 | 20 | 100 | 1863 |
| ENV | LLPIFFCLWV | 378 | 10 | 20 | 100 | 1864 |
| NUC | LLSFLPSDFF | 44 | 10 | 19 | 95 | 1865 |
| ENV | LLVLLDYQGM | 257 | 10 | 19 | 95 | 1866 |
| ENV | LLVLQAGFFL | 175 | 10 | 18 | 90 | 1867 |
| ENV | LLVPFVQWFV | 338 | 10 | 19 | 95 | 1868 |
| ENV | LPIFFCLWVY | 379 | 10 | 20 | 100 | 1869 |
| POL | LPIHTAELLA | 712 | 10 | 17 | 85 | 1070 |
| X | LPKVLHKRTL | 89 | 10 | 16 | 80 | 1871 |
| POL | LPLDKGIKPY | 123 | 10 | 20 | 100 | 1872 |
| ENV | LVLLDYQGML | 258 | 10 | 19 | 95 | 1873 |
| ENV | LVLQAGFFLL | 176 | 10 | 18 | 90 | 1874 |
| ENV | MMWYWGPSLY | 360 | 10 | 17 | 85 | 1875 |
| POL | NLLSSNLSWL | 406 | 10 | 18 | 90 | 1876 |
| ENV | NLSVPNPLGF | 15 | 10 | 15 | 75 | 1877 |
| POL | NPNKTKRWGY | 571 | 10 | 15 | 75 | 1878 |
| POL | NVSIPWTHKV | 47 | 10 | 20 | 109 | 1879 |
| POL | PIDWKVCQRI | 616 | 10 | 17 | 85 | 1880 |
| ENV | PIFFCLWVYI | 380 | 10 | 20 | 100 | 1881 |
| POL | PIHTAELLAA | 713 | 10 | 17 | 85 | 1882 |
| POL | PLDKGIKPYY | 124 | 10 | 20 | 100 | 1883 |
| POL | PLEEELPRLA | 20 | 10 | 18 | 90 | 1884 |
| ENV | PLGFFPDHQL | 10 | 10 | 19 | 95 | 1885 |
| POL | PLHPAAMPHL | 427 | 10 | 20 | 100 | 1886 |
| ENV | PLLPIFFCLW | 377 | 10 | 20 | 100 | 1887 |
| ENV | PLLVLQAGFF | 174 | 10 | 19 | 95 | 1888 |
| POL | PLPIHTAELL | 711 | 10 | 16 | 80 | 1889 |
| POL | PLSYQHFRKL | 2 | 10 | 15 | 75 | 1890 |
| POL | PLTVNEKRRL | 98 | 10 | 17 | 85 | 1891 |
| POL | PMGVGLSPFL | 505 | 10 | 16 | 80 | 1892 |
| NUC | PPNAPILSTL | 134 | 10 | 20 | 100 | 1893 |
| POL | PVNRPIDWKV | 612 | 10 | 17 | 85 | 1894 |
| NUC | QLLWFHISCL | 99 | 10 | 18 | 90 | 1895 |
| POL | RIVGLLGFAA | 624 | 10 | 18 | 90 | 1696 |
| POL | RLKLIMPARF | 106 | 10 | 15 | 75 | 1897 |
| NUC | RQALCWGEL | 56 | 10 | 18 | 90 | 1896 |
| POL | RVHFASPLHV | 818 | 10 | 15 | 75 | 1899 |
| ENV | SLLVPCQWF | 337 | 10 | 20 | 100 | 1900 |
| X | SLRGLPVCAF | 54 | 10 | 19 | 95 | 1901 |
| POL | SLTNLLSSNL | 403 | 10 | 18 | 90 | 1902 |
| NUC | SPHHTALRQA | 49 | 10 | 20 | 100 | 1903 |
| ENV | SPTVWLSVIW | 350 | 10 | 15 | 75 | 1904 |
| ENV | SVRFSWLSLL | 330 | 10 | 16 | 80 | 1905 |
| ENV | TIPQSLDSWW | 190 | 10 | 18 | 90 | 1906 |
| POL | TPARVTGGVF | 354 | 10 | 18 | 90 | 1907 |
| NUC | TPPAYRPPNA | 128 | 10 | 19 | 95 | 1908 |
| ENV | TPPHGGLLGW | 57 | 10 | 15 | 75 | 1909 |
| POL | VLGAKSVQHL | 543 | 10 | 17 | 85 | 1910 |
| X | VLGGCRHKLV | 133 | 10 | 18 | 90 | 1911 |
| ENV | VPFVQWFVGL | 340 | 10 | 19 | 95 | 1912 |
| POL | VPNLQSLTNL | 398 | 10 | 19 | 95 | 1913 |
| NUC | VQASKLCLGW | 17 | 10 | 16 | 80 | 1914 |
| POL | VVLSRKYTSF | 740 | 10 | 17 | 85 | 1915 |
| POL | WRRAFPHCL | 525 | 10 | 19 | 95 | 1916 |
| POL | WILRGTSFVY | 759 | 10 | 16 | 80 | 1917 |
| POL | WLLGCAANWI | 751 | 10 | 16 | 80 | 1918 |
| POL | WLSLDVSAAF | 414 | 10 | 19 | 95 | 1919 |
| NUC | WLWGMDIDPY | 26 | 10 | 17 | 85 | 1920 |
| ENV | WMCLRRFIIF | 237 | 10 | 19 | 95 | 1921 |
| ENV | WMMWYWGPSL | 359 | 10 | 17 | 85 | 1922 |
| POL | YLHTLWKAGI | 147 | 10 | 20 | 100 | 1923 |
| ENV | YQGMLPVCPL | 263 | 10 | 18 | 90 | 1924 |
| POL | YQHFRKLLLL | 5 | 10 | 15 | 75 | 1925 |
| POL | APFTQCGYPAL | 633 | 11 | 19 | 95 | 1926 |
| POL | AQFTSAICSVV | 516 | 11 | 19 | 95 | 1927 |
| POL | AVPNLQSLTNL | 397 | 11 | 19 | 95 | 1928 |
| ENV | CIPIPSSWAFA | 312 | 11 | 16 | 80 | 1929 |
| POL | CLAFSYMDDVV | 533 | 11 | 18 | 90 | 1930 |
| ENV | CLIFLLVLLDY | 253 | 11 | 19 | 95 | 1931 |
| ENV | CLRRFIIFLFI | 239 | 11 | 15 | 75 | 1932 |
| NUC | CPTVQASKLCL | 14 | 11 | 16 | 80 | 1933 |
| POL | CQRIVGLLGFA | 622 | 11 | 17 | 85 | 1934 |
| POL | DLNLGNLNVSI | 40 | 11 | 19 | 95 | 1935 |
| NUC | ELLSFLPSDFF | 43 | 11 | 19 | 95 | 1936 |
| ENV | FILLLCLIFLL | 248 | 11 | 16 | 80 | 1937 |
| ENV | FLFILLLCLIF | 246 | 11 | 16 | 80 | 1938 |
| ENV | FLLVLLDYQGM | 256 | 11 | 19 | 95 | 1939 |
| ENV | FPAGGSSSGTV | 130 | 11 | 15 | 75 | 1940 |
| POL | FPWLLGCAANW | 749 | 11 | 15 | 75 | 1941 |
| X | FVLGGCRHKLV | 132 | 11 | 18 | 90 | 1942 |
| POL | FVYVPSALNPA | 766 | 11 | 18 | 90 | 1943 |
| ENV | GLSPTVWLSVI | 348 | 11 | 15 | 75 | 1944 |
| POL | GPLEEELPRLA | 19 | 11 | 18 | 90 | 1945 |
| ENV | GPLLVLQAGFF | 173 | 11 | 19 | 95 | 1946 |
| POL | GPLTVNEKRRL | 97 | 11 | 17 | 85 | 1947 |
| X | HLSLRGLPVCA | 52 | 11 | 18 | 90 | 1948 |
| POL | HLYSHPIILGF | 491 | 11 | 16 | 80 | 1949 |
| ENV | IIFLFILLLCL | 244 | 11 | 16 | 80 | 1950 |
| ENV | ILLLCLIFLLV | 249 | 11 | 20 | 100 | 1951 |
| NUC | ILSTLPETIVV | 139 | 11 | 20 | 100 | 1952 |
| ENV | ILTIPQSLDSW | 188 | 11 | 18 | 90 | 1953 |
| POL | IPMGVGLSPFL | 504 | 11 | 16 | 80 | 1954 |
| POL | IVGLLGFAAPF | 625 | 11 | 18 | 90 | 1955 |
| POL | KIPMGVGLSPF | 503 | 11 | 16 | 80 | 1956 |
| POL | KLHLYSNPIIL | 489 | 11 | 16 | 80 | 1957 |
| ENV | LLCLIFLLVLL | 251 | 11 | 20 | 100 | 1958 |
| ENV | LLGWSPQAQGI | 63 | 11 | 15 | 75 | 1959 |
| ENV | LLLCLIFLLVL | 250 | 11 | 20 | 100 | 1960 |
| ENV | LLPIFFCLWVY | 378 | 11 | 20 | 100 | 1961 |
| POL | LLSSNLSWLSL | 407 | 11 | 18 | 90 | 1962 |
| ENV | LLVLLDYQGML | 257 | 11 | 19 | 95 | 1963 |
| ENV | LLVLQAGFFLL | 175 | 11 | 18 | 90 | 1964 |
| NUC | LLWFHISCLTF | 100 | 11 | 17 | 85 | 1965 |
| ENV | LPIFFCLWVYI | 379 | 11 | 20 | 100 | 1966 |
| POL | LPIHTAELLAA | 712 | 11 | 17 | 85 | 1987 |
| POL | LPLDKGIKPYY | 123 | 11 | 20 | 100 | 1968 |
| POL | LPVNRPIDWKV | 611 | 11 | 16 | 80 | 1969 |
| 644 | LQAGFFLLTRI | 178 | 11 | 16 | 80 | 1970 |
| ENV | LVPFVQWFVGL | 339 | 11 | 19 | 95 | 1971 |
| POL | MPHLLVGSSGL | 433 | 11 | 16 | 80 | 1972 |
| POL | MPLSYQHFRKL | 1 | 11 | 15 | 75 | 1973 |
| POL | NLGNLNVSPW | 42 | 11 | 19 | 95 | 1974 |
| POL | NLSWLSLDVSA | 411 | 11 | 18 | 90 | 1975 |
| POL | NPADDPSRGRL | 774 | 11 | 18 | 90 | 1976 |
| ENV | NPLGFFPDHQL | 9 | 11 | 19 | 95 | 1977 |
| POL | PIDWKVCQRV | 616 | 11 | 17 | 85 | 1978 |
| POL | PIILGFRKIPM | 496 | 11 | 16 | 80 | 1979 |
| NUC | PILSTLPETTV | 138 | 11 | 20 | 100 | 1980 |
| POL | PLHPAAMPHLL | 427 | 11 | 20 | 100 | 1981 |
| ENV | PLLPIFFCLWV | 377 | 11 | 20 | 100 | 1982 |
| ENV | PLLVLQAGFFL | 174 | 11 | 18 | 90 | 1983 |
| POL | PLPIHTAELLA | 711 | 11 | 16 | 80 | 1984 |
| POL | PLSYQHFRKLL | 2 | 11 | 15 | 75 | 1985 |
| POL | PMGVGLSPFLL | 505 | 11 | 16 | 80 | 1986 |
| NUC | PPAYRPPNAPI | 129 | 11 | 19 | 95 | 1987 |
| ENV | PQAMQWNSTTF | 106 | 11 | 16 | 80 | 1988 |
| ENV | PQSLDSWWTSL | 192 | 11 | 18 | 90 | 1989 |
| X | QLDPARDVLCL | 8 | 11 | 16 | 80 | 1990 |
| POL | QVFADATPTGW | 885 | 11 | 19 | 95 | 1991 |
| POL | RLKLIMPARFY | 106 | 11 | 15 | 75 | 1992 |
| POL | RPIDWKVCQRI | 615 | 11 | 16 | 80 | 1993 |
| NUC | RPPNAPILSTL | 133 | 11 | 20 | 100 | 1994 |
| NUC | RQAILCWGELM | 56 | 11 | 18 | 90 | 1995 |
| NUC | RQLLWFHISCL | 98 | 11 | 18 | 90 | 1996 |
| POL | RVAEDLNLGNL | 36 | 11 | 18 | 90 | 1997 |
| POL | RVHFASPLHVA | 818 | 11 | 15 | 75 | 1998 |
| POL | SIPWTHKVGNF | 49 | 11 | 20 | 100 | 1999 |
| ENV | SLDSWWTSLNF | 194 | 11 | 19 | 95 | 2000 |
| ENV | SLLVPFVQWFV | 337 | 11 | 19 | 95 | 2001 |
| NUC | SPEHCSPHHTA | 44 | 11 | 20 | 100 | 2002 |
| POL | SPFLLAQFTSA | 511 | 11 | 19 | 95 | 2003 |
| NUC | SPHHTALRQAI | 49 | 11 | 20 | 100 | 2004 |
| ENV | SPTVWLSVIWM | 350 | 11 | 15 | 75 | 2005 |
| ENV | SVRFWLSLLV | 330 | 11 | 16 | 80 | 2006 |
| POL | SVVLSRKYTSF | 739 | 11 | 17 | 85 | 2007 |
| POL | SVVRRAFPHCL | 524 | 11 | 19 | 95 | 2008 |
| POL | TPARVTGGVFL | 354 | 11 | 18 | 90 | 2009 |
| POL | TQCGYPALMPL | 636 | 11 | 19 | 95 | 2010 |
| NUC | TVQASKLCLGW | 16 | 11 | 16 | 80 | 2011 |
| ENV | VLLDYQGMLPV | 259 | 11 | 18 | 90 | 2012 |
| POL | VLSRKYTSFPW | 741 | 11 | 17 | 85 | 2013 |
| POL | VPNLQSLTNLL | 398 | 11 | 19 | 95 | 2014 |
| NUC | VQASKLCLGWL | 17 | 11 | 16 | 80 | 2015 |
| ENV | VQWFVGLSPTV | 343 | 11 | 19 | 95 | 2016 |
| POL | VVLGAKSVQHL | 542 | 11 | 16 | 80 | 2017 |
| POL | WRRAFPHCLA | 525 | 11 | 19 | 95 | 2018 |
| POL | WILRGTSFVYV | 759 | 11 | 16 | 80 | 2019 |
| POL | WLLGCAANWIL | 751 | 11 | 16 | 80 | 2020 |
| POL | WLSLDVSAAFY | 414 | 11 | 19 | 95 | 2021 |
| ENV | WLSLLVPFVQW | 335 | 11 | 20 | 100 | 2022 |
| ENV | WMCLRRFIIFL | 237 | 11 | 19 | 95 | 2023 |
| ENV | WMMWYWGPSLY | 359 | 11 | 17 | 85 | 2024 |
| POL | YLHTLWKAGIL | 147 | 11 | 20 | 100 | 2025 |
| POL | YLPLDKGIKPY | 122 | 11 | 20 | 100 | 2026 |
| POL | YPALMPLYACI | 640 | 11 | 19 | 95 | 2027 |

TABLE XV

HBV A01 Motif with Binding Information

| | | | | | | | |
|---|---|---|---|---|---|---|---|
| Conservancy | Freq. | Protein | Position | Sequence | AA | A*0101 | SEQ ID NO: |

| | | | | | | | |
|---|---|---|---|---|---|---|---|
| 100 | 20 | POL | 166 | ASFCGSPY | 8 | | 2028 |
| 90 | 18 | POL | 737 | DNSWLSRKY | 10 | 0.0001 | 2029 |
| 95 | 19 | POL | 631 | FAAPFTQCGY | 10 | 0.0680 | 2030 |
| 95 | 19 | POL | 630 | GFAAPFTQCGY | 11 | | 2031 |
| 75 | 15 | NUC | 140 | GRETVLEY | 8 | | 2032 |
| 85 | 17 | POL | 579 | GYSLNFMGY | 9 | | 2033 |
| 100 | 20 | POL | 149 | HTLWKAGILY | 10 | 0.1100 | 2034 |
| 95 | 19 | POL | 653 | KQAFTFSPTY | 10 | 0.0001 | 2035 |
| 85 | 17 | NUC | 30 | LLDTASALY | 9 | 12.0000 | 2036 |
| 95 | 19 | POL | 415 | LSLDVSAAFY | 10 | 0.0150 | 2037 |
| 75 | 15 | NUC | 137 | LTFGRETVLEY | 11 | | 2038 |
| 85 | 17 | ENV | 360 | MMWYWGPSLY | 10 | 0.0810 | 2039 |
| 75 | 15 | X | 103 | MSTTDLEAY | 9 | 0.8500 | 2040 |
| 90 | 18 | POL | 738 | NSWLSRKY | 9 | 0.0005 | 2041 |
| 100 | 20 | POL | 124 | PLDKGIKPY | 9 | | 2042 |
| 100 | 20 | POL | 124 | PLDKGIKPYY | 10 | 0.1700 | 2043 |
| 85 | 17 | POL | 797 | PTTGRTSLY | 9 | 0.2100 | 2044 |
| 100 | 20 | POL | 165 | SASFCGSPY | 9 | | 2045 |
| 95 | 19 | POL | 416 | SLDVSAAFY | 9 | 5.2000 | 2046 |
| 75 | 15 | X | 104 | STTDLEAY | 8 | | 2047 |
| 85 | 17 | POL | 798 | TTGRTSLY | 8 | | 2048 |
| 95 | 19 | POL | 414 | WLSLDVSAAFY | 11 | | 2049 |
| 85 | 17 | ENV | 359 | WMMWYWGPSLY | 11 | 0.3200 | 2050 |
| 95 | 19 | POL | 640 | YPALMPLY | 8 | | 2051 |
| 85 | 17 | POL | 580 | YSLNFMGY | 8 | | 2052 |

TABLE XVI

HBV A03 Motif With Binding

| | | | | | | | |
|---|---|---|---|---|---|---|---|
| Conservancy | Freq. | Protein | Position | Sequence | AA | A*0301 | Seq ID Num |

| | | | | | | | |
|---|---|---|---|---|---|---|---|
| 85 | 17 | POL | 721 | AACFARSR | 8 | 0.0004 | 2053 |
| 85 | 17 | POL | 721 | AACFARSRSGA | 11 | | 2054 |
| 95 | 19 | POL | 632 | AAPFTQCGY | 9 | | 2055 |
| 95 | 19 | POL | 632 | AAPFTQCGYPA | 11 | | 2056 |
| 85 | 17 | POL | 722 | ACFARSRSGA | 10 | | 2057 |
| 80 | 16 | POL | 688 | ADATPTGWGLA | 11 | | 2058 |
| 90 | 18 | POL | 776 | ADDPSRGR | 8 | | 2059 |
| 95 | 19 | POL | 529 | AFPHCLAF | 8 | | 2060 |
| 95 | 19 | POL | 529 | AFPHCLAFSY | 10 | | 2061 |
| 95 | 19 | X | 62 | AFSSAGPCA | 9 | | 2062 |
| 90 | 18 | X | 62 | AFSSAGPCALR | 11 | | 2063 |
| 95 | 19 | POL | 655 | AFTFSPTY | 8 | | 2064 |
| 95 | 19 | POL | 655 | AFTFSPTYK | 9 | 0.2600 | 2065 |
| 95 | 19 | POL | 655 | AFTFSPTYKA | 10 | | 2066 |
| 95 | 19 | POL | 655 | AFTFSPTYKAF | 11 | | 2067 |
| 80 | 16 | ENV | 180 | AGFFLLTR | 8 | | 2068 |
| 90 | 18 | X | 66 | AGPCALRF | 8 | | 2069 |
| 90 | 18 | X | 66 | AGPCALRFTSA | 11 | | 2070 |
| 95 | 19 | POL | 18 | AGPLEEELPR | 10 | 0.0004 | 2071 |
| 95 | 19 | POL | 521 | AICSWRR | 8 | −0.0002 | 2072 |
| 95 | 19 | POL | 521 | AICSVVRRA | 9 | | 2073 |
| 95 | 19 | POL | 521 | AICSWRRAF | 10 | | 2074 |
| 95 | 19 | POL | 41 | ALESPEHCSPH | 11 | | 2075 |
| 90 | 18 | POL | 772 | ALNPADDPSR | 10 | 0.0003 | 2076 |
| 85 | 17 | X | 70 | ALRFTSAR | 8 | 0.0047 | 2077 |
| 80 | 16 | ENV | 108 | AMQWNSTTF | 9 | | 2078 |
| 80 | 16 | ENV | 108 | AMQWNSTTFH | 10 | | 2079 |
| 75 | 15 | X | 102 | AMSITDLEA | 9. | | 2080 |
| 85 | 17 | NUC | 34 | ASALYREA | 8 | | 2081 |
| 100 | 20 | POL | 166 | ASFCGSPY | 8 | 0.0460 | 2082 |
| 80 | 16 | POL | 822 | ASPLHVAWR | 9 | | 2083 |
| 75 | 15 | ENV | 84 | ASTNRQSGR | 9 | 0.0009 | 2084 |
| 80 | 16 | POL | 690 | ATPTGWGLA | 9 | | 2085 |
| 80 | 16 | POL | 755 | CAANWILR | 8 | | 2086 |
| 95 | 19 | X | 61 | CAFSSAGPCA | 10 | | 2087 |
| 90 | 18 | X | 69 | CALRFTSA | 8 | | 2088 |
| 85 | 17 | X | 69 | CALRFTSAR | 9 | 0.0034 | 2089 |
| 80 | 16 | X | 6 | CCQLDPAR | 8 | | 2090 |
| 85 | 17 | POL | 723 | CFARSRSGA | 9 | | 2091 |
| 75 | 15 | POL | 607 | CFRKLPVNR | 9 | | 2092 |
| 95 | 19 | POL | 638 | CGYPALMPLY | 10 | | 2093 |
| 95 | 19 | POL | 638 | CGYPALMPLYA | 11 | | 2094 |
| 100 | 20 | ENV | 312 | CIPIPSSWA | 9 | | 2095 |
| 100 | 20 | ENV | 312 | CIPIPSSWAF | 10 | | 2096 |
| 80 | 16 | EVN | 312 | CIPIPSSWAFA | 11 | | 2097 |
| 95 | 19 | ENV | 253 | CLIFLLVLLDY | 11 | 0.0083 | 2098 |
| 90 | 18 | X | 17 | CLRPVGAESR | 10 | 0.0011 | 2099 |
| 95 | 19 | ENV | 239 | CLRRFIIF | 8 | | 2100 |
| 75 | 15 | ENV | 239 | CLRRFIIFLF | 10 | | 2101 |
| 100 | 20 | NUC | 48 | CSPHHTALR | 9 | 0.0029 | 2102 |
| 100 | 20 | NUC | 48 | CSPHHTALRQA | 11 | | 2103 |
| 95 | 19 | POL | 523 | CSVVRRAF | 8 | | 2104 |
| 95 | 19 | POL | 523 | CSVVRRAFPH | 10 | | 2105 |
| 100 | 20 | ENV | 310 | CTCIPIPSSWA | 11 | | 2106 |
| 80 | 16 | POL | 689 | DATPTGWGLA | 10 | | 2107 |
| 90 | 18 | POL | 540 | DDVVLGAK | 8 | | 2108 |
| 90 | 18 | NUC | 31 | DIDPYKEF | 8 | | 2109 |
| 90 | 18 | NUC | 31 | DIDPYKEFGA | 10 | | 2110 |
| 85 | 17 | NUC | 29 | DLLDTASA | 8 | | 2111 |
| 85 | 17 | NUC | 29 | DLLDTASALY | 10 | 0.0001 | 2112 |
| 85 | 17 | NUC | 29 | DLLDTASALYR | 11 | 0.0042 | 2113 |
| 95 | 19 | ENV | 196 | DSWWTSLNF | 9 | 0.0006 | 2114 |
| 85 | 17 | NUC | 32 | DTASALYR | 8 | 0.0004 | 2115 |
| 80 | 16 | NUC | 32 | DTASALYREA | 10 | | 2116 |
| 95 | 19 | X | 14 | DVLCLRPVGA | 10 | | 2117 |
| 95 | 19 | POL | 418 | DVSAAFYH | 8 | | 2118 |
| 90 | 18 | POL | 541 | DVVLGAKSVQH | 11 | | 2119 |
| 95 | 19 | POL | 17 | EAGPLEEELPR | 11 | −0.0009 | 2120 |
| 90 | 18 | NUC | 40 | EALESPEH | 8 | | 2121 |
| 90 | 18 | POL | 718 | ELLAACFA | 8 | | 2122 |
| 90 | 18 | POL | 718 | ELLAACFAR | 9 | 0.0002 | 2123 |
| 85 | 17 | POL | 718 | ELLAACFARSR | 11 | 0.0082 | 2124 |
| 95 | 19 | NUC | 43 | ELLSFLPSDF | 10 | | 2125 |
| 95 | 19 | NUC | 43 | ELLSFLPSDFF | 11 | | 2126 |
| 95 | 19 | NUC | 43 | ESPEHCSPH | 9 | | 2127 |
| 95 | 19 | NUC | 43 | ESPEHCSPHH | 10 | | 2128 |
| 95 | 19 | POL | 374 | ESRLVVDF | 8 | | 2129 |
| 95 | 19 | POL | 374 | ESRLVVDFSQF | 11 | | 2130 |
| 95 | 19 | NUC | 174 | ETTVVRRR | 8 | 0.0003 | 2131 |
| 80 | 16 | NUC | 174 | ETTVVRRRGR | 10 | 0.0003 | 2132 |
| 95 | 19 | POL | 631 | FAAPFTQCGY | 10 | | 2133 |
| 85 | 17 | POL | 724 | FARSRSGA | 8 | | 2134 |
| 80 | 16 | POL | 821 | FASPLHVA | 8 | | 2135 |
| 80 | 16 | POL | 821 | FASPLHVAWR | 10 | | 2136 |
| 90 | 18 | ENV | 13 | FFPDHQLDPA | 10 | | 2137 |
| 85 | 17 | ENV | 13 | FFPDHQLDPAF | 11 | | 2138 |
| 75 | 15 | NUC | 139 | FGRETVLEY | 9 | | 2139 |
| 75 | 15 | POL | 244 | FGVEPSGSGH | 10 | | 2140 |
| 95 | 19 | NUC | 122 | FGVWIRTPPA | 10 | | 2141 |
| 95 | 19 | NUC | 122 | FGVWIRTPPAY | 11 | | 2142 |
| 80 | 16 | ENV | 248 | FILLLCLIF | 9 | | 2143 |
| 80 | 16 | ENV | 246 | FLFILLLCLIF | 11 | | 2144 |
| 75 | 15 | ENV | 171 | FLGPLLVLQA | 10 | | 2145 |
| 95 | 19 | POL | 513 | FLLAQFTSA | 9 | 0.0006 | 2146 |
| 95 | 19 | POL | 562 | FLLSLGIH | 8 | | 2147 |
| 95 | 19 | ENV | 256 | FLLVLLDY | 8 | 0.0050 | 2148 |
| 100 | 20 | POL | 363 | FLVDKNPH | 8 | | 2149 |
| 95 | 19 | POL | 658 | FSPTYKAF | 8 | | 2150 |
| 95 | 19 | X | 63 | FSSAGPCA | 8 | | 2151 |
| 90 | 18 | X | 63 | FSSAGPCALR | 10 | | 2152 |
| 90 | 18 | X | 63 | FSSAGPCALRF | 11 | | 2153 |
| 100 | 20 | ENV | 333 | FSWLSLLVPF | 10 | 0.0004 | 2154 |
| 90 | 18 | POL | 536 | FSYMDDVVLGA | 11 | | 2155 |
| 95 | 19 | POL | 656 | FTFSPTYK | 8 | 0.0100 | 2156 |
| 95 | 19 | POL | 656 | FTFSPTYKA | 9 | | 2157 |
| 95 | 19 | POL | 656 | FTFSPTYKAF | 10 | 0.0004 | 2158 |
| 95 | 19 | POL | 635 | FTOCGYPA | 8 | | 2159 |
| 95 | 19 | POL | 518 | FTSAICSVVR | 10 | 0.0003 | 2160 |
| 95 | 19 | POL | 518 | FTSAICSVVRR | 11 | 0.0065 | 2161 |
| 95 | 19 | X | 132 | FVLGGCRH | 8 | | 2162 |
| 90 | 18 | X | 132 | FVLGGCRHK | 9 | 0.0430 | 2163 |
| 90 | 18 | POL | 766 | FVYVPSALNPA | 11 | | 2164 |
| 80 | 16 | POL | 754 | GCAANWILR | 9 | | 2165 |
| 95 | 19 | POL | 630 | GFAAPFTOOGY | 11 | | 2166 |
| 90 | 18 | ENV | 12 | GFFPDHOLDPA | 11 | | 2167 |
| 75 | 15 | ENV | 170 | GFLGPLLVLOA | 11 | | 2168 |
| 85 | 17 | ENV | 61 | GGLLGWSPQA | 10 | | 2169 |
| 100 | 20 | POL | 360 | GGVFLVDK | 8 | | 2170 |
| 100 | 20 | POL | 360 | GGVFLVDKNPH | 11 | | 2171 |
| 75 | 15 | POL | 567 | GIHLNPNK | 8 | | 2172 |
| 75 | 15 | POL | 567 | GIHLNPNKTK | 10 | 0.0025 | 2173 |
| 75 | 15 | POL | 567 | GIHLNPNKTKR | 11 | | 2174 |
| 85 | 17 | POL | 682 | GLCQVFADA | 9 | 0.0001 | 2175 |
| 95 | 19 | POL | 627 | GLLGFAAPF | 9 | 0.0006 | 2176 |
| 85 | 17 | ENV | 62 | GLLGWSPOA | 9 | | 2177 |
| 95 | 19 | X | 57 | GLPVCAFSSA | 10 | | 2178 |
| 95 | 19 | POL | 509 | GLSPFLLA | 8 | | 2179 |
| 95 | 19 | POL | 509 | GLSPFLLAQF | 10 | | 2180 |
| 85 | 17 | NUC | 29 | GMDIDPYK | 8 | 0.0006 | 2181 |
| 85 | 17 | NUC | 29 | GMDIDPYKEF | 10 | −0.0003 | 2182 |
| 90 | 18 | POL | 735 | GTDNSVVLSR | 10 | 0.0010 | 2183 |
| 90 | 18 | POL | 735 | GTDNSVVLSRK | 11 | 0.0140 | 2184 |
| 80 | 16 | POL | 763 | GTSFVYVPSA | 10 | | 2185 |
| 80 | 16 | POL | 245 | GVEPSGSGH | 9 | | 2186 |
| 100 | 20 | POL | 361 | GVFLVDKNPH | 10 | | 2187 |
| 80 | 16 | POL | 507 | GVGLSPFLLA | 10 | | 2188 |
| 95 | 19 | NUC | 123 | GVWIRTPPA | 9 | | 2189 |
| 95 | 19 | NUC | 123 | GVWIRTPPAY | 10 | 0.0047 | 2190 |
| 95 | 19 | NUC | 123 | GVWIRTPPAYR | 11 | 0.1900 | 2191 |
| 100 | 20 | NUC | 47 | HCSPHHTA | 8 | | 2192 |
| 100 | 20 | NUC | 47 | HCSPHHTALR | 10 | | 2193 |
| 80 | 16 | POL | 820 | HFASPLHVA | 9 | | 2194 |
| 80 | 16 | POL | 820 | HFASPLHVAWR | 11 | | 2195 |
| 95 | 19 | X | 49 | HGAHLSLR | 8 | | 2196 |
| 85 | 17 | ENV | 60 | HGGLLGWSPQA | 11 | | 2197 |
| 90 | 18 | NUC | 104 | HISCLTFGR | 9 | | 2198 |
| 75 | 15 | POL | 569 | HLNPNKTK | 8 | | 2199 |
| 75 | 15 | POL | 569 | HLNPNKTKR | 9 | | 2200 |
| 90 | 18 | X | 52 | HLSLRGLPVCA | 11 | | 2201 |
| 80 | 16 | POL | 491 | HLYSHPIILGF | 11 | | 2202 |
| 85 | 17 | POL | 715 | HTAELLAA | 8 | | 2203 |
| 85 | 17 | POL | 715 | HTAELLAACF | 10 | | 2204 |
| 85 | 17 | POL | 715 | HTAELLAACFA | 11 | | 2205 |
| 100 | 20 | POL | 149 | HTLWKAGILY | 10 | 0.0440 | 2206 |
| 100 | 20 | POL | 149 | HTLWKAGILYK | 11 | 0.5400 | 2207 |
| 95 | 19 | POL | 522 | ICSVVRRA | 8 | | 2208 |
| 95 | 19 | POL | 522 | ICSVVRRAF | 9 | | 2209 |
| 95 | 19 | POL | 522 | ICSVVRRAFPH | 11 | | 2210 |
| 90 | 18 | NUC | 32 | IDPYKEFGA | 9 | | 2211 |
| 90 | 18 | POL | 617 | IDWKVCQR | 8 | | 2212 |
| 100 | 20 | ENV | 381 | IFFCLWVY | 8 | | 2213 |
| 95 | 19 | ENV | 255 | IFLIVLLDY | 9 | | 2214 |
| 80 | 16 | POL | 734 | IGTDNSVVLSR | 11 | | 2215 |
| 100 | 20 | ENV | 249 | ILILCLIF | 8 | | 2216 |
| 80 | 16 | POL | 760 | ILRGTSFVY | 9 | 0.0440 | 2217 |
| 90 | 18 | NUC | 105 | IDCLTFGR | 8 | 0.0004 | 2218 |
| 90 | 18 | POL | 625 | IVGLLGFA | 8 | | 2219 |
| 90 | 18 | POL | 625 | IVGLLGFAA | 9 | | 2220 |
| 90 | 18 | POL | 625 | IVGLLGFAAPF | 11 | | 2221 |
| 100 | 20 | POL | 153 | KAGILYKR | 8 | 0.0002 | 2222 |
| 80 | 16 | POL | 503 | KIPMGVGLSPF | 11 | | 2223 |
| 75 | 15 | POL | 108 | KLIMPARF | 8 | | 2224 |
| 75 | 15 | POL | 108 | KLIMPARFY | 9 | | 2225 |
| 80 | 16 | POL | 610 | KLPVNRPIDWK | 11 | | 2226 |
| 85 | 17 | POL | 574 | KTKRWGYSLNF | 11 | | 2227 |
| 75 | 15 | X | 130 | KVFVLGGCR | 9 | 0.0420 | 2228 |
| 75 | 15 | X | 130 | KVFVIGGCRH | 10 | | 2229 |
| 95 | 19 | POL | 55 | KVGNFTGLY | 9 | 0.2100 | 2230 |
| 85 | 17 | POL | 720 | LAACFARSR | 9 | 0.0058 | 2231 |
| 95 | 19 | X | 16 | LCLRPVGA | 8 | | 2232 |
| 90 | 18 | X | 16 | LCLRPVGAESR | 11 | | 2233 |
| 95 | 19 | POL | 683 | LCQVFADA | 8 | | 2234 |
| 100 | 20 | POL | 125 | LDKGIKPY | 8 | | 2235 |
| 100 | 20 | POL | 125 | LDKGIKPYY | 9 | | 2236 |
| 80 | 16 | X | 9 | LDPARDVLCLR | 11 | | 2237 |
| 95 | 19 | ENV | 195 | LDSWWTSLNF | 10 | | 2238 |
| 85 | 17 | NUC | 31 | LDTASALY | 8 | | 2239 |
| 85 | 17 | NUC | 31 | LDTASALYR | 9 | 0.0004 | 2240 |
| 80 | 16 | NUC | 31 | LDTASALYREA | 11 | | 2241 |
| 95 | 19 | POL | 417 | LDVSAAFY | 8 | | 2242 |
| 95 | 19 | POL | 417 | LDVSAAFYH | 9 | | 2243 |
| 80 | 16 | ENV | 247 | LFILLLCLIF | 10 | | 2244 |
| 95 | 19 | POL | 544 | LGAKSVQH | 8 | | 2245 |
| 80 | 16 | POL | 753 | LGCAANWILR | 10 | | 2246 |
| 75 | 15 | P0L | 566 | LGIHLNPNK | 9 | | 2247 |
| 75 | 15 | POL | 566 | LGIHLNPNKTK | 11 | | 2248 |
| 95 | 19 | ENV | 172 | LGPLLVLQA | 9 | | 2249 |
| 95 | 19 | ENV | 172 | LGPLLVLQAGF | 11 | | 2250 |
| 95 | 19 | ENV | 254 | LIFLLVLLDY | 10 | 0.0022 | 2251 |
| 100 | 20 | POL | 109 | LIMPARFY | 8 | −0.0002 | 2252 |
| 90 | 18 | POL | 719 | LLAACFAR | 8 | 0.0024 | 2253 |
| 85 | 17 | POL | 719 | LLAACFARSR | 10 | | 2254 |
| 95 | 19 | POL | 514 | LLAQFTSA | 8 | | 2255 |
| 85 | 17 | NUC | 30 | LLDTASALY | 9 | 0.0013 | 2256 |
| 85 | 17 | NUC | 30 | LLDTASALYR | 10 | 0.0050 | 2257 |
| 80 | 16 | POL | 752 | LLGCAANWILR | 11 | | 2258 |
| 95 | 19 | POL | 628 | LLGFAAPF | 8 | | 2259 |
| 85 | 17 | ENV | 63 | LLGWSPQA | 8 | | 2260 |
| 100 | 20 | ETV | 378 | LLPIFFCLWVY | 11 | 0.0230 | 2261 |
| 95 | 19 | NUC | 44 | LLSFLPSDF | 9 | | 2262 |
| 95 | 19 | NUC | 44 | LLSFLPSDFF | 10 | | 2263 |
| 95 | 19 | ENV | 175 | LLVLQAGF | 8 | | 2264 |
| 95 | 19 | ENV | 175 | LLVLQAGFF | 9 | 0.0006 | 2265 |
| 100 | 20 | ENV | 336 | LLVPFVQWF | 9 | | 2266 |
| 85 | 17 | NUC | 100 | LLWFHISCLTF | 11 | | 2267 |
| 95 | 19 | NUC | 45 | LSFLPSDF | 8 | | 2268 |
| 95 | 19 | NUC | 45 | LSFLPSDFF | 9 | 0.0006 | 2269 |
| 95 | 19 | POL | 415 | LSLDVSAA | 8 | | 2270 |
| 95 | 19 | POL | 415 | LSLDVSAAF | 9 | 0.0004 | 2271 |
| 95 | 19 | POL | 415 | LSLDVSAAFY | 10 | | 2272 |
| 95 | 19 | POL | 415 | LSLDVSAAFYI | 11 | | 2273 |
| 75 | 15 | POL | 564 | LSLGIHLNPNK | 11 | | 2274 |
| 100 | 20 | ENV | 336 | LSLLVPFVQWF | 11 | | 2275 |
| 95 | 19 | X | 53 | LSLRGLPVCA | 10 | | 2276 |
| 95 | 19 | X | 53 | LSLRGLPVCAF | 11 | | 2277 |
| 95 | 19 | POL | 510 | LSPFLLAQF | 9 | | 2278 |
| 9 5 85 | 17 | POL | 742 | LSRKYTSF | 8 | | 2279 |
| 95 | 19 | NUC | 169 | LSTLPETTVVR | 11 | −0.0009 | 2280 |
| 75 | 15 | ENV | 16 | LSVPNPLGF | 9 | | 2281 |
| 100 | 20 | POL | 412 | LSWLSLDVSA | 10 | 0.0048 | 2282 |
| 100 | 19 | POL | 412 | LSWLSLDVSAA | 11 | | 2283 |
| 95 | 15 | POL | 3 | LSYQIIFRK | 8 | | 2284 |
| 75 | 15 | NUC | 137 | LTFGRETVLEY | 11 | | 2285 |
| 85 | 17 | POL | 99 | LTVNEKRR | 8 | −0.0002 | 2286 |
| 95 | 19 | ENV | 176 | LVLQAGFF | 8 | | 2287 |
| 100 | 20 | ENV | 339 | LVPFVQWF | 8 | 0.0028 | 2288 |
| 90 | 18 | NUC | 119 | LVSFGVWIR | 9 | | 2289 |
| 100 | 20 | POL | 377 | LWDFSQF | 8 | 0.0016 | 2290 |
| 100 | 20 | POL | 377 | LWDFSQFSR | 10 | | 2291 |
| 95 | 19 | ENV | 238 | MCLRRFIIF | 9 | | 2292 |
| 75 | 15 | ENV | 238 | MCLRRFIIFLF | 11 | | 2293 |
| 90 | 18 | POL | 539 | MDDWLGA | 8 | | 2294 |
| 90 | 18 | POL | 539 | MDDWLGAK | 9 | | 2295 |
| 90 | 18 | NUC | 30 | MDIDPYKEF | 9 | | 2296 |
| 90 | 18 | NUC | 30 | MDIDPYKEFGA | 11 | | 2297 |
| 80 | 16 | POL | 506 | MGVGLSPF | 8 | | 2298 |
| 80 | 16 | POL | 506 | MGVGLSPFLLA | 11 | | 2299 |
| 85 | 17 | ENV | 360 | MMWYWGPSLY | 10 | 0.0500 | 2300 |
| 80 | 16 | X | 103 | MSTTDLEA | 8 | | 2301 |
| 75 | 15 | X | 103 | MSTTDLEAY | 9 | 0.0008 | 2302 |
| 75 | 15 | X | 103 | MSTTDLEAYF | 10 | | 2303 |
| 75 | 15 | X | 103 | MSTTDLEAYFK | 11 | | 2304 |
| 95 | 19 | POL | 561 | NFLLSLGIH | 9 | | 2305 |
| 90 | 18 | NUC | 75 | NLEDPASR | 8 | −0.0002 | 2306 |
| 95 | 19 | POL | 45 | NLNVSIPWTH | 10 | | 2307 |
| 95 | 19 | POL | 45 | NLNVSIPWTHK | 11 | −0.0009 | 2308 |
| 95 | 15 | ENV | 15 | NLSVPNPLGF | 10 | | 2309 |
| 90 | 18 | POL | 411 | NLSWLSLDVSA | 11 | | 2310 |
| 75 | 15 | ENV | 215 | NSQSPTSNH | 9 | | 2311 |
| 90 | 18 | POL | 738 | NSVVLSRK | 8 | 0.0006 | 2312 |
| 90 | 18 | POL | 738 | NSVVLSRKY | 9 | 0.0020 | 2313 |
| 100 | 20 | POL | 47 | NVSIPWTH | 8 | | 2314 |
| 100 | 20 | POL | 47 | NVSIPWTHK | 9 | 0.0820 | 2315 |
| 90 | 18 | POL | 775 | PADDPSRGR | 9 | 0.0008 | 2316 |
| 95 | 19 | POL | 641 | PALMPLYA | 8 | | 2317 |
| 75 | 15 | X | 145 | PAPCNFFTSA | 10 | | 2318 |
| 80 | 16 | X | 11 | PARDVLCLR | 9 | 0.0002 | 2319 |
| 90 | 18 | POL | 355 | PARVTGGVF | 9 | | 2320 |
| 75 | 15 | ENV | 83 | PASTNRQSGR | 10 | | 2321 |
| 95 | 19 | NUC | 130 | PAYRPPNA | 8 | | 2322 |
| 90 | 18 | X | 68 | PCALRFTSA | 9 | | 2323 |
| 85 | 17 | X | 68 | PCALRFTSAR | 10 | | 2324 |
| 75 | 15 | X | 147 | PCNFFTSA | 8 | | 2325 |
| 95 | 19 | ENV | 15 | PDHQLDPA | 8 | | 2326 |
| 90 | 18 | ENV | 15 | PDHQLDPAF | 9 | | 2327 |
| 95 | 19 | POL | 512 | PFLLAQFTSA | 10 | | 2328 |
| 95 | 19 | POL | 634 | PFTQOGYPA | 9 | | 2329 |
| 100 | 20 | ENV | 233 | PGYRMMCLR | 9 | 0.0008 | 2330 |
| 95 | 19 | ENV | 233 | PGYRWMCLRR | 10 | 0.0048 | 2331 |
| 95 | 19 | ENV | 233 | PGYRWMCLRRF | 11 | | 2332 |
| 90 | 18 | POL | 616 | PIDWKVCQR | 9 | 0.0002 | 2333 |
| 100 | 20 | ENV | 380 | PIFFCLWVY | 9 | 0.0011 | 2334 |
| 85 | 17 | POL | 713 | PIHTAELLA | 9 | | 2335 |
| 85 | 17 | POL | 713 | PIHTAELLAA | 10 | | 2336 |
| 80 | 16 | POL | 496 | PIILGFRK | 8 | | 2337 |
| 100 | 20 | ENV | 314 | PIPSSWAF | 8 | | 2338 |
| 80 | 16 | ENV | 314 | PIPSSWAFA | 9 | | 2339 |
| 100 | 20 | POL | 124 | PLDKGIKPY | 9 | 0.0001 | 2340 |
| 100 | 20 | POL | 124 | PLDKGIKPYY | 10 | 0.0002 | 2341 |
| 95 | 19 | POL | 20 | PLEEELPR | 8 | 0.0002 | 2342 |
| 90 | 16 | POL | 20 | PLEEELPRLA | 10 | | 2343 |
| 90 | 19 | ENV | 10 | PLGFFPDH | 8 | | 2344 |
| 100 | 20 | POL | 427 | PLHPAAMPH | 9 | 0.0012 | 2345 |
| 95 | 19 | ENV | 174 | PLLVLQAGF | 9 | | 2346 |
| 95 | 19 | ENV | 174 | PLLVLQAGFF | 10 | | 2347 |
| 80 | 16 | POL | 711 | PLPIHTAELLA | 11 | | 2348 |
| 100 | 20 | POL | 2 | PLSYQHFR | 8 | −0.0002 | 2349 |
| 75 | 15 | POL | 2 | PLSYQHFRK | 9 | 0.0011 | 2350 |
| 85 | 17 | POL | 98 | PLTVNEKR | 8 | 0.0002 | 2351 |
| 85 | 17 | POL | 98 | PLTVNEKRR | 9 | 0.0008 | 2352 |
| 80 | 16 | POL | 505 | PMGVGLSPF | 9 | | 2353 |
| 85 | 17 | POL | 797 | PTTGRTSLY | 9 | 0.0001 | 2354 |
| 85 | 17 | POL | 797 | PTTGRTSLYA | 10 | | 2355 |
| 95 | 19 | X | 59 | PVCAFSSA | 8 | | 2356 |
| 90 | 18 | X | 20 | PVGAESRGR | 9 | 0.0002 | 2357 |
| 85 | 17 | POL | 612 | PVNRPIDWK | 9 | 0.0310 | 2358 |
| 95 | 19 | POL | 654 | QAFTFSPTY | 9 | 0.0030 | 2359 |
| 95 | 19 | POL | 654 | QAFTFSPTYK | 10 | 0.0450 | 2360 |
| 95 | 19 | POL | 654 | QAFTFSPTYKA | 11 | | 2361 |
| 80 | 16 | ENV | 179 | QAGFRLLTR | 9 | | 2362 |
| 80 | 16 | ENV | 107 | QAMQWNSTTF | 10 | | 2363 |
| 80 | 16 | ENV | 107 | QAMQWNSTTFH | 11 | | 2364 |
| 95 | 19 | POL | 637 | QCGYPALMPLY | 11 | | 2365 |
| 95 | 19 | POL | 517 | QFTSAICSVVR | 11 | | 2366 |
| 75 | 15 | NUC | 169 | QSPRRRRSQSR | 11 | | 2367 |
| 80 | 16 | POL | 189 | QSSGILSR | 8 | | 2368 |
| 95 | 19 | POL | 528 | RAFPHCLA | 8 | | 2369 |
| 95 | 19 | POL | 528 | RAFPHCLAF | 9 | 0.0015 | 2370 |
| 95 | 19 | POL | 528 | RAFPHCIAFSY | 11 | 0.1200 | 2371 |
| 85 | 17 | NUC | 28 | RDLLDTASA | 9 | | 2372 |
| 85 | 17 | NUC | 28 | RDLLDTASALY | 11 | | 2373 |
| 95 | 19 | X | 13 | RDVLCLRPVGA | 11 | | 2374 |
| 100 | 20 | ENV | 332 | RFSWLSLLVPF | 11 | | 2375 |
| 95 | 19 | X | 56 | RGLPVCAF | 8 | | 2376 |
| 95 | 19 | X | 56 | RGLPVCAFSSA | 11 | | 2377 |
| 100 | 20 | NUC | 152 | RGRSPRRR | 8 | | 2378 |
| 80 | 16 | POL | 762 | RGTSFVYVPSA | 11 | | 2379 |
| 90 | 18 | POL | 624 | RNGLLGF | 8 | | 2380 |
| 90 | 18 | POL | 624 | RIVGLLGFA | 9 | | 2381 |
| 90 | 18 | POL | 624 | RIVGLLGFAA | 10 | | 2382 |
| 75 | 15 | POL | 106 | RLKLIMPA | 8 | | 2383 |
| 75 | 15 | POL | 106 | RLKIMPAR | 9 | 0.0950 | 2384 |
| 75 | 15 | POL | 106 | RLKLIMPARF | 10 | | 2385 |
| 75 | 15 | POL | 106 | RLKLIMPARFY | 11 | | 2386 |
| 75 | 15 | X | 128 | RLKVFVLGGCR | 11 | | 2387 |
| 95 | 19 | POL | 376 | RLVVDFSQF | 9 | 0.0006 | 2388 |
| 95 | 19 | POL | 376 | RLVVDFSQFSR | 11 | 0.2800 | 2389 |
| 95 | 19 | NUC | 163 | RSPRRRTPSPR | 11 | −0.0007 | 2390 |
| 75 | 15 | NUC | 167 | RSQSPRRR | 8 | | 2391 |
| 75 | 15 | NUC | 167 | RSQSPRRRR | 9 | | 2392 |
| 90 | 18 | POL | 353 | RTPARVTGGVF | 11 | | 2393 |
| 95 | 19 | NUC | 127 | RTPPAYRPPNA | 11 | | 2394 |
| 95 | 19 | NUC | 188 | RTPSPRRR | 8 | −0.0002 | 2395 |
| 95 | 19 | NUC | 188 | RTPSPRRPR | 9 | 0.0054 | 2396 |
| 95 | 16 | POL | 818 | RVHFASPLH | 9 | | 2397 |
| 75 | 15 | POL | 818 | RVHFASPLHVA | 11 | | 2398 |
| 100 | 20 | POL | 357 | RVTGGVFIVDK | 11 | 0.0190 | 2399 |
| 90 | 18 | X | 65 | SAGPCALR | 8 | −0.0002 | 2400 |
| 90 | 18 | X | 65 | SAGPCALRF | 9 | −0.0003 | 2401 |
| 95 | 19 | POL | 520 | SAICSVVR | 8 | −0.0002 | 2402 |
| 95 | 19 | POL | 520 | SAICSVVRR | 9 | 0.0058 | 2403 |
| 95 | 19 | POL | 520 | SAICSWRRA | 10 | | 2404 |
| 95 | 19 | POL | 520 | SAICSWRRAF | 11 | | 2405 |
| 95 | 18 | POL | 771 | SALNPADDPSR | 11 | −0.0004 | 2406 |
| 90 | 20 | POL | 165 | SASFCGSPY | 9 | | 2407 |
| 100 | 18 | NUC | 121 | SFGVWIRTPPA | 11 | | 2408 |
| 90 | 19 | NUC | 46 | SFLPSDFF | 8 | | 2409 |
| 95 | 15 | POL | 748 | SFPWLGCA | 9 | | 2410 |
| 75 | 15 | POL | 740 | SFPWLLGCAA | 10 | | 2411 |
| 75 | 16 | POL | 765 | SFVWPSA | 8 | | 2412 |
| 80 | 20 | POL | 49 | SIPWTHKVGNF | 11 | | 2413 |
| 100 | 19 | ENV | 194 | SLDSWWTSINF | 11 | | 2414 |
| 95 | 19 | POL | 416 | SLDVSAAF | 8 | | 2415 |
| 95 | 19 | POL | 416 | SIQVSAAFY | 9 | 0.0016 | 2416 |
| 95 | 19 | POL | 416 | SLDVSAAFYH | 10 | | 2417 |
| 75 | 15 | POL | 565 | SIGIHLNPNK | 10 | | 2418 |
| 100 | 20 | ENV | 337 | SLLVPFVQWF | 10 | | 2419 |
| 95 | 19 | X | 54 | SLRGLPVCA | 9 | | 2420 |
| 95 | 19 | X | 54 | SLRGLPVCAF | 10 | 0.0004 | 2421 |
| 95 | 18 | X | 64 | SSAGPCALR | 9 | 0.0080 | 2422 |
| 90 | 18 | X | 64 | SSAGPCALRF | 10 | −0.0003 | 2423 |
| 90 | 19 | NUC | 170 | STIPETTVVR | 10 | 0.0007 | 2424 |
| 95 | 19 | NUC | 170 | STLPETTWRR | 11 | 0.0150 | 2425 |
| 95 | 16 | ENV | 85 | STNRQSGR | 8 | | 2426 |
| 80 | 15 | X | 104 | STTDLEAY | 8 | | 2427 |
| 75 | 15 | X | 104 | STTDLEAYF | 9 | | 2428 |
| 75 | 15 | X | 104 | STTDLEAYFK | 10 | 0.0066 | 2429 |
| 75 | 15 | ENV | 17 | SVPNPLGF | 8 | | 2430 |
| 90 | 18 | POL | 739 | SVVLSRKY | 8 | −0.0002 | 2431 |
| 85 | 17 | POL | 739 | SVVLSRKYTSF | 11 | | 2432 |
| 95 | 19 | POL | 524 | SVVRRAFPH | 9 | 0.1100 | 2433 |
| 85 | 17 | POL | 716 | TAELLAACF | 9 | | 2434 |
| 85 | 17 | POL | 716 | TAELLAACFA | 10 | | 2435 |
| 85 | 17 | POL | 716 | TAELLAACFAR | 11 | 0.0006 | 2436 |
| 80 | 16 | NUC | 33 | TASALYREA | 9 | | 2437 |
| 100 | 20 | ENV | 311 | TCIPIPSSWA | 10 | | 2438 |
| 100 | 20 | ENV | 311 | TCIPIPSSWAF | 11 | | 2439 |
| 80 | 16 | X | 106 | TDLEAYFK | 8 | | 2440 |
| 90 | 18 | POL | 736 | TDNSVVLSR | 9 | | 2441 |
| 90 | 18 | POL | 736 | TDNSVVLSRK | 10 | 0.0006 | 2442 |
| 90 | 18 | POL | 736 | TDNSVVLSRKY | 11 | | 2443 |
| 75 | 15 | NUC | 138 | TFGRETVLEY | 10 | | 2444 |
| 95 | 19 | POL | 657 | TFSPTYKA | 8 | | 2445 |
| 95 | 19 | POL | 857 | TFSPTYKAF | 9 | | 2446 |
| 100 | 20 | POL | 359 | TGGVFLVQK | 9 | 0.0007 | 2447 |
| 85 | 17 | POL | 799 | TGRTSLYA | 6 | | 2448 |
| 95 | 19 | NUC | 171 | TLPETTVVR | 9 | 0.0008 | 2449 |
| 95 | 19 | NUC | 171 | TLPETTVVRR | 10 | 0.0007 | 2450 |
| 95 | 19 | NUC | 171 | TLPETTVVRRR | 11 | 0.0005 | 2451 |
| 100 | 20 | POL | 150 | TLWKAGILY | 9 | 0.1300 | 2452 |
| 100 | 20 | P0L | 150 | TLWKAGILYK | 10 | 5.3000 | 2453 |
| 100 | 20 | POL | 150 | TLWKAGILYKR | 11 | 0.0082 | 2454 |
| 95 | 19 | POL | 519 | TSAICSVVR | 9 | 0.0005 | 2455 |
| 95 | 19 | POL | 519 | TSAICSVVRR | 10 | 0.0018 | 2456 |
| 95 | 19 | POL | 519 | TSAICSVVRRA | 11 | | 2457 |
| 75 | 15 | POL | 747 | TSFPWLLGCA | 10 | | 2458 |
| 75 | 15 | POL | 747 | TSFPWLLGCAA | 11 | | 2459 |
| 80 | 16 | POL | 764 | TSFVYVPSA | 9 | | 2460 |
| 75 | 15 | X | 105 | TTDLEAYF | 8 | | 2461 |
| 75 | 15 | X | 105 | TTDLEAVFK | 9 | 0.0006 | 2462 |
| 85 | 17 | POL | 798 | TTGRTSLY | 8 | 0.0004 | 2463 |
| 85 | 17 | POL | 798 | TTGRTSLYA | 9 | | 2464 |
| 75 | 15 | ENV | 278 | TTSTGPCK | 8 | | 2465 |
| 80 | 16 | NUC | 175 | TTVVRRRGR | 9 | 0.0008 | 2466 |
| 80 | 16 | NUC | 176 | TVVRRRGR | 8 | 0.0003 | 2467 |
| 80 | 16 | NUC | 176 | TVVRRRGRSPR | 11 | | 2468 |
| 95 | 19 | X | 60 | VCAFSSAGPCA | 11 | | 2469 |
| 85 | 17 | POL | 621 | VCQRNGLLGF | Ii | | 2470 |
| 100 | 20 | POL | 379 | VDFSQFSR | 8 | | 2471 |
| 100 | 20 | POL | 362 | VFLVDKNPH | 9 | | 2472 |
| 80 | 16 | X | 131 | VFVLGGCR | 8 | | 2473 |
| 80 | 16 | X | 131 | VFVLGGCRH | 9 | | 2474 |
| 75 | 15 | X | 131 | VFVLGGCRHK | 10 | | 2475 |
| 95 | 19 | X | 21 | VGAESRGR | 8 | | 2476 |
| 95 | 19 | POL | 626 | VGLLGFAA | 8 | | 2477 |
| 95 | 19 | POL | 626 | VGLLGFAAPF | 10 | | 2478 |
| 80 | 16 | POL | 508 | VGLSPFLLA | 9 | | 2479 |
| 80 | 16 | POL | 508 | VGLSPFLLAQF | 11 | | 2480 |
| 95 | 19 | POL | 56 | VGNFTGLY | 8 | | 2481 |
| 85 | 17 | POL | 96 | VGPLTVNEK | 9 | 0.0007 | 2482 |
| 85 | 17 | POL | 96 | VGPLTVNEKR | 10 | | 2483 |
| 85 | 17 | POL | 96 | VGPLTVNEKRR | 11 | | 2484 |
| 95 | 19 | X | 15 | VLCLRPVGA | 9 | | 2485 |
| 95 | 19 | POL | 543 | VLGAKSVQH | 9 | | 2486 |
| 90 | 18 | X | 133 | VLGGCRHK | 8 | 0.0150 | 2487 |
| 80 | 16 | ENV | 177 | VLQAGFFLLTR | 11 | | 2488 |
| 85 | 17 | POL | 741 | VLSRKYTSF | 9 | | 2489 |
| 90 | 18 | NUC | 120 | VSFGVWIR | 8 | 0.0040 | 2490 |
| 100 | 20 | POL | 48 | VSIPWTHK | 8 | 0.0130 | 2491 |
| 100 | 20 | POL | 358 | VTGGVFLVDK | 10 | 0.0390 | 2492 |
| 100 | 20 | POL | 378 | VVDFSQFSR | 9 | 0.0015 | 2493 |
| 90 | 18 | POL | 542 | VVLGAKSVQH | 10 | | 2494 |
| 85 | 17 | POL | 740 | VVLSRKYTSF | 10 | 0.0004 | 2495 |
| 95 | 19 | POL | 525 | VVRRAFPH | 8 | | 2496 |
| 95 | 19 | POL | 525 | VVRRAFPHCLA | 11 | | 2497 |
| 80 | 16 | NUC | 177 | WRRRGRSPR | 10 | 0.0027 | 2498 |
| 80 | 16 | NUC | 177 | WRRRGRSPRR | 11 | | 2499 |
| 90 | 18 | NUC | 102 | WFHISCLTF | 9 | | 2500 |
| 90 | 18 | NUC | 102 | WFHISCLTFGR | 11 | | 2501 |
| 85 | 17 | NUC | 28 | WGMDIDPY | 8 | | 2502 |
| 85 | 17 | NUC | 28 | WGMDIDPYK | 9 | −0.0003 | 2503 |
| 85 | 17 | NUC | 28 | WGMDIDPYKEF | 11 | | 2504 |
| 85 | 17 | POL | 578 | WGYSLNFMGY | 10 | | 2505 |
| 80 | 16 | POL | 759 | WILRGTSF | 8 | | 2506 |
| 80 | 16 | POL | 759 | WILRGTSFVY | 10 | 0.0076 | 2507 |
| 95 | 19 | NUC | 125 | WIRTPPAY | 8 | −0.0002 | 2508 |
| 95 | 19 | NX | 125 | WIRTPPAYR | 9 | 0.0008 | 2509 |
| 90 | 18 | POL | 314 | WLQFRNSK | 8 | −0.0002 | 2510 |
| 100 | 20 | POL | 414 | WLSLDVSA | 8 | | 2511 |
| 95 | 19 | POL | 414 | WLSLDVSAA | 9 | | 2512 |
| 95 | 19 | POL | 414 | WLSLDVSAAF | 10 | | 2513 |
| 95 | 19 | POL | 414 | WISLDVSAAFY | 11 | 0.0034 | 2514 |
| 100 | 20 | ENV | 335 | WLSLLVPF | 8 | | 2515 |
| 85 | 17 | NUC | 26 | WLWGMDIDPY | 10 | 0.0002 | 2516 |
| 85 | 17 | NUC | 26 | WLWGMDIDPYK | 11 | 0.0030 | 2517 |
| 95 | 19 | ENV | 237 | WMCLRRFIIF | 10 | 0.0004 | 2518 |
| 85 | 17 | ENV | 359 | WMMWYWGPSLY | 11 | 0.0009 | 2519 |
| 100 | 20 | POL | 52 | WTHKVGNF | 8 | | 2520 |
| 100 | 20 | POL | 147 | YLHTLWKA | 8 | | 2521 |
| 100 | 20 | POL | 122 | YLPLDKGIK | 9 | 0.0001 | 2522 |
| 100 | 20 | POL | 122 | YLPLDKGIKPY | 11 | −0.0004 | 2523 |
| 90 | 18 | NUC | 118 | YLVSFGVWIR | 10 | 0.0005 | 2524 |
| 90 | 18 | POL | 538 | YMDDVVLGA | 9 | 0.0001 | 2525 |
| 90 | 18 | POL | 538 | YMDDWLGAK | 10 | 0.0330 | 2526 |
| 80 | 16 | POL | 493 | YSHPIILGF | 9 | | 2527 |
| 80 | 18 | POL | 493 | YSHPIILGFR | 10 | | 2528 |
| 80 | 16 | POL | 493 | YSHIPIILGFR | 11 | | 2529 |
| | | | | K | | | |
| 85 | 17 | POL | 580 | YSLNFMGY | 8 | −0.0002 | 2530 |
| 75 | 15 | POL | 746 | YTSFPWLLGCA | 11 | | 2531 |
| 90 | 18 | POL | 768 | YVPSALNPA | 9 | | 2532 |

TABLE XVII

A11 Motif With Binding Information

| | | | | | | | |
|---|---|---|---|---|---|---|---|
| Conservancy | Frequency | Protein | Position | Sequence | AA | A*1101 | Seq ID Num |

| | | | | | | |
|---|---|---|---|---|---|---|
| 85 | 17 | POL | 721 | AACFARSR | 8 | 2533 |
| 95 | 19 | POL | 632 | AAPFTQCGY | 9 | 2534 |
| 90 | 18 | | 778 | ADDPSRGR | 8 | 2535 |
| 95 | 19 | POL | 529 | AFPHCLAFSY | 10 | 2538 |
| 90 | 19 | X | 62 | AFSSAGPCALR | 11 | 2537 |
| 95 | 19 | POL | 655 | AFTFSPTY | 8 | 2538 |
| 95 | 19 | POL | 655 | AFTFSPTYK | 9 | 2539 |
| 80 | 16 | ENV | 180 | AGFFLLTR | 8 | 2540 |
| 95 | 19 | POL | 18 | AGPLEEELPR | 10 | 2541 |
| 95 | 19 | POL | 521 | AICSVVRR | 8 | 2542 |
| 95 | 19 | NUC | 41 | ALESPEHCSPH | 11 | 2543 |
| 90 | 18 | POL | 772 | ALNPADDPSR | 10 | 2544 |
| 85 | 17 | X | 70 | ALRFTSAR | 8 | 2545 |
| 80 | 16 | ENV | 108 | AMQWNSTTFH | 10 | 2546 |
| 80 | 8 | POL | 166 | AFCGSPY | 8 | 2547 |
| 80 | 16 | POL | 822 | ASPLHVAWR | 9 | 2548 |
| 75 | 15 | ENV | 84 | ASTNRQSGR | 9 | 2549 |
| 80 | 16 | POL | 755 | CAANWILR | 8 | 2550 |
| 85 | 17 | X | 69 | CALRFTSAR | 9 | 2551 |
| 80 | 16 | X | 6 | CCQLDPAR | 8 | 2552 |
| 75 | 15 | POL | 607 | CFRKLPVNR | 9 | 2553 |
| 95 | 19 | POL | 638 | CGYPALMPLY | 10 | 2554 |
| 95 | 19 | ENV | 253 | CLIFLLVLLDY | 11 | 2555 |
| 90 | 18 | X | 17 | CLRPVGAESR | 10 | 2556 |
| 100 | 20 | NUC | 48 | CSPHHTALR | 9 | 2557 |
| 95 | 19 | POL | 523 | CSVVRRAFPH | 10 | 2558 |
| 90 | 18 | POL | 540 | DDVVLGAK | 8 | 2559 |
| 85 | 17 | NUC | 29 | DLLDTASALY | 10 | 2560 |
| 85 | 17 | NUC | 29 | DLLDTASALYR | 11 | 2561 |
| 90 | 18 | POL | 737 | DNSVVLSR | 8 | 2562 |
| 90 | 18 | POL | 787 | DNSVVLSRK | 9 | 2562 |
| 90 | 18 | POL | 737 | DNSVVLSRKY | 10 | 2533 |
| 85 | 17 | NUC | 32 | DTASALYR | 8 | 2534 |
| 95 | 19 | POL | 418 | DVSAAFYH | 8 | 2535 |
| 90 | 18 | POL | 541 | DVVLGAKSVQH | 11 | 2536 |
| 95 | 19 | POL | 17 | EAGPLEEELPR | 11 | 2537 |
| 90 | 18 | NUC | 40 | EALESPEH | 8 | 2538 |
| 90 | 18 | POL | 718 | ELLAACFAR | 9 | 2539 |
| 85 | 17 | POL | 718 | ELLAACFARSR | 11 | 2540 |
| 95 | 19 | NUC | 43 | ESPEHCSPH | 9 | 2541 |
| 95 | 19 | NUC | 43 | ESPEHCSPHH | 10 | 2542 |
| 95 | 19 | NUC | 174 | ETTVVRRR | 8 | 2543 |
| 80 | 16 | NUC | 174 | ETTWRRRGR | 10 | 2544 |
| 95 | 19 | POL | 631 | FAAPFTQCGY | 10 | 2545 |
| 80 | 16 | POL | 821 | FASPLHVAWR | 10 | 2546 |
| 75 | 15 | NUC | 139 | FGRETVLEY | 9 | 2547 |
| 75 | 15 | POL | 244 | FGVEPSGSGH | 10 | 2548 |
| 95 | 19 | NUC | 122 | FGVWIRTPPAY | 11 | 2549 |
| 95 | 19 | POL | 562 | FLLSLGIH | 8 | 2550 |
| 95 | 19 | ENV | 256 | FLLVLLDY | 8 | 2551 |
| 100 | 20 | POL | 363 | FLVDKVPH | 8 | 2552 |
| 90 | 18 | X | 83 | FSSAGPCALR | 10 | 2553 |
| 95 | 19 | POL | 656 | FTFSPTYK | 8 | 2554 |
| 95 | 19 | POL | 518 | FTSAICSVVR | 10 | 2555 |
| 95 | 19 | POL | 518 | FTSAICSVVRR | 11 | 2556 |
| 95 | 19 | X | 132 | FVLGGCRH | 6 | 2557 |
| 90 | 18 | X | 132 | FVLGGCRHK | 9 | 2558 |
| 80 | 16 | POL | 754 | GCAANWILR | 9 | 2559 |
| 95 | 19 | POL | 630 | GFAAPFTQCGY | 11 | 2560 |
| 100 | 20 | POL | 360 | GGVFLVDK | 8 | 2561 |
| 100 | 20 | POL | 360 | GGVFLVDKNPH | 11 | 2562 |
| 75 | 15 | POL | 567 | GIHLNPNK | 8 | 2563 |
| 75 | 15 | POL | 567 | GIHLNPNKTK | 10 | 2564 |
| 75 | 15 | POL | 567 | GIHLNPNKTKR | 11 | 2565 |
| 85 | 17 | NUC | 29 | GMDIDPYK | 8 | 2566 |
| 95 | 19 | POL | 44 | GNLNVSIPWTH | 11 | 2567 |
| 90 | 18 | POL | 735 | GTDNSVVLSR | 10 | 2568 |
| 90 | 18 | POL | 735 | GTDNSVVLSRK | 11 | 2569 |
| 80 | 16 | POL | 245 | GVEPSGSGH | 9 | 2570 |
| 100 | 20 | POL | 361 | GVFLVDKNPH | 10 | 2571 |
| 95 | 19 | NUC | 123 | GVWIRTPPAY | 10 | 2572 |
| 95 | 19 | NUC | 123 | GVWIRTPPAYR | 11 | 2573 |
| 100 | 20 | NUC | 47 | HCSPHHTALR | 10 | 2574 |
| 80 | 16 | POL | 820 | HFASPLHVAWR | 11 | 2575 |
| 95 | 19 | X | 49 | HGAHLSLR | 8 | 2576 |
| 90 | 18 | NUC | 104 | HISCLTFGR | 9 | 2577 |
| 75 | 15 | POL | 569 | HLNPNKTK | 8 | 2578 |
| 75 | 15 | POL | 569 | HLNPNKTKR | 9 | 2579 |
| 100 | 20 | POL | 149 | HTLWKAGILY | 10 | 2580 |
| 100 | 20 | POL | 149 | HTLWKAGILYK | 11 | 2581 |
| 95 | 19 | POL | 522 | ICSVVRRAFPH | 11 | 2582 |
| 90 | 18 | POL | 617 | IDWKVCQR | 8 | 2583 |
| 100 | 20 | ENV | 381 | IFFCLWVY | 8 | 2584 |
| 95 | 19 | ENV | 255 | IFLLVLLDY | 9 | 2585 |
| 80 | 16 | POL | 734 | IGTDNSVVLSR | 11 | 2588 |
| 80 | 16 | POL | 760 | ILRGTSFVY | 9 | 2587 |
| 90 | 18 | NUC | 105 | ISCLTFGR | 8 | 2588 |
| 100 | 20 | POL | 153 | KAGILYKR | 8 | 2589 |
| 75 | 15 | POL | 108 | KLIMPARFY | 9 | 2590 |
| 80 | 16 | POL | 610 | KLPVNRPIDWK | 11 | 2591 |
| 75 | 15 | X | 130 | KVFVLGGCR | 9 | 2592 |
| 75 | 15 | X | 130 | KVFVLGGCRH | 10 | 2593 |
| 95 | 19 | POL | 55 | KVGNFTGLY | 9 | 2594 |
| 85 | 17 | POL | 720 | LAACFARSR | 9 | 2595 |
| 90 | 18 | X | 16 | LCLRPVGAESR | 11 | 2596 |
| 100 | 20 | POL | 125 | LDKGIKPY | 8 | 2597 |
| 100 | 20 | POL | 125 | LDKGIKPYY | 9 | 2598 |
| 80 | 16 | X | 9 | LDPARDVLCLR | 11 | 2599 |
| 85 | 17 | NUC | 31 | LDTASALY | 8 | 2600 |
| 85 | 17 | NUC | 31 | LDTASALYR | 9 | 2601 |
| 95 | 19 | POL | 417 | LDVSAAFY | 8 | 2802 |
| 95 | 19 | POL | 417 | LDVSAAFYH | 9 | 2603 |
| 95 | 19 | POL | 544 | LGAKSVQH | 8 | 2604 |
| 80 | 16 | POL | 753 | LGCAANWILR | 10 | 2605 |
| 75 | 15 | POL | 566 | LGIHLNPNK | 9 | 2606 |
| 75 | 15 | POL | 566 | LGIHLNPNKTK | 11 | 2607 |
| 95 | 19 | ENV | 254 | LIFLLVLLDY | 10 | 2608 |
| 100 | 20 | POL | 109 | LIMPARFY | 8 | 2609 |
| 90 | 18 | POL | 719 | LLAACFAR | 8 | 2610 |
| 85 | 17 | POL | 719 | LLAACFARSR | 10 | 2811 |
| 85 | 17 | NUC | 30 | LLDTASALY | 9 | 2812 |
| 85 | 17 | NUC | 30 | LLDTASALYR | 10 | 2613 |
| 80 | 16 | POL | 752 | LLGCAANWILR | 11 | 2814 |
| 100 | 20 | ENV | 378 | LLPIFFCLWVY | 11 | 2615 |
| 90 | 18 | POL | 773 | LNPADDPSR | 9 | 2616 |
| 90 | 18 | POL | 773 | LNPADDPSRGR | 11 | 2617 |
| 75 | 15 | POL | 570 | LNPNKTKR | 8 | 2618 |
| 75 | 15 | POL | 570 | LNPNKTKRWGY | 11 | 2819 |
| 95 | 19 | POL | 46 | LNVSIPWTH | 11 | 2620 |
| 95 | 19 | POL | 46 | LNVSIPWTHK | 10 | 2621 |
| 95 | 19 | POL | 415 | LSLDVSAAFY | 10 | 2622 |
| 95 | 19 | POL | 415 | LSLDVSAAFYH | 11 | 2623 |
| 75 | 15 | POL | 564 | LSLGIHLNPNK | 11 | 2624 |
| 95 | 19 | NUC | 169 | LSTLPETTVVR | 11 | 2825 |
| 75 | 15 | POL | 3 | LSYQHFRK | 8 | 2626 |
| 75 | 15 | NUC | 137 | LTFGRETVLEY | 11 | 2627 |
| 85 | 17 | POL | 99 | LTVNEKRR | 8 | 2628 |
| 90 | 18 | NUC | 119 | LVSFGVWIR | 9 | 2629 |
| 100 | 20 | POL | 377 | LVVDFSQFSR | 10 | 2630 |
| 90 | 18 | POL | 539 | MDDVVLGAK | 9 | 2631 |
| 85 | 17 | ENV | 360 | MMWYWGPSLY | 10 | 2632 |
| 75 | 15 | X | 103 | MSTTDLEAY | 9 | 2633 |
| 75 | 15 | X | 103 | MSTTDLEAYFK | 11 | 2634 |
| 95 | 19 | POL | 561 | NFLLSLGIH | 9 | 2635 |
| 90 | 18 | NUC | 75 | NLEDPASR | 8 | 2636 |
| 95 | 19 | POL | 45 | NLNVSIPWTH | 10 | 2637 |
| 95 | 19 | POL | 45 | NLNVSIPWTHK | 11 | 2638 |
| 75 | 15 | ENV | 215 | NSQSPTSNH | 9 | 2639 |
| 90 | 18 | POL | 738 | NSVVLSRK | 8 | 2640 |
| 90 | 16 | POL | 736 | NSVVLSRKY | 9 | 2641 |
| 100 | 20 | POL | 47 | NVSIPWTH | 8 | 2642 |
| 100 | 20 | POL | 47 | NVSIPWTHK | 9 | 2643 |
| 90 | 18 | POL | 775 | PADDPSRGR | 9 | 2644 |
| 80 | 16 | X | 11 | PARDVLCLR | 9 | 2645 |
| 75 | 15 | ENV | 83 | PASTNRQSGR | 10 | 2646 |
| 85 | 17 | X | 68 | PCALRFTSAR | 10 | 2647 |
| 100 | 20 | ENV | 233 | PGYRWMCLR | 9 | 2648 |
| 95 | 19 | ENV | 233 | PGYRWMCLRR | 10 | 2649 |
| 90 | 18 | POL | 616 | PIDWKVCQR | 9 | 2650 |
| 100 | 20 | ENV | 360 | PIFFCLWVY | 9 | 2651 |
| 80 | 16 | POL | 496 | PILGFRK | 8 | 2652 |
| 100 | 20 | POL | 124 | PLDKGIKPY | 9 | 2653 |
| 100 | 20 | POL | 124 | PLDKGIKPTY | 10 | 2654 |
| 95 | 19 | POL | 20 | PLEEELPR | 8 | 2655 |
| 95 | 19 | POL | 10 | PLGFFPDH | 8 | 2658 |
| 100 | 20 | POL | 427 | PLHPAAMPH | 9 | 2657 |
| 100 | 20 | POL | 2 | PLSYQHFR | 8 | 2858 |
| 75 | 15 | POL | 2 | PLSYQHFRK | 9 | 2659 |
| 85 | 17 | POL | 98 | PLTVNEKR | 8 | 2660 |
| 85 | 17 | POL | 98 | PLTVNEKRR | 9 | 2661 |
| 75 | 15 | POL | 572 | PNKTKRWGY | 9 | 2662 |
| 85 | 17 | POL | 797 | PTTGRTSLY | 9 | 2663 |
| 90 | 18 | X | 20 | PVGAESRGR | 9 | 2664 |
| 85 | 17 | POL | 612 | PVNRPIDWK | 9 | 2685 |
| 95 | 19 | PCI | 654 | QAFTFSPTY | 9 | 2666 |
| 95 | 19 | POL | 654 | QAFTFSPTYK | 10 | 2687 |
| 80 | 16 | ENV | 179 | QAGFFLLTR | 9 | 2668 |
| 80 | 16 | ENV | 107 | QAMQWNSTTFH | 10 | 2669 |
| 95 | 19 | POL | 637 | QCGYPALMPLY | 11 | 2670 |
| 95 | 19 | POL | 517 | QFTSAICSVVR | 11 | 2671 |
| 75 | 15 | NUC | 169 | QSPRRRRSQSR | 11 | 2672 |
| 80 | 16 | POL | 189 | QSSGILSR | 8 | 2673 |
| 95 | 19 | POL | 528 | RAFPHCLAFSY | 11 | 2674 |
| 85 | 17 | NUC | 28 | RDLLDTASALY | 11 | 2675 |
| 100 | 20 | NUC | 152 | RGRSPRRR | 8 | 2676 |
| 75 | 15 | POL | 106 | RLKLIMPAR | 9 | 2677 |
| 75 | 15 | POL | 106 | RLKLIMPARFY | 11 | 2678 |
| 75 | 15 | X | 128 | RLKVFVLGGCR | 11 | 2679 |
| 95 | 19 | POL | 376 | RLVVDFSQFSR | 11 | 2680 |
| 95 | 19 | NUC | 183 | RSPRRRTPSPR | 11 | 2681 |
| 75 | 15 | NUC | 167 | RSQSPRRR | 8 | 2682 |
| 75 | 15 | NUC | 167 | RSQSPRRR | 9 | 2683 |
| 95 | 19 | NUC | 188 | RTPSPRRR | 8 | 2684 |
| 95 | 19 | NUC | 188 | RTPSPRRR | 9 | 2685 |
| 80 | 16 | POL | 818 | RVHFASPLH | 9 | 2686 |
| 100 | 20 | POL | 357 | RVTGGVFLVDK | 11 | 2687 |
| 90 | 18 | X | 64 | SAGPCALR | 8 | 2688 |
| 95 | 19 | POL | 520 | SAICSVVR | 8 | 2689 |
| 95 | 19 | POL | 520 | SAICSVVRR | 9 | 2690 |
| 90 | 18 | POL | 771 | SALNPADDPSR | 11 | 2691 |
| 100 | 20 | POL | 165 | SASFQGSPY | 9 | 2692 |
| 95 | 19 | POL | 416 | SLDVSAAFY | 9 | 2693 |
| 95 | 19 | POL | 416 | SLDVSAAFYH | 10 | 2694 |
| 75 | 15 | POL | 565 | SLGIHLNPNK | 10 | 2695 |
| 90 | 18 | X | 641 | SSAGPCALR | 9 | 2696 |
| 95 | 19 | NUC | 170 | STLPETTVVR | 10 | 2697 |
| 95 | 19 | NUC | 170 | STLPETTVVRR | 11 | 2698 |
| 80 | 16 | ENV | 85 | STNRQSGR | 8 | 2699 |
| 75 | 15 | X | 104 | STTDLEAY | 8 | 2700 |
| 75 | 15 | X | 104 | STTDLEAYFK | 10 | 2701 |
| 90 | 18 | POL | 739 | SVVLSRKY | 8 | 2702 |
| 95 | 19 | POL | 524 | SVVRRAFPH | 9 | 2703 |
| 85 | 17 | POL | 716 | TAELLAACFAR | 11 | 2704 |
| 80 | 16 | X | 106 | TDLEAYFK | 8 | 2705 |
| 90 | 18 | POL | 736 | TDNSVVLSR | 9 | 2706 |
| 90 | 18 | POL | 736 | TDNSVVLSRK | 10 | 2707 |
| 90 | 18 | POL | 736 | TDNSVVLSRKY | 11 | 2708 |
| 75 | 15 | NUC | 138 | TFGRETVLEY | 10 | 2709 |
| 100 | 20 | POL | 359 | TGGVFLVDK | 9 | 2710 |
| 95 | 19 | NUC | 171 | TLPETTVVR | 9 | 2711 |
| 95 | 19 | NUC | 171 | TLPETTVVRR | 10 | 2712 |
| 95 | 19 | NUC | 171 | TLPETTVVRRR | 11 | 2713 |
| 100 | 20 | POL | 150 | TLWKAGILY | 9 | 2714 |
| 100 | 20 | POL | 150 | TLWKAGILYK | 10 | 2715 |
| 100 | 20 | POL | 150 | TLWKAGILYKR | 11 | 2716 |
| 95 | 19 | POL | 560 | TNFLLSLGIH | 10 | 2717 |
| 95 | 19 | POL | 519 | TSAICSVVR | 9 | 2718 |
| 95 | 19 | POL | 519 | TSAICSVVRR | 10 | 2719 |
| 75 | 15 | X | 105 | TTDLEAYFK | 9 | 2720 |
| 85 | 17 | POL | 798 | TTGRTSLY | 8 | 2721 |
| 75 | 15 | ENV | 278 | TTSTGPCK | 8 | 2722 |
| 80 | 16 | NUC | 175 | TTVVRRRGR | 9 | 2723 |
| 80 | 16 | NUC | 176 | TVVRRRGR | 8 | 2724 |
| 80 | 16 | NUC | 178 | TVVRRRGRSPR | 11 | 2725 |
| 100 | 20 | POL | 379 | VDFSQFSR | 8 | 2726 |
| 100 | 20 | POL | 362 | VFLVDKNPH | 9 | 2727 |
| 80 | 16 | X | 131 | VFVLGGCR | 8 | 2728 |
| 80 | 16 | X | 131 | VFVLGGCRH | 9 | 2729 |
| 75 | 15 | X | 131 | VFVLGGCRHK | 10 | 2730 |
| 95 | 19 | X | 21 | VGAESRGR | 8 | 2731 |
| 95 | 19 | POL | 56 | VGNFTGLY | 8 | 2732 |
| 85 | 17 | POL | 96 | VGPLTVNEK | 9 | 2733 |
| 85 | 17 | POL | 96 | VGPLTVNEKR | 10 | 2734 |
| 85 | 17 | POL | 96 | VGPLTVNEKRR | 11 | 2735 |
| 95 | 19 | POL | 543 | VLGAKSVQH | 9 | 2736 |
| 90 | 18 | X | 133 | VLGGCRH | 8 | 2737 |
| 80 | 16 | ENV | 177 | VLQAGFFLLTR | 11 | 2738 |
| 85 | 17 | POL | 613 | VNRPIDWK | 8 | 2739 |
| 90 | 18 | NUC | 120 | VSFGVWIR | 8 | 2740 |
| 100 | 20 | POL | 48 | VSIPWTHK | 8 | 2741 |
| 100 | 20 | POL | 358 | VTGGVFLVDK | 10 | 2742 |
| 100 | 20 | POL | 378 | VVDFSQFSR | 9 | 2743 |
| 90 | 18 | POL | 542 | VVLGAKSVQH | 10 | 2744 |
| 95 | 19 | POL | 525 | VVRRAFPH | 8 | 2745 |
| 80 | 16 | NUC | 177 | VVRRRGRSPR | 10 | 2746 |
| 80 | 16 | NUC | 177 | VRRRGRSPRR | 11 | 2747 |
| 90 | 18 | NUC | 102 | WFHISCLTFGR | 11 | 2748 |
| 85 | 17 | NUC | 28 | WGMDIDPY | 8 | 2749 |
| 85 | 17 | NUC | 28 | WGMDIDPYK | 9 | 2750 |
| 85 | 17 | POL | 578 | WGYSLNFMGY | 10 | 2751 |
| 80 | 16 | POL | 759 | WILRGTSFVY | 10 | 2752 |
| 95 | 19 | NUC | 125 | WIRTPPAY | 8 | 2753 |
| 95 | 19 | NUC | 125 | WIRTPPAYR | 9 | 2754 |
| 90 | 18 | POL | 314 | WLQFRNSK | 8 | 2755 |
| 95 | 19 | POL | 414 | WLSLDVSAAFY | 11 | 2756 |
| 85 | 17 | NUC | 26 | WLWGMDIDPY | 10 | 2757 |
| 85 | 17 | NUC | 26 | WLWGMDIDPYK | 11 | 2758 |
| 85 | 17 | ENV | 359 | WMMWYWGPSLY | 11 | 2759 |
| 100 | 20 | POL | 122 | YLPDKGIK | 9 | 2760 |
| 100 | 20 | POL | 122 | YLPLDKGIKPY | 11 | 2761 |
| 90 | 18 | NUC | 118 | YLVSFGVWIR | 10 | 2782 |
| 90 | 18 | POL | 538 | YMDDVVLGAK | 10 | 2783 |
| 80 | 16 | POL | 493 | YSHPIILGFR | 10 | 2764 |
| 60 | 16 | POL | 493 | YSHPIILGFRK | 11 | 2765 |
| 85 | 17 | POL | 580 | YSLNFMGY | 8 | 2766 |

TABLE XVIII

HBV A24 Motif With Binding Information

| | | | | | | | | |
|---|---|---|---|---|---|---|---|---|
| | | | | | SEQ ID | | | |
| Conservancy | Freq. | Protein | Position | Sequence | NO: | AA | Filed | A*2401 |

| | | | | | | | | |
|---|---|---|---|---|---|---|---|---|
| 95 | 19 | POL | 529 | AFPHCLAF | 2767 | 8 | | |
| 95 | 19 | X | 82 | AFSSAGPCAL | 2768 | 10 | | 0.0012 |
| 90 | 18 | POL | 535 | AFSYMDDVVL | 2769 | 10 | | 0.0009 |
| 95 | 19 | POL | 655 | AFTFSPTYKAF | 2770 | 11 | | |
| 80 | 16 | ENN | 108 | AMQWNSTTF | 2771 | 9 | | |
| 100 | 20 | NUC | 131 | AYRPPNAPI | 2772 | 9 | — | 0.0310 |
| 100 | 20 | NUC | 131 | AYRPPNAPIL | 2773 | 10 | — | 0.0042 |
| 75 | 15 | POL | 807 | CFRKLPVNRPI | 2774 | 11 | | |
| 85 | 17 | POL | 618 | DWKVCQRI | 2775 | 8 | | |
| 85 | 17 | POL | 618 | DWKVCQRPIVGL | 2776 | 11 | | |
| 90 | 18 | ENV | 262 | DYQGMLPVCPL | 2777 | 11 | | 0.0002 |
| 90 | 18 | NUC | 117 | EYLVSFGVW | 2778 | 9 | — | |
| 90 | 18 | NUC | 117 | EYLVSFGVWI | 2779 | 10 | | |
| 100 | 20 | ENV | 382 | FFCLWVYI | 2780 | 8 | | |
| 80 | 16 | ENV | 182 | FFLLTRIL | 2781 | 8 | | |
| 80 | 16 | ENV | 182 | FFLLTRILTI | 2782 | 10 | | |
| 85 | 17 | ENV | 13 | FFPDHQLDPAF | 2783 | 11 | | |
| 80 | 16 | ENV | 181 | GFFLLTRI | 2784 | 8 | | |
| 80 | 16 | ENV | 181 | GFFLLTRIL | 2785 | 9 | | |
| 80 | 16 | ENV | 181 | GFFLLTRILTI | 2786 | 11 | | |
| 95 | 19 | ENV | 12 | GFFPDHQL | 2787 | 8 | | |
| 75 | 15 | ENV | 170 | GFLGPLLVL | 2788 | 9 | | |
| 80 | 16 | POL | 500 | GFRKIPMGVGL | 2789 | 11 | | |
| 85 | 17 | NUC | 29 | GMDIDPYKEF | 2790 | 10 | | |
| 90 | 18 | ENV | 265 | GMLPVCPL | 2791 | 8 | | |
| 85 | 17 | NUC | 25 | GWLWGMDI | 2792 | 8 | | |
| 85 | 17 | ENV | 65 | GWSPQAQGI | 2793 | 9 | | 0.0024 |
| 85 | 17 | ENV | 65 | GWSPQAQGIL | 2794 | 10 | | 0.0003 |
| 95 | 19 | POL | 639 | GYPALMPL | 2795 | 8 | | |
| 95 | 19 | ENV | 234 | GYRWMCLRRF | 2796 | 10 | — | 0.0007 |
| 95 | 19 | ENV | 234 | GYRWMCLRRFI | 2797 | 11 | | |
| 75 | 15 | POL | 579 | GYSLNFMGYVI | 2798 | 11 | | |
| 80 | 16 | POL | 820 | HFASPLHVAW | 2799 | 10 | | |
| 75 | 15 | POL | 7 | HFRKLLL | 2800 | 8 | | |
| 100 | 20 | POL | 146 | HYLHTLWKAGI | 2801 | 11 | | |
| 100 | 20 | ENV | 381 | IFFCLWVYI | 2802 | 9 | | 0.0087 |
| 80 | 16 | ENV | 245 | IFLFILLL | 2803 | 8 | | |
| 80 | 16 | ENV | 245 | IFLFILLLCL | 2804 | 10 | | |
| 80 | 16 | ENV | 245 | IFLFILLLCLI | 2805 | 11 | | |
| 85 | 17 | ENV | 358 | IWMMWYWGPSL | 2806 | 11 | | 0.0004 |
| 95 | 19 | POL | 395 | KFAVPNLQSL | 2807 | 10 | | 0.0020 |
| 100 | 20 | POL | 121 | KYLPLDKGI | 2808 | 9 | | |
| 85 | 17 | POL | 745 | KYTSFPWL | 2809 | 8 | | |
| 85 | 17 | POL | 745 | KYTSFPWLL | 2810 | 8 | — | 5.3000 |
| 80 | 16 | ENV | 247 | LFILLLCL | 2811 | 8 | | |
| 80 | 16 | ENV | 247 | LFILLLCLI | 2812 | 9 | | |
| 80 | 16 | ENV | 247 | LFILLLCLIF | 2813 | 10 | | |
| 80 | 16 | ENV | 247 LFILLLCLIFL | 2814 | 11 | | | |
| 95 | 19 | POL | 643 | LMPLYACI | 2815 | 8 | | |
| 90 | 18 | NUC | 101 | LWFHISCL | 2816 | 8 | | |
| 85 | 17 | NUC | 101 | LWFHISCLTF | 2817 | 10 | | |
| 80 | 16 | POL | 492 | LYSHPIIL | 2818 | 8 | | |
| 80 | 16 | POL | 492 | LYSHPIILGF | 2819 | 10 | — | 1.1000 |
| 85 | 17 | ENV | 360 | MMWYWGPSL | 2820 | 9 | — | 0.0060 |
| 85 | 17 | ENV | 361 | MWYWGPSL | 2821 | 8 | | 0.0005 |
| 95 | 19 | POL | 561 | NFLLSLGI | 2822 | 8 | | |
| 95 | 19 | POL | 561 | NFLLSLGIHL | 2823 | 10 | | 0.0099 |
| 80 | 16 | POL | 758 | NWILRGTSF | 2824 | 9 | | |
| 95 | 19 | POL | 512 | PFLLAQFTSAI | 2825 | 11 | | |
| 95 | 19 | POL | 634 | PFTQCGYPAL | 2826 | 10 | | 0.0002 |
| 95 | 19 | ENV | 341 | PFVQWFVGL | 2827 | 9 | | 0.0003 |
| 80 | 16 | POL | 505 | PMGVGLSPF | 2828 | 9 | | |
| 80 | 16 | POL | 505 | PMGVGLSPFL | 2829 | 10 | | |
| 80 | 16 | POL | 505 | PMGVGLSPFLL | 2830 | 11 | | |
| 80 | 16 | POL | 750 | PWLLGCAANW | 2831 | 10 | | |
| 80 | 16 | POL | 750 | PWLLGCAANWI | 2832 | 11 | | |
| 100 | 20 | POL | 51 | PWTHKVGNF | 2833 | 9 | — | 0.0290 |
| 95 | 19 | ENV | 344 | QWFVGLSPTVW | 2834 | 11 | | |
| 75 | 15 | ENV | 242 | RFIIFLFI | 2835 | 8 | | |
| 75 | 15 | ENV | 242 | RFIIFLFIL | 2836 | 9 | | |
| 75 | 15 | ENV | 242 | RFIIFLFILL | 2837 | 10 | | |
| 75 | 15 | ENV | 242 | RFIIFIFILLL | 2838 | 11 | | |
| 100 | 20 | ENV | 332 | RFSWLSLL | 2839 | 8 | | |
| 100 | 20 | ENV | 332 | RFSWLSLLVPF | 2840 | 11 | | |
| 85 | 17 | POL | 577 | RWGYSLNF | 2841 | 8 | | |
| 95 | 19 | ENV | 236 | RWMCLRRF | 2842 | 8 | | |
| 95 | 19 | ENV | 236 | RWMCLRRFI | 2843 | 9 | — | 0.0710 |
| 95 | 19 | ENV | 236 | RWMCLRRFII | 2844 | 10 | — | 1.1000 |
| 95 | 19 | ENV | 236 | RWMCLRRFIIF | 2845 | 11 | | |
| 100 | 20 | POL | 167 | SFCGSPYSW | 2846 | 9 | — | 0.0710 |
| 95 | 19 | NUC | 46 | SFLPSDFF | 2847 | 8 | | |
| 80 | 16 | POL | 765 | SFVYVPSAL | 2848 | 9 | | |
| 95 | 19 | POL | 413 SWLSLDVSAAF | 2849 | 11 | | | |
| 100 | 20 | ENV | 334 | SWLSLLVPF | 2850 | 9 | — | 0.3900 |
| 95 | 19 | POL | 392 | SWPKFAVPNL | 2851 | 10 | — | 5.6000 |
| 100 | 20 | ENV | 197 | SWWTSLNF | 2852 | 8 | | |
| 95 | 19 | ENV | 197 | SWWTSLNFL | 2853 | 9 | — | 0.3800 |
| 90 | 18 | POL | 537 | SYMDDVVL | 2854 | 8 | | |
| 75 | 15 | POL | 4 | SYQHFRKL | 2855 | 8 | | |
| 75 | 15 | POL | 4 | SYQHFRKLL | 2856 | 9 | | 0.0051 |
| 75 | 15 | POL | 4 | SYQHFRKLLL | 2857 | 10 | — | 0.0660 |
| 75 | 15 | POL | 4 | SYQHFRKLLLL | 2858 | 11 | | |
| 75 | 15 | NUC | 138 | TFGRETVL | 2859 | 8 | | |
| 75 | 15 | NUC | 138 | TFGRETVLEYL | 2860 | 11 | | |
| 95 | 19 | POL | 857 | TFSPTYKAF | 2861 | 9 | | 0.0060 |
| 95 | 19 | POL | 657 | TFSPTYKAFL | 2862 | 10 | | 0.0043 |
| 95 | 19 | POL | 686 | VFADATPTGW | 2863 | 10 | — | 0.0180 |
| 75 | 15 | X | 131 | VFVLGGCRHKL | 2864 | 11 | | |
| 90 | 18 | NUC | 102 | WFHISCLTF | 2865 | 9 | — | 0.0300 |
| 95 | 19 | ENV | 345 | WFVGLSPTVW | 2866 | 10 | — | 0.0120 |
| 95 | 19 | ENV | 345 | WFVGLSPTVWL | 2867 | 11 | | |
| 95 | 19 | ENV | 237 | WMCLRRFI | 2868 | 8 | | |
| 95 | 19 | ENV | 237 | WMCLRRFII | 2869 | 9 | — | |
| 95 | 19 | RNV | 237 | WMCLRRFIIF | 2870 | 10 | | 0.0013 |
| 95 | 19 | ENV | 237 | WMCLRRFIIFL | 2871 | 11 | | |
| 85 | 17 | ENV | 359 | WMMWYWGPSL | 2872 | 10 | — | |
| 95 | 19 | ENV | 198 | WWTSLNFL | 2873 | 8 | | |

TABLE XIXa

| HBV DR-SUPER MOTIF | | | | | | | | | |
|---|---|---|---|---|---|---|---|---|---|
| | Core SEQ | Core | | Core | Exemplary | Exemplary | Position in HBV | Exemplary Sequence | Exemplary Sequence |
| Protein | ID NO: | Sequence | Core Freq. | Conservancy (%) | SEQ ID NO: | Sequence | Poly-Protein | Frequency | Conservancy (%) |
| POL | 2874 | FAAPFTQCG | 19 | 95 | 3021 | LLGFAAPFTQCGYPA | 628 | 19 | 95 |
| POL | 2875 | FADATPTGW | 19 | 95 | 3022 | CQVFADATPTGWGLA | 684 | 16 | 80 |
| POL | 2876 | FAVPNLQSL | 19 | 95 | 3023 | WPKFAVPNLQSLTNL | 393 | 19 | 95 |
| NUC | 2877 | FGRETVLEY | 15 | 75 | 3024 | CLTFGRETVLEYLVS | 136 | 14 | 70 |
| POL | 2876 | FGVEPSSGS | 15 | 75 | 3025 | RRSFGVEPSGSGHID | 252 | 6 | 30 |
| NUC | 2879 | FHISCLTFG | 18 | 90 | 3026 | LLWFHISCLTFGRET | 100 | 17 | 85 |
| NUC | 2880 | FHLCLIISC | 16 | 80 | 3027 | MQLFHLCLIISCSCP | 1 | 10 | 50 |
| ENV | 2881 | FILLLCLIF | 16 | 80 | 3028 | IFLFILLLCLIFLLV | 245 | 18 | 80 |
| ENV | 2882 | FLFILLLCL | 16 | 80 | 3029 | FIIFLFILLLCLIFL | 243 | 16 | 80 |
| ENV | 2883 | FLGPLLVLQ | 15 | 75 | 3030 | TSGFLGPLLVLQAGF | 168 | 15 | 75 |
| ENV | 2884 | FLLTRILTI | 16 | 80 | 3031 | AGFFLLTRILTIPQS | 180 | 16 | 80 |
| ENV | 2885 | FLLVLLDYQ | 19 | 95 | 3032 | CLIFLLVLLDYQGML | 253 | 19 | 95 |
| ENV | 2886 | FPAGGSSSG | 15 | 75 | 3033 | GLYFPAGGSSSGTVN | 127 | 11 | 55 |
| ENV | 2887 | FPDHQLDPA | 18 | 90 | 3034 | LGFFPDHQLDPAFGA | 22 | 9 | 45 |
| POL | 2888 | FPHCLAFSY | 19 | 95 | 3035 | RRAFPHCLAFSYMDD | 527 | 19 | 95 |
| POL | 2889 | FRKIPMGVG | 16 | 80 | 3036 | ILGFRKIPMGVGLSP | 498 | 13 | 65 |
| POL | 2890 | FRKLPVNRP | 16 | 80 | 3037 | KQCFRKLPVNRPIDW | 616 | 9 | 45 |
| X | 2891 | FSSAGPCAL | 19 | 95 | 3038 | VCAFSSAGPCALRFT | 60 | 18 | 90 |
| ENV | 2892 | FSWLSLLVP | 20 | 100 | 3039 | SVRFSWLSLLVPFVQ | 330 | 16 | 80 |
| POL | 2893 | FTFSPTYKA | 19 | 95 | 3040 | KQAFTFSPTYKAFLC | 653 | 12 | 60 |
| POL | 2894 | FTGLYSSTV | 18 | 90 | 3041 | VGNFTGLYSSTVPVF | 56 | 11 | 55 |
| POL | 2895 | FTSAICSVV | 19 | 95 | 3042 | LAQFTSAICSVVRRA | 515 | 19 | 95 |
| ENV | 2896 | FVGLSPTVW | 19 | 95 | 3043 | VQWFVGLSPTVWLSV | 343 | 14 | 70 |
| X | 2897 | FVLGGCRHK | 18 | 90 | 3044 | LKVFVLGGCRHKLVC | 129 | 14 | 70 |
| ENV | 2898 | FVQWFVGLS | 19 | 95 | 3045 | LVPFVQWFVGLSPTV | 339 | 19 | 95 |
| POL | 2899 | FVYVPSALN | 18 | 90 | 3046 | GTSFVYVPSALNPAD | 763 | 16 | 80 |
| POL | 2900 | IDWKVCQRI | 17 | 85 | 3047 | NRPIDWKVCQRIVGL | 614 | 16 | 80 |
| ENV | 2901 | IFLFILLLC | 16 | 80 | 3048 | RFIIFLFILLLCLIF | 242 | 15 | 75 |
| ENV | 2902 | IFLLVLLDY | 19 | 95 | 3049 | LCLIFLLVLLDYQGM | 252 | 19 | 95 |
| POL | 2903 | IGTDNSVVL | 16 | 80 | 3050 | AKLIGTDNSVVLSRK | 731 | 13 | 65 |
| POL | 2904 | IHTAELLAA | 17 | 85 | 3051 | PLPIHTAELLAACFA | 711 | 16 | 80 |
| ENV | 2905 | IIFIFILLI | 16 | 80 | 3052 | RRFIIFLFILLLCLI | 241 | 15 | 75 |
| ENV | 2906 | ILLLCLIFL | 20 | 100 | 3053 | FLFILLLCLIFLLVL | 246 | 16 | 80 |
| POL | 2907 | ILRGTSFVY | 16 | 80 | 3054 | ANWILRGTSFVYVPS | 757 | 16 | 80 |
| NUC | 2908 | ILSTLPETT | 20 | 100 | 3055 | NAPILSTLPETTVVR | 165 | 19 | 95 |
| ENV | 2909 | IPIPSSWAF | 20 | 100 | 3056 | CTCIPIPSSWAFARF | 321 | 8 | 40 |
| NUC | 2910 | IRTPPAYRP | 19 | 95 | 3057 | GVWIRTPPAYRPPNA | 123 | 19 | 95 |
| POL | 2911 | LAACFARSR | 17 | 85 | 3058 | AELLAACFARSRSGA | 717 | 16 | 80 |
| POL | 2912 | LAFSYMDDV | 18 | 90 | 3059 | PHCLAFSYMDDVVLG | 531 | 18 | 90 |
| POL | 2913 | LAQFTSAIC | 19 | 95 | 3060 | PFLLAQFTSAICSVV | 512 | 19 | 95 |
| NUC | 2914 | LCLGWLWGM | 17 | 85 | 3061 | ASKLCLGWLWGMDID | 19 | 17 | 85 |
| ENV | 2915 | LCLIFLLVL | 20 | 100 | 3062 | ILLLCLIFLLVLLDY | 249 | 19 | 95 |
| X | 2916 | LCLRPVGAE | 19 | 95 | 3063 | RDVLCLRPVGAESRG | 13 | 18 | 90 |
| POL | 2917 | LCQVFADAT | 19 | 95 | 3064 | RPGLCQVFADATPTG | 680 | 11 | 55 |
| ENV | 2918 | LDSWWTSLN | 19 | 95 | 3065 | PQSLDSWWTSLNFLG | 192 | 17 | 85 |
| NUC | 2919 | LDTASALYR | 17 | 85 | 3086 | RDLLDTASALYREAL | 28 | 18 | 80 |
| POL | 2920 | LDVSAAFYH | 19 | 95 | 3067 | WLSLDVSAAFYHIPL | 425 | 11 | 55 |
| ENV | 2921 | LDYQGMLPV | 18 | 90 | 3088 | LVLLDYQGMLPVCPL | 258 | 18 | 90 |
| POL | 2922 | LEEELPRLA | 18 | 90 | 3069 | AGPLEEELPRLADEG | 18 | 13 | 65 |
| ENV | 2923 | LFILLLCLI | 16 | 80 | 3070 | IIFLFILLLCLIFLL | 244 | 16 | 80 |
| POL | 2924 | LGAKSVQHL | 17 | 95 | 3071 | DVVLGAKSVQHLESL | 541 | 16 | 80 |
| POL | 2925 | LGFAAPPTQ | 19 | 95 | 3072 | VGLLGFAAPFTQCGY | 626 | 19 | 95 |
| POL | 2926 | LGFRKIPMG | 19 | 95 | 3073 | PIILGFRKIPMGVGL | 496 | 13 | 85 |
| POL | 2927 | LGNLNVSIP | 19 | 95 | 3074 | DLNLGNLNVSIPWTH | 40 | 19 | 95 |
| ENV | 2928 | LGPLLVLQA | 19 | 95 | 3075 | SGFLGPLLVLQAGFF | 169 | 15 | 75 |
| POL | 2929 | LHPAAMPHL | 20 | 100 | 3076 | HLPLHPAAMPHLLVG | 425 | 9 | 45 |
| ENV | 2930 | LIFLLVLLD | 19 | 95 | 3077 | LLCIFLLVLLDYQG | 251 | 19 | 95 |
| POL | 2931 | LKLIMPARF | 15 | 75 | 3078 | KRRLKLIMPARFYPN | 104 | 7 | 35 |
| X | 2932 | LKVFVLGGC | 15 | 75 | 3079 | EIRLKVFVLGGCRHK | 126 | 13 | 65 |
| POL | 2933 | LLAQFTSAI | 19 | 95 | 3080 | SPFLLAQFTSAICSV | 511 | 19 | 95 |
| NUC | 2934 | LLDTASALY | 17 | 85 | 3081 | IRDLLDTASALYREA | 56 | 9 | 45 |
| POL | 2935 | LLGCAANWI | 18 | 80 | 3082 | FPWLLGCAANWILRG | 749 | 15 | 75 |
| POL | 2936 | LLGFAAPFT | 19 | 95 | 3083 | IVGLLGFAAPFTQCG | 625 | 18 | 90 |
| ENV | 2937 | LLGWSPQAQ | 17 | 85 | 3084 | HGGLLGWSPQAQGIL | 60 | 15 | 75 |
| ENV | 2938 | LLLCLIFLL | 20 | 100 | 3085 | LFILLLCLIFLLVLL | 247 | 16 | 80 |
| NUC | 2939 | LLSFPSDF | 19 | 95 | 3086 | SVELLSFLPSDFFPS | 41 | 11 | 55 |
| POL | 2940 | LLSLGIHLN | 19 | 95 | 3087 | TNFLLSLGIHLNPNK | 560 | 15 | 75 |
| POL | 2941 | LLSSNLSWL | 18 | 90 | 3088 | LTNLLSSNLSWLSLD | 404 | 18 | 90 |
| ENV | 2942 | LLTRILTIP | 16 | 80 | 3089 | GFFLLTRILTIPQSL | 181 | 16 | 80 |
| ENV | 2943 | LLVLQAGFF | 19 | 95 | 3090 | LGPLLVLQAGFFLLT | 172 | 18 | 90 |
| ENV | 2944 | LLVPFVQWF | 20 | 100 | 3091 | WLSLLVPFVQWFVGL | 335 | 19 | 95 |
| NUC | 2945 | LLWFHISCL | 18 | 90 | 3092 | IRQLLWFHISCLTFG | 126 | 13 | 85 |
| POL | 2946 | LMPLYACIQ | 19 | 95 | 3093 | YPALMPLYACIQSKQ | 640 | 11 | 55 |
| POL | 2947 | LNLGNLNVS | 19 | 95 | 3094 | AEDLNLGNLNVSIPW | 38 | 19 | 95 |
| POL | 2948 | LNPNKTKRW | 15 | 75 | 3095 | GIHLNPNKTKRWGYS | 567 | 15 | 75 |
| POL | 2949 | LNRRVAEDL | 17 | 85 | 3096 | DEGLNRRVAEDLNLG | 30 | 12 | 60 |
| POL | 2950 | LNVSIPWTH | 19 | 95 | 3097 | LGNLNVSPWTHKVG | 43 | 19 | 95 |
| NUC | 2951 | LPETTVVRR | 19 | 95 | 3098 | LSTLPETTVVRRRGR | 169 | 16 | 80 |
| ENV | 2952 | LPIFFCLWV | 20 | 100 | 3099 | LPLLPIFFCLWVYIZ | 376 | 13 | 65 |
| POL | 2953 | LPIHTAELL | 17 | 85 | 3100 | VAPLPIHTAELLAAC | 709 | 9 | 45 |
| POL | 2954 | LPVNRPIDW | 16 | 80 | 3101 | FRKLPVNRPIDWKVC | 608 | 15 | 75 |
| POL | 2955 | LQFRNSKPC | 18 | 90 | 3102 | CWWLQFRNSKPCSDY | 312 | 10 | 50 |
| X | 2956 | LRGLPVCAF | 19 | 95 | 3103 | HLSLRGLPVCAFSSA | 52 | 18 | 90 |
| X | 2957 | LRPVGAESR | 18 | 90 | 3104 | VLCLRPVGAESRGRP | 15 | 18 | 90 |
| NUC | 2958 | LRQAILCWG | 18 | 90 | 3105 | HTALRQAILCWGELM | 52 | 18 | 90 |
| ENV | 2959 | LRRFIIFLF | 15 | 75 | 3106 | WMCLRRFIIFLFILL | 237 | 15 | 75 |
| NUC | 2960 | LSFLPSDFF | 19 | 95 | 3107 | VELLSFLPSDFFPSI | 42 | 10 | 50 |
| POL | 2961 | LSLDVSAAF | 19 | 95 | 3108 | LSWLSLDVSAAFYHI | 423 | 11 | 55 |
| ENV | 2962 | LSLLVPFVQ | 20 | 100 | 3109 | FSWLSLLVPPVQWFV | 333 | 19 | 95 |
| X | 2963 | LSLRGLPVC | 19 | 3110 | GAHLSLRGLPVCAFS | 50 | 18 | 90 | |
| POL | 2964 | LSPFLLAQF | 19 | 95 | 3111 | GVGLSPFLLAQFTSA | 507 | 18 | 80 |
| POL | 2965 | LSRKYTSFP | 17 | 85 | 3112 | SVVLSRKYTSFPWLL | 739 | 17 | 85 |
| POL | 2966 | LSSNLSWLS | 18 | 90 | 3113 | TNLLSSNLSWLSLDV | 405 | 18 | 90 |
| ENV | 2967 | LSVPNPLGF | 15 | 75 | 3114 | GTNLSVPNPLGFFPD | 13 | 14 | 70 |
| POL | 2968 | LSWLSLDVS | 20 | 100 | 3115 | SSNLSWLSLDVSAAF | 409 | 17 | 85 |
| ENV | 2969 | LTIPQSLDS | 18 | 90 | 3116 | TRILTIPQSLDSWWT | 186 | 15 | 75 |
| POL | 2970 | LTNLLSSNL | 18 | 90 | 3117 | LQSLTNLLSSNLSWL | 401 | 18 | 90 |
| ENV | 2971 | LTRILTIPQ | 16 | 80 | 3118 | FFLLTRILTIPQSLD | 182 | 15 | 75 |
| POL | 2972 | LVDKNPHNT | 20 | 100 | 3119 | GVFLVDKNPHNTTES | 372 | 11 | 55 |
| NUC | 2973 | LVSFGVWIR | 18 | 90 | 3120 | LEYLVSFGVWIRTPP | 145 | 14 | 70 |
| POL | 2974 | LVVDSQFS | 20 | 100 | 3121 | ESRLVVDFSQFSRGN | 374 | 45 | |
| NUC | 2975 | LWFHISCLT | 17 | 85 | 3122 | RQLLWFHISCLTFGR | 98 | 17 | 85 |
| NUC | 2976 | LWGMDIDPY | 17 | 85 | 3123 | LGWLWGMDIDPYKEF | 24 | 17 | 85 |
| POL | 2977 | LWKAGILYK | 20 | 100 | 3124 | LHTLWKAGILYKRET | 148 | 18 | 90 |
| NUC | 2978 | LYREALESP | 17 | 85 | 3125 | ASALYREALESPEHC | 34 | 17 | 85 |
| POL | 2979 | LYSHPIILG | 16 | 80 | 3126 | KLHLYSHPIILGFRK | 489 | 16 | 80 |
| POL | 2980 | MDDVVLGAK | 18 | 90 | 3127 | FSYMDDVVLGAKSVQ | 536 | 18 | 90 |
| POL | 2981 | MGVGLSPFL | 18 | 80 | 3128 | KIPMGVGLSPFLLAQ | 503 | 16 | 80 |
| POL | 2982 | MPHLLVGSS | 17 | 85 | 3129 | PAAMPHLLVGSSGLS | 430 | 8 | 40 |
| ENV | 2983 | MQWNSTTFH | 16 | 80 | 3130 | PQAMQWNSTTFHQTL | 106 | 8 | 40 |
| X | 2984 | MSTTDLEAY | 15 | 75 | 3131 | LSAMSTTDLEAYFKD | 100 | 9 | 45 |
| ENV | 2985 | MWYWGPSLY | 17 | 85 | 3132 | IWMMWYWGPSLYNIL | 389 | 9 | 45 |
| X | 2986 | VCAFSSAGP | 19 | 95 | 3133 | GLPVCAFSSAGPCAL | 57 | 18 | 90 |
| POL | 2987 | VCQRIVGLL | 17 | 85 | 3134 | DWKVCQRIVGLLGFA | 618 | 17 | 85 |
| POL | 2988 | VFADATPTG | 19 | 95 | 3135 | LCQVFADATPTGWGL | 683 | 19 | 95 |
| ENV | 2989 | VGLSPTVWL | 19 | 95 | 3136 | QWFVGLSPTVWLSVI | 344 | 14 | 70 |
| POL | 2990 | VGPLTVNEK | 17 | 85 | 3137 | QQYVGPLTVNEKRRL | 93 | 8 | 40 |
| POL | 2991 | VHFASPLHV | 16 | 80 | 3138 | PDRVHFASPLHVAWR | 816 | 12 | 60 |
| X | 2992 | VLCLRPVGA | 19 | 95 | 3139 | ARDVLCLRPVGAESR | 12 | 14 | 70 |
| POL | 2993 | VLGAKSVQH | 19 | 95 | 3140 | DDVVLGAKSVQHLES | 540 | 16 | 80 |
| X | 2994 | VLHKRTLGL | 17 | 85 | 3141 | LPKVLHKRTLGLSAM | 89 | 11 | 55 |
| POL | 2995 | VPNLQSLTN | 19 | 95 | 3142 | KFAVPNLQSLTNLLS | 395 | 19 | 95 |
| NUC | 2996 | VQASKICLG | 16 | 80 | 3143 | CPTVQASKLCLGWLW | 14 | 15 | 75 |
| ENV | 2997 | VRFSWLSLL | 16 | 80 | 3144 | WASVRFSWLSLLVPF | 328 | 13 | 65 |
| POL | 2998 | VRRAFPHCL | 19 | 95 | 3145 | CSVVRRAFPHCLAFS | 523 | 19 | 95 |
| POL | 2999 | VSIPWTHKV | 20 | 100 | 3146 | NLNVSIPWTHKVGNF | 45 | 19 | 95 |
| NUC | 3000 | VWIATPPAY | 19 | 95 | 3147 | SFGVWIRTPPAYRPP | 121 | 18 | 90 |
| POL | 3001 | VYVPSALNP | 18 | 90 | 3148 | TSFVYVPSALNPADD | 764 | 18 | 80 |
| NUC | 3002 | WFHISCLTF | 18 | 90 | 3149 | QLLWFHISCLTFGRE | 99 | 17 | 85 |
| ENV | 3003 | WFVGLSPTV | 19 | 95 | 3150 | FVQWFVGLSPTVWLS | 342 | 19 | 95 |
| POL | 3004 | WILRGTSFV | 16 | 80 | 3151 | AANWILRGTSFVYVP | 756 | 14 | 70 |
| NUC | 3005 | WIRTPPAYR | 19 | 95 | 3152 | FGVWIRTPPAYRPPN | 122 | 19 | 95 |
| POL | 3006 | WKAGILYKR | 20 | 100 | 3153 | HTLWKAGILYKRETT | 149 | 18 | 90 |
| POL | 3007 | WLLGCAANW | 16 | 80 | 3154 | SFPWLLGCAANWILR | 748 | 15 | 75 |
| POL | 3008 | WLSLDVSAA | 19 | 95 | 3155 | NLSWLSLDVSAAFYH | 411 | 17 | 85 |
| ENV | 3009 | WLSLLVPFV | 20 | 100 | 3156 | RFSWLSLLVPFVQWF | 332 | 20 | 100 |
| POL | 3010 | WPKFAVPNL | 19 | 95 | 3157 | RVSWPKFAVPNLQSL | 390 | 11 | 55 |
| POL | 3011 | YMDDVVLGA | 18 | 90 | 3158 | AFSYMDDVVLGAKSV | 535 | 18 | 90 |
| POL | 3012 | YPALMPLYA | 19 | 95 | 3159 | QCGYPALMPLYACIQ | 637 | 19 | 95 |
| ENV | 3013 | YQGMLPVCP | 18 | 3160 | LLDYQGMILPVCPLIP | 260 | 10 | 50 | |
| NUC | 3014 | YRPPNAPIL | 20 | 100 | 3161 | PPAYRPPNAPILSTL | 129 | 19 | 95 |
| ENV | 3015 | YRWMCLRRF | 19 | 95 | 3162 | CPGYRWMCLRRFIIF | 232 | 19 | 95 |
| POL | 3016 | YSHPIILGF | 16 | 80 | 3163 | LHLYSHPIILGFRKI | 490 | 16 | 80 |
| POL | 3017 | YSLNFMGYV | 15 | 75 | 3164 | RWGYSLNFMGYVIGS | 588 | 11 | 55 |
| POL | 3018 | YVPSALNPA | 18 | 90 | 3165 | SFVYVPSALNPADDP | 765 | 16 | 80 |
| ENV | 3019 | FFCLWVYIZ | 20 | | | | 382 | | |
| ENV | 3020 | MGTNLSVPN | 15 | | | | 12 | | |

TABLE XIXB

| HBV DR-SUPER MOTIF With Binding Data | | | | | | | | | | | | | | | | |
|---|---|---|---|---|---|---|---|---|---|---|---|---|---|---|---|---|
| Core | | | | | | | | | | | | | | | | |
| SEQ ID | | SEQ ID | | | | | | | | | | | | | | |
| NO: | Core Sequence | NO: | Exemplary Sequence | DR1 | DR2wβ1 DR2wβ2 | DR3 | DR4w4 | DR4w15 | DR5w11 | DR5w12 | DR6w19 | DR7 | DR8W2 | DR9 | Drw53 | |
| 2874 | FAAPFTQCG | 3021 | LLGFAAPFTQCGYPA | | | | | | | | | | | | | |
| 2875 | FADATPTGW | 3022 | CQVFADATPTGWGLA | | | | | | 0.2800 | | | | | | | |
| 2876 | FAVPNLQSL | 3023 | WPKFAVPNLQSLTNL | 0.0007 | | 0.0013 | | 0.0023 | | 0.0002 | | | 0.0008 | | | 0.0180 |
| 2877 | FGRETVLEY | 3024 | CLTFGRETVLEYLVS | | | | | | | | | | | | | |
| 2878 | FGVEPSGSG | 3025 | RRSFGVEPSGSGHID | | | | | | | | | | | | | |
| 2879 | FHISCLTFG | 3026 | LLWFHISCLTFGRET | | | | | | | | | | | | | |
| 2880 | FHLCLIISC | 3027 | MQLFHLCLIISCSCP | | | | | | | | | | | | | |
| 2881 | FILILCLIF | 3028 | IFLFILLLCLIFLLV | 0.0005 | | | | 0.0041 | | | | | 0.0018 | | | |
| 2882 | FLFILLLCL | 3029 | FIIFLFILLLCLIFL | | | | | | | | | | | | | |
| 2883 | FLGPLLVLQ | 3030 | TSGFLGPLLVLQAGF | | | | | | | | | | | | | |
| 2884 | FLLTRILTI | 3031 | AGFFLLTRILTIPQS | 4.6000 | 0.0420 | 0.0190 | 0.0040 | 5.3000 | 0.1500 | 3.6000 | 0.0700 | 0.3700 | 3.1000 | 0.2600 | 1.3000 | |
| 2885 | FLLVLLDYQ | 3032 | CLIFLLVLLDYQGML | | | | | | | | | | | | | |
| 2888 | FPAGGSSSG | 3033 | GLYFPAGGSSSGTVN | | | | | | | | | | | | | |
| 2887 | FPDHQLDPA | 3034 | LGFFPDHQLDPAFGA | | | | | | | | | | | | | |
| 2888 | FPHCLAFSY | 3035 | RRAFPHCLAFSYMDD | 0.0010 | | 0.0010 | | −0.0009 | | 0.0010 | | | 0.0017 | | | |
| 2889 | FRKIPMGVG | 3036 | ILGFRKIPMGVGLSP | | | | | | | | | | | | | |
| 2890 | FRKLPVNRP | 3037 | KQCFRKLPVNRPIDW | 1.5000 | 0.0022 | 0.0210 | −0.0006 | 1.2000 | 0.8500 | 0.0130 | 0.0013 | 0.0043 | 0.4000 | 0.0580 | 0.0250 | |
| 2891 | FSSAGPCAL | 3038 | VCAFSSAGPCALRFT | 0.2100 | | 0.2600 | | 0.0023 | | 0.0003 | | | 0.0200 | | | 0.0150 |
| 2892 | FSWLSLLVP | 3039 | SVRFSWLSLLVPFVQ | 0.9000 | | | | 0.0099 | | | | | 0.0037 | | | |
| 2893 | FTFSPTYKA | 3040 | KQAFTFSPTYKAFLC | 0.5300 | 0.2400 | 0.1400 | 0.0090 | 1.1000 | 0.2200 | 0.2400 | 0.0024 | 0.0200 | 0.3300 | 0.1200 | 0.5400 | |
| 2894 | FTGLYSSTV | 3041 | VGNFTGLYSSTVPVF | 1.7000 | 0.0100 | 0.0016 | | 0.0140 | 0.1700 | 0.0035 | | 0.0580 | 0.5800 | 0.0044 | 0.3100 | |
| 2895 | FTSAICSVV | 3042 | LAQFTSAICSVVRRA | 0.0120 | 0.0065 | 0.1500 | −0.0009 | 0.0150 | 0.0280 | 0.0076 | 0.0091 | 0.0010 | 0.0280 | 0.0150 | 0.0880 | 0.0190 |
| 2896 | FVGLSPTVW | 3043 | VQWFVGLSPTVWLSV | | | | | | | | | | | | | |
| 2897 | FVLGGCRHK | 3044 | LKVFVLGGCRHKLVC | | | | | | | | | | | | | |
| 2898 | FVQWFVGLS | 3045 | LVPFVQWFVGLSPTV | 0.0130 | 0.6900 | 0.0140 | −0.0013 | 0.1500 | 1.4000 | 0.3800 | 0.66000.0018 | 0.0092 | 0.6600 | 2.5000 | 2.6000 | |
| 2899 | FVYVPSALN | 3046 | GTSFVYVPSALNPAD | 0.3500 | 0.0140 | 0.0500 | −0.0008 | 0.3800 | 0.4100 | 0.0470 | −0.0001 | 0.0001 | 0.2700 | 0.0610 | 0.3400 | |
| 2900 | IDWKVCQRI | 3047 | NRPIDWKVCQRIVGL | | | | | | | | | | | | | |
| 2901 | IFLFILLLC | 3048 | RFIIFLFILLLCLIF | | | | | | | | | | | | | |
| 2902 | IFLLVLLDY | 3049 | LCLIFLLVLLDYQGM | 0.0016 | | 0.0060 | | 0.0230 | | 0.0017 | | | 0.0044 | | | |
| 2903 | IGTDNSVVL | 3050 | AKLIGTDNSVVLSRK | | | | | | | | | | | | | |
| 2904 | IHTAELLAA | 3051 | PLPIHTAELLAACFA | 0.0046 | | | | 0.0490 | | | | | −0.0003 | | | |
| 2905 | IIFLFILLL | 3052 | RRFIIFLFILLLCLI | | | | | | | | | | | | | |
| 2908 | ILLLCLIFL | 3053 | FLFILLLCLIFLLVL | | | | | | | | | | | | | |
| 2907 | ILRGTSFVY | 3054 | ANWILRGTSFVYVPS | | | | | | | | | | | | | |
| 2908 | ILSTLPETT | 3055 | NAPILSTLPETTVVR | 0.0009 | | 0.0009 | | −0.0007 | | −0.0002 | | | 0.0005 | | | 0.1600 |
| 2909 | IPIPSSWAF | 3056 | CTCIPIPSSWAFARF | | | | | | | | | | | | | |
| 2910 | IRTPPAYRP | 3057 | GVWIRTPPAYRPPNA | 0.3700 | 0.0420 | 7.2000 | 0.0120 | 3.4000 | 0.5700 | 0.4800 | 0.0140 | −0.0004 | 0.2200 | 0.5300 | 0.0450 | |
| 2911 | LAACFARSR | 3058 | AELLAACFARSRSGA | | | | | | | | | | | | | |
| 2912 | LAFSYMDDV | 3059 | PHCLAFSYMDDVVLG | | | | | | | | | | | | | |
| 2913 | LAQFTSAIC | 3060 | PFLLAQFTSAICSVV | 0.1800 | 0.0270 | 0.0042 | −0.0013 | 0.0800 | 0.1200 | 0.0120 | 0.0016 | 0.0800 | 0.0770 | 0.0580 | 0.0590 | |
| 2914 | LCLGWLWGM | 3061 | ASKLCLGWLWGMDID | 0.0002 | | −0.0005 | | 0.0017 | | −0.0002 | | | 0.0013 | | | 0.0010 |
| 2915 | LCLIFLLVL | 3062 | ILLLCLIFLLVLLDY | 0.0026 | | 0.0069 | | 0.0320 | | 0.0018 | | | 0.0047 | | | |
| 2916 | LCLRPVGAE | 3063 | RDVLCLRPVGAESRG | | | | | | | | | | | | | |
| 2917 | LCQVFADAT | 3064 | RPGLCQVFADATPTG | | | | | | | | | | | | | |
| 2918 | LDSWWTSLN | 3085 | PQSLDSWWTSLNFLG | | | | | | | | | | | | | |
| 2919 | LDTASALYR | 3066 | RDLLDTASALYREAL | 0.0001 | | | | 0.0092 | | | | | 0.0770 | | | |
| 2920 | LDVSAAFYH | 3087 | WLSLDVSAAFYHIPL | | | | | | | | | | | | | |
| 2921 | LDYQGMLPV | 3088 | LVLLDYQGMLPVCPL | 0.0034 | | | −0.0013 | | | | | 0.0011 | | | | |
| 2922 | LEEELPRLA | 3089 | AGPLEEELPRLADEG | | | | 0.0022 | | | | | | | | | |
| 2923 | LFILLLCLI | 3070 | IIFLFILLLCLIFLL | | | | | | | | | | | | | |
| 2924 | LGAKSVQHL | 3071 | DVVLGAKSVQHLESL | | | | | | | | | | | | | |
| 2925 | LGFAAPFTQ | 3072 | VGLLGFAAPFTQCGY | 0.0470 | 0.3100 | 0.0008 | | −0.0014 | | −0.0004 | | −0.0001 | 0.0014 | | 0.5700 | |
| 2926 | LGFRKIPMG | 3073 | PIILFRKIPMGVGL | | | | | | | | | | | | | |
| 2927 | LGNLNVSIP | 3074 | DLNLGNLNVSIPWTH | 0.0038 | | | | 0.0240 | | | | | 0.0010 | | | |
| 2928 | LGPLLVLQA | 3075 | SGFLGPLLVLQAGFF | | | | | | | | | | | | | |
| 2929 | LHPAAMPHL | 3076 | HLPLHPAAMPHLLVG | | | | | | | | | | | | | |
| 2930 | LIFLLVLLD | 3077 | LLCLIFLLVLLDYQG | | | | | | | | | | | | | |
| 2931 | LKLIMPARF | 3078 | KRRLKLIMPARFYPN | | | | | | | | | | | | | |
| 2932 | LKVFVLGGC | 3079 | EIRLKVFVLGGCRHK | | | | | | | | | | | | | |
| 2933 | LLAQFTSAI | 3080 | SPFLLAQFTSAICSV | 0.1200 | 0.0200 | 0.0085 | −0.0013 | 0.0740 | 0.0190 | −0.0002 | −0.0013 | 0.0540 | 0.0330 | 0.0014 | 0.0380 | 0.2000 |
| 2934 | LLDTASALY | 3081 | IRDLLDTASALYREA | | | | | | | | | | | | | |
| 2935 | LLGCAANWI | 3082 | FPWLLGCAANWILRG | | | | | | | | | | | | | |
| 2936 | LLGFAAPFT | 3083 | IVGLLGFAAPFTQCG | 0.0200 | | −0.0005 | | −0.0007 | | −0.0002 | | | 0.0009 | | | 0.0067 |
| 2937 | LLGWSPQAQ | 3084 | HGGLLGWSPQAQGIL | | | | | | | | | | | | | |
| 2938 | LLLCLIFLL | 3085 | LFILLLCLIFLLVLL | | | | | | | | | | | | | |
| 2939 | LLSFLPSDF | 3088 | SVELLSFLPSDFFPS | | | | | | | | | | | | | |
| 2940 | LLSLGIHLN | 3087 | TNFLLSLGIHLNPNK | 3.5000 | 0.0410 | 0.1200 | | 0.0220 | 0.0360 | 0.0053 | | 0.0160 | 0.2200 | 0.0032 | 0.3800 | |
| 2941 | LLSSNLSWL | 3088 | LTNLLSSNLSWLSLD | 0.0010 | | 0.0083 | | 0.0160 | | 0.0013 | | | 0.0019 | | | 0.0200 |
| 2942 | LLTRILTIP | 3089 | GFFLLTRILTIPQSL | 0.4300 | 0.0150 | 0.0110 | | 3.1000 | 0.4500 | 2.3000 | | 0.0780 | 3.5000 | 1.6000 | 0.5500 | |
| 2943 | LLVLQAGFF | 3090 | LGPLLVLQAGFFLLT | | | | | | | | | | | | | |
| 2944 | LLVPFVQWF | 3091 | WLSLLVPFVQWFVGL | | | | | | | | | | | | | |
| 2945 | LLWFHISCL | 3092 | IRQLLWFHISCLTFG | | | | | | | | | | | | | |
| 2946 | LMPLYACIQ | 3093 | YPALMPLYACIQSKQ 0.2400 | | | | 0.0014 | | | | | 0.0011 | | | | |
| 2947 | LNLGNLNVS | 3094 | AEDLNLGNLNVSIPW | 0.0001 | | −0.0005 | | −0.0007 | | −0.0002 | | | −0.0003 | | | 0.0170 |
| 2948 | LNPNKTKRW | 3095 | GHLNPNKTKRWGYS | | | | | | | | | | | | | |
| 2949 | LNRRVAEDL | 3098 | DEGLNRRVAEDLNLG | | | | | | | | | | | | | |
| 2950 | LNVSIPWTH | 3097 | LGNLNVSIPWTHKVG | | | | | | | | | | | | | |
| 2951 | LPETTVVRR | 3098 | LSTLPETTVVRRRGR | | | | | | | | | | | | | |
| 2952 | LPIFFCLWV | 3099 | LPLLPIFFCLWVYIZ | | | | | | | | | | | | | |
| 2953 | LPIHTAELL | 3100 | VAPLPIHTAELLAAC | | | | | | | | | | | | | |
| 2954 | LPVNRPIDW | 3101 | FRKLPVNRPIDWKVC | | | | | | | | | | | | | |
| 2955 | LQFRNSKPC | 3102 | CWWLQFRNSKPCSDY | | | | | | | | | | | | | |
| 2956 | LRGLPVCAF | 3103 | HLSLRGLPVCAFSSA | 1.3000 | | | | 0.0028 | | | | | 0.0130 | | | |
| 2957 | LRPVGAESR | 3104 | VLCLRPVGAESRGRP | | | | | | | | | | | | | |
| 2958 | LRDAILCWG | 3105 | HTALRQAILCWGELM | | | | | | | | | | | | | |
| 2959 | LRRFIIFLF | 3106 | WMCLRRFIIFLFILL | | | | | | | | | | | | | |
| 2960 | LSFLPSDFF | 3107 | VELLSFLPSDFFPSI | | | | | | | | | | | | | |
| 2961 | LSLDVSAAF | 3108 | LSWLSLDVSAAFYHI | | | | | | | | | | | | | |
| 2962 | LSLLVPFVQ | 3109 | FSWLSLLVPFVQWFV | | | | | | | | | | | | | |
| 2963 | LSLRGLPVC | 3110 | GAHLSLRGLPVCAFS | 0.7899 | | 0.0042 | −0.0041 | 0.0011 | | 0.0025 | | | 0.0077 | | | 0.0150 |
| 2964 | LSPFLLADF | 3111 | GVGLSPFLLAQFTSA | | | | | | | | | | | | | |
| 2965 | LSRKYTSFP | 3112 | SVVLSRKYTSFPWLL | 0.0005 | | 0.0057 | 0.2100 | −0.0016 | | 0.5300 | | | 0.0130 | | | |
| 2966 | LSSNLSWLS | 3113 | TNLLSSNLSWLSLDV | 0.0016 | | −0.0005 | | 0.1300 | | 0.0006 | | | 0.0019 | | | 0.0410 |
| 2967 | LSVPNPLGF | 3114 | GTTNLSVPNPLGFFPD | | | | | | | | | | | | | |
| 2968 | LSWLSLDVS | 3115 | SSNLSWLSLDVSAAF | 0.1400 | 0.0030 | −0.0005 | 1.5000 | 0.2700 | | 0.0046 | 0.0180 | 0.1000 | 0.0039 | 0.0480 | 0.0110 | 6.2000 |
| 2969 | LTIPQSLDS | 3116 | TRILTIPQSLDSWWT | | | | | | | | | | | | | |
| 2970 | LTNLLSSNL | 3117 | LQSLTNLLSSNLSWL | 2.5000 | 0.4400 | 0.0200 | −0.0013 | 4.8000 | 0.8100 | 0.0680 | 0.7500 | 0.0260 | 0.1500 | 0.0880 | 0.1100 | |
| 2971 | LTRILTIPQ | 3118 | FFLLTRILIPQSLD | | | | | | | | | | | | | |
| 2972 | LVDKNPHNT | 3119 | GVFLVDKNPHNTTES | | | | | | | | | | | | | |
| 2973 | LVSFGVWIR | 3120 | LEYLVSFGVWIRTPP | | | | | | | | | | | | | |
| 2974 | LVVDFSQFS | 3121 | ESRLVVDFSQFSRGN | 0.0007 | 0.0074 | −0.0010 | 2.6000 | | | −0.0004 | | 0.0040 | −0.0014 | 0.0029 | | |
| 2975 | LWFHISCLT | 3122 | RQLLWFHISCLTFGR | 0.0002 | | 0.0009 | | 0.0140 | | 0.0011 | | | 0.0061 | | | 0.0096 |
| 2976 | LWGMDIDPY | 3123 | LGWLWGMDIDPYKEF | 0.0004 | | 0.0006 | 0.02000.0280 | | −0.0002 | | | 0.0004 | | | 0.0430 | |
| 2977 | LWKAGILYK | 3124 | LHTLWKAGILYKRET | | | | | | | | | | | | | |
| 2978 | LYREALESP | 3125 | ASALYREALESPEHC | | | | | | | | | | | | | |
| 2979 | LYSHPIILG | 3126 | KLHLYSHPIILGFRK | | | | | | | | | | | | | |
| 2980 | MDDVVLGAK | 3127 | FSYMDDVVLGASVQ | | | | | | | | | | | | | |
| 2981 | MGVGLSPFL | 3128 | KIPMGVGLSPFLLAQ | | | | | | | | | | | | | |
| 2982 | MPHLLVGSS | 3129 | PAAMPHLLVGSSGLS | | | | | | | | | | | | | |
| 2983 | MQWNSTTFH | 3130 | PQAMQWNSTTFHQTL | 0.0012 | | | | 0.0300 | | | | | 0.1200 | | | |
| 2984 | MSTTDLEAY | 3131 | LSAMSTTDLEAYFKD | | | | | | | | | | | | | |
| 2985 | MWYWGPSLY | 3132 | IWMMWYWGPSLYNIL | | | | | | | | | | | | | |
| 2986 | VCAFSSAGP | 3133 | GLPVCAFSSAGPCAL | | | | | | | | | | | | | |
| 2987 | VCQRIVGLL | 3134 | DWKVCQRIVGLLGFA | 0.0120 | | −0.0026 | | 0.0030 | | 0.2500 | | | 0.0018 | | | 0.0130 |
| 2988 | VFADATPTG | 3135 | LCQVFADATPTGWGL | 0.0020 | | | | 0.9600 | | | | | 0.0013 | | | |
| 2989 | VGLSPVWL | 3136 | QWFVGLSPTWLSVI | | | | | | | | | | | | | |
| 2990 | VGPLTVNEK | 3137 | QQYVGPLTVNEKRRL | | | | | | | | | | | | | |
| 2991 | VHFASPLHV | 3138 | PDRVHFASPLHVAWR | 0.0610 | 0.0290 | 0.0008 | | 0.0008 | 0.0054 | 0.0008 | | 0.0190 | 0.0810 | 0.0035 | 0.2400 | |
| 2992 | VLCLRPVGA | 3139 | ARDVLCLRPVGAESR | | | | | | | | | | | | | |
| 2993 | VLGAKSVQH | 3140 | DDWLGAKSVQHLES | | | | | | | | | | | | | |
| 2994 | VLHKRTLGL | 3141 | LPKVLHKRTLGLSAM | | | | | | | | | | | | | |
| 2995 | VPNLQSLTN | 3142 | KFAVPNLQSLTNLLS | 0.0180 | 0.0005 | −0.0003 | | 0.1300 | | 0.0043 | | 0.0088 | −0.0003 | | 0.0056 | |
| 2996 | VQASKLCLG | 3143 | CPTVQASKLCLGWLW | | | | | | | | | | | | | |
| 2997 | VRFSWLSLL | 3144 | WASVRFSWLSLLVPF | | | | | | | | | | | | | |
| 2998 | VRRAFPHCL | 3145 | CSVVRRAFPHCLAFS | 0.1000 | 0.1024 | 0.0770 | 0.00320.0016 | −0.0022 | 0.0008 | −0.0013 | 0.0540 | 0.0590 | 0.0250 | 1.2000 | 0.0460 | |
| 2999 | VSIPWTHKV | 3146 | NLNVSIPWTHKVGNF | 0.0001 | | −0.0005 | −0.0041 | −0.0007 | | −0.0002 | | | 0.0005 | | | 0.0009 |
| 3000 | VWIRTPPAY | 3147 | SFGVWIRTPPAYRPP | 0.0094 | 0.0110 | 0.4300 | −0.0009 | 0.0780 | 0.0630 | 0.0260 | 0.0071 | 0.0002 | 0.0240 | 0.2500 | 0.0800 | 0.0016 |
| 3001 | VYVPSALNP | 3148 | TSFVYVPSALNPADD | | | | | | | | | | | | | |
| 3002 | WFHISCLTF | 3149 | QLLWFHSCLTFGRE | | | | | | | | | | | | | |
| 3003 | WFVGLSPTV | 3150 | FVQWFVGLSPTVWLS | 0.4700 | 0.0035 | 0.0160 | −0.0013 | 0.0130 | | 0.0072 | 0.0021 | 0.0190 | 0.0690 | 0.0180 | 0.0410 | 0.0044 |
| 3004 | WILRGTSFV | 3151 | AANWILRGTSFVYVP | 0.0920 | 0.0240 | 0.0061 | 0.00230.0510 | 0.0250 | 0.0140 | 0.3700 | 0.0250 | 0.5800 | 0.2500 | 0.2700 | | |
| 3005 | WIRTPPAYR | 3152 | FGVWIRTPPAYRPPN | | | | | | | | | | | | | |
| 3006 | WKAGILYKR | 3153 | HTLWKAGILYKRETT | | | | | | | | | | | | | |
| 3007 | WLLGCAANW | 3154 | SFPWLLGCAANWLR | | | | | | | | | | | | | |
| 3008 | WLSLDVSAA | 3155 | NLSWLSLDVSAAFYH | 0.1400 | 0.0003 | −0.0005 | 1.3000 | 0.2900 | | 0.0033 | 0.0022 | 0.0330 | 0.0041 | 0.0150 | 0.0620 | 2.4000 |
| 3009 | WLSLLVPFV | 3156 | RFSWLSLLVPFVQWF | 0.0430 | | 0.0009 | | −0.0007 | | 0.0002 | | | 0.0005 | | | 0.0031 |
| 3010 | WPKFAVPNL | 3157 | RVSWPKFAVPNLQSL | | | | | | | | | | | | | |
| 3011 | YMDDVVLGA | 3158 | AFSYMDDVVLGAKSV | 0.0027 | | −0.0005 | 0.0130 | 2.9000 | | 0.0006 | | | −0.0003 | | | −0.0005 |
| 3012 | YPALMPLYA | 3159 | QCGYPALMPLYACIQ | 0.0062 | | 0.0018 | | 0.0068 | | 0.0023 | | | 0.0006 | | | |
| 3013 | YQGMLPVCP | 3160 | LLDYQGMLPVCPLIP | | | | | | | | | | | | | |
| 3014 | YRPPNAPIL | 3161 | PPAYRPPNAPILSTL | 0.0056 | | −0.0005 | | 0.0038 | | 0.0022 | | | 0.0024 | | | 0.0015 |
| 3015 | YRWMCLRRF | 3162 | CPGYRWMCLRRFIIF | | | | | | | | | | | | | |
| 3016 | YSHPIILGF | 3163 | LHLYSHPILGFRKI | 0.0220 | 0.0340 | 0.0400 | 0.0040 | 0.8800 | 0.1600 | 0.0410 | 0.0310 | 0.0002 | 0.0006 | 0.0610 | 0.0490 | |
| 3017 | YSLNFMGYV | 3164 | RWGYSLNFMGYVIGS | | | | | | | | | | | | | |
| 3018 | YVPSALNPA | 3165 | SFVYVPSALNPADDP | | | | | | | | | | | | | |
| 3019 | FFCLWWIZ | | | | | | | | | | | | | | | |
| 3020 | MGTNLSVPN | | | | | | | | | | | | | | | |

TABLE XXa

HBV DR-3A Motif

| | | | | | | | |
|---|---|---|---|---|---|---|---|
| | Core SEQ | Core | | Core | Exemplary | Exemplary | Position in |
| Protein | ID NO: | Sequence | Core Freq. | Conservancy (%) | SEQ ID NO: | Sequence | Poly-Protein |

| | | | | | | | |
|---|---|---|---|---|---|---|---|
| ENV | 3166 | FFPDHQLDP | 19 | 95 | 3181 | PLGFFPQLDPAFG | 10 |
| NUC | 3167 | FGRETVLEY | 15 | 75 | 3182 | CLTFGRETVLEYLVS | 136 |
| POL | 3168 | FGVEPSGSG | 15 | 75 | 3183 | RRSFGVEPSGSGHD | 241 |
| POL | 3189 | FLVDKNPHN | 20 | 100 | 3184 | GGVFLVDKNPHNTTE | 360 |
| POL | 3170 | IGTDNSVVL | 16 | 80 | 3185 | AKLIGTDNSVVLSRK | 731 |
| POL | 3171 | LEEELPRLA | 18 | 90 | 3186 | AGPLEEELPRLADEG | 18 |
| POL | 3172 | LPLDKGIKP | 20 | 100 | 3187 | TKYLPLDKGIKYYP | 120 |
| POL | 3173 | LSLDVSAAF | 19 | 95 | 3188 | LSWLSLDVSAAFYHI | 412 |
| POL | 3174 | LVVDFSDFS | 20 | 100 | 3189 | ESRLVVDFSQFSRGN | 374 |
| NUC | 3175 | LYREALESP | 17 | 85 | 3190 | ASALYREALESPEHC | 34 |
| NUC | 3176 | MDIDPYKFE | 17 | 85 | 3191 | LWGMDIDPYKEFGAS | 27 |
| POL | 3177 | VAEDLNLGN | 20 | 100 | 3192 | NRRVAEDLNLGNLNV | 34 |
| POL | 3178 | VFADATPTG | 19 | 95 | 3193 | LCQVFADATPTGWGL | 683 |
| ENV | 3179 | VLLDYQGML | 19 | 95 | 3194 | FLLVLLDYQGMLPVC | 256 |
| POL | 3180 | YMDDVVLGA | 18 | 90 | 3195 | AFSYMDDVVLGAKSV | 535 |

| | | |
|---|---|---|
| | Exemplary Sequence | Exemplary Sequence |
| Protein | Frequency | Consrvancy (%) |

| | | |
|---|---|---|
| ENV | 9 | 95 |
| NUC | 14 | 75 |
| POL | 6 | 75 |
| POL | 11 | 100 |
| POL | 13 | 80 |
| POL | 13 | 90 |
| POL | 20 | 100 |
| POL | 11 | 95 |
| POL | 9 | 100 |
| NUC | 17 | 85 |
| NUC | 1 | 85 |
| POL | 17 | 100 |
| POL | 19 | 95 |
| ENV | 18 | 95 |
| POL | 18 | 90 |

TABLE XXb

HCV DR 3A Motif

| | | | | | | | | | | | |
|---|---|---|---|---|---|---|---|---|---|---|---|
| Core SEQ | Core | SEQ ID | Exemplary | | | | | | | | |
| ID NO: | Sequence | NO: | Sequence | DR1 | DR2w2β1 | DR2w2β2 | DR3 | DR4w4 | DR4w15 | DR5w11 | DR5w12 |

| | | | | | | | | | |
|---|---|---|---|---|---|---|---|---|---|
| 3166 | FFPDHQLDP | 3181 | PLGFFPDHQLDPAFG | | | | | | |
| 3167 | FGRETVLEY | 3182 | CLTFGRETVLEYLVS | | | | | | |
| 3168 | FGVEPSGSG | 3183 | RRSGFVEPSGSGHD | | | | | | |
| 3189 | FLYDKNPHN | 3184 | GGVFLVDKNPHNTTE | | | | 0.0790 | | |
| 3170 | IGTDNSVVL | 3185 | AKLIGTDNSVVLSRK | | | | | | |
| 3171 | LEEELPRLA | 3186 | AGLEEELPRLADEG | | | | 0.0022 | | |
| 3172 | LPLDKGIKP | 3187 | TKYLPLDKGKPYYP | | | | −0.0017 | | |
| 3173 | LSLDVSAAF | 3186 | LSWLSLDVSAAFYHI | | | | | | |
| 3174 | LVVDFSQFS | 3189 | ESRLVVDFSQFSRGN | 0.0007 | 0.0074 | −0.0010 | 2.8000 | | 0.0004 |
| 3175 | LYREALESP | 3190 | ASALYREALESPEHC | | | | | | |
| 3176 | MDIDPYKEF | 3191 | LWGMDIDPYKEFGAS | | | | | | |
| 3177 | VAEDLNLGN | 3192 | NRRVAEDLNLGNLNV | | | | 0.1400 | | |
| 3178 | VFADATPTG | 3193 | LCQVFADATPTGWGL | 0.0020 | | | | 0.9600 | |
| 3179 | VLLDYQGML | 3194 | FLLVLLDYQGMLPVC | | | | 0.0170 | | |
| 3180 | YMDDVVLGA | 3195 | AFSYMDDVVLGAKSV | 0.0027 | | −0.0005 | 0.0130 | 2.9000 | 0.0006 |

| | | | | | |
|---|---|---|---|---|---|
| Core SEQ | | | | | |
| ID NO: | DR8w19 | DR7 | DR8W2 | DR9 | DRW53 |

| | | | | |
|---|---|---|---|---|
| 3166 | | | | |
| 3167 | | | | |
| 3168 | | | | |
| 3189 | | | | |
| 3170 | | | | |
| 3171 | | | | |
| 3172 | | | | |
| 3173 | | | | |
| 3174 | 0.4000 | −0.0014 | 0.0029 | |
| 3175 | | | | |
| 3176 | | | | |
| 3177 | | | | |
| 3178 | | 0.0013 | | |
| 3179 | | | | |
| 3180 | | −0.0003 | | −0.0005 |

TABLE XXc

HBV DR-3B Motif

| | | | | | | | | | |
|---|---|---|---|---|---|---|---|---|---|
| | Core | | | Core | | | Position In | Exemplary | |
| | SEQ | Core | Core | Conservancy | | Exemplary | HBV | Sequence | Exemplary |
| Protein | ID NO: | Sequence | Freq. | (%) | SEQ ID NO: | Sequence | Poly-Protein | Frequency | Sequence |

| | | | | | | | | | |
|---|---|---|---|---|---|---|---|---|---|
| X | 3196 | AHLSLRGLP | 18 | 90 | 3202 | DNGAHLSLRGLPVCA | 48 | 18 | 90.00 |
| POL | 3197 | FSPTYKAFL | 19 | 95 | 3203 | AFTFSPTYKAFLCKQ | 655 | 11 | 55.00 |
| POL | 3198 | IPWTHKVGN | 20 | 100 | 3204 | NVSIPWTHKVGNFTG | 47 | 20 | 100.00 |
| POL | 3199 | LTVNEKRRL | 17 | 85 | 3205 | VGPLTVNEKRRLKLI | 98 | 12 | 60.00 |
| X | 3200 | VGAESRGRP | 19 | 95 | 3206 | LRPVGAESRGPVSG | 18 | 7 | 35.00 |
| POL | 3201 | VVLSRKYTS | 18 | 90 | 3207 | DNSVVLSRKYTSFPW | 737 | 17 | 85.00 |

TABLE XXd

HBV DR-3B Motif With Binding Information

| | | | | | | | | | | | |
|---|---|---|---|---|---|---|---|---|---|---|---|
| Core SEQ | Core | SEQ ID | | | | | | | | | |
| ID NO: | Sequence | NO: | Exemplary Sequence | DR1 | DR2w2β1 | DR2w2β2 | DR3 | DR4w4 | DR4w15 | DR5w11 | DR5w12 |

| | | | | | | | | |
|---|---|---|---|---|---|---|---|---|
| 3196 | AHLSLRGLP | 3202 | DHGAAHLSLRGLPVCA | | | | | |
| 3197 | FSPTYKAFL | 3203 | AFTFSPTYKAFLCKQ | | | | 0.0035 | |
| 3198 | IPWTHKVGN | 3204 | NVSIPWTHKVGNFTG | | | | | |
| 3199 | LTVNEKRRL | 3205 | VGPLTVNEKRRLKLI | 0.0006 | 0.0022 | 0.0047 | 2.2000 | 0.0030 |
| 3200 | VGAESRGRP | 3206 | LRPVGAESRGRPVSG | | | | −0.0017 | |
| 3201 | VVLSRKYTS | 3207 | DNSVVLSRKYTSFPW | | | | | |

TABLE XXI
Population coverage with combined HLA Supertypes

| | PHENOTYPIC FREQUENCY | | | | | |
|---|---|---|---|---|---|---|
| | | North | | | | |
| | | American | | | | |
| HLA-SUPERTYPES | Caucasian | Black | Japanese | Chinese | Hispanic | Average |
| a. Individual Supertypes | | | | | | |
| A2 | 45.8 | 39.0 | 42.4 | 45.9 | 43.0 | 43.2 |
| A3 | 37.5 | 42.1 | 45.8 | 52.7 | 43.1 | 44.2 |
| B7 | 38.6 | 52.7 | 48.8 | 35.5 | 47.1 | 44.7 |
| A1 | 47.1 | 16.1 | 21.8 | 14.7 | 26.3 | 25.2 |
| A24 | 23.9 | 38.9 | 58.6 | 40.1 | 38.3 | 40.0 |
| B44 | 43.0 | 21.2 | 42.9 | 39.1 | 39.0 | 37.0 |
| B27 | 28.4 | 26.1 | 13.3 | 13.9 | 35.3 | 23.4 |
| B62 | 12.6 | 4.8 | 36.5 | 25.4 | 11.1 | 18.1 |
| B58 | 10.0 | 25.1 | 1.6 | 9.0 | 5.9 | 10.3 |
| b. Combined Supertypes | | | | | | |
| A2, A3, B7 | 83.0 | 86.1 | 87.5 | 88.4 | 86.3 | 86.2 |
| A2, A3, B7, A24, B44, A1 | 99.5 | 98.1 | 100.0 | 99.5 | 99.4 | 99.3 |
| A2, A3, B7, A24, B44, A1, | 99.9 | 99.6 | 100.0 | 99.8 | 99.9 | 99.8 |
| B27, B62, B58 | | | | | | |

TABLE XXII
HBV ANALOGS

| | | | | | | | | | | |
|---|---|---|---|---|---|---|---|---|---|---|
| | | | | A2 | A3 | | B7 | 1* | | |
| | | Fixed | A1 | Super | Super | A24 | Super | Anchor | | SEQ ID |
| AA | Sequence | Nomen | Motif | Motif | Motif | Motif | Motif | Fixer | Analog | NO: |

| | | | | | | | | | | |
|---|---|---|---|---|---|---|---|---|---|---|
| 10 | CILLLCLIFL | | N | Y | N | N | N | No | A | 3208 |
| 9 | RMTGGVFLV | VM2.V9 | N | Y | N | N | N | 1 | A | 3209 |
| 9 | LMPFVQWFV | VM2.V9 | N | Y | N | N | N | 1 | A | 3210 |
| 9 | RLTGGVFLV | VL2.V9 | N | Y | N | N | N | 1 | A | 3211 |
| 9 | GLCQVFADV | L2.AV9 | N | Y | N | N | N | 1 | A | 3212 |
| 9 | WLLRGTSFV | IL2.V9 | N | Y | N | N | N | 1 | A | 3213 |
| 9 | NLGNLNVSV | L2.IV9 | N | Y | N | N | N | 1 | A | 3214 |
| 9 | YLPSALNPV | VL2.AV9 | N | Y | N | N | N | 1 | A | 3215 |
| 9 | GLWIRTPPV | VL2.AV9 | N | Y | N | N | N | 1 | A | 3216 |
| 9 | RLSWPKFAV | VL2.V9 | N | Y | N | N | N | 1 | A | 3217 |
| 9 | ILGLLGFAV | VL2.AV9 | N | Y | N | N | N | 1 | A | 3218 |
| 9 | RMLTIPQSV | IM2.LV9 | N | Y | N | N | N | 1 | A | 3219 |
| 9 | SLDSWWTSV | L2.LV9 | N | Y | N | N | N | 1 | A | 3220 |
| 10 | FMLLLCLIFL | IM2.L10 | N | Y | N | V | N | 1 | A | 3221 |
| 10 | LMLQAGFFLV | VM2.LV | N | Y | N | N | N | 1 | A | 3222 |
| 10 | SMLSPFLPLV | IM2.LV1 | N | Y | N | N | N | 1 | A | 3223 |
| 10 | LMLLDYQGMV | VM2.LV | N | Y | N | N | N | 1 | A | 3224 |
| 10 | FLGLSPTVWV | VL2.LV1 | N | Y | N | N | N | 1 | A | 3225 |
| 8 | FPMMPHL | | N | N | N | N | Y | A | 3226 | |
| 8 | HPFAMPHL | | N | N | N | N | Y | A | 3227 | |
| 8 | HPAAMPHI | | N | N | N | N | Y | A | 3228 | |
| 8 | FMFSPTYK | | N | N | Y | N | N | A | 3229 | |
| 8 | FVFSPTYK | | N | N | Y | N | N | A | 3230 | |
| 9 | FLLTRILTV | L2.IV9 | N | Y | N | N | N | 1 | A | 3231 |
| 9 | ALMPLYACV | L2.IV9 | N | Y | N | N | N | 1 | A | 3232 |
| 9 | LLAQFTSAV | L2.IV9 | N | Y | N | N | N | 1 | A | 3233 |
| 9 | LLPFVQWFV | VL2.V9 | N | Y | N | N | N | 1 | A | 3234 |
| 9 | FLLAQFTSV | L2.AV9 | N | Y | N | N | N | 1 | A | 3235 |
| 9 | KLHLYSHPV | L2.IV9 | N | Y | N | N | N | 1 | A | 3236 |
| 9 | KLFLYSHPI | | N | Y | N | N | N | No | A | 3237 |
| 9 | LLSSLSWV | L2.LV9 | N | Y | N | N | N | 1 | A | 3238 |
| 9 | FLLSLGIHV | L2.LV9 | N | Y | N | N | N | 1 | A | 3239 |
| 9 | MMWYWGPSV | M2.LV9 | N | Y | N | N | N | 1 | A | 3240 |
| 9 | VLQAGFFLV | L2.LV9 | N | Y | N | N | N | 1 | A | 3241 |
| 9 | PLLPIFFCV | L2.LV9 | N | Y | N | N | N | 1 | A | 3242 |
| 9 | FLLPIFFCL | | N | Y | N | N | N | No | A | 3243 |
| 9 | VLLDYQGMV | L2.LV9 | N | Y | N | N | N | 1 | A | 3244 |
| 9 | YMFDVVLGA | | N | Y | N | N | N | No | A | 3245 |
| 9 | GLLGWSPQV | L2.AV9 | N | Y | N | N | N | 1 | A | 3246 |
| 9 | FPAAMPHLL | | N | N | N | N | Y | | A | 3247 |
| 9 | HPFAMPHLL | | N | N | N | N | Y | | A | 3248 |
| 9 | HPAAMPHLI | | N | N | N | N | Y | | A | 3249 |
| 9 | FPVCAFSSA | | N | N | N | N | Y | | A | 3250 |
| 9 | LPFCAFSSA | | N | N | N | N | Y | | A | 3251 |
| 9 | LPVCAFSSI | | N | N | N | N | Y | | A | 3252 |
| 9 | FPALMPLYA | | N | N | N | N | Y | | A | 3253 |
| 9 | YPFLMPLYA | | N | N | N | N | Y | | A | 3254 |
| 9 | YPALMPLYI | | N | N | N | N | Y | | A | 3255 |
| 9 | FPSRGRLGL | | N | N | N | N | Y | | A | 3256 |
| 9 | DPFRGRLGL | | N | N | N | N | Y | | A | 3257 |
| 9 | DPSRGRLGI | | N | N | N | N | Y | | A | 3258 |
| 9 | SMICSVVRR | | N | N | Y | N | N | | A | 3259 |
| 9 | SVICSVVRR | | N | N | Y | N | N | | A | 3260 |
| 9 | KVGNFTGLK | | N | N | Y | N | N | | A | 3261 |
| 9 | KVNGFTGLR | | N | N | Y | N | N | | A | 3262 |
| 9 | WFFSQFSR | | N | N | Y | N | N | | A | 3263 |
| 9 | SVNRPIDWK | | N | N | Y | N | N | | A | 3264 |
| 9 | TLWKAGILK | | N | N | Y | N | N | | A | 3265 |
| 9 | TLWKAGILR | | N | N | Y | N | N | | A | 3266 |
| 9 | TMWKAGILY | | Y | N | Y | N | N | | A | 3267 |
| 9 | TVWKAGILY | | N | N | Y | N | N | | A | 3288 |
| 9 | RMYLHTLWK | | N | N | Y | N | N | | A | 3269 |
| 9 | RVYLHTLWK | | N | N | Y | N | N | | A | 3270 |
| 9 | AMTFSPTYK | | N | N | Y | N | N | | A | 3271 |
| 9 | SVVRRAFPR | | N | N | Y | N | N | | A | 3272 |
| 9 | SVVRRAFPK | | N | N | Y | N | N | | A | 3273 |
| 9 | SAIXSVVRR | | N | N | Y | N | N | | A | 3274 |
| 9 | LPVXAFSSA | | N | N | N | N | Y | | A | 3275 |
| 10 | FLLAQFTSAV | L2.LV10 | N | Y | N | N | N | 1 | A | 3276 |
| 10 | YLFTWKAGI | | N | Y | N | N | N | No | A | 3277 |
| 10 | YLLTLWKAGI | | N | Y | N | N | N | No | A | 3278 |
| 10 | LLFYQGMILPV | | N | Y | N | N | N | No | A | 3279 |
| 10 | LLLYQGMILPV | | N | Y | N | N | N | No | A | 3280 |
| 10 | LLVLQAGFFV | L2.LV10 | N | Y | N | N | N | 1 | A | 3281 |
| 10 | ILLLCLIFLV | L2.LV10 | N | Y | N | N | N | 1 | A | 3282 |
| 10 | FPFCLAFSYM | | N | N | N | N | Y | | A | 3283 |
| 10 | FPHCLAFSYI | | N | N | N | N | Y | | A | 3284 |
| 10 | FPARVTGGVF | | N | N | N | N | Y | | A | 3285 |
| 10 | TPFRVTGGVF | | N | N | N | N | Y | | A | 3286 |
| 10 | TPARVTGGVI | | N | N | N | N | Y | | A | 3287 |
| 10 | FPCALRFTSA | | N | N | N | N | Y | | A | 3288 |
| 10 | GPFALRFTSA | | N | N | N | N | Y | | A | 3289 |
| 10 | GPCALRFTSI | | N | N | N | N | Y | | A | 3290 |
| 10 | FPAAMPHLLV | | N | N | N | N | Y | | A | 3291 |
| 10 | HPFAMPHLLV | | N | N | N | N | Y | | A | 3292 |
| 10 | HPAAMPHLLI | | N | N | N | N | Y | | A | 3293 |
| 10 | QMFTFSPTYK | | N | N | Y | N | N | | A | 3294 |
| 10 | QVFTFSPTYK | | N | N | Y | N | N | | A | 3295 |
| 10 | TMWKAGILYK | | N | N | Y | N | N | | A | 3296 |
| 10 | TVWKAGILYK | | N | N | Y | N | N | | A | 3297 |
| 10 | VMGGVFLVDK | | N | N | Y | N | N | | A | 3298 |
| 10 | VVGGVFLVDK | | N | N | Y | N | N | | A | 3299 |
| 10 | SMLPETTVVR | | N | N | Y | N | N | | A | 3300 |
| 10 | SVLPETTVVR | | N | N | Y | N | N | | A | 3301 |
| 10 | TMPETTVVRR | | N | N | Y | N | N | | A | 3302 |
| 10 | TVPETTVVRR | | N | N | Y | N | N | | A | 3303 |
| 10 | HTLWKAGILK | | N | N | Y | N | N | | A | 3304 |
| 10 | HTLWKAGILR | | N | N | Y | N | N | | A | 3305 |
| 10 | HMLWKAGILY | | Y | N | Y | N | N | | A | 3306 |
| 10 | HVLWKAGILY | | N | N | Y | N | N | | A | 3307 |
| 10 | GMDNSVVLSR | | N | N | Y | N | N | | A | 3308 |
| 10 | GVDNSVVLSR | | N | N | Y | N | N | | A | 3309 |
| 10 | GTFNSVVLSR | | N | N | Y | N | N | | A | 3310 |
| 10 | YMFDVVLGAK | | N | N | Y | N | N | | A | 3311 |
| 10 | MMWYWGPSLK | | N | N | Y | N | N | | A | 3312 |
| 10 | MMWYWGPSLR | | N | N | Y | N | N | | A | 3313 |
| 9 | ILLLXLIFL | | N | Y | N | N | N | | A | 3314 |
| 9 | LLLXLIFLL | | N | Y | N | N | N | | A | 3315 |
| 9 | LLXLIFLLV | | N | Y | N | N | N | | A | 3316 |
| 9 | PLLPIFFXL | | N | Y | N | N | N | | A | 3317 |
| 9 | ALMPLYAXI | | N | Y | N | N | N | | A | 3318 |
| 9 | GLXQVFADA | | N | Y | N | N | N | | A | 3319 |
| 9 | HISXLTFGR | | N | N | Y | N | N | | A | 3320 |
| 9 | FVLGGXRHK | | N | N | Y | N | N | | A | 3321 |
| 10 | FILLLXLIFL | | N | Y | N | N | N | | A | 3322 |
| 10 | ILLLXLIFLL | | N | Y | N | N | N | | A | 3323 |
| 10 | LLLXLIFLLV | | N | Y | N | N | N | | A | 3324 |
| 10 | LLPIFFXLWV | | N | Y | N | N | N | | A | 3325 |
| 10 | QLLWFHISXL | | N | Y | N | N | N | | A | 3326 |
| 10 | LLGXAANWIL | | N | Y | N | N | N | | A | 3327 |
| 10 | TSAIXSVVAR | | N | N | V | N | N | | A | 3328 |
| 10 | GYRWMXLRRF | | N | N | N | Y | N | | A | 3329 |
| 10 | GPXALRFTSA | | N | N | N | N | Y | | A | 3330 |
| 10 | FPHXLAFSVM | | N | N | N | N | Y | | A | 3331 |
| 11 | HMLWKAGILYK | | N | N | Y | N | N | | A | 3332 |
| 11 | HVLWKAGILYK | | N | N | Y | N | N | | A | 3333 |
| 11 | SMLPETTVVRR | | N | N | Y | N | N | | A | 3334 |
| 11 | SVLPETTVVRR | | N | N | Y | N | N | | A | 3335 |
| 11 | GMDNSVVLSRK | | N | N | Y | N | N | | A | 3336 |
| 11 | GVDNSVVLSRK | | N | N | Y | N | N | | A | 3337 |
| 11 | GTFNSVVLSRK | | N | N | Y | N | N | | A | 3338 |
| 8 | MPLSYQHI | | N | N | N | N | Y | | A | 3339 |
| 8 | LPIFFCLI | | N | N | N | N | Y | | A | 3340 |
| 8 | SPFLLAQI | | N | N | N | N | Y | | A | 3341 |
| 8 | YPALMPLI | | N | N | N | N | Y | | A | 3342 |
| 8 | VPSALNPI | | N | N | N | N | Y | | A | 3343 |
| 9 | LPIFFCLWI | | N | N | N | N | Y | | A | 3344 |
| 9 | LPIHTAELI | | N | N | N | N | Y | | A | 3345 |
| 10 | VPFVQWFVGI | | N | N | N | N | Y | | A | 3346 |
| 11 | NPLGFFPDHQI | | N | N | N | N | Y | | A | 3347 |
| 11 | LPIHTAELLAI | | N | N | N | N | Y | | A | 3348 |
| 9 | FLPSVFPSA | L2.FV5 | N | Y | N | N | N | Rev3 | A | 3349 |
| 10 | YLHTLWKAGV | L2.IV10 | N | Y | N | N | N | 1 | A | 3350 |
| 11 | STLPETYVVRR | | N | N | Y | N | N | | A | 3351 |
| 9 | YMDDVVLGV | M2.AV9 | N | Y | N | N | N | 1 | A | 3352 |
| 9 | FPIPSSWAF | | N | N | N | N | Y | | A | 3353 |
| 9 | IPITSSWAF | | N | N | N | N | Y | | A | 3354 |
| 9 | IPILSSWAF | | N | N | N | N | Y | | A | 3355 |
| 9 | FPVCLAFSY | | N | N | N | N | Y | | A | 3356 |
| 9 | FPHCLAFAY | | N | N | N | N | Y | | A | 3357 |
| 9 | FPHCLAFSL | | N | N | N | N | Y | | A | 3358 |
| 9 | IPIPMSWAF | | N | N | N | N | Y | | A | 3359 |
| 9 | FPHCLAFAL | | N | N | N | N | Y | | A | 3360 |
| 10 | FLPSZFFPSV | | N | Y | N | N | N | No | A | 3361 |
| 10 | FLPSZFFPSV | | N | Y | N | N | N | No | A | 3362 |
| 9 | IPFPSSWAF | | N | N | N | N | Y | | A | 3363 |
| 9 | IPIPSSWAI | | N | N | N | N | Y | | A | 3364 |
| 9 | FPFCLAFSY | | N | N | N | N | Y | | A | 3365 |
| 9 | FPHCLAFSA | | N | N | N | N | Y | | A | 3366 |
| 9 | FPHCLAFSA | | N | N | N | N | Y | | A | 3367 |
| 10 | FQPSDYFPSV | | N | Y | N | N | N | Rev | A | 3368 |
| 9 | YLLTRILTI | | N | Y | N | N | N | | A | 3369 |
| 9 | FLYTRILTI | | N | Y | N | N | N | | A | 3370 |
| 9 | FLLTYILTI | | N | Y | N | N | N | | A | 3371 |
| 9 | FLLTRILYI | | N | Y | N | N | N | | A | 3372 |
| 11 | FLPSDFFPSVR | | N | N | Y | N | N | | A | 3373 |
| 9 | FLPSDFFPS | | N | N | N | N | N | | A | 3374 |
| 8 | FLPSDFFP | | N | N | N | N | N | | A | 3375 |
| 10 | FLPSDFFPSI | L2.V110 | N | Y | N | N | N | Rev | A | 3376 |
| 10 | FLPSDYFPSV | | N | Y | N | N | N | No | A | 3377 |
| 12 | YSFLSDFFPSV | | N | N | N | N | N | | A | 3378 |
| 10 | YNMGLKFRQL | | N | N | N | N | N | | A | 3379 |
| 9 | NMGLKYRQL | | N | Y | N | Y | N | No | A | 3380 |
| 10 | FLPS(X)YFPSV | | N | N | N | N | N | | A | 3381 |
| 10 | FLPSD(X)FPSV | | N | N | N | N | N | | A | 3382 |
| 11 | FLPSDLLPSVR | | N | N | N | N | N | | A | 3383 |
| 12 | FLPSDFFPSVRD | | N | N | N | N | N | | A | 3384 |
| 12 | LSFLPSDFFPSV | | N | N | N | N | N | | A | 3385 |
| 11 | SFLPSDFFPSV | | N | N | N | N | N | | A | 3386 |
| 8 | PSDFFPSV | | N | N | N | N | N | | A | 3387 |
| 9 | FLMSYFPSV | | N | Y | N | N | N | No | A | 3388 |
| 9 | FLPSYFPSV | L2.FY5. | N | Y | N | N | N | 3 | A | 3389 |
| 10 | FLMSDYFPSV | | N | Y | N | N | N | No | A | 3390 |
| 11 | CILLLCLIFLL | | N | Y | N | N | N | No | A | 3391 |
| 10 | FLPNDFFPSA | L2.SN4. | N | Y | N | N | N | Rev | A | 3392 |
| 10 | FLPDDFFPSA | L2.SD4. | N | Y | N | N | N | Rev | A | 3393 |
| 10 | FLPNDFFPSV | | N | Y | N | N | N | No | A | 3394 |
| 10 | FLPSDFFPSA | L2.VA10 | N | Y | N | N | N | Rev | A | 3395 |
| 10 | FLPDDFFPSV | | N | Y | N | N | N | No | A | 3396 |
| 10 | FLPPADFFPSV | | N | Y | N | N | N | No | A | 3397 |
| 10 | FLPVDFFPSV | | N | Y | N | N | N | No | A | 3398 |
| 10 | FLPADFFPSI | L2.SA4. | N | Y | N | N | N | Rev | A | 3399 |
| 10 | FLPVDFFPSI | L2.SV4. | N | Y | N | N | N | Rev | A | 3400 |
| 10 | FLPSDAFPSV | | N | Y | N | N | N | No | A | 3401 |
| 10 | FLPSAFFPSV | | N | Y | N | N | N | No | A | 3402 |
| 10 | FLPSDFAPSV | | N | Y | N | N | N | No | A | 3403 |
| 10 | FLPSDFFASV | | N | Y | N | N | N | No | A | 3404 |
| 10 | FLPSDFFPAV | | N | Y | N | N | N | No | A | 3405 |
| 10 | FLASDFFPSV | | N | Y | N | N | N | No | A | 3406 |
| 10 | FAPSDFFPSV | LA2.V10 | N | Y | N | N | N | Rev | A | 3407 |
| 10 | ALPSSFFPSV | | N | Y | N | N | N | No | A | 3408 |
| 10 | YLPSDFFPSV | | Y | N | N | N | No | A | 3409 | |
| 10 | FMPSDFFPSV | LM2.V1 | N | Y | N | N | N | 1 | A | 3410 |
| 10 | FLKSDFFPSV | | N | Y | N | N | N | No | A | 3411 |
| 10 | FLPSEFFPSV | | N | Y | N | N | N | No | A | 3412 |
| 10 | FLPSDFYPSV | | N | Y | N | N | N | No | A | 3413 |
| 10 | FLPSDFFKSV | | N | Y | N | N | N | No | A | 3414 |
| 10 | FLPSDFFPKV | | N | Y | N | N | N | No | A | 3415 |
| | FLPSDFFPSV(CONH2) | | | | | | | | | 3416 |
| | VLEYLVSFGV(NH2) | | | | | | | | | 3417 |
| | ATVELLSFLPSDFFPSV-NH2 | | | | | | | | | 3418 |
| | TVELLSFLPSDFFPSV-NH2 | | | | | | | | | 3419 |
| | VELLSFLPSDFFPSV-NH2 | | | | | | | | | 3420 |
| | ELLSFLPSDFFPSV-NH2 | | | | | | | | | 3421 |
| | LLSFLPSDFFPSV-NH2 | | | | | | | | | 3422 |
| | LSFLPSDFFPSV-NH2 | | | | | | | | | 3423 |
| | SFLPSDFFPSV-NH2 | | | | | | | | | 3424 |
| | FLPSDFFPSV-NH2 | | | | | | | | | 3425 |
| | LPSDFFPSV-NH2 | | | | | | | | | 3426 |
| | PSDFFPSV-NH2 | | | | | | | | | 3427 |
| | FLPSDFFPS-NH2 | | | | | | | | | 3428 |
| | FLPSDFFP-NH2 | | | | | | | | | 3429 |
| | FLPSDFF-NH2 | | | | | | | | | 3430 |
| | ALPSDFFPSV-NH2 | | | | | | | | | 3431 |
| | SLNFLGGTTV(NH2) | | | | | | | | | 3432 |
| | FLPSDFFPSVR-NH2 | | | | | | | | | 3433 |
| | ALFKDWEEL | | | | | | | | | 3434 |
| | VLGGSRHKL | | | | | | | | | 3435 |
| | KIKESFRKL | | | | | | | | | 3436 |
| | ALMPLYASI | | | | | | | | | 3437 |
| | FLSKQYLNL | | | | | | | | | 3438 |
| | LLGSAANWI | | | | | | | | | 3439 |
| | NNLNNLNVSI | | | | | | | | | 3440 |
| | IIKKSEQFV | | | | | | | | | 3441 |
| | ALSLIVNLL | | | | | | | | | 3442 |
| | RIPRTPRSV | | | | | | | | | 3443 |
| | | | | | | | | | | 3444 |
| | | | | | | | | | | 3445 |

TABLE XXIII

Immunogenicity of HLBY-derived peptides

Immunogenicity

| | | | | | | | | | |
|---|---|---|---|---|---|---|---|---|---|
| Supermotif | Peptide | Sequence | SEQ ID NO: | Protein | XRN | primary | transgenic | patients | overall¹ |

| | | | | | | | | | | |
|---|---|---|---|---|---|---|---|---|---|---|
| A2 supermotif | 924.07 | FLPSDFFPSV | 3492 | HBV core 18 | 5 | 10/10 | 6/6 | 25/32^{a} | + | |
| | 1069.06 | LLVPFVQWFV | 3493 | HBVV env 338 | 5 | 3/4 | 6/9 | | + | |
| | 1147.13 | FLLAQFTSAI | 3494 | HBV pol 513 | 5 | | 0/3 | | unk | |
| | 1090.77 | YMDDVVLGV | 3495 | HBV pol 538 | 5 | | 9/9 | | + | |
| | 777.03 | FLLTRILTI | 3496 | HBV env 183 | 4 | | | 14/23^{a} | + | |
| | 927.15 | ALMPLYACI | 3497 | HBV pol 642 | 4 | 10/12 | 3/5 | 2/15^{a} | + | |
| | 1013.01 | WLSLLVPFV | 3498 | HBV env 335 | 4 | 2/6 | 5/9 | 23/29^{a} | + | |
| | 1069.05 | LLAQFTSAI | 3499 | HBV pol 504 | 4 | 0/4 | 0/5 | | unk | |
| | 1132.01 | LVPFVQWFV | 3500 | HBV env 339 | 4 | 0/3 | 0/4 | | unk | |
| | 1147.14 | VLLDYQGMLPV | 3501 | HBV env 259 | 4 | 4/4 | 6/6 | | + | |
| | 927.41 | LLSSNLSWL | 3502 | HBV pol 992 | 3 | 0/4 | 0/3 | | unk | |
| | 927.42 | NLSWLSLDV | 3503 | HBV pol 411 | 3 | | 2/8 | | + | |
| | 927.46 | KLHLYSHPI | 3504 | HBV pol 489 | 3 | 0/4 | 4/6 | | + | |
| | 1069.07 | FLLAQFTSA | 3505 | HBV pol 503 | 3 | 1/2 | 0/3 | | + | |
| | 1168.02 | GLSRYVARL | 3506 | HBV pol 455 | 3 | | | 9/13^{a} | + | |
| A2 supermotif | 927.11 | FLLSLGIHL | 3507 | HBV pol 562 | 2 | 15/22 | 12/13 | 9/15^{a} | + | |
| | 927.47 | HLYSHPIIL | 3508 | HBV pol 1076 | 2 | | 10/14 | | + | |
| | 1039.03 | MMWYWGPSL | 3509 | HBV env 360 | 2 | 3/4 | 0/4 | | + | |
| | 1069.12 | YLHTLWKAGV | 3510 | HBV pol 147 | 2 | 2/4 | | | + | |
| | 1137.02 | LLDYQGMLPV | 3511 | HBV env 260 | 2 | 1/2 | 0/4 | | + | |
| | 1142.07 | GLLGWSPQA | 3512 | HBV env 62 | 2 | 3/4 | 5/6 | | + | |
| | 1.0573 | ILRGTSFVYV | 3513 | HBV pol 773 | 1 | | | 3/7^{b} | + | |
| | 1013.14 | VLQAGFFLL | 3514 | HBV env 177 | 1 | 0/4 | 5/12 | | + | |
| | 1069.10 | LLPIFFCLWV | 3515 | HBV env 378 | I | 3/3 | 0/4 | 2/5^{c} | | |
| | 1069.13 | PLLPIFFCL | 3516 | HBV env 377 | 1 | 0/4 | 7/12 | | + | |
| | 1090.06 | LLVLQAGFFL | 3517 | HBV env 175 | 1 | 1/5 | 0/4 | | + | |
| | 1090.12 | YLVSFGVWI | 3518 | HBV nuc 118 | 1 | 9/9 | | | + | |
| | 1.0518 | GLSPTVWLSV | 3519 | HBV eny 338 | 1 | | | 3/9^{c} | + | |
| | 1090.14 | YMDDVVLGA | 3520 | HBV pol 538 | 1 | 2/7 | 2/5 | 2/7^{b} | + | |
| A3 supermotif | 1147.16 | HTLWKAGILYK | 3521 | HBV POL 149 | 5 | 0/6 | 3/3 | 1/22 | + | |
| | 1083.01 | STLPETTVVRR | 3522 | HBV core 141 | 4 | 3/5 | 6/6 | 8/32 | + | |
| | 1150.51 | GSTHVSWPK | 3523 | HBV pol 398 | 4 | | 3/6 | | + | |
| | 1.02.19 | FVLGGCRHK | 3524 | HBV adr “X” 1550 | 3 | 0/4 | | unk | | |
| | 1069.16 | NVSIPWTHK | 3525 | HBV pol 47 | 3 | 0/8 | 0/3 | 1/21 | + | |
| | 1069.20 | LVVDFSQFSR | 3526 | HBV pol 388 | 3 | 0/4 | 6/6 | 1/22 | + | |
| | 1090.10 | QAFTFSPTYK | 3527 | HBV pol 665 | 3 | 3/6 | 0/3 | 3/21 | + | |
| | 1090.11 | SAICSVVRR | 3528 | HBV pok 531 | 3 | 1/4 | | 2/22 | + | |
| A3 supermotif | 1069.15 | TLWKAGILYK | 3529 | HBV pol 150 | 2 | 3/8 | 0/3 | 5/28 | + | |
| | 1142.05 | KVGNFTGLY | 3530 | HBV adr POL 629 | 2 | | 0/3 | 2/22 | + | |
| B7 supermotif | 1147.05 | FPHCLAFSYM | 3531 | HBV POL 530 | 5 | 1/3 | | 0/12 | + | |
| | 988.05 | LPSDFFPSV | 3532 | HBV core 19-27 | 4 | | | 2/16 | + | |
| | 1145.04 | IPIPSSWAF | 3533 | HBV ENV 313 | 4 | 0/4 | | 1/12 | + | |
| | 1147.02 | HPAAMPHLL | 3534 | HBV POL 429 | 4 | 0/5 | | 0/12 | unk | |
| | 1147.06 | LPVCAFSSA | 3535 | HBV X 58 | 4 | 1/4 | | | + | |
| | 1147.08 | YPALMPLYA | 3536 | HBV POL 640 | 4 | | | 0/12 | unk | |
| | 1145.08 | FPHCLAFSYM | 3537 | HBV POL 541 | 3 | 0/4 | | | unk | |
| B7 supermotif | 1147.04 | TPARVTGGVF | 3538 | HBV POL 354 | 2 | | | | 2/12 | + |

Immunogenicity evaluation derived from primary cultures, acute patients (a-Bertoni et al, J Clin Invest 100:503, b-Rehermann et al., J. Clin. Invest 97:1655, c-Nayersina et al., J. Immunol 150:4659) or transgenic mice. A positive assessment (+) is assigned when responders have been noted in one of these systems. Unk = unknown

TABLE XXIV

MHC-peptide binding assays: cell lines and radiolabeled ligands.

Radiolabeled peptide

| | | | | | | |
|---|---|---|---|---|---|---|
| | | | | | | SEQ ID |
| Species | Antigen | Allele | Cell line | Source | Sequence | NO: |

A. Class I binding assays

| | | | | | | |
|---|---|---|---|---|---|---|
| Human | A1 | A*0101 | Steinlin | Hu. J chain 102-110 | YTAVVPLVY | 3539 |
| | A2 | A*0201 | JY | HBVc 18-27 F6 -> Y | FLPSDYFPSV | 3540 |
| | A2 | A*0202 | P815 | HBVc 18-27 F6 -> Y | FLPSDYFPSV | 3540 |
| | | | (transfected) | | | |
| | A2 | A0203 | FUN | HBVc 18-27 F6 -> Y | FLPSDYFPSV | 3540 |
| | A2 | A0206 | CLA | HBVc 18-27 F6 -> Y | FLPSDYFPSV | 3540 |
| | A2 | A*0207 | 721.221 | HBVc 18-27 F6 -> Y | FLPSDYFPSV | 3540 |
| | | | (transfected) | | | |
| | A3 | | GM3107 | non-natural (A3CON1) | KVFPYALINK | 3541 |
| | All | | BVR | non-natural (A3CON1) | KVFPYALINK | 3541 |
| | A24 | A*2402 | KAS116 | non-natural (A24CON1) | AYLDNYNKF | 3542 |
| | A31 | A*3101 | SPACH | non-natural (A3CON1) | KVFPYALINK | 3541 |
| | A33 | A*3301 | LWAGS | non-natural (A3CON1) | KVFPYALINK | 3541 |
| | A28/68 | A*6801 | C1R | HBVc 141-151 T7 -> Y | STLPETYVVRR | 3543 |
| | A28/68 | A*6802 | AMAT | HBV pol 646-654 G4 -> A | FTQAGYPAL | 3544 |
| | B7 | B*0702 | GM3 107 | A2 sigal seq. 5-13 (L7 -> Y) | APRTLVYLL | 3545 |
| | B8 | B*0801 | Steinlin | HIV gp 586-593 Y1 -> F, Q5 -> Y | FLKDYQLL | 3546 |
| | B27 | B*2705 | LG2 | R 60s | FRYNGLIHR | 3547 |
| | B35 | B*3501 | CIR, BVR | non-natural (B35CON2) | FPFKYAAAF | 3548 |
| | B35 | B*3502 | TISI | non-natural (B35CON2) | FPFKYAAAF | 3548 |
| | B35 | B*3503 | EHM | non-natural (B35CON2) | FPFKYAAAF | 3548 |
| | B44 | B*4403 | PITOUT | EF-1 G6 -> Y | AEMGKYSFY | 3549 |
| | B51 | | KAS116 | non-natural (B35CON2) | FPFKYAAAF | 3550 |
| | B53 | B*5301 | AMAT | non-natural (B3SCON2) | FPFKYAAAF | 3550 |
| | B54 | B*5401 | KT3 | non-natural (B3SCON2) | FPFKYAAAF | 3550 |
| | Cw4 | Cw*0401 | C1R | non-natural (C4CON) | QYDDAVYKL | 3551 |
| | Cw6 | Cw*0602 | 721.221 | non-natural (C6CON1) | YRHDGGNVL | 3552 |
| | | | transfected | | | |
| | Cw7 | Cw*0702 | 721.221 | non-natural (C6CON1) | YRHDGGNVL | 3552 |
| | | | transfected | | | |
| Mouse | D^{b} | | EL4 | Adenovirus EIA P7 -> Y | SGPSNTYPEI | 3553 |
| | K^{b} | | EL4 | VSV NP 52-59 | RGYVFQGL | 3554 |
| | D^{d} | | P815 | HIV-IIIB ENV G4 -> Y | RGPYRAFVTI | 3555 |
| | K^{d} | | P815 | non-natural (KdCON1) | KFNPMLKTYI | 3556 |
| | L^{d} | | P815 | HBVs 28-39 | IPQSLDSYWTSL | 3557 |

B. Class II binding assays

| | | | | | | |
|---|---|---|---|---|---|---|
| Human | DR1 | DRB1*0101 | LG2 | HA Y307-319 | YPKYVKQNTLKLAT | 3558 |
| | DR2 | DRB1*1501 | L466.1 | MBP 88-102Y | VVHFFKNIVTPRTPPY | 3559 |
| | DR2 | DRB1*1601 | L242.5 | non-natural (760.16) | YAAFAAAKTAAAFA | 3560 |
| | DR3 | DRB1*0301 | MAT | MT 65kD Y3-13 | YKTIAFDEEARR | 3561 |
| | DR4w4 | DRBP*0401 | Preiss | non-natural (717.01) | YARFQSQTTLKQKT | 3562 |
| | DR4w10 | DRBP*0402 | YAR | non-natural (717.10) | YARFQRQTTLKAAA | 3563 |
| | DR4w14 | DRB1*0404 | BIN 40 | non-natural (717.01) | YARFQSQTTLKQKT | 3562 |
| | DR4w15 | DRB1*0405 | KT3 | non-natural (717.01) | YARFQSQTTLKQKT | 3562 |
| | DR7 | DRB1*0701 | Pitout | Tet. tox. 830-843 | QYIKANSKFIGITE | 3564 |
| | DR8 | DRB1*0802 | OLL | Tet. tox. 830-843 | QYIKANSKFIGITE | 3564 |
| | DR8 | DRB1*0803 | LUY | Tet. tox. 830-843 | QYIKANSKFIGITE | 3564 |
| | DR9 | DRB1*0901 | HID | Tet. tox. 830-843 | QYIKANSKFIGITE | 3564 |
| | DR11 | DRB1*1101 | Sweig | Tet. tox. 830-843 | QYIKANSKFIGITE | 3564 |
| | DR12 | DRB1*1201 | Herluf | unknown eluted peptide | EALIHQLKENPYVLS | 3565 |
| | DR13 | DRB1*1302 | H0301 | Tet. tox. 830-843 S->A | QYIKANAKFIGITE | 3566 |
| | DR51 | DRB5*0101 | GM3107 or | Tet. tox. 830-843 | QYIKANAKFIGITE | 3566 |
| | | | L416.3 | | | |
| | DR51 | DRB5*0201 | L255.1 | HA 307-3 19 | PKYVKQNTLKLAT | 3567 |
| | DR52 | DRB3*0101 | MAT | Tet. tox. 830-843 | NGQIGNDPNRDIL | 3568 |
| | DR53 | DRB4*0101 | L257.6 | non-natural (717.01) | YARFQSQTTLKQKT | 3569 |
| | DQ3.1 | QAI*0301/DQBI*03( | PF | non-natural (ROIV) | AHAAHAAHAAhAAHAA | 3570 |
| Mouse | IA^{b} | | DB27.4 | non-natural (ROW) | AHAAHAAHAAHAAHAA | 3570 |
| | IA^{d} | | A20 | non-natural (ROW) | AHAAHAAHAAHAAHAA | 3570 |
| | IA^{k} | | CH-12 | HEL46-61 | YNTDGSTDYGILQINSR | 3571 |
| | IA | | LS102.9 | non-natural (ROIV) | AHAAHAAHAAHAAHAA | 3570 |
| | IA | | 91.7 | non-natural (ROIV) | AHAAHAAHAAHAAHAA | 3570 |
| | IE^{d} | | A20 | Lambda repressor 12-26 | YLEDARRKKAIYEKKK | 3572 |
| | IE^{k} | | CH-12 | Lambda repressor 12-26 | YLEDARRXKAIYEKKK | 3572 |

TABLE XXV
Monoclonal antibodies used in MHC purifi

| Monoclonal antibody | Specificity |
|---|---|
| W6/32 | HLA-class I |
| B123.2 | HLA-B and C |
| IVD12 | HLA-DQ |
| LB3.1 | HLA-DR |
| M1/42 | H-2 class I |
| 28-14-8S | H-2 D^{b} and L^{d} |
| 34-5-8S | H-2 D^{d} |
| B8-24-3 | H-2 K^{b} |
| SF1-1.1.1 | H-2 K^{d} |
| Y-3 | H-2 K^{b} |
| 10.3.6 | H-2 IA^{k} |
| 14.4.4 | H-2 IE^{d}, IE^{K} |
| MKD6 | H-2 IA^{d} |
| Y3JP | H-2 IA^{b}, IA^{s}, IA^{u} |

TABLE XXVI

in vitro binding of conserved HBV-derived peptides to
HLA-A2-supertype alleles.

| | |
|---|---|
| A2-supertype | |
| binding capacity (IC50 nM) | Alleles |

| | | | | | | | | | | | | |
|---|---|---|---|---|---|---|---|---|---|---|---|---|
| Peptide | AA | Molecule | 1st Pos | Sequence | SEQ ID NO: | Consv.¹ | A*0201 | A*0202 | A*0203 | A*0206 | A*6802 | bound² |

| | | | | | | | | | | | | |
|---|---|---|---|---|---|---|---|---|---|---|---|---|
| 924.07 | 10 | Core | 18 | FLPSDFFPSV | 3492 | 95 | 2.5 | 2.1 | 6.0 | 3.0 | 36 | 5 |
| 1069.06 | 10 | ENV | 349 | LLVPFVQWFV | 3493 | 95 | 7.5 | 11 | 5.9 | 13 | 286 | 5 |
| 1147.13 | 10 | POL | 524 | FLLAQFTSA | 3494 | 95 | 24 | 134 | 1.4 | 34 | 455 | 5 |
| 1013.0102 | 9 | ENV | 346 | WLSLLVPFV | 3498 | 100 | 4.6 | 113 | 1.4 | 10 | 1290 | 4 |
| 777.03 | 9 | ENV | 183 | FLLTRILTI | 3496 | 80 | 9.8 | 100 | 1.3 | 19 | —³ | 4 |
| 927.15 | 9 | POL | 653 | ALMPLYACI | 3497 | 95 | 10 | 126 | 3.0 | 160 | 851 | 4 |
| 1069.05 | 9 | POL | 525 | LLAQFTSAI | 3499 | 95 | 50 | 16 | 3.0 | 1538 | 51 | 4 |
| 1132.01 | 9 | ENV | 350 | LVPFVQWFV | 3500 | 95 | 119 | 287 | 2083 | 463 | 14 | 4 |
| 1147.14 | 11 | ENV | 259 | VLLDYQGMLPV | 3501 | 90 | 8.6 | 20 | 2.0 | 13 | 2353 | 4 |
| 1090.77 | 9 | POL | 538(a) | YMDDVVLGV | 3495 | 90 | 5.1 | 90 | 6.7 | 71 | 1905 | 4 |
| 1069.073 | 9 | POL | 524 | FLLAQFTSA | 3505 | 95 | 6.0 | 1654 | 9.1 | 39 | 870 | 3 |
| 927.46 | 9 | POL | 500 | KLHLYSHPI | 3504 | 95 | 72 | 126 | 3.7 | 627 | 26667 | 3 |
| 927.42 | 9 | POL | 422 | NLSWLSLDV | 3503 | 90 | 77 | 843 | 16 | 2313 | 404 | 3 |
| 1168.02 | 9 | POL | 455 | GLSRYVARL | 3506 | 90 | 79 | 391 | 18 | 12333 | — | 3 |
| 927.41 | 9 | POL | 418 | LLSSNLSWL | 3502 | 90 | 455 | 55 | 2.6 | 1370 | 4000 | 3 |
| 1039.031 | 9 | ENV | 360 | MMWYWGPSL | 3509 | 85 | 5.6 | 5375 | 833 | 112 | 3636 | 2 |
| 927.11 | 9 | POL | 573 | FLLSLGIHL | 3507 | 95 | 7.7 | 4300 | 1000 | 34 | 11429 | 2 |
| 1142.07 | 9 | ENV | 73 | GLLGWSPQA | 3512 | 85 | 13 | 14333 | 286 | 1429 | — | 2 |
| 927.47 | 9 | POL | 502 | HLYSHPIIL | 3508 | 80 | 23 | 14333 | 11 | 2176 | 755 | 2 |
| 1137.02 | 10 | ENV | 271 | LLDYQGMLPV | 3511 | 90 | 51 | — | 500 | 552 | — | 2 |
| 1069.09 | 9 | ENV | 270 | VLLDYQGML | 3573 | 95 | 1144 | — | 476 | 4111 | - | 2 |
| 1069.14 | 10 | NUC | 168 | ILSTLPETTV | 3574 | 100 | 238 | 506 | 130 | 1194 | 5970 | 2 |
| 1069.11 | 10 | POL | 147 | YLHTLWKAGI | 3575 | 100 | 313 | 8600 | 18 | 4000 | 1250 | 2 |
| 1142.01 | 9 | NUC | 129 | LLWFHISCL | 3576 | 90 | 385 | 21500 | 238 | 1194 | 4082 | 2 |
| 1090.12 | 9 | NUC | 147 | YLVSFGVWI | 3538 | 90 | 13 | | | | | 1 |
| 1.0538 | 10 | ENV | 359 | GLSPTVWLSV | 3519 | 75 | 18 | | | | | 1 |
| 1013.1402 | 9 | ENV | 177 | VLQAGFFLL | 3514 | 95 | 33 | 2389 | 3704 | 1947 | 6349 | 1 |
| 1069.13 | 9 | ENV | 388 | PLLPIFFCL | 3516 | 100 | 77 | — | 5556 | 3364 | 8511 | 1 |
| 1069.10 | 10 | ENV | 389 | LLPIFFCLWV | 3515 | 100 | 156 | 5375 | 667 | 5000 | — | 1 |
| 1090.06 | 10 | ENV | 175 | LLVLQAGFFL | 3517 | 90 | 161 | 1162 | 2222 | 2467 | 3636 | 1 |
| 1.0895 | 10 | ENV | 248 | FILLLCLIFL | 3577 | 80 | 179 | | | | | 1 |
| 927.24 | 9 | POL | 770 | WILRGTSFV | 3578 | 80 | 185 | | | | | 1 |
| 1090.14 | 9 | POL | 538 | YMDDVVLGA | 3520 | 90 | 200 | — | 4167 | — | — | 1 |
| 3.0205 | 10 | ENV | 171 | FLGPLLVLQA | 3579 | 75 | 263 | | | | | 1 |
| 1069.08 | 10 | ENV | 260 | ILLLCLIFLL | 3580 | 100 | 263 | — | — | 2846 | 26667 | 1 |
| 1.0573 | 10 | POL | 773 | ILRGTSFVYV | 3581 | 80 | 313 | | | | | 1 |

¹Frequency of entire sequence amongst isolales scanned.
²Number of supertpe alleles bound. Peptides binding 3 or more alleles are considered degenerate.
³A dash (—) indicates IC50

TABLE XXVII

in vitro binding of conserved HBV-derived peptides to
HLA-A3-supertype alleles.

| | |
|---|---|
| A3-supertype | |
| binding capacity (IC50 nM) | Alleles |

| | | | | | | | | | | | | |
|---|---|---|---|---|---|---|---|---|---|---|---|---|
| Peptide | AA | Molecule | 1st Pos | Sequence | SEQ ID NO: | Consv.¹ | A*03 | A*11 | A*3101 | A*3301 | A*6801 | bound |

| | | | | | | | | | | | | |
|---|---|---|---|---|---|---|---|---|---|---|---|---|
| 26.0535 | 11 | X NUC FUS | 299 | GVWIRTPPAYR | 3582 | 95 | 58 | 35 | 3.0 | 40 | 12 | 5 |
| 1147.16 | 11 | pol | 149 | HTLWKAGILYK | 3583 | 100 | 20 | 14 | 486 | 403 | 42 | 5 |
| 26.0539 | 11 | POL | 376 | RLVVDFSQFSR | 3584 | 95 | 39 | 2.0 | 7.0 | 24 | 1.0 | 5 |
| 26.0149 | 9 | X | 69 | CALRFTSAR | 3585 | 85 | 3235 | 261 | 12 | 3.6 | 11 | 4 |
| 1.0993 | 9 | X | 130 | KVFVLGGCR | 3586 | 75 | 262 | 73 | 30 | 408 | 2667 | 4 |
| 26.0153 | 9 | X | 64 | SSAGPCALR | 3587 | 90 | 1375 | 43 | 55 | 181 | 11 | 4 |
| 1083.01 | 11 | Core | 141 | STLPETTVVRR | 3588 | 95 | 733 | 4.0 | 180 | 181 | 26 | 4 |
| 20.0130 | 9 | pol | 655 | AFTFSPTYK | 3589 | 95 | 42 | 150 | 3103 | 13182 | 296 | 3 |
| 26.0008 | 8 | POL | 656 | FTFSPTYK | 3590 | 95 | 193 | 136 | 1286 | 1000 | 73 | 3 |
| 1.0219 | 9 | X | 1550 | FVLGGCRHK | 3591 | 80 | 169 | 316 | 1500 | 744 | 103 | 3 |
| 1069.20 | 10 | POL | 388 | LVVDFSQFSR | 3592 | 100 | 6875 | 17 | 692 | 126 | 16 | 3 |
| 1069.16 | 9 | POL | 47 | NVSIPWTHK | 3593 | 100 | 134 | 105 | —³ | 2900 | 250 | 3 |
| 1090.10 | 10 | POL | 665 | QAFTFSPTYK | 3594 | 95 | 244 | 11 | 18000 | 5088 | 6.7 | 3 |
| 1090.11 | 9 | POL | 531 | SAICSVVRR | 3595 | 95 | 1897 | 29 | 1200 | 446 | 21 | 3 |
| 20.0131 | 9 | pol | 524 | SVVRRAFPH | 3596 | 95 | 100 | 10 | 621 | — | 500 | 3 |
| 26.0545 | 11 | X NUC FUS | 318 | TLPETTVVRRR | 3597 | 95 | 22000 | 375 | 2951 | 408 | 13 | 3 |
| 26.0023 | 8 | X NUC FUS | 296 | VSFGVWIR | 3598 | 90 | 2750 | 207 | 240 | 1074 | 222 | 3 |
| 1142.05 | 9 | POL | 55 | KVGNFTGLY | 3599 | 95 | 52 | 353 | — | — | — | 2 |
| 1142.06 | 9 | POL | 623 | PVNRPIDWK | 3600 | 85 | 355 | 43 | — | — | 8889 | 2 |
| 1.0975 | 9 | POL | 106 | RLKLIMPAR | 3601 | 75 | 116 | — | 5.8 | 592 | — | 2 |
| 1.0562 | 10 | POL | 576 | SLGIHLNPNK | 3602 | 75 | 55 | 77 | | | | 2 |
| 1069.21 | 10 | NUC | 170 | STLPETTVVR | 3603 | 95 | 15714 | 100 | 2250 | 1208 | 320 | 2 |
| 1069.22 | 10 | NUC | 171 | TLPETTVVRR | 3604 | 95 | 15714 | 261 | — | 2417 | 182 | 2 |
| 1069.15 | 10 | POL | 150 | TLWKAGILYK | 3605 | 100 | 2.1 | 17 | 3529 | 29000 | 615 | 2 |
| 1.0215. | 9 | X | 105 | TTDLEAYFK | 3606 | 75 | 18333 | 6.5 | — | 24167 | 471 | 2 |
| 1069.17 | 10 | POL | 369 | VTGGVFLVDK | 3607 | 100 | 282 | 65 | — | — | 3636 | 2 |
| 1069.19 | 9 | POL | 389 | VVDFSQFSR | 3608 | 100 | 7333 | 80 | 13846 | 1706 | 242 | 2 |
| 26.0026 | 8 | POL | 168 | ASFCGSPY | 3609 | 100 | 239 | 26 | — | — | 20000 | 2 |
| 26.0549 | 11 | ENV | 389 | LLPIFFCLWVY | 3610 | 100 | 478 | 10000 | 2609 | 644 | 82 | 2 |
| 26.0550 | 11 | POL | 528 | RAFPHCLAFSY | 3611 | 95 | 92 | 15 | 667 | 26364 | 2667 | 2 |
| 1090.04 | 10 | POL | 746 | GTDNSVVLSR | 3612 | 90 | 11000 | 143 | 6000 | 15263 | 10000 | 1 |
| 1069.04 | 10 | POL | 149 | HTLWICAGILY | 3613 | 100 | 250 | 7500 | — | 8529 | 6667 | 1 |
| 1.0205 | 9 | POL | 771 | ILRGTSFVY | 3614 | 80 | 250 | — | — | — | — | 1 |
| 1090.08 | 9 | NUC | 148 | LYSFGVWIR | 3615 | 90 | 3929 | 500 | | | | 1 |
| 1039.01 | 10 | ENV | 360 | MMWYWGPSLY | 3616 | 85 | 220 | 7500 | — | — | 26667 | 1 |
| 1.0584 | 10 | X | 104 | STTDLEAYFK | 3617 | 75 | 1667 2.2 | | | | 1 | |
| 1147.17 | 11 | pol | 735 | GTDNSVVLSRK | 3618 | 90 | 786 | 11 | — | — | — | 1 |
| 1147.18 | 11 | pol | 357 | RVTGGVFLVDK | 3619 | 100 | 578 | 207 | — | — | — | 1 |
| 1099.03 | 9 | POL | 150 | TLWKAGILY | 3620 | 100 | 85 | 7500 | — | — | — | 1 |
| 3090.15 | 10 | POL | 549 | YMDDVVLGAK | 3621 | 90 | 333 | 1395 | — | — | — | 1 |
| 26.0024 | 8 | POL | 50 | VSIPWTHK | 3622 | 100 | 846 | 353 | 5806 | 22308 | 20000 | 1 |

¹Frequency of entire sequence amongst isolates scanned.
2. Number of supeelpe alleles bound. Peptides binding 3 or more alleles are considered degenerate.
³A dash (—) indicates IC50

TABLE XXVIII

in vitro binding of conserved HBV-derived peptides to
HLA-B7 supertype alleles.

| | |
|---|---|
| B7-supertype binding capacity (IC50 nM) | Alleles |

| | | | | | | | | | | | | |
|---|---|---|---|---|---|---|---|---|---|---|---|---|
| Peptide | AA | Molecule | 1st Pos | Sequence | SEQ ID NO: | Consv.¹ | B*0702 | B*3501 | B*5101 | B*5301 | B*5401 | bound² |

| | | | | | | | | | | | | |
|---|---|---|---|---|---|---|---|---|---|---|---|---|
| 1147.05 | 10 | POL | 541 | FPHCLAFSYM | 3623 | 95 | 56 | 33 | 61 | 118 | 208 | 5 |
| 1145.04 | 9 | ENV | 324 | IPIPSSWAF | 3624 | 100 | 42 | 2.6 | 2.3 | 12 | 2941 | 4 |
| 1147.02 | 9 | POL | 440 | HPAAMPHLL | 3625 | 100 | 56 | 267 | 500 | 186 | 833 | 4 |
| 1147.06 | 9 | X | 58 | LPVCAFSSA | 3626 | 95 | 115 | 101 | 500 | 10333 | 0.53 | 4 |
| 1147.08 | 9 | POL | 651 | YPALMPLYA | 3627 | 95 | 306 | 150 | 162 | 664 | 0.63 | 4 |
| 988.05 | 9 | CORE | 19 | LPSDFFPSV | 3628 | 95 | 1774 | 343 | 9.0 | 120 | 4.8 | 4 |
| 1145.08 | 9 | POL | 541 | FPHCLAFSY | 3629 | 95 | —³ | 14 | 83 | 17 | 503 | 3 |
| 19.0014 | 8 | POL | 640 | YPALMPLY | 3630 | 190 | 13750 | 28 | 13 | 207 | 1786 | 3 |
| 26.0570 | 11 | pol | 640 | YPALMPLYACI | 3631 | 95 | 1375 | — | 117 | 291 | 143 | 3 |
| 1147.04 | 10 | POL | 365 | TPARVTGGVF | 3632 | 90 | 17 | 72 | 939 | 16667 | 2 | |
| 15.0034 | 9 | ENV | 390 | LPIFFCLWV | 3633 | 100 | — | — | 57 | 2325 | 53 | 2 |
| 20.0140 | 9 | POL | 723 | LPIHTAELL | 3634 | 85 | 1375 | 114 | 1058 | 30 | 20000 | 2 |
| 19.0006 | 8 | ENV | 340 | VPFVQWFV | 3635 | 95 | 5500 | — | 0.29 | 91 | 2 | |
| 19.0007 | 8 | ENV | 379 | LPIFFCLW | 3636 | 100 | — | — | 153 | 66 | 2857 | 2 |
| 19.0010 | 8 | POL | 1 | MPLSYQHF | 3637 | 100 | — | 742 | 458 | 251 | 526 | 2 |
| 19.0011 | 8 | POL | 429 | HPAAMPHL | 3638 | 100 | 85 | 18000 | 18 | 2514 | 625 | 2 |
| 19.0012 | 8 | POL | 511 | SPFLLAQF | 3639 | 95 | 10 | 8000 | 306 | 10333 | 1075 | 2 |
| 26.0566 | 11 | poL | 511 | SPFLLAQFTSA | 3640 | 95 | 67 | — | — | — | 0.83 | 2 |
| 1147.01 | 9 | POL | 789 | DPSRGRLGL | 3641 | 90 | 458 | — | — | — | — | 1 |
| 16.0182 | 10 | X | 67 | GPCALRFTSA | 3642 | 90 | 61 | — | — | — | 2857 | |
| 20.0273 | 10 | POL | 440 | HPAAMPHLLV | 3643 | 85 | 344 | 3600 | 705 | 664 | 588 | 1 |
| 15.0030 | 9 | ENV | 191 | IPQSLDSWW | 3644 | 90 | — | — | 27500 | 62 | — | 1 |
| 15.0210 | 10 | POL | 123 | LPLDKGIKPY | 3645 | 100 | — | 248 | 27500 | — | — | 1 |
| 16.0006 | 9 | ENV | 25 | FPDHQLDPA | 3646 | 90 | — | 8000 | — | — | 12 | 1 |
| 16.0177 | 10 | ENV | 324 | IPIPSSWAFA | 3647 | 80 | 4231 | 3000 | 6643 | | 22 | 1 |
| 16.0180 | 10 | POL | 644 | APFTQCGYPA | 3648 | 95 | 1897 | — | | — | 7.1 | 1 |
| 16.0181 | 10 | POL | 723 | LPIHTAELLA | 3649 | 85 | 3056 | 6545 | | 5813 | 30 | 1 |
| 19.0003 | 8 | ENV | 173 | OPLLVLQA | 3650 | 95 | 18333 | — | 500 | — | 1538 | 1 |
| 19.0005 | 8 | ENV | 313 | IPIPSSWA | 3651 | 100 | 13750 | 18000 | 2895 | — | 167 | 1 |
| 19.0009 | 8 | NUC | 133 | RPPNAPIL | 3652 | 100 | 724 | — | 196 | — | — | 1 |
| 19.0015 | 8 | POL | 659 | SPTYKAFL | 3653 | 95 | 14 | — | 2895 | — | — | 1 |
| 19.0016 | 8 | POL | 769 | VPSALNPA | 3654 | 90 | 5000 | — | 786 | — | 10 | 1 |
| 26.0554 | 11 | pol | 633 | APFTQCGYPAL | 3655 | 95 | 24 | 7200 | 13750 | — | 1075 | 1 |
| 26.0359 | 11 | pol | 712 | LPIHTAELLAA | 3656 | 85 | 611 | 2667 | — | 775 | 3.6 | 1 |
| 26.0561 | 11 | pol | 774 | NPADDPSRGRL | 3657 | 90 | 458 | — | — | — | — | 1 |
| 26.0564 | 11 | Core | 133 | RPPNAPILSTL | 3658 | 100 | 42 | — | 3056 | — | — | 1 |
| 26.0567 | 11 | Core | 49 | SPHHTALRQAI | 3659 | 100 | 9.5 | — | 13750 | 18600 | — | 1 |
| 26.0568 | 11 | pol | 354 | TPARVTGGVFL | 3660 | 90 | 58 | — | — | 18600 | 20000 | 1 |

¹Frequency of entire sequence amongst isolates scanned.
²Number of supertype alleles bound. Peptides binding 3 or more alleles are considered degenerate.
A dash (—) indicates IC50

TABLE XXIX

HBV derived A1- and A24- motif containing peptides

| | | | | | | |
|---|---|---|---|---|---|---|
| | | | | | | HLA-A*0101 |
| Peptide | Molecule | Position | Sequence | SEQ ID NO: | Conserv. | binding (IC50 nM) |

a. A1-motif peptides

| | | | | | | |
|---|---|---|---|---|---|---|
| 1069.01 | Core | 59 | LLDTASALY | 3661 | 75 | 2.1 |
| 1.0519 | Core | 419 | DLLDTASALY | 3662 | 75 | 2.3 |
| 1069.02 | pol | 427 | SLDVSAAFY | 3663 | 95 | 4.8 |
| 2.0239 | | 1000 | LSLDVSAAFY | 3664 | 95 | 6.0 |
| 2.0126 | | 1521 | MSTTDLEAY | 3665 | 75 | 29 |
| 1039.06 | ENV | 359 | WMMWYWGPSLY | 3666 | 85 | 78 |
| 1090.14 | pol | 538 | YMDDVVLGA | 3667 | 90 | 96 |
| 1090.09 | pol | 808 | PTTGRTSLY | 3668 | 85 | 119 |
| 1069.03 | pol | 124 | PLDKGIKPYY | 3669 | 100 | 147 |
| 1069.08 | env | 249 | ILLLCLIFLL | 3670 | 100 | 192 |
| 1069.04 | pol | 149 | HTLWKAGILY | 3671 | 100 | 381 |
| 1039.01 | | 360 | MMWYWGPSLY | 3672 | 85 | 309 |
| 1.0774 | Core | 416 | WLWGMDIDPY | 3673 | 75 | 309 |
| 20.0254 | pol | 631 | FAAPFTQCGY | 3674 | 95 | 368 |
| 1.0166 | pol | 629 | KVGNFTGLY | 3675 | 95 | 368 |

| | | | | | | |
|---|---|---|---|---|---|---|
| | | | | | | HLA-A*2402 |
| Peptide | Molecule | Position | Sequence | SEQ ID NO: | Conserv. | binding (IC50 nM) |

b. A24- motif peptides

| | | | | | | |
|---|---|---|---|---|---|---|
| 20.0271 | POL | 392 | SWPKFAVPNL | 3676 | 95 | 2.1 |
| 1069.23 | POL | 745 | KYTSFPWLL | 3677 | 85 | 2.3 |
| 2.0181 | POL | 492 | LYSHPIILGF | 3678 | 80 | 11 |
| 20.0269 | ENV | 236 | RWMCLRRFII | 3679 | 95 | 11 |
| 20.0136 | ENV | 334 | SWLSLLVPF | 3680 | 100 | 31 |
| 20.0137 | ENV | 197 | SWWTSLNFL | 3681 | 95 | 32 |
| 20.0135 | ENV | 236 | RWMCLRRFI | 3682 | 95 | 169 |
| 20.0139 | POL | 167 | SFCGSPYSW | 3683 | 100 | 169 |
| 2.0173 | POL | 4 | SYQHFRKLLL | 3684 | 75 | 182 |
| 2.0060 | | 1224 | GYPALMPLY | 3685 | 95 | 245 |
| 13.0129 | NUC | 117 | EYLVSFGVWI | 3686 | 90 | 353 |
| 1090.02 | core | 131 | AYRPPNAPI | 3687 | 90 | 387 |
| 13.0073 | NUC | 102 | WFHISCLTF | 3688 | 80 | 400 |
| 20.0138 | POL | 51 | PWTHKVGNF | 3689 | 100 | 414 |

A dash indicates IC50 nM

TABLE XXXa

Immunogenicity of HBV-derived A2-supermotif cross-reactive peptides

Immunogenicity

| | | | | | | | | |
|---|---|---|---|---|---|---|---|---|
| Peptide | Sequence | SEQ ID NO: | Protein | XRN | primary | transgenic | patients | overall¹ |

| | | | | | | | | |
|---|---|---|---|---|---|---|---|---|
| 924.07 | FLPSDPFPSV | 3690 | HBV core 18 | 5 | 10/10 | 6/6 | 25/32^{a} | + |
| 1069.06 | LLVPFVQWFV | 3691 | HBV env 338 | 5 | 3/4 | 6/9 | | + |
| 1147.13 | FLLAQFTSAI | 3692 | HBV pol 513 | 5 | | 0/3 | | − |
| 1090.77 | YMDDVVLGV | 3693 | HBV pol 538 | 5 | | 9/9 | | + |
| 777.03 | FLLTRILTI | 3694 | HBV env 183 | 4 | | | 14/23^{a} | + |
| 927.15 | ALMPLYACI | 3695 | HBV pol 642 | 4 | 10/12 | 3/5 | 2/15^{a} | + |
| 1013.01 | WLSLLVPFV | 3696 | HBV env 335 | 4 | 2/6 | 5/9 | 23/29^{a} | + |
| 1069.05 | LLAQFTSAI | 3697 | HBV pol 504 | 4 | 0/4 | 0/5 | | − |
| 1132.01 | LVPFVQWFV | 3698 | HBV env 339 | 4 | 0/3 | 0/4 | | − |
| 1147.14 | VLLDYQGMLPV | 3699 | HBV env 259 | 4 | 4/4 | 6/6 | | + |
| 927.41 | LLSSNLSWL | 3700 | HBV pol 992 | 3 | 0/4 | 0/3 | | − |
| 927.42 | NLSWLSLDV | 3701 | HBV pol 411 | 3 | | 2/8 | | + |
| 927.46 | KLHLYSHPI | 3702 | HBV pol 489 | 3 | 0/4 | 4/6 | | + |
| 1069.07 | FLLAQFTSA | 3703 | HBV pol 503 | 3 | 1/2 | 0/3 | | + |
| 1168.02 | GLSRYVARL | 3704 | HBV pol 455 | 3 | | | 9/13^{a} | + |

Immunogenicity evaluation derived from primary cultures, acute patients (a-Bertoni et al, J Clin Invest 100:503, b-Rehermann et al., J. Clin. Invest 97:1655, c-Nayersina et al., J Immunol 150:4659) or transgenic mice. A positive assessment (+) is assigned when responders have been noted in one of these systems.

TABLE XXXb

Immunogenicity of non-crossreactive HBV A2-supermotif peptides

Immunogenicity

| | | | | | | | | |
|---|---|---|---|---|---|---|---|---|
| Peptide | Sequence | SEQ ID NO: | Protein | XRN | primary | transgenic | patients | overall¹ |

| | | | | | | | | |
|---|---|---|---|---|---|---|---|---|
| 927.11 | FLLSLGIHL | 3705 | HBV pol 562 | 2 | 15/22 | 12/13 | 9/15^{a} | + |
| 927.47 | HLYSHPIIL | 3706 | HBV pol 1076 | 2 | | 10/14 | | + |
| 1039.03 | MMWYWGPSL | 3707 | HBV env 360 | 2 | 3/4 | 0/4 | | + |
| 1069.12 | YLHTLWKAGV | 3708 | HBV pol 147 | 2 | 2/4 | | | + |
| 1137.02 | LLDYQGMLPV | 3709 | HBV env 260 | 2 | 1/2 | 0/4 | | + |
| 1142.07 | GLLGWSPQA | 3710 | HBV env 62 | 2 | 3/4 | 5/6 | | + |
| 1.0573 | ILRGTSFVYV | 3711 | HBV pol 773 | 1 | | | 3/7^{b} | + |
| 1013.14 | VLQAGFFLL | 3712 | HBV env 177 | 1 | 0/4 | 5/12 | | + |
| 1069.10 | LLPIFFCLWV | 3713 | HBV env 378 | 1 | 3/3 | 0/4 | 2/5^{c} | + |
| 1069.13 | PLLPIFFCL | 3714 | HBV env 377 | 1 | 0/4 | 7/12 | | + |
| 1090.06 | LLVLQAGFFL | 3715 | HBV env 175 | 1 | 1/5 | 0/4 | | + |
| 1090.12 | YLVSFGVWI | 3716 | HBV nuc 118 | 1 | 9/9 | | | + |
| 1.0518 | GLSPTVWLSV | 3717 | HBV env 338 | 1 | | | 3/9^{c} | + |
| 1090.14 | YMDDVVLGA | 3718 | HBV pol 538 | 1 | 2/7 | 2/5 | 2/7^{b} | + |

Immunogenicity evaluation derived from primary cultures, acute patients (a-Bertoni et al, J Clin Invest 100:503, b-Rehermann et al., J. Clin. Invest 97:1655, c-Nayersina et al., J Immunol 150:4659) or transgenic mice. A positive assessment (+) is assigned when responders have been noted in one of these systems.

TABLE XXXc
Cross-recognition of HBV pol 538 and a Lamivudine induced
pol 538 variant by CTL induced with a pol 538 analog^{a}.

| | Day 6 CTL response (ΔLU) | |
|---|---|---|
| | HBV pol 538 | HBV pol 538 mutant |
| Stimulating peptide | (YMDDVVLGA)^{b} | (YVDDVVLGA) |
| HBV pol 538 | 27.8 | 54.2 |
| HBV pol 538 mutant | 35.3 | 27.9 |

^{a}CTLs were induced using the 1090.77 analog of HBV pol 538 (peptide 1090.14). 1090.77 was encoded in the DNA minigene pEP2.AOS.
^{b}Values shown represent the geometric mean of ΔLU from 2 independent cultures. Peptides loaded onto target cells were 1090.14 (HBV pol 538) or 1353.02 (a Lamivudine induced mutant of pol 538).

TABLE XXXIa

Immunogenicity of HBV-derived A3-supermotif cross-reactive peptides

Immunogenicity

| | | | | | | | | |
|---|---|---|---|---|---|---|---|---|
| Peptide | Sequence | SEQ ID NO: | Protein | XRN | primary | transgenic | patients | overall¹ |

| | | | | | | | | |
|---|---|---|---|---|---|---|---|---|
| 1147.16 | HTLWKAGILYK | 3719 | HBV POL 149 | 5 | 0/6 | 3/3 | 1/22 | + |
| 1083.01 | STLPETTVVRR | 3720 | HBV core 141 | 4 | 3/5 | 6/6 | 8/32 | + |
| 1150.51 | GSTHVSWPK | 3721 | HBV pol 398 | 4 | | 3/6 | | + |
| 1.0219 | FVLGGCRHK | 3722 | HBV adr “X” 1550 | 3 | 0/4 | | | − |
| 1069.16 | NVSIPWTHK | 3723 | HBV pol 47 | 3 | 0/8 | 0/3 | 1/21 | + |
| 1069.20 | LVVDFSQFSR | 3724 | HBV pol 388 | 3 | 0/4 | 6/6 | 1/22 | + |
| 1090.10 | QAFTFSPTYK | 3725 | HBV pol 665 | 3 | 3/6 | 0/3 | 3/21 | + |
| 1090.11 | SAICSVVRR | 3726 | HBV pol 531 | 3 | 1/4 | | 2/22 | + |

¹Immunogenicity evaluation derived from primary cultures, Bertoni et al, J Clin Invest 100:503 or transgenic mice. A positive assessment (+) is assigned when responders have been noted in one of these systems. A negative assessment (−) indicates that no responders when examined.

TABLE XXXIb

Immunogenicity of non-crossreactive HBV A3-supermotif peptides

Immunogenicity

| | | | | | | | | |
|---|---|---|---|---|---|---|---|---|
| Peptide | Sequence | SEQ ID NO: | Protein | XRN | primary | transgenic | patients | overall¹ |

| | | | | | | | | |
|---|---|---|---|---|---|---|---|---|
| 1069.15 | TLWKAGILYK | 3727 | HBV pol 150 | 2 | 3/8 | 0/3 | 5/28 | + |
| 1142.05 | KVGNFTGLY | 3728 | HBV adr POL 629 | 2 | | 0/3 | 2/22 | + |

¹Immunogenicity evaluation derived from primary cultures, Bertoni et al, J Clin Invest 100:503 or transgenic mice. A positive assessment (+) is assigned when responders have been noted in one of these systems. A negative assessment (−) indicates that no responders when examined.

TABLE XXXIIa

Immunogenicity of HBV B7-supermotif cross-reactive peptides

Immunogenicity

| | | | | | | | | |
|---|---|---|---|---|---|---|---|---|
| Peptide | Sequence | SEQ ID NO: | Protein | XRN | primary | transgenic | patients | overall¹ |

| | | | | | | | |
|---|---|---|---|---|---|---|---|
| 1147.05 | FPHCLAFSYM | 3729 | KBV POL 530 | 5 | 1/3 | 0/12 | + |
| 988.05 | LPSDFFPSV | 3730 | HBV core 19-27 | 4 | | 2/16 | + |
| 1145.04 | IPIPSSWAF | 3731 | HBV ENV 313 | 4 | 0/4 | 1/12 | + |
| 1147.02 | HPAAMPHLL | 3732 | HBV POL 429 | 4 | 0/5 | 0/12 | - |
| 1147.06 | LPVCAFSSA | 3733 | HBV X 58 | 4 | 1/4 | | + |
| 1147.08 | YPALMPLYA | 3734 | HBV POL 640 | 4 | | 0/12 | - |
| 1145.08 | FPHCLAFSY | 3735 | HBV POL 541 | 3 | 0/4 | | − |

¹Immunogenicity evaluation derived from primary cultures, Bertoni et al, J Clin Invest 100:503 or transgenic mice. A positive assessment (+) is assigned when responders have been noted in one of these systems. A negative assessment (−) indicates that no responders when examined.

TABLE XXXIIb:

Immunogenicity of non-crossreactive HBV B7-supermotif peptides

Immunogenicity

| | | | | | | | | |
|---|---|---|---|---|---|---|---|---|
| Peptide | Sequence | SEQ ID NO: | Protein | XRN | primary | transgenic | patients | overall¹ |

| | | | | | | |
|---|---|---|---|---|---|---|
| 1147.04 | TPARVTGGVF | 3736 | HBV POL 354 | 2 | 2/12 | + |

¹Immunogenicity evaluation derived from primary cultures, Bertoni et al, J Clin Invest 100:503 or transgenic mice. A positive assessment (+) is assigned when responders have been noted in one of these systems. A negative assessment (−) indicates that no responders when examined.

TABLE XXXIII

Candidate HBV-derived HTL epitopes

| | |
|---|---|
| Selection | Conservancy |

| | | | | | | | |
|---|---|---|---|---|---|---|---|
| criteria | Peptide | Mol | 1st Pos | Core | Total | Sequence | SEQ ID NO: |

| | | | | | | | |
|---|---|---|---|---|---|---|---|
| DR-supermotif | F107.01 | ENV | 249 | 100 | 95 | ILLLCLIFLLVLLDY | 3737 |
| | F107.02 | ENV | 252 | 95 | 95 | LCLIFLLVLLDYQGM | 3738 |
| | 1280.17 | ENV | 258 | 90 | 90 | LVLLDYQGMLPVCPL | 3739 |
| | 1186.22 | ENV | 332 | 100 | 100 | RFSWLSLLVPFVQWF | 3740 |
| | 1186.15 | ENV | 339 | 95 | 95 | LVPFVQWFVGLSPTV | 3741 |
| | 1186.06 | ENV | 342 | 95 | 95 | FVQWFVGLSPTVWLS | 3742 |
| | 1186.03 | NUC | 19 | 85 | 85 | ASKLCLGWLWGMDID | 3743 |
| | 1186.12 | NUC | 24 | 85 | 85 | LGWLWGMDIDPYKEF | 3744 |
| | 857.02 | NUC | 50 | | 90 | PHHTALRQAILCWGELMTLA | 3745 |
| | 1186.23 | NUC | 98 | 85 | 85 | RQLLWFHISCLTFGR | 3746 |
| | 27.0279 | NUC | 117 | | 90 | EYLVSFGVWIRTPPA | 3747 |
| | 27.0280 | NUC | 123 | 95 | 95 | GVWIRTPPAYRPPNA | 3748 |
| | 1186.20 | NUC | 129 | 100 | 95 | PPAYRPPNAPTLSTL | 3749 |
| | 1186.16 | NUC | 136 | 100 | 95 | NAPILSTLPETTVVR | 3750 |
| | 1186.01 | POL | 38 | 95 | 95 | AEDLNLGNLNVSIPW | 3751 |
| | 1186.17 | POL | 45 | 100 | 95 | NLNVSIPWTHKVGNF | 3752 |
| | 27.0281 | POL | 145 | 100 | 100 | RHYLHTLWKAGILYK | 3753 |
| | 1280.13 | POL | 406 | 95 | 95 | KFAVPNLQSLTNLLS | 3754 |
| | 27.0283 | POL | 409 | | 85 | VPNLQSLTNLLSSNL | 3755 |
| | F107.03 | POL | 412 | 90 | 90 | LQSLTNLLSSNLSWL | 3756 |
| | 1186.28 | POL | 416 | 90 | 90 | TNLLSSNLSWLSLDV | 3757 |
| | 1186.27 | POL | 420 | 100 | 85 | SSNLSWLSLDVSAAF | 3758 |
| | F107.04 | POL | 523 | 95 | 95 | PFLLAQFTSAICSVV | 3759 |
| | 1186.10 | POL | 526 | 95 | 95 | LAQFTSAICSVVRRA | 3760 |
| | 1186.04 | POL | 534 | 95 | 95 | CSVVRRAFPHCLAFS | 3761 |
| | F107.05 | POL | 538 | 95 | 95 | RRAFPHCLAFSYMDD | 3762 |
| | 1186.02 | POL | 546 | 90 | 90 | AFSYMDDVVLGAKSV | 3763 |
| | 1186.05 | POL | 629 | 85 | 85 | DWKVCQRIVGLLGFA | 3764 |
| | 1280.21 | POL | 637 | 95 | 95 | VGLLGFAAPFTQCGY | 3765 |
| | 27.0278 | POL | 643 | | 95 | AAPFTQCGYPALMPL | 3766 |
| | 1186.21 | POL | 648 | 95 | 95 | QCGYPALMPLYACIQ | 3767 |
| | 1280.14 | POL | 694 | 95 | 95 | LCQVFADATPTGWGL | 3768 |
| | 27.0282 | POL | 750 | 85 | 85 | SVVLSRKYTSFPWLL | 3769 |
| | | X | 13 | 95 | 90 | RDVLCLRPVGAESRG | 3770 |
| | 1186.07 | X | 50 | 95 | 90 | GAHLSLRGLPVCAFS | 3771 |
| | 1186.29 | X | 60 | 95 | 90 | VCAFSSAGPCALRFT | 3772 |
| Algorithm | 1280.20 | ENV | 330 | 100 | 80 | SVRFSWLSLLVPFVQ | 3773 |
| | 1280.19 | NUC | 28 | 85 | 80 | RDLLDTASALYREAL | 3774 |
| | 1298.02 | POL | 56 | 90 | 55 | VGNFTGLYSSTVPVF | 3775 |
| | 1298.03 | POL | 571 | 95 | 75 | TNFLLSLGIHLNPNK | 3776 |
| | 1298.05 | POL | 651 | 95 | 55 | YPALMPLYACIQSKQ | 3777 |
| | 1298.06 | POL | 664 | 95 | 60 | KQAFTFSPTYKAFLC | 3778 |
| | 1280.181 | POL | 722 | 85 | 80 | PLPIHTAELLAACFA | 3779 |
| | 1280.09 | POL | 774 | 90 | 80 | GTSFVYVPSALNPAD | 3780 |
| DR3-motif | 795.05 | ENV | 10 | | 95 | PLGFFPDHQLDP | 3781 |
| | 35.0090 | ENV | 312 | 95 | 90 | FLLVLLDYQGMLPVC | 3782 |
| | CF-03 | NUC | 28 | 85 | 80 | RDLLDTASALYREALESPEH | 3783 |
| | 35.0091 | POL | 18 | 90 | 65 | AGPLEEELPRLADEG | 3784 |
| | 35.0092 | POL | 34 | 100 | 85 | NRRVAEDLNLGNLNV | 3785 |
| | 35.0093 | POL | 96 | 85 | 60 | VGPLTVNEKRRLKLI | 3786 |
| | 35.0094 | POL | 120 | 100 | 100 | TKYLPLDKGIKPYYP | 3787 |
| | 35.0095 | POL | 371 | 100 | 55 | GGVFLVDKNPHNTTE | 3788 |
| | 35.0096 | POL | 385 | 100 | 45 | ESRLVVDFSQFSRGN | 3789 |
| | 1186.18 | POL | 422 | 95 | 85 | NLSWLSLDVSAAFYH | 3790 |
| | 35.0099 | POL | 666 | 95 | 55 | AFTFSPTYKAFLCKQ | 3791 |
| | 35.0101 | X | 18 | 95 | 35 | LRPVGAESRGRPVSG | 3792 |
| Lower | 799.01 | ENV | 11 | 80 | 75 | PLLVLQAGFFLLTRILTIPQ | 3793 |
| conservancy | 799.02 | ENV | 31 | 95 | | SLDSWWTSLNPLGGTTVCLG | 3794 |
| or miscellaneous 799.04 | ENV | 71 | 95 | 75 | GYRWMCLRRFIIFLFILLLC | 3795 | |
| | 1298.01 | ENV | 137 | 80 | 40 | PQAMQWNSTTFHQTL | 3796 |
| | 1280.06 | ENV | 180 | 80 | 80 | AGFFLLTRILTIPQS | 3797 |
| | 1280.11 | ENV | 245 | 80 | 80 | IFLFILLLCLIFLLV | 3798 |
| | CF-08 | NUC | 120 | | 90 | VSFGVWIRTPPAYRPPNAPI | 3799 |
| | 1186.25 | NUC | 121 | 95 | 90 | SFGVWIRTPPAYRPP | 3800 |
| | 1280.15 | POL | 501 | 80 | 80 | LHLYSHIPIIIGFRKI | 3803 |
| | 1298.04 | POL | 618 | 80 | 45 | KQCFRKLPVNRPIDW | 3802 |
| | 1298.07 | POL | 767 | 80 | 70 | AANWILRGTSFVYVP | 3803 |
| | 1298.08 | POL | 827 | 80 | 60 | PDRVHFASPLHVAWR | 3804 |

TABLE XXXIV
HLA-DR screening panels

| Screening | | | Representative Assay | | Phenotypic Frequencies | | | | | |
|---|---|---|---|---|---|---|---|---|---|---|
| Panel | Antigen | Alleles | Allele | Alias | Cauc. | Blk. | Jpn. | Chn. | Hisp. | Avg. |
| Primary | DR1 | DRB1*0101-03 | DRB1*0101 | (DR1) | 18.5 | 8.4 | 10.7 | 4.5 | 10.1 | 10.4 |
| | DR4 | DRB1*0401-12 | DRB1*0401 | (DR4w4) | 23.6 | 6.1 | 40.4 | 21.9 | 29.8 | 24.4 |
| | DR7 | DRB1*0701-02 | DRB1*0701 | (DR7) | 26.2 | 11.1 | 1.0 | 15.0 | 16.6 | 14.0 |
| | Panel total | | | | 59.6 | 24.5 | 49.3 | 38.7 | 51.1 | 44.6 |
| Secondary | DR2 | DRB1*1501-03 | DRB1*1501 | (DR2w2 β1) | 19.9 | 14.8 | 30.9 | 22.0 | 15.0 | 20.5 |
| | DR2 | DRB5*0101 | DRB5*0101 | (DR2w2 β2) | — | — | — | — | — | — |
| | DR9 | DRB1*09011, 09012 | DRB1*0901 | (DR9) | 3.6 | 4.7 | 24.5 | 19.9 | 6.7 | 11.9 |
| | DR13 | DRB1*1301-06 | DRB1*1302 | (DR6w19) | 21.7 | 16.5 | 14.6 | 12.2 | 10.5 | 15.1 |
| | Panel total | | | | 42.0 | 33.9 | 61.0 | 48.9 | 30.5 | 43.2 |
| Tertiary | DR4 | DRB1*0405 | DRB1*0405 | (DR4w15) | — | — | — | — | — | — |
| | DR8 | DRB1*0801-5 | DRB1*0802 | (DR8w2) | 5.5 | 10.9 | 25.0 | 10.7 | 23.3 | 15.1 |
| | DR11 | DRB1*1101-05 | DRB1*1101 | (DR5w11) | 17.0 | 18.0 | 4.9 | 19.4 | 18.1 | 15.5 |
| | Panel total | | | | 22.0 | 27.8 | 29.2 | 29.0 | 39.0 | 29.4 |
| Quarternary | DR3 | DRB1*0301-2 | DRB1*0301 | (DR3w17) | 17.7 | 19.5 | 0.4 | 7.3 | 14.4 | 11.9 |
| | DR12 | DRB1*1201-02 | DRB1*1201 | (DR5w12) | 2.8 | 5.5 | 13.1 | 17.6 | 5.7 | 8.9 |
| | Panel total | | | | 20.2 | 24.4 | 13.5 | 24.2 | 19.7 | 20.4 |

TABLE XXXV

HBV-derived cross-reactive HLA-DR binding peptides

| | |
|---|---|
| Conservancy | HLA-DR binding capacity (IC50 nM) |

| | | | | | | | | | | | | |
|---|---|---|---|---|---|---|---|---|---|---|---|---|
| Peptide | Mol | 1st Pos | Core | Total | Sequence | SEQ ID NO: | DR1 | DR2w2β1 | DR2w2β2 | DR3 | DR4w4 | DR4w15 |

| | | | | | | | | | | | | |
|---|---|---|---|---|---|---|---|---|---|---|---|---|
| F107.03 | POL | 412 | 90 | 90 | LQSLTNLLSSNLSWL | 3805 | 2.0 | 21 | 1000 | —^{a} | 9.4 | 47 |
| 1293.06 | POL | 664 | 95 | 60 | KQAFTFSPTYKAFLC | 3806 | 9.4 | 38 | 143 | — | 41 | 173 |
| 1280.06 | ENV | 180 | 80 | 80 | AGFFLLTRILTTPQS | 3807 | 1.1 | 217 | 1053 | — | 8.5 | 253 |
| 1280.09 | POL | 774 | 90 | 80 | GTSFVYVPSALNPAD | 3808 | 14 | 650 | 400 | — | 118 | 93 |
| 11186.25 | NUC | 121 | 95 | 90 | SFGVWIRTPPAYRPP | 3809 | 532 | 827 | 47 | — | 577 | 603 |
| 27.0280 | NUC | 123 | 95 | 95 | GVWIRTPPAYRPPNA | 3810 | 14 | 217 | 2.8 | — | 13 | 67 |
| CF-08 | NUC | 120 | | 90 | VSFGVWIRTPPAYRPPNAPI | 3811 | 192 | | 105 | | 300 | |
| 27.0281 | POL | 145 | 100 | 100 | RHYLHTLWKAGILYK | 3812 | 17 | 5.4 | 35 | — | 2250 | 1462 |
| 1186.15 | ENV | 339 | 95 | 95 | LVPFVQWFVGLSPTV | 3813 | 385 | 13 | 1429 | — | 300 | 27 |
| 1280.15 | POL | 501 | 80 | 80 | LHLYSHPIILGFRKI | 3814 | 227 | 268 | 500 | — | 66 | 238 |
| F107.04 | POL | 523 | 95 | 95 | PFLLAQFTSAICSVV | 3815 | 28 | 337 | 4762 | — | 563 | 317 |
| 1298.04 | POL | 618 | 80 | 45 | KQCFRKLPVNRPIDW | 3816 | 3.3 | 4136 | 952 | — | 38 | 45 |
| 1298.07 | POL | 767 | 80 | 70 | AANWILRGTSFVYVP | 3817 | 54 | 379 | 3279 | — | 882 | 1520 |
| 857.02 | NUC | 50 | | 90 | PHHTALRQAILCWGELMTLA | 3818 | 70 | 9.1 | 211 | — | 85 | |

| | |
|---|---|
| HLA-DR binding capacity (IC50 nM) | Total DR |

| | | | | | | |
|---|---|---|---|---|---|---|
| Peptide | DR5w11 | DR6 | DR7 | DR8 | DR9 | alleles bound |

| | | | | | | |
|---|---|---|---|---|---|---|
| F107.03 | 294 | 135 | 167 | 557 | 682 | 10 |
| 1293.06 | 83 | 175 | 76 | 408 | 139 | 10 |
| 1280.06 | 5.6 | 9.5 | 8.1 | 188 | 58 | 9 |
| 1280.09 | 426 | — | 93 | 803 | 221 | 9 |
| 11186.25 769 | 17500 | 1042 | 196 | 938 | 8 | |
| 27.0280 | 42 | — | 114 | 92 | 1667 | 8 |
| CF-08 | 426 | | 124 | | | 5 |
| 27.0281 | 42 | 745 | 61 | 27 | 174 | 8 |
| 1186.15 | 53 | 1944 | 2717 | 74 | 30 | 7 |
| 1280.15 | 488 | 17500 | — | 803 | 1531 | 7 |
| F107.04 | 1667 | 44 | 325 | 845 | 1271 | 7 |
| 1298.04 | 1538 | 814 | 63 | 845 | 3000 | 7 |
| 1298.07 | 1429 | 140 | 43 | 196 | 278 | 7 |
| 857.02 | 263 | 193000 | 676 | 196 | 2273 | 7 |

^{a}A dash (-) indicates IC50 nM > 20.000.

TABLE XXXVI

HBV-derived DR3-binding peptides

Conservancy

| | | | | | | | |
|---|---|---|---|---|---|---|---|
| Peptide | Mol | 1st Pos | Core | Total | Sequence | +HC,3o SEQ ID NO: | DR3 |

| | | | | | | | |
|---|---|---|---|---|---|---|---|
| 1280.14* | POL | 694 | 95 | 95 | LCQVFADATPTGWGL | 3819 | 67 |
| 35.0096 | POL | 385 | 100 | 45 | ESRLVVDFSQFSRGN | 3820 | 115 |
| 35.0093 | POL | 96 | 85 | 60 | VGPLTVNEKRRLKLI | 3821 | 136 |
| 1186.27 | POL | 420 | 100 | 85 | SSNLSWLSLDVSAAF | 3822 | 200 |
| 1186.18 | POL | 422 | 95 | 85 | NLSWLSLDVSAAFYH | 3823 | 231 |

*tested as peptide 35.0100

TABLE XXXVIIa

HBV Preferred CTL Epitopes

| | | | | |
|---|---|---|---|---|
| Peptide | Sequence | SEQ ID NO: | Protein | HLA |

| | | | | | |
|---|---|---|---|---|---|
| 924.07 | FLPSDFFPSV | 3824 | core 18 | A2 | |
| 777.03 | FLLTRILTI | 3825 | env 183 | A2 | |
| 927.15 | ALMPLYACI | 3826 | pol 642 | A2 | |
| 1013.01 | WLSLLVPFV | 3827 | env335 | A2 | |
| 1090.77 | YMDDVVLGV | 3828 | pol 538 | A2/A1 | |
| 1168.02 | GLSRYVARL | 3829 | pol 455 | A2 | |
| 927.11 | FLLSLGIHL | 3830 | pol 562 | A2 | |
| 1069.10 | LLPIFFCLWV | 3831 | env 378 | A2 | |
| 1069.06 | LLVPGVQWFV | 3832 | env 338 | A2 | |
| 1147.16 | HTLWKAGILYK | 3833 | pol 149 | A3/A1 | |
| 1083.01 | STLPETTVVRR | 3834 | core 141 | A3 | |
| 1069.16 | NVSIPWTHK | 3835 | pol 47 | A3 | |
| 1069.20 | LVVDFSQFSR | 3836 | pol 388 | A3 | |
| 1090.10 | QAFTFSPTYK | 3837 | pol 665 | A3 | |
| 1090.11 | SAICSVVRR | 3838 | pol 531 | A3 | |
| 1142.05 | KVGNFTGLY | 3839 | pol 629 | A3/A1 | |
| 1147.05 | FPHCLAFSYM | 3840 | pol 530 | B7 | |
| 988.05 | LPSDFFPSV | 3841 | core 19 | B7 | |
| 1145.04 | IPIPSSWAF | 3842 | env 313 | B7 | |
| 1147.02 | HPAAMPHLL | 3843 | pol 429 | B7 | |
| 26.0570 | YPALMPLYACI | 3844 | pol 640 | B7 | |
| 1147.04 | TPARVTGGVF | 3845 | pol | 354 | B7 |
| 1.0519 | DLLDTASALY | 3846 | core 419 | A1 | |
| 2.0239 | LSLDVSAAFY | 3847 | pol 1000 | A1 | |
| 1039.06 | WMMWYWGPSLY | 3848 | env 359 | A1 | |
| 20.0269 | RWMCLRRFII | 3849 | env 236 | A24 | |
| 20.0136 | SWLSLLVPF | 3850 | env 334 | A24 | |
| 20.0137 | SWWTSLNFL | 3851 | env 197 | A24 | |
| 13.0129 EYLVSFGVWI | 3852 | core 117 | A24 | | |
| 1090.02 | AYRPPNAPI | 3853 | core 131 | A24 | |
| 13.0073 | WFHISCLTF | 3854 | core 102 | A24 | |
| 20.0271 | SWPKFAVPNL | 3855 | pol 392 | A24 | |
| 1069.23 | KYTSFPWLL | 3856 | pol 745 | A24 | |
| 2.0181 | LYSHPIILGF | 3857 | pol 492 | A24 | |

TABLE XXXVIIb

HBV Preferred HTL epitopes

| | |
|---|---|
| Selection | Conservancy |

| | | | | | | | |
|---|---|---|---|---|---|---|---|
| Criteria | Peptide | Mol | 1st Pos | Core | Total | SEQ ID NO: | Sequence |

| | | | | | | | |
|---|---|---|---|---|---|---|---|
| DR supermotif | F107.03 | POL | 412 | 90 | 90 | 3838 | LQSLTNLLSSNLSWL |
| | 1298.06 | POL | 664 | 95 | 60 | 3859 | KQAFTFSPTYKAFLC |
| | 1280.06 | ENV | 180 | 80 | 80 | 3860 | AGFFLLTRILTIPQS |
| | 1280.09 | POL | 774 | 90 | 80 | 3861 | GTSFVYVPSALNPAD |
| | CF-08 | CORE | 120 | | 90 | 3862 | VSFGVWIRTPPAYRPPNAPI |
| | 27.0281 | POL | 145 | 100 | 100 | 3863 | RHYLHTHLWKAGILYK |
| | 1186.15 | ENV | 339 | 95 | 95 | 3864 | LVPFVQWFVGLSPTV |
| | 1280.15 | POL | 501 | 80 | 80 | 3865 | LHLYSHPIILGFRKI |
| | F107.04 | POL | 523 | 95 | 95 | 3866 | PFLLAQFTSAICSVV |
| | 1298.04 | POL | 618 | 80 | 45 | 3867 | KQCFRKLPVNRPIDW |
| | 1298.07 | POL | 767 | 80 | 70 | 3868 | AANWILRGTSFVYVP |
| | 857.02 | CORE | 50 | | 90 | 3869 | PHHTALRQAILCWGELMTLA |
| DR3 motif | 1280.14 | POL | 694 | 95 | 95 | 3870 | LCQVFADATPTGWGL |
| | 35.0096 | POL | 385 | 100 | 45 | 3871 | ESRLVVDFSQFSRGN |
| | 35.0093 | POL | 96 | 85 | 60 | 3872 | VGPLTVNEKRRLKLI |
| | 1186.27 | POL | 420 | 100 | 85 | 3873 | SSNLSWLSLDVSAAF |

TABLE XXXVIII
Estimated population coverage by a panel of HBV derived HTL epitopes

| | | Representative | No. of | Population coverage (phenotypic frequency) | | | | | |
|---|---|---|---|---|---|---|---|---|---|
| Antigen | Alleles | assay | epitopes² | Cauc. | Blk. | Jpn. | Chn. | Hisp. | Avg. |
| DR1 | DRB1*0101-03 | DR1 | 12 | 18.5 | 8.4 | 10.7 | 4.5 | 10.1 | 10.4 |
| DR2 | DRB1*1501-03 | DR2w2 β1 | 11 | 19.9 | 14.8 | 30.9 | 22.0 | 15.0 | 20.5 |
| DR2 | DRB5*0101 | DR2w2 β2 | 8 | — | — | — | — | — | — |
| DR3 | DRB1*0301-2 | DR3 | 4 | 17.7 | 19.5 | 0.40 | 7.3 | 14.4 | 11.9 |
| DR4 | DRB1*0401-12 | DR4w4 | 11 | 23.6 | 6.1 | 40.4 | 21.9 | 29.8 | 24.4 |
| DR4 | DRB1*0401-12 | DR4w15 | 9 | — | — | — | — | — | — |
| DR7 | DRB1*0701-02 | DR7 | 9 | 26.2 | 11.1 | 1.0 | 15.0 | 16.6 | 14.0 |
| DR8 | DRB1*0801-5 | DR8w2 | 7 | 5.5 | 10.9 | 25.0 | 10.7 | 23.3 | 15.1 |
| DR9 | DRB1*09011, 09012 | DR9 | 10 | 3.6 | 4.7 | 24.5 | 19.9 | 6.7 | 11.9 |
| DR11 | DRB1*1101-05 | DR5w11 | 11 | 17.0 | 18.0 | 4.9 | 19.4 | 18.1 | 15.5 |
| DR13 | DRB1*1301-06 | DR6w19 | 7 | 21.7 | 16.5 | 14.6 | 12.2 | 10.5 | 15.1 |
| Total¹ | | | | 98.5 | 95.1 | 97.1 | 91.3 | 94.3 | 95.1 |

¹Total opulation coverage has been adjusted to acount for the presence of DRX in many ethnic populations. It has been assumed that the range of specificities represented by DRX alleles will mirror those of previously characterized HLA-DR alleles. The proportion of DRX incorporated under each motif is representative of the frequency of the motif in the remainder of the population. Total coverage has not been adjusted to account for unknown gene types.
²Number of epitopes represents a minimal estimate, considering only the epitopes shown in Table 12. Additional alleles possibly bound by nested epitopes have not been accounted.

1-37. (canceled)

38. A minigene construct comprising a polynucleic acid encoding the following epitopes: WLSLLVPFV (SEQ ID NO: 551), HTLWKAGILYK (SEQ ID NO: 605), FLPSDFFPSV (SEQ ID NO: 3492), STLPETTVVRR (SEQ ID NO: 3522), and GLSRYVARL (SEQ ID NO: 3704), wherein the minigene does not encode a wild-type full length protein from Hepatitis B Virus (HBV).

39. The minigene construct of claim 38, which further comprises one or a plurality of spacer nucleic acids.

40. The minigene construct of claim 38, which further comprises a member selected from the group consisting of:
(1) at least one cytotoxic T lymphocyte (CTL) epitope;
(2) at least one helper T lymphocyte (HTL) epitope; and
(3) a nucleic acid encoding at least one of the epitopes of Table XXXVIIa or Table XXXVIIb.

41. The minigene of claim 38, further comprising a nucleic acid encoding the epitope YMDDVVLGV (SEQ ID NO: 3828) or YMDDVVLGA (SEQ ID NO: 564).

42. The minigene construct of claim 40, wherein the at least one HTL epitope is a PADRE® epitope.

43. The minigene construct of claim 38, further comprising a signal sequence.

44. A vector comprising the minigene of claim 38.

45. The vector of claim 44, which is selected from the group consisting of a plasmid, a viral vector, and a bacterial vector.

46. The vector of claim 45, wherein the viral vector is vaccinia virus.

47. The vector of claim 46, which is a recombinant MVA.

48. A composition comprising the minigene of claim 38, and a pharmaceutical excipient.

49. The composition of claim 48, wherein the pharmaceutical excipient comprises an adjuvant.

50. The composition of claim 48, further comprising a member selected from the group consisting of:
(1) a liposome, wherein the epitopes are on or within the liposome; and
(2) an antigen presenting cell, wherein the epitopes are on or within the antigen presenting cell.

51. The composition of claim 50, wherein the epitopes are joined to a lipid.

52. The composition of claim 50, wherein the antigen presenting cell is a dendritic cell.

53. The composition according to claim 48, which is a vaccine composition.

54. A composition comprising the vector of claim 44, and a pharmaceutical excipient.

55. A method of inducing an immune response against Hepatitis B Virus (HBV) comprising administering the composition of claim 48.

56. A method of treating and/or preventing HBV comprising administering the composition of claim 48.

57. The method of claim 56, comprising the use of a prime boost protocol, wherein the prime boost protocol comprises administration of a boosting agent.

58. The method of claim 57, wherein the boosting agent comprises the minigene.

59. A polyepitopic peptide comprising the following epitopes: WLSLLVPFV (SEQ ID NO: 551), HTLWKAGILYK (SEQ ID NO: 605), FLPSDFFPSV (SEQ ID NO: 3492), STLPETTVVRR (SEQ ID NO: 3522), and GLSRYVARL (SEQ ID NO: 3704), wherein the polyepitopic peptide is not a wild-type full length protein from Hepatitis B Virus (HBV).

60. A polyepitopic peptide according to claim 59, whereby the epitopes are linked by a spacer molecule.

61. The polyepitopic peptide of claim 59, which further comprises a member selected from the group consisting of:
(1) at least one cytotoxic T lymphocyte (CTL) epitope;
(2) at least one helper T lymphocyte (HTL) epitope; and
(3) at least one of the epitopes of Table XXXVIIa or Table XXXVIIb.

62. The polyepitopic peptide of claim 59, further comprising the epitope YMDDVVLGV (SEQ ID NO: 3828) or YMDDVVLGA (SEQ ID NO: 564).

63. The polyepitopic peptide of claim 59, wherein the at least one HTL epitope is a PADRE® epitope.

64. The polyepitopic peptide of claim 59, further comprising a signal sequence.

65. A composition comprising the polyepitopic peptide of claim 59, and a pharmaceutical excipient.

66. A method of inducing an immune response against Hepatitis B Virus (HBV) comprising administering the composition of claim 65.

67. A method of treating and/or preventing HBV comprising administering the composition of claim 65.

68. The method of claim 67, comprising the use of a prime boost protocol, wherein the prime boost protocol comprises administration of a boosting agent.

69. The method of claim 68, wherein the boosting agent comprises the polyepitopic peptide.
